(19) United States
(12) Patent Application Publication
(10) Pub. No.: US 2016/0194725 A1
(43) Pub. Date: Jul. 7, 2016

(54) MOLECULAR IDENTIFICATION OF ALLERGY CAUSING MITES BY PCR

(71) Applicant: ALK-ABELLÓ A/S, Hørsholm (DK)

(72) Inventors: Pedro HERNÀNDEZ-CRESPO, Madrid (ES); Beatriz BEROIZ, Madrid (ES); Pedro CASTAÑERA, Madrid (ES); Félix ORTEGO, Madrid (ES); Maria José CHAMORRO SALILLAS, Madrid (ES); Manuel LOMBARDERO VEGA, Madrid (ES); Carmen ARTEAGA VÁZQUEZ, Madrid (ES)

(73) Assignee: ALK-Ablló S/A, Hørsholm (DK)

(21) Appl. No.: 14/905,211

(22) PCT Filed: Jul. 16, 2014
(86) PCT No.: PCT/EP2014/065276

§ 371 Date: Jan. 14, 2016

(30) Foreign Application Priority Data
Jul. 16, 2013 (EP) 13176734.5

(51) Int. Cl. C12Q 1/68

(52) U.S. Cl. CPC C12Q 1/6888; C12Q 2600/16

(57) ABSTRACT

The present invention relates to novel methods for the identification of specific mite species in a sample, such as mass reared sample or an environmental sample. The invention further relates to nucleic acid molecules encoding the structural ribosomal RNA elements (rRNA) as well as to the non-functional RNA situated between such structural ribosomal RNAs of specific mite species and its use for designing primers for use in the method.

### FIELD OF THE INVENTION

The present invention relates to novel nucleic acid sequences of specific Astigmata mite species, corresponding to nuclear ribosomal DNA (rDNA) that codes for ribosomal RNA. The invention further relates to the use of such sequences or fragments thereof in methods for the identification of the specific mite species in biological samples such as mass reared cultures, purified fractions from the cultures, house dust and other environmental samples.

### BACKGROUND OF THE INVENTION

Mites of the suborder Astigmata are recognised as important respiratory allergy causing elements. The most relevant species belong to the families Pyroglyphidae (Dermatophagoides and Euroglyphus), Acaridae (Acarus and Tyrophagus) and Glycyphagidae (Blomia, Glycyphagus and Lepidoglyphus). Allergen avoidance, drug therapy and immunotherapy are the main strategies currently conducted to reduce the allergic disease caused by mites, the latter being the only disease-modifying approach and the most promising to counteract allergy. The current immunotherapy involves administration to the patient of allergenic extracts in a suitable delivery form. In the case of mites, the extracts are produced from mass reared cultures of the relevant mites. Species identification and avoidance of cross contamination in mite cultures are key factors in the standardisation of allergen production. Furthermore, there is a regulatory requirement to certify the identity/purity/lack of cross-contamination of the mite cultures for preparing medical grade allergen extracts.

Also identification of mite populations in environmental samples from patients' houses is useful in order to monitor the risk of allergen exposure and for diagnostic purposes.

Mite species identification in mass reared cultures and in environmental samples has traditionally been based on morphological identification such as described in Spieksma 1990. The method is reliable but it can only be performed on samples of adult stages of intact mites and demands a high level of expertise. Morphological identification is time-consuming, represents an increased cost for the industry and cannot be applied on purified mite fractions downstream in the production process. Morphological identification of mite species in environmental samples can be challenging since the number of intact mites present may be quite low or even non existing.

Various molecular methods have been suggested to analyse phylogenetic relationships within phylogenetic orders of microorganisms, fungi, mites and ticks. In order to conduct phylogenetic studies, one or more suitable molecular markers must be identified. Cruickshank 2002 describes in a review article suitable properties of molecular markers and suggests nine possible molecular markers mainly selected within mitochondrial genes (mtDNA) and nuclear ribosomal genes (rDNA). Accordingly, the highly conserved regions of ribosomal DNA (18S rDNA, 5.8S rDNA and 28S rDNA), mitochondrial genes (cytochrome oxidase, 12S and 16S rDNA) and internal transcribed spacer regions of the rDNA (ITS1 and ITS2) have been proposed for phylogenetic studies.

Navajas 1999 assessed the usefulness of the molecular markers ITS1, ITS2 and 5.8S gene of rDNA for phylogenetic analysis and identification of species within Phytoseiidae mites. The entire ITS1-5.8S-ITS2 region was amplified with PCR (Polymerase Chain Reaction) using universal primers generated from the 18S and the 28S regions of rDNA. Each PCR-product was sequenced and aligned in order to determine the phylogenetic relationship. Navajas concluded that the level of DNA variation within a new group cannot be predicted and therefore preliminary assessment is necessary in order to identify suitable molecular markers for a species or a group of species. For Phytosiidae mites, ITS1 was longer than ITS2 and had much more sequence variation. ITS2 was considered too short to be of value in taxonomic studies and ITS1 was considered too variable, and 5.8S in combination with ITS2 was not considered giving adequate specificity within the group.

Noge 2005 used the ITS2 region of rDNA as molecular marker in order to make a phylogenetic analysis of 73 mite species. The primers for the PCR amplification were generated from the highly conserved regions flanking the ITS2 region (one in the 5.8S region and one in the 28S region). Three clones of each PCR-product were sequenced and aligned in order to determine the phylogenetic relationship.

Suarez-Martinez 2005 used mitochondrial 12S rRNA as a molecular marker in order to identify the four representative Astigmata mites Dermatophagoides pteronyssinus, Glycyphagus privatus, Aleuroglyphus ovatus and Blomia tropicalis. All species were amplified using one universal forward primer and one universal reverse primer generated from the rRNA 12S marker. Each PCR-product was sequenced and aligned in order to determine the phylogenetic relationship and to identify variants.

Some techniques for molecular identification of mite species in environmental samples have been proposed, such as PCR methods ((Restriction Fragment Length Polymorphism (RFLP), Amplified Fragment Length Polymorphism (AFLP), multiplexPCR)) and arrays.

Wong 2011 successfully identified Dermatophagoides pteronyssinus, Dermatophagoides farinae, Blomia tropicalis, Tyrophagus putrescentiae, Aleuroglyphus ovatus and Glycycometus malaysiensis in house dust using the ITS2 region of rDNA as molecular marker in a RFLP PCR. The primers were generated from the highly conserved regions flanking the ITS2 region (one in the 5.8S region and one in the 28S region). After amplification, identification was performed by digesting the PCR products with a combination of restriction enzymes specific for the mite to be identified and separating the restriction fragments with SDS-PAGE. The restriction fragment size pattern was used to identify the mite species in question. Wong suggests isolating single mites if there are several different mites present in the same dust sample.

JP2007-202462, JP2008-35773 and JP2009-171986 all disclose various aspects of the same invention. The invention regards an array system based on nucleic acid hybridisation for detection or differentiation of mites and fungi in house dust samples as well as nucleic acid probes for use in the microarray. In brief, the entire ITS1-5.8S-ITS2 regions of mites and fungi were amplified from dust samples using mite-specific primers (SEQ ID NOs: 56 and 57) and fungi-specific primers (SEQ ID NOs: 58 and 59) all generated from the 18S and the 28S regions of the rDNA. The amplification of mites and fungi could be performed in a “1-tube PCR” in which mite-specific primers and fungi-specific primers were both added to the same tube. For each mite and fungus to be detected, nucleic acid probes were amplified from pure samples of the mite or fungus in question using primers generated either from each end of the ITS1 or from each end of the ITS2. The resulting probes of the invention thus correspond to either the ITS1 or the ITS2 of the species in question or to fragments thereof or to the complements thereof. In the microarray, each well identifies an ITS1 region of one species or an ITS2 region of the species or the complements thereof such that detection of one species uses four wells.

Thet-em 2012 designed a multiplex PCR using ITS2 and Cox I as molecular markers to identify Dermatophagoides pteronyssinus, Dermatophagoides farinae and Blomia tropicalis in house dust. Species specific primers for Dermatophagoides pteronyssinus and Dermatophagoides farinae were generated from the ITS2 region of rDNA. Species specific primers for Blomia tropicalis were generated from the Cox I gene of mitochondrial DNA.

None of these methods use primers for mite species identification designed on the ITS1 region. Further the methods all require a set of primers per DNA sequence to amplify. In the cases where the amplicons are large, it is necessary to subject the amplicons to various restriction enzymes and analyze the resulting patterns of the size distribution of the fragments obtained in order to identify the exact species by the molecular sizes of the amplicons. Most of the methods are only suitable to identify a single species in a sample.

Accordingly, fairly large quantities of samples are still necessary in order to identify several species in a sample using these methods. Finally, most of the methods include a first step of non-specific amplification using mite specific primers and a second step of quite complex processing of the amplicon to allow species identification (restriction enzymes in RFLP-PCR, sequencing or binding to probes in arrays).

Kumar et al 1999 developed a PCR multiplex technique for identifying Cecidophyopsis mites using species specific differences in rDNA ITS-1 sequences. Four PCR primers derived from ITS-1 were used for the simultaneous amplification (multiplex PCR) of interspecifically variable simple sequence repeats (vSSRs). The primers consist of two forward primers designed in 18S (M1) and in a first conserved area of ITS1 (M3) respectively and the two reverse primers are designed in the 5.8S (M4) and a second conserved area of ITS1 (M2). None of the primers are species specific. They are all mite specific (or common to all the mites) and amplify amplicons S1, S2 and S3 in all mite species. The differentiation between mite species is done by comparing the pattern of S1+S2+S3 to known patterns of mite species. Since some of the amplicons differentiate by only 1 bp, it is necessary to use polyacrylamide gels. Mites were identified by electrophoresing PCR products on polyacrylamide gels alongside those obtained from plasmids containing ITS copies of known mite species. The article mentions that “the patterns were not discernable in agarose gels”.

There is still a need for more simple and robust methods for the identification of one or more mite species in mite cultures for preparing allergenic extracts for diagnostic, prophylactic and therapeutic purposes as well as in house dust.

### OBJECT OF THE INVENTION

The inventors of the present invention have designed a method based on molecular markers in order to facilitate identification or certification of mite species in mass reared mite cultures or purified mite fractions thereof or in environmental samples. DNA markers have the advantage of neither requiring a given developmental stage, nor intact individuals for the morphological analysis or requiring special training of the staff. Further, the method is advantageous for performing routine mite species identification or certification of a large number of samples, since the method has low requirements to sample quality and quantity and it reduces the time and skills necessary to perform the identification of mite species in comparison to the morphological identification. A DNA marker appropriate for the species certification in the production of allergenic extracts should identify the mite species in either whole mite cultures or purified fractions of mites of mite bodies or mite faeces.

The inventors found the full-length ITS1, 5.8S sub-unit and ITS2 sequences of the rDNA from thirteen Astigmata species belonging to genera Dermatophagoides, Euroglyphus, Acarus, Tyrophagus, Glycyphagus, Lepidoglyphus and Blomia (families Pyroglyphidae, Acaridae, Glycyphagidae and Echymopididae). Based on the sequences obtained, a singleplex-PCR and multiplex-PCR method were developed to identify 10 of those species which are recognised as important respiratory allergy causing agents. Despite polymorphism and high variability in the ITS1 region, the inventors showed that a singleplex or a multiplex PCR method using primers designed on the ITS1 region of Astigmata mite species provides a simple, robust and reliable method of Astigmata mite species identification. In the multiplex PCR method, the primers may be combined for the simultaneous identification of multiple Astigmata mite species. The system can be used for species certification in mite cultures and purified fractions thereof (bodies and faeces) used for the industrial production of allergenic extracts. Finally, the system has been optimised for the detection of Astigmata mite species in environmental samples by introducing an optional preamplification step.

It is an object of the invention to provide sequence information for the full-length ITS1, 5.8S sub-unit and ITS2 sequences for specific Astigmata species providing new molecular markers for mite species identification or certification, as well as methods for the identification, detection, discrimination or differentiation of one or more different Astigmata mite species. It is also an object of the invention to use the sequence information both to design primers which are unique to a specific species (species specific) and to design primers which are specific to all Astigmata mite species (mite specific). It is a further object of the invention to provide singleplex and multiplex methods which are simple to perform and robust yet highly accurate.

### SUMMARY OF THE INVENTION

It has been found by the present inventors that the full-length ITS1, 5.8S sub-unit and ITS2 sequences of the rDNA from the specific Astigmata mite species may be used for the identification, detection or discrimination of these specific mite species.

So in a first aspect, the present invention relates to a method for the identification of one or more different Astigmata mite species in a sample, the method comprising the steps of:

a) obtaining DNA from the sample;
b) amplifying, such as by PCR, a region of the rDNA of each of the mite species to be identified using
i. one or more, such as 1, 2, 3, 4, 5, 6, 7, 8, 9 or 10 first primers each first primer specifically hybridising to the ITS1 sequence of the rDNA of each of the mite species to be identified, or the complementary sequence thereof, and
ii. one or more, such as one, second primers specifically hybridising to a sequence selected from any of the 18S, 5.8S or 28S sequences of the rDNA of the mite species to be identified, or the complementary sequence thereof,
   to produce an amplicon specific to the mite species to be identified, and;
   c) identifying the mite species by evaluating a characteristic of the amplicon.

It is to be understood in b) i. that “each first primer specifically hybridising to each of the ITS1 sequence of the rDNA of the mite species to be identified” means that each first primer hybridizes to only one sequence of a specific Astigmata mite species to be identified. Accordingly, if different species are present in a sample, each first primer will only hybridize to one specific species and not to the others. In a specific embodiment, the first primer is designed so that in addition to hybridizing to only one of the different Astigmata mite species to be identified, it will not hybridize to the ITS1 of any other known Astigmata mite species. Accordingly the first primer will only hybridize to the ITS1 of one specific known Astigmata mite species and will not be able to hybridize to identify any other known Astigmata mite species present in the sample or not.

It is to be understood in c) that when several mite species are present, several different amplicons are produced, each being specific for one particular mite species to be identified.

The sample may be any Astigmata mite containing sample such as a sample of a mass reared mite culture, a purified fraction thereof or an environmental sample. In particular, this method enables the identification of mite species in purified fractions of mass reared mite cultures.

Obtaining DNA from a sample is to be understood as extracting DNA according to methods known in the art, such as described in the examples, and in a form suitable for the subsequent amplification step.

The first primers may be forward primers and the second primers may be reverse primers or the opposite.

This method is highly sensitive, simple to perform, robust and provides a high degree of accuracy in identification of mite species in samples.

The polymerase chain reaction (PCR) is a biochemical technology in molecular biology to amplify a single or a few copies of a piece of DNA across several orders of magnitude, generating from thousands to millions of copies of a particular DNA sequence. The technology is well known to the person skilled in the art.

In brief, the method relies on thermal cycling, consisting of cycles of repeated heating and cooling of the reaction for DNA melting and enzymatic replication of the DNA. Primers (short DNA fragments) containing sequences complementary to the target region along with a DNA polymerase (after which the method is named) are key components to enable selective and repeated amplification. As PCR progresses, the DNA generated is itself used as a template for replication, setting in motion a chain reaction in which the DNA template is exponentially amplified. PCR can be extensively modified to perform a wide array of genetic manipulations.

Almost all PCR applications employ a heat-stable DNA polymerase, such as Taq polymerase, an enzyme originally isolated from the bacterium Thermus aquaticus. This DNA polymerase enzymatically assembles a new DNA strand from DNA building-blocks, the nucleotides, by using single-stranded DNA as a template and DNA oligonucleotides (also called DNA primers), which are required for initiation of DNA synthesis. The vast majority of PCR methods use thermal cycling, i.e., alternately heating and cooling the PCR sample through a defined series of temperature steps. In the first step, the two strands of the DNA double helix are physically separated at a high temperature in a process called DNA melting. In the second step, the temperature is lowered and the two DNA strands become templates for DNA polymerase to selectively amplify the target DNA. The selectivity of PCR is achieved by the use of primers that are complementary to the DNA region targeted for amplification under specific thermal cycling conditions.

The designing of primers and the optimization of the PCR conditions are key factors for the specificity and efficiency of the PCR as the skilled person will know. The temperature of the PCR should be optimised in accordance with the melting temperature of the primers (Tm, a measure of the stability of the duplex formed by hybridisation of the primer with their complementary sequence).

Various software tools are available to propose theoretical primers for a target DNA, or guidelines in textbooks may be followed. The composition of a primer affects the melting temperature and the ability of the primer to hybridise to a target DNA, and especially the 3′ end of the primer should have exact complementarity to the target DNA.

In an embodiment, the one (or more) first primers is a species specific primer. In a further embodiment, the one (or more) second primers is one common primer specific to Astigmata mites. In a preferred embodiment, the one (or more) first primers is species specific and the one (or more) second primers is one common primer specific to Astigmata mites. Such embodiment has the advantage that the number of different primers used may be reduced if several Astigmata mite species are to be identified in a single assay, such as in a multiplex-PCR. Furthermore, when the second primer is one common primer for several mite species to be identified, such that the amplicons produced has one common starting or ending point, it becomes more straight forward to design primers for the other end which result in amplicons of significantly different sizes for each species. By significantly different is meant that the sizes differ by at least 15 bp. This difference in size ensures that an agarose gel can be used to separate the amplicons by electrophoresis. An agarose gel has the advantage of not being influenced by the sequence of the amplicons and therefore it is insensitive to polymorphisms within the amplicons. It differentiates only by by size. In comparison polyacrylamide gels are sensitive to to sequence variation such as polymorphisms which may affect the resolution of the electrophoresis on an polyacrylamide gel. So the separation in a polyacrylamide gel depends on both the nature and the length of the sequence.

Multiplex-PCR may be useful in identifying the presence of different Astigmata mite species in a sample, such as in environmental samples as well as in certifying the purity and lack of cross-contamination in a single species culture.

In some embodiments, the method may be preceded by a preamplification step. This is advantageous if the sample has a low content of rDNA such as in environmental samples.

In a second aspect, the present invention relates to an isolated nucleic acid molecule at least about 80% identical to a nucleic acid sequence selected from the list consisting of SEQ ID NOs:1-100 or fragment thereof, or complementary sequence thereof.

In some embodiments, the nucleic acid molecule is a polynucleotide.

These sequences provide new sequence information which is useful in designing new primers or probes for the identification, detection, discrimination or differentiation of different mite species in a sample. Also the sequence information provided confirms the phylogenetic relationship of the Astigmata mites identified.

Whenever used herein and in some embodiments, the phrase “at least about 80% identical to” refers to a sequence of at least about 81% identical to, such as at least about 82% identical to, such as at least about 83% identical to, such as at least about 84% identical to, such as at least about 85% identical to, such as at least about 86% identical to, such as at least about 87% identical to, such as at least about 88% identical to, such as at least about 89% identical to, such as at least about 90% identical to, such as at least about 91% identical to, such as at least about 92% identical to, such as at least about 93% identical to, such as at least about 94% identical to, such as at least about 95% identical to, such as at least about 96% identical to, such as at least about 97% identical to, such as at least about 98% identical to, such as at least about 99% identical to, such as about 100% identical to a nucleic acid sequence selected from the list consisting of SEQ ID NO:1-100 or fragment thereof, or complementary sequence thereof.

In some embodiments, the isolated nucleic acid molecule is at least about 80% identical to a nucleic acid sequence selected from the list consisting of SEQ ID NO:1-100 or fragment thereof.

In some embodiments, the isolated nucleic acid molecule is at least about 80% identical to a complementary sequence of a nucleic acid sequence selected from the list consisting of SEQ ID NO:1-100 or fragment thereof.

In a further aspect, the present invention relates to a composition comprising nucleic acid molecules of one or more, such as 1, 2, 3, 4, 5, 6, 7, 8, 9, or 10 different species in the Astigmata suborder, the nucleic acid molecules being at least about 80% identical to a nucleic acid sequence selected from the list consisting of SEQ ID NO:1-100 or fragment thereof, or complementary sequence thereof.

In some embodiments, the composition according to the present invention comprises nucleic acid molecules of at least 2, such as at least 3, such as at least 4, such as at least 5, such as at least 6, such as at least 7, such as at least 8, such as at least 9, such as 10 different species in the Astigmata suborder at least about 80% identical to a nucleic acid sequence selected from the list consisting of SEQ ID NO:1-100 or fragment thereof, or complementary sequence thereof.

In some embodiments, the composition according to the present invention comprises sequences to detect, discriminate, or identify two or more, such as 2, 3, 4, 5, 6, 7, 8, 9, or 10 different species selected from the list consisting of Tyrophagus fanetzhangorum, Lepidoglyphus destructor, Glycyphagus domesticus, Dermatophagoides pteronyssinus, Tyrophagus putrescentiae, Blomia tropicalis, Euroglyphus maynei, Dermatophagoides micro ceras, Acarus siro, and Dermatophagoides farinae.

In some embodiments, the composition according to the present invention further comprises a nucleic acid molecule at least about 80% identical to 5.8S in a sequence selected from any one of SEQ ID NOs:1-100, or the complementary sequence thereof, or fragment thereof, such as Rast5.8, such as a nucleic acid sequence defined by SEQ ID NO:111, or the complementary sequence thereof.

Accordingly, the composition may in one embodiment comprise first and second primers designed on the ITS1 sequence of the Astigmata mite species to be identified. In a specific embodiment, the first primers are designed on the ITS1 sequence and the second primer(s) is/are designed on the 5.8S sequence. Such composition has the advantage that the number of different primers used may be reduced if several Astigmata mite species are to be identified in a single assay. As will be clear to the skilled person the total amount of forward primers must equal the total amount of revers primers.

In a further aspect, the present invention relates to the use of one or more nucleic acid molecules at least about 80% identical to a nucleic acid sequence independently selected from the list consisting of SEQ ID NOs:1-111 or fragment thereof, or complementary sequence thereof, for the detection, discrimination, or identification of one or more, such as 1, 2, 3, 4, 5, 6, 7, 8, 9, or 10 different specific species of the Astigmata suborder. In some embodiments, the one or more nucleic acid molecule is/are a nucleic acid molecule according to the present invention. In some embodiments, the nucleic acid molecule is as defined herein, or is part of a composition according to present invention.

In some embodiments, the isolated nucleic acid molecule is as defined herein and comprising ITS1, to design a primer which is unique to a specific Astigmata mite species. In some embodiments, the use is of an isolated nucleic acid molecule as defined herein and comprising 5.8S or 18S to design a primer which specifically hybridises to any of the rDNA of the Astigmata mite species of Tyrophagus fanetzhangorum, Lepidoglyphus destructor, Glycyphagus domesticus, Dermatophagoides pteronyssinus, Tyrophagus putrescentiae, Blomia tropicalis, Euroglyphus maynei, Dermatophagoides microceras, Acarus siro and Dermatophagoides farinae.

In order to design a species specific primer, based on the sequence of ITS1, to be useful for species identification, the following steps may be performed:

1. Align all know sequences of ITS1 from Astigmata mite species. Given the intra-individual and the intra-specific polymorphism, it is recommendable to include in the analysis more than one sequences from each species that could represent natural polymorphism.
2. Select from the alignment the regions full-filling two requisites: high intra-specific conservation and low inter-specific conservation (this regions can be defined as “species specific”)
3. Select, among the regions in point 2, those containing sequences that could be appropriate for the design of primers following the standard roles for primer design (e.g. 18-32 consecutive nucleotides containing more than 40% of G/C against A/T; no self-complementariness, etc.)
4. Design primers having a relative high Tm (for example, using the “bases stacking method”, Tm could be between 52 and 56), between 18 and 30 bps (18-23 recommendable) and a good quality considering GC composition, complexity (polyX and triplet repetitions), 3′ stability and self dimers (The software AmplifX v1.4.4 ([Nicolas Jullien 2001-2007] or any other software may be use for primer design.
5. Once the primers are designed, select the primers not showing a high similarity (mainly at their 3′ end) to known sequences of other organisms (the analysis may be performed by BLASTN against public databases). The primers selected at this point would be good candidates for PCRs, however, selection must continue in order to select the primers that could be suitable for a PCR.

Species specific direct sense primers should be combined with an appropriate reverse sense primer that should be based on conserved regions of the rDNA, preferably it should be an Astigmata-specific primer. Thus, primers should be selected to:

i. show no complementariness with the reverse primer or primers to be used in the PCR reaction.
ii. show no complementariness with the other primers to be used in a multiplex PCR

Finally, the combinations of primers forward-reverse in a PCR should be designed to obtain amplicons of different size when amplifying DNA from different species.

In a further aspect, the present invention relates to amplicons obtained by the method according to the invention.

In a further aspect, the present invention relates to a molecular size marker composition for use in the method according to the invention comprising one or more polynucleotides, such as DNA of a size (in base pairs) corresponding to one or more amplicons obtained by the method according to the invention. A size corresponding to the size of the amplicons means the exact sizes of the amplicons +−30, 20 or 10 base pairs.

Such composition may be useful when comparing the size of the amplicon of the mite to be detected with the molecular markers. When the reference nucleotide has nearly the same size as the amplicon to be evaluated, it is easier to compare with the eye and thus to identify the species.

In a further aspect, the present invention relates to a method for the identification of one or more different Astigmata mite species in a sample, the method comprising the steps of:

a) Obtaining DNA from the sample;
b) Amplifying a region of the rDNA of the each of the mite species to be identified using
i. a first primer, each primer specifically hybridising to the ITS1 sequence of the rDNA of each of the mite species to be identified, or the complementary sequence thereof, and
ii. a second primer specifically hybridising to a sequence selected from 18S, 5.8S and 28S sequences of the rDNA of the mite species to be identified, or the complementary sequence thereof,
   to produce an amplicon specific to the mite species to be identified, and;
   c) identifying the mite species by evaluating the molecular size of the amplicon.

In some embodiments, the method according to the present invention is performed using one or more sets of a forward and a reverse primers, wherein at least one of said primers of a set is specific for said species and identical to a sequence at least about 80% identical to a nucleic acid sequence selected from the list consisting of SEQ ID NOs:1-100 or fragment thereof, or complementary sequence thereof.

In some embodiments, the method according to the present invention is performed with primers of a composition according to the present invention.

In some embodiments, the method according to the present invention further comprises a step after step a) of amplification, such as by PCR, of any rDNA component in said sample, such as by use of primer pairs specific to 18S, 5.8S or 28S sequences.

Such preamplification may be useful if the samples have a low content of rDNA material to be identified such as when only a few or even only one mite is present, for instance in environmental samples.

In a further aspect, the present invention relates to a kit of parts comprising:
a) A composition according to the invention; and
b) A molecular size marker, such as a molecular size marker composition as defined herein.

In some embodiments, the kit comprises a pair of primers specific to 18S, 5.8S or 28S sequences suitable for amplification, such as by PCR, of any rDNA component in a sample. In some embodiments, the kit further comprises an extraction solution and/or an instruction manual.

In a further aspect, the present invention relates to a method for the preparation of a certified specimen of an Astigmata mite culture or of a purified fraction thereof, wherein the identity of one or more specific species in the Astigmata suborder in said sample is known, the method comprising the steps of

a) Obtaining DNA from a sample of the culture or purified fraction;
b) Detecting a nucleic acid molecule specific for said species, said sequence being identical to a nucleic acid sequence at least about 80% identical to a nucleic acid sequence selected from the list consisting of SEQ ID NOs:1-100 or fragment thereof, or complementary sequence thereof;
c) Identifying said specific species in the Astigmata suborder based on the detection of a nucleic acid molecule specific for said species;
d) Obtaining said specimen, wherein the identity of one or more specific species in the Astigmata suborder in said specimen is known from step c).

In some embodiments, step b) is performed using PCR on the rDNA with one or more set of a forward and a reverse primer, wherein at least one of said primers of a set is specific for said species and identical to a sequence at least about 80% identical to a nucleic acid sequence selected from the list consisting of SEQ ID NOs:1-100 or fragment thereof, or complementary sequence thereof.

In some embodiments, the PCR is performed with primers of a composition as defined herein.

In some embodiments, step b) is preceded by a preamplification step, such as by PCR, wherein the rDNA of all Astigmata mite species in the sample is amplified using a first primer specifically hybridising to the 18S sequence of the rDNA and a second primer specifically hybridising to a sequence selected from the 5.8S or 28S sequences of the rDNA.

In some embodiments, the one or more specific species in the Astigmata suborder is selected from the list consisting of: Tyrophagus fanetzhangorum, Lepidoglyphus destructor, Glycyphagus domesticus, Dermatophagoides pteronyssinus, Tyrophagus putrescentiae, Blomia tropicalis, Euroglyphus maynei, Dermatophagoides microceras, Acarus siro and Dermatophagoides farinae.

In a further aspect, the present invention relates to a mite culture or a purified fraction prepared according to this method, such as a preparation of a certified mite culture or of a certified purified fraction.

Instead of or as a supplement to using species-specific ITS1 derived first primers in step b) i., it is equally possible to perform a molecular amplification which includes the presence of a detectable probe, which has the same hybridization characteristics as the above-defined first primer (i.e. that it hybridises specifically with a part of the ITS1 sequence (or its complementary sequence) of one single species of Astigmata; obviously, the probe must have a nucleic acid sequence that matches part of the amplicon obtained. Such a probe is particularly useful in embodiments of qPCR or real-time PCR, where a signal from the specific probe can be detected/recorded after conclusion of each amplification cycle, as is well-known in the art. Such a probe can e.g. be in the form of a nucleic acid sequence equipped with a fluorescent probe and a matching quencher, where the quencher is released when the probe is incorporated into an amplicon by a DNA polymerase.

Therefore, only probes that ultimately are present in an amplicon will fluoresce, providing a precise quantitative measure of the amount of amplicon from each cycle by methods well-known in the art. Particularly high specificity can be obtained if both the first primer and such a probe fulfil the hybridisation requirements for a first primer defined herein—but as mentioned it will be possible to use a more generic primer as a first primer, if a specific probe is included. In embodiments where several species are to be determined, the fluorescent probes used to detect each species can each be uniquely labelled so as to fluoresce at different wavelengths; hence, in multiplex amplifications, the relative quantities of different amplicons can be determined by correlating to the relative fluorescence intensities at the relevant wavelengths.

### LEGENDS TO THE FIGURES

FIG. 1. One step Multiplex-PCR analysis of DNA extracted from mites cultures provided by ALK-ABELLÓ. Each lane is from left: M (100 bp DNA Ladder (Promega)). Ma: (Marker adapted for identification of allergy-causing mites), T. fanetzhangorum (Tf), Lepidoglyphus destructor (Ld), Glycyphagusdomesticus (Gd), D. pteronyssinus (Dp), Tyrophagus putrescentiae (Tp), Ma, Blomia tropicalis (Bt), Euroglyphus maynei (Em), Dermatophagoides microceras (Dm), Acarus siro (As), D. farinae (Df), Ma, and M. (see Example 4).

FIG. 2. Two steps Multiplex-PCR analysis of DNA extracted from mites cultures provided by ALK-ABELLÓ. Each lane is from left: M (100 bp DNA Ladder (Promega)). Ma: (Marker adapted for identification of allergy-causing mites), T. fanetzhangorum (Tf), Lepidoglyphus destructor (Ld), Glycyphagusdomesticus (Gd), D. pteronyssinus (Dp), Tyrophagus putrescentiae (Tp), Ma, Blomia tropicalis (Bt), Euroglyphus maynei (Em), Dermatophagoides microceras (Dm), Acarus siro (As), D. farinae (Df), Ma, and M. (see Example 4).

FIG. 3. Ma Marker. DNA ladder prepared from nucleotides of by sizes corresponding to the amplicons produced in Example 2 for Tyrophagus fanetzhangorum (Tf), Lepidoglyphus destructor (Ld), Glycyphagus domesticus (Gd), Dermatophagoides pteronyssinus (Dp), Tyrophagus putrescentiae (Tp), Blomia tropicalis (Bt), Euroglyphus maynei (Em), Dermatophagoides microceras (Dm), Acarus siro (As), Dermatophagoides farinae (Df).

FIG. 4. Representation of the primers of Example 2 and Example 3, step 3

FIG. 5. Representation of the primers of Example 3, step 2 (preamplification)

FIG. 6. One step Multiplex-PCR analysis of DNA extracted from mite cultures provided by ALK-ABELLÓ. Each lane is from left: 100 bp DNA Ladder (Promega), T. fanetzhangorum (Tf), Lepidoglyphus destructor (Ld), Glycyphagusdomesticus (Gd), D. pteronyssinus (Dp), Tyrophagus putrescentiae (Tp), Blomia tropicalis (Bt), Euroglyphus maynei (Em), Dermatophagoides microceras (Dm), Acarus siro (As), D. farinae (Df), and 100 bp DNA Ladder (Promega).

### DETAILED DISCLOSURE OF THE INVENTION

### Definitions

When terms such as “one”, “a” or “an” are used in this disclosure they mean “at least one”, or “one or more” unless otherwise indicated. Further, the term “comprising” is intended to mean “including” and thus allows for the presence of other constituents, features, conditions, or steps than those explicitly recited.

The term “purified fraction” of a mass reared culture refers to a fraction of the culture, which is of mite origin, for instance mite bodies (body fraction) or mite faeces (faeces fraction). The purified fractions may be obtained from a mite culture by any fractionation method, such as by sieving or otherwise separating the sample. The predominant content of the purified fraction is bodies or faeces of one or more specific mite species compared to other constituents of the culture, such a nutrients and waste products.

The term “identification” as used herein refers to the mere detection or determination of the presence of one or more specific Astigmata mite species in a sample, the identification of the specific Astigmata mite species, as well as the ability to discriminate between one or more different specific Astigmata mite species in a sample. For example, identification of a mite species can refer to determining which phylogenetic genus, species, or subspecies an individual mite belongs.

The term “Ribosomal DNA” or “rDNA”, as used herein refers to a DNA sequence that codes for ribosomal RNA, such as the ribosomal RNA of Astigmata mite species. Ribosomes are assemblies of proteins and rRNA molecules that translate mRNA molecules to produce proteins. rDNA of eukaryotes including mites consists of a tandem repeat of a unit segment, an operon, containing the elements 18S, ITS1, 5.8S, ITS2, and 28S.

The term “Internal transcribed spacer 1 (ITS1)” or ITS1 as used herein refers to the nucleic acid sequence, such as in any one of SEQ ID NOs:1-100 situated between the nucleic acid sequences encoding the structural ribosomal RNAs 18S rRNA and 5.8S rRNA. Accordingly, ITS1 is defined by having boundaries to 18S (5′ AGGATCATTA 3′) and to 5.8S (5′, CTGYYAGTGG 3′).

The term “Internal transcribed spacer 2 (ITS2)” or ITS2 as used herein refers to the nucleic acid sequence, such as in any one of SEQ ID NOs:1-100 situated between the nucleic acid sequences encoding the structural ribosomal RNAs 5.8S rRNA and 28S rRNA. Accordingly, ITS2 is defined by having boundaries to 5.8S (5′ TGAGCGTCGT 3′) and to 28S (5′ CGACCTCAG 3′).

The term “5.8S” as used herein refers to the nucleic acid sequence, such as in any one of SEQ ID NOs:1-100 situated between ITS1 and ITS2, such as the nucleic acid sequences encoding the structural ribosomal RNAs with boundaries 5′, CTGYYAGTGG 3′ and 5′ TGAGCGTCGT 3′ of SEQ ID NO:1-100.

The term “28S” as used herein refers to the nucleic acid sequence encoding the structural ribosomal 28S RNAs just downstream of ITS2 having boundaries (5′ CGACCTCAG 3′) of SEQ ID NO:1-100.

The term “18S” as used herein refers to the nucleic acid sequence encoding the structural ribosomal 18S RNAs just upstream of ITS1 having boundaries (5′ AGGATCATTA 3′) of SEQ ID NO:1-100.

As used herein the term “first primer” refers to a primer in a set of primers used in the amplification, such as by PCR, of a rDNA fragment. The first primer may be the forward primer or the reverse primer relative to the “second primer”. It follows that the term “second primer” also refers to a primer in a set of primers used in the amplification, such as by PCR, of a ribosomal DNA

An “isolated” molecule is a molecule that is the predominant species in the composition wherein it is found with respect to the class of molecules to which it belongs (i.e. it makes up at least about 50% of the type of molecule in the composition and typically will make up at least about 70%, at least about 80%, at least about 85%, at least about 90%, at least about 95%, or more of the species of molecule, e.g., nucleotide or peptide, in the composition). Commonly, a composition of a nucleic acid molecule will exhibit 98%-99% homogeneity for nucleic acid molecules in the context of all present nucleic acid species in the composition or at least with respect to substantially active nucleic acid species in the context of the proposed use.

The term “specifically hybridising to” refers to primers or probes which, under suitable conditions, specifically hybridise with the relevant nucleic acids. Said suitable conditions are preferably stringent hybridisation conditions as defined below. In a preferred embodiment, a probe hybridises only with one nucleic acid, e.g. a rDNA for one particular mite species clone. For example, a primer that “specifically hybridizes” to an ITS1 sequence or a “specific primer” describes a primer that hybridizes to only one mite species in a sample of multiple mite species. Likewise, an amplicon “specific for” a given mite species describes an amplicon that is present (or amplified from) only one mite species to be identified hi a sample comprising multiple mite species. Alternatively, it is preferred that a probe hybridises with several nucleic acid clones of the same type of mite species.

“Stringent hybridisation conditions” include conditions comprising e.g.: overnight incubation at 65° C. in 4×SSC (600 mM sodium chloride, 60 mM sodium citrate), followed by a washing step at 65° C. in 0.1×SSC for 1 hour. Alternatively, it is possible to incubate at 42° C. in a solution that contains 50% formamide, 5×SSC (750 mM sodium chloride, 75 mM sodium citrate), 50 mM sodium phosphate (pH 7.6), 5×Denhardt's solution, 10% dextrane sulphate and 20 μg/ml denatured, sheared DNA from salmon sperm, followed by washing steps in 0.1×SSC of 5 to 20 minutes at approximately 65° C. These hybridisation conditions are known to the person skilled in the art as highly stringent hybridisation conditions. Unless otherwise stated, the term “Sequence identity” for nucleotides as used herein refers to the sequence identity calculated as 100−(n_{ref}−n_{dif})·100/n_{ref}, wherein n_{dif} is the total number of non-identical nucleotides in the two sequences when aligned and wherein n_{ref} is the number of residues in one of the sequences.

Unless otherwise stated, the number of residues n_{ref} and the alignment are made only in the length of the shortest sequence. Accordingly, if a short primer is compared with the sequence of a longer DNA sequence, only the sequence of the overlap or corresponding regions thereof is compared. Hence, the nucleic acid sequence GCATACCGTGTTGAAGCAGG will have a sequence identity of 80% with the sequence AAATACCGTGTTGAAGCAAA (n_{dif}=4 and n_{ref}=20). The alignment may be be done direct-direct or direct reverse. The alignment showing the maximum similarity should be used.

In some embodiments, the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (BLASTN, BLASTX and TBLASTX, GAP, BESTFIT, FASTA, and TFASTA in the Wisconsin Genetics Software Package, Genetics Computer Group). The BLAST algorithm (Altschul et al., 1990, Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

Sequence identity analysis includes database search and alignment. Examples of public databases include the DNA Database of Japan (DDBJ) (on the World Wide Web at ddbj.nig.acjp/); Genebank (on the World Wide Web at ncbi.nlm.nih.gov/Web/Search/Index.htlm); and the European Molecular Biology Laboratory Nucleic Acid Sequence Database (EMBL) (on the World Wide Web at ebi.ac.uk/ebi_docs/embl_db/embl-db.html). Other appropriate databases include dbEST (on the World Wide Web at ncbi.nlm.nih.gov/dbEST/index.html), Swissprot (on the World Wide Web at ebi.ac.uk/ebi_docs/swisprot db/swisshome.html), PIR (on the World Wide Web at nbrt.georgetown.edu/pir/) and The Institute for Genome Research (on the World Wide Web at tigr.org/tdb/tdb.html).

A number of different search algorithms have been developed, one example of which are the suite of programmes referred to as BLAST programmes. There are five implementations of BLAST, three designed for nucleotide sequences queries (BLASTN, BLASTX and TBLASTX) and two designed for protein sequence queries (BLASTP and TBLASTN) (Coulson, Trends in Biotechnology 12:76-80 (1994); Birren et al., Genome Analysis 1, Cold Spring Harbor Laboratory Press, Cold Spring Harbor, N.Y. 543-559 (1997)).

BLASTN takes a nucleotide sequence (the query sequence) and its reverse complement and searches them against a nucleotide sequence database. BLASTN was designed for speed, not maximum sensitivity and may not find distantly related coding sequences. BLASTX takes a nucleotide sequence, translates it in three forward reading frames and three reverse complement reading frames and then compares the six translations against a protein sequence database. BLASTX is useful for sensitive analysis of preliminary (single-pass) sequence data and is tolerant of sequencing errors (Gish and States, Nature Genetics 3:266-272 (1993), the entirety of which is herein incorporated by reference). BLASTN and BLASTX may be used in concert for analyzing EST data (Coulson, Trends in Biotechnology 12:76-80 (1994); Birren et al., Genome Analysis 1:543-559 (1997)).

Given a coding nucleotide sequence and the protein it encodes, it is often preferable to use the protein as the query sequence to search a database because of the greatly increased sensitivity to detect more subtle relationships. This is due to the larger alphabet of proteins (20 amino acids) compared with the alphabet of nucleic acid sequences (4 bases), where it is far easier to obtain a match by chance. In addition, with nucleotide alignments, only a match (positive score) or a mismatch (negative score) is obtained, but with proteins, the presence of conservative amino acid substitutions can be taken into account. Here, a mismatch may yield a positive score if the non-identical residue has physical/chemical properties similar to the one it replaced. Various scoring matrices are used to supply the substitution scores of all possible amino acid pairs. A general purpose scoring system is the BLOSUM62 matrix (Henikoff and Henikoff, Proteins 17:49-61 (1993), the entirety of which is herein incorporated by reference), which is currently the default choice for BLAST programmes. BLOSUM62 is tailored for alignments of moderately diverged sequences and thus may not yield the best results under all conditions. Altschul, J. Mol. Biol. 36:290-300 (1993), the entirety of which is herein incorporated by reference, describes a combination of three matrices to cover all contingencies. This may improve sensitivity, but at the expense of slower searches. In practice, a single BLOSUM62 matrix is often used but others (PAM40 and PAM250) may be attempted when additional analysis is necessary. Low PAM matrices are directed at detecting very strong but localized sequence similarities, whereas high PAM matrices are directed at detecting long but weak alignments between very distantly related sequences.

Homologues in other organisms are available that can be used for comparative sequence analysis. Multiple alignments are performed to study similarities and differences in a group of related sequences. CLUSTAL W is a multiple sequence alignment package that performs progressive multiple sequence alignments based on the method of Feng and Doolittle, J. Mol. Evol. 25:351-360 (1987), the entirety of which is herein incorporated by reference. Each pair of sequences is aligned and the distance between each pair is calculated; from this distance matrix, a guide tree is calculated and all of the sequences are progressively aligned based on this tree. A feature of the program is its sensitivity to the effect of gaps on the alignment; gap penalties are varied to encourage the insertion of gaps in probable loop regions instead of in the middle of structured regions. Users can specify gap penalties, choose between a number of scoring matrices, or supply their own scoring matrix for both pairwise alignments and multiple alignments. CLUSTAL W for UNIX and VMS systems is available at: ftb.ebi.ac.uk. Another program is MACAW (Schuler et al., Proteins Struct. Func. Genet. 9:180-190 (1991), the entirety of which is herein incorporated by reference, for which both Macintosh and Microsoft Windows versions are available. MACAW uses a graphical interface, provides a choice of several alignment algorithms and is available by anonymous ftp at: ncbi.nlm.nih.gov (directory/pub/macaw).

### SPECIFIC EMBODIMENTS OF THE INVENTION

As described above the present invention relates to a method for the identification of one or more different Astigmata mite species in a sample, the method comprising the steps of:

a) obtaining DNA from the sample;
b) amplifying, such as by PCR, a region of the rDNA of each of the mite species to be identified using
i. one or more, such as 1, 2, 3, 4, 5, 6, 7, 8, 9, or 10 first primers specifically hybridising to the ITS1 sequence of the rDNA of the mite species to be identified, or the complementary sequence thereof, and
ii. one or more, such as one, second primers specifically hybridising to a sequence selected from any of the 18S, 5.8S or 28S sequences of the rDNA of the mite species to be identified, or the complementary sequence thereof,
   to produce an amplicon specific to the mite species to be identified, and;
   c) identifying the mite species by evaluating a characteristic of the amplicon.

In some embodiments under step b), the amplicon produced has a molecular size which is characteristic of the specific mite species to be identified.

In some embodiments under step c), the mite species is identified by evaluating the molecular size of the amplicon which is characteristic of the mite species to be identified.

However, the amplicons may also be characterised by sequencing the amplicon and identifying the mite species by comparing to SEQ ID NO's:1-100.

In some embodiments, less than 13, such as 10, such as 8, such as 6, such as 5, such as 3 different Astigmata mites are identified.

In some embodiments under step b), two or more amplicons specific to the mite species to be identified are produced, which amplicons differ in length by at least 15 bp, such as 16, 17, 18, 19, 20, 21, 22, 23, 24, 25, 26, 27, 28, 29, or 30 bp.

In some embodiments, the second primer is 90%, such as 91, 92, 93, 94, 95, 96, 97, 98, 99, or 100% identical to at least 15 consecutive nucleotides of said sequence of any of the Astigmata mite species to be identified.

In some embodiments, the one or more first primers used in step b) i. contains at least 3, such as 4, 5 or 6 consecutive nucleotides in the 3′ end with exact complementarity to any ITS1 sequence of the mite species to be identified.

In some embodiments, the one or more first primers used in step b) i. is at least about 70%, such as 76, 77, 78, 79, 80, 81, 82, 83, 84, 85, 86, 87, 88, 89, 90, 91, 92, 93, 94, 95, 96, 97, 98, 99% identical to the sequence of any corresponding part of the ITS1 sequence or a complementary part thereof of the mite species to be identified.

In some embodiments, the method is for the identification of at least 2, 3, 4, 5, 6, 7, 8, 9, 10, 11, or 12, or more different Astigmata mite species in the sample.

In some embodiments, step c) is performed by comparing the molecular size(s) of the amplicon(s) to the molecular sizes of reference nucleotides of a molecular marker composition, the sizes of the reference nucleotides spanning the relevant base pair interval.

Reference nucleotide compositions are commercially available. An example is the Thermo Scientific GeneRuler 100 bp DNA Ladder. It contains reference nucleotides of 1000, 900, 800, 700, 600, 500, 400, 300, 200, 100 bp. It is suitable for both agarose gels and polyacrylamide gels. Another DNA ladder is available from Promega. The ladder is dissolved in buffer and electroforesed together with the DNA sample to be analysed. When reading amplicon sizes using such a classic DNA ladder, the amplicon size is conveniently estimated by comparing by eye the distance travelled by the amplicon with the distance travelled by the reference nucleotides of the ladder (having steps of 100 bp).

In some embodiments, the sizes of the reference nucleotides correspond to the sizes of the amplicons characteristic of the mite species to be identified. An advantage of using such reference nucleotides of the sizes of the amplicons to be identified, is that it becomes easier to compare the sizes of the amplicons with the sizes of the reference nucleotides. Especially by eye.

Electrophoresing a reference nucleotide composition together with the sample on a gel enables identification of each Astigmata mite species present in the sample directly from the result of the electrophoresis by comparing the sample result with the reference nucleotide composition. No intermediate step is necessary, such as sequencing the amplicon or evaluating the band pattern of multiple amplicons per mite species to be identified.

In some embodiments, step b) is preceded by a preamplification step, such as by PCR, wherein the rDNA containing the ITS1 region of all Astigmata mite species in the sample is amplified using a first primer specifically hybridising to the 18S sequence of the rDNA and a second primer specifically hybridising to a sequence selected from the 5.8S and 28S sequences of the rDNA.

In some embodiments, the sample is an environmental sample.

In some embodiments, the sample is from a mass reared culture or a purified fraction thereof.

In some embodiments, the sample is from a mass reared culture or a purified fraction thereof wherein a preamplification step according to claim 10 is not conducted.

In some embodiments, two or more first primers are used, each primer specifically hybridising to the ITS1 sequences of one mite species to be identified, or the complementary sequence thereof, and not cross hybridising to other mite species to be identified.

In some embodiments, the first primer is designed on two or more, such as 2, 3, 4, 5, 6, 7, 8, 9, or 10 groups of sequences identified by any one of SEQ ID NOs:1-10, SEQ ID NOs:11-20, SEQ ID NOs:21-30, SEQ ID NOs:31-40, SEQ ID NOs:41-50, SEQ ID NOs:51-60, SEQ ID NOs:61-70, SEQ ID NOs:71-80, SEQ ID NOs:81-90, and SEQ ID NOs:91-10.

In some embodiments, the first primer referred to in b) i. comprises a sequence at least about 70%, such as 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to the ITS1 of a sequence selected from any one of SEQ ID NOs:1-100, or the complementary sequence thereof, or a fragment thereof.

In some embodiments, the first primer is at least about 6, 7, 8, 9, 10, 11, 12, 13, 14, 15, 16, 17, 18, 19, 20, 21, 22, 23, 24 or 25 contiguous nucleotides in length.

In some embodiments, the first primer is not more than about 70, 60, 50, 40, 30, 25, 23, 20 contiguous nucleotides in length.

In some embodiments, the first primer comprises a sequence at least about 70%, such as 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to a nucleic acid sequence selected from the list consisting of SEQ ID NO:101, 102, 103, 104, 105, 106, 107, 108, 109, 110, 122, 123, and 124, or the complementary sequence thereof, or fragment thereof, or complementary sequence thereof.

In some embodiments, the first primer consists of a sequence at least about 70%, such as 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to a nucleic acid sequence selected from the list consisting of SEQ ID NO:101, 102, 103, 104, 105, 106, 107, 108, 109, 110, 122, 123, and 124, or the complementary sequence thereof, or fragment thereof.

In some embodiments, the second primer, comprises a nucleic acid sequence at least about 70%, such as 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to a fragment of 5.8S in a sequence selected from any one of SEQ ID NOs:1-100, or the complementary sequence thereof, such as Rast5.8, such as a nucleic acid sequence defined by SEQ ID NO:111 or the complementary sequence thereof, or fragment thereof.

In some embodiments, the second primer, comprises a nucleic acid sequence at least about 70%, such as 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical to a fragment of 18S in a sequence selected from any one of SEQ ID NOs:1-100, or the complementary sequence thereof, such as FRibNav, such as a nucleic acid sequence defined by SEQ ID NO:121 or the complementary sequence thereof, or fragment thereof.

In some embodiments, the one or more different species in the Astigmata suborder is/are selected from the group consisting of: Tyrophagus fanetzhangorum, Lepidoglyphus destructor, Glycyphagus domesticus, Dermatophagoides pteronyssinus, Tyrophagus putrescentiae, Blomia tropicalis, Euroglyphus maynei, Dermatophagoides microceras, Acarus siro and Dermatophagoides farinae.

As mentioned above, the present invention relates to an isolated nucleic acid molecule at least about 80% identical to a nucleic acid sequence selected from the list consisting of SEQ ID NOs:1-100 or fragment thereof, or complementary sequence thereof.

In some embodiments, the isolated nucleic acid molecule is at least about 6, 7, 8, 9, 10, 11, 12, 13, 14, 15, 16, 17, 18, 19, 20, 21, 22, 23, 24 or 25 contiguous nucleotides in length.

In some embodiments, the isolated nucleic acid molecule according to the invention is at least 7, such as at least 8, such as at least 9, such as at least 10, such as at least 11, such as at least 12, such as at least 13, such as at least 14, such as at least 15, such as at least 16, such as at least 17, such as at least 18, such as at least 19, such as at least 20, such as at least 21, such as at least 22, such as at least 23, such as at least 24, such as at least 25, such as at least 26, such as at least 27, such as at least 28, such as at least 29, such as at least 30, such as at least 31, such as at least 32, such as at least 33, such as at least 34, such as at least 35, such as at least 36, such as at least 37, such as at least 38, such as at least 39, such as at least 40, such as at least 41, such as at least 42, such as at least 43, such as at least 44, such as at least 45, such as at least 46, such as at least 47, such as at least 48, such as at least 49, such as at least 50, such as at least 51, such as at least 52, such as at least 53, such as at least 54, such as at least 55, such as at least 56, such as at least 57, such as at least 58, such as at least 59, such as at least 60, such as at least 61, such as at least 62, such as at least 63, such as at least 64, such as at least 65, such as at least 66, such as at least 67, such as at least 68, such as at least 69, such as at least 70, such as at least 71, such as at least 72, such as at least 73, such as at least 74, such as at least 75, such as at least 76, such as at least 77, such as at least 78, such as at least 79, such as at least 80, such as at least 81, such as at least 82, such as at least 83, such as at least 84, such as at least 85, such as at least 86, such as at least 87, such as at least 88, such as at least 89, such as at least 90, such as at least 91, such as at least 92, such as at least 93, such as at least 94, such as at least 95, such as at least 96, such as at least 97, such as at least 98, such as at least 99, such as at least 100, such as at least 101, such as at least 102, such as at least 103, such as at least 104, such as at least 105, such as at least 106, such as at least 107, such as at least 108, such as at least 109, such as at least 110, such as at least 111, such as at least 112, such as at least 113, such as at least 114, such as at least 115, such as at least 116, such as at least 117, such as at least 118, such as at least 119, such as at least 120, such as at least 121, such as at least 122, such as at least 123, such as at least 124, such as at least 125, such as at least 126, such as at least 127, such as at least 128, such as at least 129, such as at least 130, such as at least 131, such as at least 132, such as at least 133, such as at least 134, such as at least 135, such as at least 136, such as at least 137, such as at least 138, such as at least 139, such as at least 140, such as at least 141, such as at least 142, such as at least 143, such as at least 144, such as at least 145, such as at least 146, such as at least 147, such as at least 148, such as at least 149, such as at least 150, such as at least 151, such as at least 152, such as at least 153, such as at least 154, such as at least 155, such as at least 156, such as at least 157, such as at least 158, such as at least 159, such as at least 160, such as at least 161, such as at least 162, such as at least 163, such as at least 164, such as at least 165, such as at least 166, such as at least 167, such as at least 168, such as at least 169, such as at least 170, such as at least 171, such as at least 172, such as at least 173, such as at least 174, such as at least 175, such as at least 176, such as at least 177, such as at least 178, such as at least 179, such as at least 180, such as at least 181, such as at least 182, such as at least 183, such as at least 184, such as at least 185, such as at least 186, such as at least 187, such as at least 188, such as at least 189, such as at least 190, such as at least 191, such as at least 192, such as at least 193, such as at least 194, such as at least 195, such as at least 196, such as at least 197, such as at least 198, such as at least 199, such as at least 200, such as at least 201, such as at least 202, such as at least 203, such as at least 204, such as at least 205, such as at least 206, such as at least 207, such as at least 208, such as at least 209, such as at least 210, such as at least 211, such as at least 212, such as at least 213, such as at least 214, such as at least 215, such as at least 216, such as at least 217, such as at least 218, such as at least 219, such as at least 220, such as at least 221, such as at least 222, such as at least 223, such as at least 224, such as at least 225, such as at least 226, such as at least 227, such as at least 228, such as at least 229, such as at least 230, such as at least 231, such as at least 232, such as at least 233, such as at least 234, such as at least 235, such as at least 236, such as at least 237, such as at least 238, such as at least 239, such as at least 240, such as at least 241, such as at least 242, such as at least 243, such as at least 244, such as at least 245, such as at least 246, such as at least 247, such as at least 248, such as at least 249, such as at least 250, such as at least 251, such as at least 252, such as at least 253, such as at least 254, such as at least 255, such as at least 256, such as at least 257, such as at least 258, such as at least 259, such as at least 260, such as at least 261, such as at least 262, such as at least 263, such as at least 264, such as at least 265, such as at least 266, such as at least 267, such as at least 268, such as at least 269, such as at least 270, such as at least 271, such as at least 272, such as at least 273, such as at least 274, such as at least 275, such as at least 276, such as at least 277, such as at least 278, such as at least 279, such as at least 280, such as at least 281, such as at least 282, such as at least 283, such as at least 284, such as at least 285, such as at least 286, such as at least 287, such as at least 288, such as at least 289, such as at least 290, such as at least 291, such as at least 292, such as at least 293, such as at least 294, such as at least 295, such as at least 296, such as at least 297, such as at least 298, such as at least 299, such as at least 300, such as at least 301, such as at least 302, such as at least 303, such as at least 304, such as at least 305, such as at least 306, such as at least 307, such as at least 308, such as at least 309, such as at least 310, such as at least 311, such as at least 312, such as at least 313, such as at least 314, such as at least 315, such as at least 316, such as at least 317, such as at least 318, such as at least 319, such as at least 320, such as at least 321, such as at least 322, such as at least 323, such as at least 324, such as at least 325, such as at least 326, such as at least 327, such as at least 328, such as at least 329, such as at least 330, such as at least 331, such as at least 332, such as at least 333, such as at least 334, such as at least 335, such as at least 336, such as at least 337, such as at least 338, such as at least 339, such as at least 340, such as at least 341, such as at least 342, such as at least 343, such as at least 344, such as at least 345, such as at least 346, such as at least 347, such as at least 348, such as at least 349, such as at least 350, such as at least 351, such as at least 352, such as at least 353, such as at least 354, such as at least 355, such as at least 356, such as at least 357, such as at least 358, such as at least 359, such as at least 360, such as at least 361, such as at least 362, such as at least 363, such as at least 364, such as at least 365, such as at least 366, such as at least 367, such as at least 368, such as at least 369, such as at least 370, such as at least 371, such as at least 372, such as at least 373, such as at least 374, such as at least 375, such as at least 376, such as at least 377, such as at least 378, such as at least 379, such as at least 380, such as at least 381, such as at least 382, such as at least 383, such as at least 384, such as at least 385, such as at least 386, such as at least 387, such as at least 388, such as at least 389, such as at least 390, such as at least 391, such as at least 392, such as at least 393, such as at least 394, such as at least 395, such as at least 396, such as at least 397, such as at least 398, such as at least 399, such as at least 400, such as at least 401, such as at least 402, such as at least 403, such as at least 404, such as at least 405, such as at least 406, such as at least 407, such as at least 408, such as at least 409, such as at least 410, such as at least 411, such as at least 412, such as at least 413, such as at least 414, such as at least 415, such as at least 416, such as at least 417, such as at least 418, such as at least 419, such as at least 420, such as at least 421, such as at least 422, such as at least 423, such as at least 424, such as at least 425, such as at least 426, such as at least 427, such as at least 428, such as at least 429, such as at least 430, such as at least 431, such as at least 432, such as at least 433, such as at least 434, such as at least 435, such as at least 436, such as at least 437, such as at least 438, such as at least 439, such as at least 440, such as at least 441, such as at least 442, such as at least 443, such as at least 444, such as at least 445, such as at least 446, such as at least 447, such as at least 448, such as at least 449, such as at least 450, such as at least 451, such as at least 452, such as at least 453, such as at least 454, such as at least 455, such as at least 456, such as at least 457, such as at least 458, such as at least 459, such as at least 460, such as at least 461, such as at least 462, such as at least 463, such as at least 464, such as at least 465, such as at least 466, such as at least 467, such as at least 468, such as at least 469, such as at least 470, such as at least 471, such as at least 472, such as at least 473, such as at least 474, such as at least 475, such as at least 476, such as at least 477, such as at least 478, such as at least 479, such as at least 480, such as at least 481, such as at least 482, such as at least 483, such as at least 484, such as at least 485, such as at least 486, such as at least 487, such as at least 488, such as at least 489, such as at least 490, such as at least 491, such as at least 492, such as at least 493, such as at least 494, such as at least 495, such as at least 496, such as at least 497, such as at least 498, such as at least 499, such as at least 500, such as at least 501, such as at least 502, such as at least 503, such as at least 504, such as at least 505, such as at least 506, such as at least 507, such as at least 508, such as at least 509, such as at least 510, such as at least 511, such as at least 512, such as at least 513, such as at least 514, such as at least 515, such as at least 516, such as at least 517, such as at least 518, such as at least 519, such as at least 520, such as at least 521, such as at least 522, such as at least 523, such as at least 524, such as at least 525, such as at least 526, such as at least 527, such as at least 528, such as at least 529, such as at least 530, such as at least 531, such as at least 532, such as at least 533, such as at least 534, such as at least 535, such as at least 536, such as at least 537, such as at least 538, such as at least 539, such as at least 540, such as at least 541, such as at least 542, such as at least 543, such as at least 544, such as at least 545, such as at least 546, such as at least 547, such as at least 548, such as at least 549, such as at least 550, such as at least 551, such as at least 552, such as at least 553, such as at least 554, such as at least 555, such as at least 556, such as at least 557, such as at least 558, such as at least 559, such as at least 560, such as at least 561, such as at least 562, such as at least 563, such as at least 564, such as at least 565, such as at least 566, such as at least 567, such as at least 568, such as at least 569, such as at least 570, such as at least 571, such as at least 572, such as at least 573, such as at least 574, such as at least 575, such as at least 576, such as at least 577, such as at least 578, such as at least 579, such as at least 580, such as at least 581, such as at least 582, such as at least 583, such as at least 584, such as at least 585, such as at least 586, such as at least 587, such as at least 588, such as at least 589, such as at least 590, such as at least 591 contiguous nucleotides in length.

In some embodiments, the isolated nucleic acid molecule according to the invention is not more than 999 contiguous nucleotides, such as not more than 998, such as not more than 997, such as not more than 996, such as not more than 995, such as not more than 994, such as not more than 993, such as not more than 992, such as not more than 991, such as not more than 990, such as not more than 989, such as not more than 988, such as not more than 987, such as not more than 986, such as not more than 985, such as not more than 984, such as not more than 983, such as not more than 982, such as not more than 981, such as not more than 980, such as not more than 979, such as not more than 978, such as not more than 977, such as not more than 976, such as not more than 975, such as not more than 974, such as not more than 973, such as not more than 972, such as not more than 971, such as not more than 970, such as not more than 969, such as not more than 968, such as not more than 967, such as not more than 966, such as not more than 965, such as not more than 964, such as not more than 963, such as not more than 962, such as not more than 961, such as not more than 960, such as not more than 959, such as not more than 958, such as not more than 957, such as not more than 956, such as not more than 955, such as not more than 954, such as not more than 953, such as not more than 952, such as not more than 951, such as not more than 950, such as not more than 949, such as not more than 948, such as not more than 947, such as not more than 946, such as not more than 945, such as not more than 944, such as not more than 943, such as not more than 942, such as not more than 941, such as not more than 940, such as not more than 939, such as not more than 938, such as not more than 937, such as not more than 936, such as not more than 935, such as not more than 934, such as not more than 933, such as not more than 932, such as not more than 931, such as not more than 930, such as not more than 929, such as not more than 928, such as not more than 927, such as not more than 926, such as not more than 925, such as not more than 924, such as not more than 923, such as not more than 922, such as not more than 921, such as not more than 920, such as not more than 919, such as not more than 918, such as not more than 917, such as not more than 916, such as not more than 915, such as not more than 914, such as not more than 913, such as not more than 912, such as not more than 911, such as not more than 910, such as not more than 909, such as not more than 908, such as not more than 907, such as not more than 906, such as not more than 905, such as not more than 904, such as not more than 903, such as not more than 902, such as not more than 901, such as not more than 900, such as not more than 899, such as not more than 898, such as not more than 897, such as not more than 896, such as not more than 895, such as not more than 894, such as not more than 893, such as not more than 892, such as not more than 891, such as not more than 890, such as not more than 889, such as not more than 888, such as not more than 887, such as not more than 886, such as not more than 885, such as not more than 884, such as not more than 883, such as not more than 882, such as not more than 881, such as not more than 880, such as not more than 879, such as not more than 878, such as not more than 877, such as not more than 876, such as not more than 875, such as not more than 874, such as not more than 873, such as not more than 872, such as not more than 871, such as not more than 870, such as not more than 869, such as not more than 868, such as not more than 867, such as not more than 866, such as not more than 865, such as not more than 864, such as not more than 863, such as not more than 862, such as not more than 861, such as not more than 860, such as not more than 859, such as not more than 858, such as not more than 857, such as not more than 856, such as not more than 855, such as not more than 854, such as not more than 853, such as not more than 852, such as not more than 851, such as not more than 850, such as not more than 849, such as not more than 848, such as not more than 847, such as not more than 846, such as not more than 845, such as not more than 844, such as not more than 843, such as not more than 842, such as not more than 841, such as not more than 840, such as not more than 839, such as not more than 838, such as not more than 837, such as not more than 836, such as not more than 835, such as not more than 834, such as not more than 833, such as not more than 832, such as not more than 831, such as not more than 830, such as not more than 829, such as not more than 828, such as not more than 827, such as not more than 826, such as not more than 825, such as not more than 824, such as not more than 823, such as not more than 822, such as not more than 821, such as not more than 820, such as not more than 819, such as not more than 818, such as not more than 817, such as not more than 816, such as not more than 815, such as not more than 814, such as not more than 813, such as not more than 812, such as not more than 811, such as not more than 810, such as not more than 809, such as not more than 808, such as not more than 807, such as not more than 806, such as not more than 805, such as not more than 804, such as not more than 803, such as not more than 802, such as not more than 801, such as not more than 800, such as not more than 799, such as not more than 798, such as not more than 797, such as not more than 796, such as not more than 795, such as not more than 794, such as not more than 793, such as not more than 792, such as not more than 791, such as not more than 790, such as not more than 789, such as not more than 788, such as not more than 787, such as not more than 786, such as not more than 785, such as not more than 784, such as not more than 783, such as not more than 782, such as not more than 781, such as not more than 780, such as not more than 779, such as not more than 778, such as not more than 777, such as not more than 776, such as not more than 775, such as not more than 774, such as not more than 773, such as not more than 772, such as not more than 771, such as not more than 770, such as not more than 769, such as not more than 768, such as not more than 767, such as not more than 766, such as not more than 765, such as not more than 764, such as not more than 763, such as not more than 762, such as not more than 761, such as not more than 760, such as not more than 759, such as not more than 758, such as not more than 757, such as not more than 756, such as not more than 755, such as not more than 754, such as not more than 753, such as not more than 752, such as not more than 751, such as not more than 750, such as not more than 749, such as not more than 748, such as not more than 747, such as not more than 746, such as not more than 745, such as not more than 744, such as not more than 743, such as not more than 742, such as not more than 741, such as not more than 740, such as not more than 739, such as not more than 738, such as not more than 737, such as not more than 736, such as not more than 735, such as not more than 734, such as not more than 733, such as not more than 732, such as not more than 731, such as not more than 730, such as not more than 729, such as not more than 728, such as not more than 727, such as not more than 726, such as not more than 725, such as not more than 724, such as not more than 723, such as not more than 722, such as not more than 721, such as not more than 720, such as not more than 719, such as not more than 718, such as not more than 717, such as not more than 716, such as not more than 715, such as not more than 714, such as not more than 713, such as not more than 712, such as not more than 711, such as not more than 710, such as not more than 709, such as not more than 708, such as not more than 707, such as not more than 706, such as not more than 705, such as not more than 704, such as not more than 703, such as not more than 702, such as not more than 701, such as not more than 700, such as not more than 699, such as not more than 698, such as not more than 697, such as not more than 696, such as not more than 695, such as not more than 694, such as not more than 693, such as not more than 692, such as not more than 691, such as not more than 690, such as not more than 689, such as not more than 688, such as not more than 687, such as not more than 686, such as not more than 685, such as not more than 684, such as not more than 683, such as not more than 682, such as not more than 681, such as not more than 680, such as not more than 679, such as not more than 678, such as not more than 677, such as not more than 676, such as not more than 675, such as not more than 674, such as not more than 673, such as not more than 672, such as not more than 671, such as not more than 670, such as not more than 669, such as not more than 668, such as not more than 667, such as not more than 666, such as not more than 665, such as not more than 664, such as not more than 663, such as not more than 662, such as not more than 661, such as not more than 660, such as not more than 659, such as not more than 658, such as not more than 657, such as not more than 656, such as not more than 655, such as not more than 654, such as not more than 653, such as not more than 652, such as not more than 651, such as not more than 650, such as not more than 649, such as not more than 648, such as not more than 647, such as not more than 646, such as not more than 645, such as not more than 644, such as not more than 643, such as not more than 642, such as not more than 641, such as not more than 640, such as not more than 639, such as not more than 638, such as not more than 637, such as not more than 636, such as not more than 635, such as not more than 634, such as not more than 633, such as not more than 632, such as not more than 631, such as not more than 630, such as not more than 629, such as not more than 628, such as not more than 627, such as not more than 626, such as not more than 625, such as not more than 624, such as not more than 623, such as not more than 622, such as not more than 621, such as not more than 620, such as not more than 619, such as not more than 618, such as not more than 617, such as not more than 616, such as not more than 615, such as not more than 614, such as not more than 613, such as not more than 612, such as not more than 611, such as not more than 610, such as not more than 609, such as not more than 608, such as not more than 607, such as not more than 606, such as not more than 605, such as not more than 604, such as not more than 603, such as not more than 602, such as not more than 601, such as not more than 600, such as not more than 599, such as not more than 598, such as not more than 597, such as not more than 596, such as not more than 595, such as not more than 594, such as not more than 593, such as not more than 592, such as not more than 591, such as not more than 590, such as not more than 589, such as not more than 588, such as not more than 587, such as not more than 586, such as not more than 585, such as not more than 584, such as not more than 583, such as not more than 582, such as not more than 581, such as not more than 580, such as not more than 579, such as not more than 578, such as not more than 577, such as not more than 576, such as not more than 575, such as not more than 574, such as not more than 573, such as not more than 572, such as not more than 571, such as not more than 570, such as not more than 569, such as not more than 568, such as not more than 567, such as not more than 566, such as not more than 565, such as not more than 564, such as not more than 563, such as not more than 562, such as not more than 561, such as not more than 560, such as not more than 559, such as not more than 558, such as not more than 557, such as not more than 556, such as not more than 555, such as not more than 554, such as not more than 553, such as not more than 552, such as not more than 551, such as not more than 550, such as not more than 549, such as not more than 548, such as not more than 547, such as not more than 546, such as not more than 545, such as not more than 544, such as not more than 543, such as not more than 542, such as not more than 541, such as not more than 540, such as not more than 539, such as not more than 538, such as not more than 537, such as not more than 536, such as not more than 535, such as not more than 534, such as not more than 533, such as not more than 532, such as not more than 531, such as not more than 530, such as not more than 529, such as not more than 528, such as not more than 527, such as not more than 526, such as not more than 525, such as not more than 524, such as not more than 523, such as not more than 522, such as not more than 521, such as not more than 520, such as not more than 519, such as not more than 518, such as not more than 517, such as not more than 516, such as not more than 515, such as not more than 514, such as not more than 513, such as not more than 512, such as not more than 511, such as not more than 510, such as not more than 509, such as not more than 508, such as not more than 507, such as not more than 506, such as not more than 505, such as not more than 504, such as not more than 503, such as not more than 502, such as not more than 501, such as not more than 500, such as not more than 499, such as not more than 498, such as not more than 497, such as not more than 496, such as not more than 495, such as not more than 494, such as not more than 493, such as not more than 492, such as not more than 491, such as not more than 490, such as not more than 489, such as not more than 488, such as not more than 487, such as not more than 486, such as not more than 485, such as not more than 484, such as not more than 483, such as not more than 482, such as not more than 481, such as not more than 480, such as not more than 479, such as not more than 478, such as not more than 477, such as not more than 476, such as not more than 475, such as not more than 474, such as not more than 473, such as not more than 472, such as not more than 471, such as not more than 470, such as not more than 469, such as not more than 468, such as not more than 467, such as not more than 466, such as not more than 465, such as not more than 464, such as not more than 463, such as not more than 462, such as not more than 461, such as not more than 460, such as not more than 459, such as not more than 458, such as not more than 457, such as not more than 456, such as not more than 455, such as not more than 454, such as not more than 453, such as not more than 452, such as not more than 451, such as not more than 450, such as not more than 449, such as not more than 448, such as not more than 447, such as not more than 446, such as not more than 445, such as not more than 444, such as not more than 443, such as not more than 442, such as not more than 441, such as not more than 440, such as not more than 439, such as not more than 438, such as not more than 437, such as not more than 436, such as not more than 435, such as not more than 434, such as not more than 433, such as not more than 432, such as not more than 431, such as not more than 430, such as not more than 429, such as not more than 428, such as not more than 427, such as not more than 426, such as not more than 425, such as not more than 424, such as not more than 423, such as not more than 422, such as not more than 421, such as not more than 420, such as not more than 419, such as not more than 418, such as not more than 417, such as not more than 416, such as not more than 415, such as not more than 414, such as not more than 413, such as not more than 412, such as not more than 411, such as not more than 410, such as not more than 409, such as not more than 408, such as not more than 407, such as not more than 406, such as not more than 405, such as not more than 404, such as not more than 403, such as not more than 402, such as not more than 401, such as not more than 400, such as not more than 399, such as not more than 398, such as not more than 397, such as not more than 396, such as not more than 395, such as not more than 394, such as not more than 393, such as not more than 392, such as not more than 391, such as not more than 390, such as not more than 389, such as not more than 388, such as not more than 387, such as not more than 386, such as not more than 385, such as not more than 384, such as not more than 383, such as not more than 382, such as not more than 381, such as not more than 380, such as not more than 379, such as not more than 378, such as not more than 377, such as not more than 376, such as not more than 375, such as not more than 374, such as not more than 373, such as not more than 372, such as not more than 371, such as not more than 370, such as not more than 369, such as not more than 368, such as not more than 367, such as not more than 366, such as not more than 365, such as not more than 364, such as not more than 363, such as not more than 362, such as not more than 361, such as not more than 360, such as not more than 359, such as not more than 358, such as not more than 357, such as not more than 356, such as not more than 355, such as not more than 354, such as not more than 353, such as not more than 352, such as not more than 351, such as not more than 350, such as not more than 349, such as not more than 348, such as not more than 347, such as not more than 346, such as not more than 345, such as not more than 344, such as not more than 343, such as not more than 342, such as not more than 341, such as not more than 340, such as not more than 339, such as not more than 338, such as not more than 337, such as not more than 336, such as not more than 335, such as not more than 334, such as not more than 333, such as not more than 332, such as not more than 331, such as not more than 330, such as not more than 329, such as not more than 328, such as not more than 327, such as not more than 326, such as not more than 325, such as not more than 324, such as not more than 323, such as not more than 322, such as not more than 321, such as not more than 320, such as not more than 319, such as not more than 318, such as not more than 317, such as not more than 316, such as not more than 315, such as not more than 314, such as not more than 313, such as not more than 312, such as not more than 311, such as not more than 310, such as not more than 309, such as not more than 308, such as not more than 307, such as not more than 306, such as not more than 305, such as not more than 304, such as not more than 303, such as not more than 302, such as not more than 301, such as not more than 300, such as not more than 299, such as not more than 298, such as not more than 297, such as not more than 296, such as not more than 295, such as not more than 294, such as not more than 293, such as not more than 292, such as not more than 291, such as not more than 290, such as not more than 289, such as not more than 288, such as not more than 287, such as not more than 286, such as not more than 285, such as not more than 284, such as not more than 283, such as not more than 282, such as not more than 281, such as not more than 280, such as not more than 279, such as not more than 278, such as not more than 277, such as not more than 276, such as not more than 275, such as not more than 274, such as not more than 273, such as not more than 272, such as not more than 271, such as not more than 270, such as not more than 269, such as not more than 268, such as not more than 267, such as not more than 266, such as not more than 265, such as not more than 264, such as not more than 263, such as not more than 262, such as not more than 261, such as not more than 260, such as not more than 259, such as not more than 258, such as not more than 257, such as not more than 256, such as not more than 255, such as not more than 254, such as not more than 253, such as not more than 252, such as not more than 251, such as not more than 250, such as not more than 249, such as not more than 248, such as not more than 247, such as not more than 246, such as not more than 245, such as not more than 244, such as not more than 243, such as not more than 242, such as not more than 241, such as not more than 240, such as not more than 239, such as not more than 238, such as not more than 237, such as not more than 236, such as not more than 235, such as not more than 234, such as not more than 233, such as not more than 232, such as not more than 231, such as not more than 230, such as not more than 229, such as not more than 228, such as not more than 227, such as not more than 226, such as not more than 225, such as not more than 224, such as not more than 223, such as not more than 222, such as not more than 221, such as not more than 220, such as not more than 219, such as not more than 218, such as not more than 217, such as not more than 216, such as not more than 215, such as not more than 214, such as not more than 213, such as not more than 212, such as not more than 211, such as not more than 210, such as not more than 209, such as not more than 208, such as not more than 207, such as not more than 206, such as not more than 205, such as not more than 204, such as not more than 203, such as not more than 202, such as not more than 201, such as not more than 200, such as not more than 199, such as not more than 198, such as not more than 197, such as not more than 196, such as not more than 195, such as not more than 194, such as not more than 193, such as not more than 192, such as not more than 191, such as not more than 190, such as not more than 189, such as not more than 188, such as not more than 187, such as not more than 186, such as not more than 185, such as not more than 184, such as not more than 183, such as not more than 182, such as not more than 181, such as not more than 180, such as not more than 179, such as not more than 178, such as not more than 177, such as not more than 176, such as not more than 175, such as not more than 174, such as not more than 173, such as not more than 172, such as not more than 171, such as not more than 170, such as not more than 169, such as not more than 168, such as not more than 167, such as not more than 166, such as not more than 165, such as not more than 164, such as not more than 163, such as not more than 162, such as not more than 161, such as not more than 160, such as not more than 159, such as not more than 158, such as not more than 157, such as not more than 156, such as not more than 155, such as not more than 154, such as not more than 153, such as not more than 152, such as not more than 151, such as not more than 150, such as not more than 149, such as not more than 148, such as not more than 147, such as not more than 146, such as not more than 145, such as not more than 144, such as not more than 143, such as not more than 142, such as not more than 141, such as not more than 140, such as not more than 139, such as not more than 138, such as not more than 137, such as not more than 136, such as not more than 135, such as not more than 134, such as not more than 133, such as not more than 132, such as not more than 131, such as not more than 130, such as not more than 129, such as not more than 128, such as not more than 127, such as not more than 126, such as not more than 125, such as not more than 124, such as not more than 123, such as not more than 122, such as not more than 121, such as not more than 120, such as not more than 119, such as not more than 118, such as not more than 117, such as not more than 116, such as not more than 115, such as not more than 114, such as not more than 113, such as not more than 112, such as not more than 111, such as not more than 110, such as not more than 109, such as not more than 108, such as not more than 107, such as not more than 106, such as not more than 105, such as not more than 104, such as not more than 103, such as not more than 102, such as not more than 101, such as not more than 100, such as not more than 99, such as not more than 98, such as not more than 97, such as not more than 96, such as not more than 95, such as not more than 94, such as not more than 93, such as not more than 92, such as not more than 91, such as not more than 90, such as not more than 89, such as not more than 88, such as not more than 87, such as not more than 86, such as not more than 85, such as not more than 84, such as not more than 83, such as not more than 82, such as not more than 81, such as not more than 80, such as not more than 79, such as not more than 78, such as not more than 77, such as not more than 76, such as not more than 75, such as not more than 74, such as not more than 73, such as not more than 72, such as not more than 71, such as not more than 70, such as not more than 69, such as not more than 68, such as not more than 67, such as not more than 66, such as not more than 65, such as not more than 64, such as not more than 63, such as not more than 62, such as not more than 61, such as not more than 60, such as not more than 59, such as not more than 58, such as not more than 57, such as not more than 56, such as not more than 55, such as not more than 54, such as not more than 53, such as not more than 52, such as not more than 51, such as not more than 50, such as not more than 49, such as not more than 48, such as not more than 47, such as not more than 46, such as not more than 45, such as not more than 44, such as not more than 43, such as not more than 42, such as not more than 41, such as not more than 40, such as not more than 39, such as not more than 38, such as not more than 37, such as not more than 36, such as not more than 35, such as not more than 34, such as not more than 33, such as not more than 32, such as not more than 31, such as not more than 30, such as not more than 29, such as not more than 28, such as not more than 27, such as not more than 26, such as not more than 25 contiguous nucleotides in length.

In some embodiments, the isolated nucleic acid molecule is not more than about 1200, 1100, 1000, 900, 800, 700, 600, 500, 400, 300, 200, 100, 90, 80, 70, 60, 50, 40, 30, or 20 contiguous nucleotides in length.

In some embodiments, the isolated nucleic acid molecule is specific for Tyrophagus fanetzhangorum. In some embodiments, the isolated nucleic acid molecule is specific for Lepidoglyphus destructor. In some embodiments, the isolated nucleic acid molecule is specific for Glycyphagus domesticus. In some embodiments, the isolated nucleic acid molecule is specific for Dermatophagoides pteronyssinus. In some embodiments, the isolated nucleic acid molecule is specific for Tyrophagus putrescentiae. In some embodiments, the isolated nucleic acid molecule is specific for Blomia tropicalis. In some embodiments, the isolated nucleic acid molecule is specific for Euroglyphus maynei. In some embodiments, the isolated nucleic acid molecule is specific for Dermatophagoides microceras. In some embodiments, the isolated nucleic acid molecule is specific for Acarus siro. In some embodiments, the isolated nucleic acid molecule is specific for Dermatophagoides farinae.

In some embodiments, the isolated nucleic acid molecule comprises a sequence at least about 80% identical to the internal transcribed spacer 1 (ITS1) of a sequence selected from any one of SEQ ID NOs:1-100, or the complementary sequence thereof, or fragment thereof.

In some embodiments, the isolated nucleic acid molecule comprises a sequence at least about 80% identical to the internal transcribed spacer 2 (ITS2) of a sequence selected from any one of SEQ ID NOs:1-100, or the complementary sequence thereof, or fragment thereof.

In some embodiments, the isolated nucleic acid molecule comprises a sequence at least about 80% identical to the internal transcribed spacer 1 (ITS1) and internal transcribed spacer 2 (ITS2) of the same sequence selected from any one of SEQ ID NOs:1-100, or the complementary sequence thereof, or fragment thereof.

In some embodiments the isolated nucleic acid molecule is comprising a sequence at least about 80% identical to a nucleic acid sequence selected from the list consisting of SEQ ID NO:101, 102, 103, 104, 105, 106, 107, 108, 109, 110, 122, 123, and 124, or the complementary sequence thereof, or fragment thereof, or complementary sequence thereof.

In some embodiments, the isolated nucleic acid molecule is consisting of a sequence at least about 80% identical to a nucleic acid sequence selected from the list consisting of SEQ ID NO:101, 102, 103, 104, 105, 106, 107, 108, 109, 110, 122, 123, and 124, or the complementary sequence thereof, or fragment thereof.

In some embodiments, the isolated nucleic acid molecule is comprising a nucleic acid sequence at least about 80% identical to 5.8S in a sequence selected from any one of SEQ ID NOs:1-100, or the complementary sequence thereof, such as Rast5.8, such as a nucleic acid sequence defined by SEQ ID NO:111 or the complementary sequence thereof, or fragment thereof.

In some embodiments, the isolated nucleic acid molecule is comprising a nucleic acid sequence at least about 80% identical to 18S in a sequence selected from any one of SEQ ID NOs:1-100, or the complementary sequence thereof, such as FRibNav, such as a nucleic acid sequence defined by SEQ ID NO:121 or the complementary sequence thereof, or fragment thereof.

Sequences:

Clon_DM1
(SEQ ID NO: 1)
Clon_DM21
(SEQ ID NO: 2)
Clon_DM6
(SEQ ID NO: 3)
Clon_DM20
(SEQ ID NO: 4)
Clon_DM9
(SEQ ID NO: 5)
Clon_DM12
(SEQ ID NO: 6)
Clon_DM7
(SEQ ID NO: 7)
Clon_DM11
(SEQ ID NO: 8)
Clon_DM14
(SEQ ID NO: 9)
Clon_DMA
(SEQ ID NO: 10)
Clon_DF1
(SEQ ID NO: 11)
Clon_DF6
(SEQ ID NO: 12)
Clon_DF4
(SEQ ID NO: 13)
Clon_DF26
(SEQ ID NO: 14)
Clon_DF4_50
(SEQ ID NO: 15)
Clon_DF19
(SEQ ID NO: 16)
Clon_DF3
(SEQ ID NO: 17)
Clon_DF5
(SEQ ID NO: 18)
Clon_DF2
(SEQ ID NO: 19)
Clon_DF7
(SEQ ID NO: 20)
Clon_AS15
(SEQ ID NO: 21)
Clon_AS14
(SEQ ID NO: 22)
Clon_AS20
(SEQ ID NO: 23)
Clon_AS13
(SEQ ID NO: 24)
Clon_AS10
(SEQ ID NO: 25)
Clon_AS11
(SEQ ID NO: 26)
Clon_AS2
(SEQ ID NO: 27)
Clon_AS12
(SEQ ID NO: 28)
Clon_AS1
(SEQ ID NO: 29)
Clon_AS16
(SEQ ID NO: 30)
Clon_BT8
(SEQ ID NO: 31)
Clon_BT9
(SEQ ID NO: 32)
Clon_BT16
(SEQ ID NO: 33)
Clon_BT3
(SEQ ID NO: 34)
Clon_BT14
(SEQ ID NO: 35)
Clon_BT17
(SEQ ID NO: 36)
Clon_BT13
(SEQ ID NO: 37)
Clon_BT1
(SEQ ID NO: 38)
Clon_BT10
(SEQ ID NO: 39)
Clon_BT15
(SEQ ID NO: 40)
Clon_TPA1_20
(SEQ ID NO: 41)
Clon_TPA1_22
(SEQ ID NO: 42)
Clon_TPA1_29
(SEQ ID NO: 43)
Clon_TPA1_28
(SEQ ID NO: 44)
Clon_TPA1_26
(SEQ ID NO: 45)
Clon_TPA1_21
(SEQ ID NO: 46)
Clon_TPA1_36
(SEQ ID NO: 47)
Clon_TPA1_27
(SEQ ID NO: 48)
Clon_TPA1_23
(SEQ ID NO: 49)
Clon_TPA1_1
(SEQ ID NO: 50)
Clon_TF22
(SEQ ID NO: 51)
Clon_TF24
(SEQ ID NO: 52)
Clon_TF3
(SEQ ID NO: 53)
Clon_TF2
(SEQ ID NO: 54)
Clon_TF23
(SEQ ID NO: 55)
Clon_TF1
(SEQ ID NO: 56)
Clon_TF4
(SEQ ID NO: 57)
Clon_TF7
(SEQ ID NO: 58)
Clon_TF15
(SEQ ID NO: 59)
Clon_TF14
(SEQ ID NO: 60)
Clon_DP8
(SEQ ID NO: 61)
Clon_DP1
(SEQ ID NO: 62)
Clon_DP7
(SEQ ID NO: 63)
Clon_DP3
(SEQ ID NO: 64)
Clon_DP6
(SEQ ID NO: 65)
Clon_DP9
(SEQ ID NO: 66)
Clon_DP2
(SEQ ID NO: 67)
Clon_DP4
(SEQ ID NO: 68)
Clon_DP10
(SEQ ID NO: 69)
Clon_DP5
(SEQ ID NO: 70)
Clon_EM4
(SEQ ID NO: 71)
Clon_EM21
(SEQ ID NO: 72)
Clon_EM2
(SEQ ID NO: 73)
Clon_EM23
(SEQ ID NO: 74)
Clon_EM3
(SEQ ID NO: 75)
Clon_EM24
(SEQ ID NO: 76)
Clon_EM22
(SEQ ID NO: 77)
Clon_EM1
(SEQ ID NO: 78)
Clon_EM6
(SEQ ID NO: 79)
Clon_EM5
(SEQ ID NO: 80)
Clon_GD1
(SEQ ID NO: 81)
Clon_GD10
(SEQ ID NO: 82)
Clon_GD2
(SEQ ID NO: 83)
Clon_GD5
(SEQ ID NO: 84)
Clon_GD3
(SEQ ID NO: 85)
Clon_GD12
(SEQ ID NO: 86)
Clon_GD7
(SEQ ID NO: 87)
Clon_GD9
(SEQ ID NO: 88)
Clon_GD8
(SEQ ID NO: 89)
Clon_GD13
(SEQ ID NO: 90)
Clon_LD5
(SEQ ID NO: 91)
Clon_LD13
(SEQ ID NO: 92)
Clon_LD14
(SEQ ID NO: 93)
Clon_LD1
(SEQ ID NO: 94)
Clon_LD11
(SEQ ID NO: 95)
Clon_LD3
(SEQ ID NO: 96)
Clon_LD2
(SEQ ID NO: 97)
Clon_LD12
(SEQ ID NO: 98)
Clon_LD8
(SEQ ID NO: 99)
Clon_LD15
(SEQ ID NO: 100)
Primer Sequences (5′-3′):
Forward (first) primers:
F1Tf_824
(SEQ ID NO: 101)
GACAGAAGCTGAAAGCCGT (Tyrophagus fanetzhangorum)
F1Ld_608
(SEQ ID NO: 102)
GATGTTCGAATCAATTGCTAGTG (Lepidoglyphus destructor)
F1Gd_567
(SEQ ID NO: 103)
GCATACCGTGTTGAAGCAGG (Glycyphagus domesticus)
F1Dp_501
(SEQ ID NO: 104)
GATCGACTGGCAATTGTTGAC
(Dermatophagoides pteronyssinus)
F1Tp_474
(SEQ ID NO: 105)
CGCCATTTGACACAGTACC (Tyrophagus putrescentiae)
F1Bt_419
(SEQ ID NO: 106)
TGTGTGTGGGGGATTTTGC (Blomia tropicalis)
F1Em_384
(SEQ ID NO: 107)
GAGCCTGACAATTATCAATGTGC (Euroglyphus maynei)
F1Dm_304
(SEQ ID NO: 108)
CGGGATGAACGTGTGGATG (Dermatophagoides microceras)
F1As_234
(SEQ ID NO: 109)
GTCGGTTACGGTCAAACG (Acarus siro)
F1Df_159
(SEQ ID NO: 110)
GAAACAATTGAATTGTGATTCTGC (Dermatophagoides farinae)
Reverse universal (second) primer:
RAst5.85
(SEQ ID NO: 111)
5′-TGCGTTCGAAWGTCGAGT-3′, W = T or A
Forward universal (second) primer:
FRibNav
(SEQ ID NO: 121)
5′-AGAGGAAGTAAAAGTCGTAACAAG-3′
Reverse (first) primers:
R1Dp_181
(SEQ ID NO: 122)
GCTTTCAATAACCTCATCAGTGTC (Dermatophagoides
pteronyssinus)
R1Bt_347
(SEQ ID NO: 123)
CCATCACTAAAGGACAGAACCGC (Blomia tropicalis)
R1Df_419
(SEQ ID NO: 124)
CTCCAGCAATCGAATTATGCTC (Dermatophagoides farinae)
Sequences and CLUSTAL W 2.1 multiple sequence alignment.

ITS1 and ITS2 are defined herein by the boundaries of ITS1 and ITS2 to the conserved sequences of 18s (in bold), 5.8s (2nd sequence in bold), and 28s (3rd sequence in bold). Accordingly, ITS1 is defined by the sequences having 18s with the sequence 5′-AGGATCATTA-3′ in the 5′ terminal of ITS1, and 5.8s with the sequence 5′-CTGYYAGTGG-3′ in the 3′ terminal of ITS1 (the sequences of 18s and 5.8s not included). ITS2 is defined by the sequences having 5.8s with the sequence 5′ TGAGCGTCGT 3′ in the 5′ terminal of ITS2, and 28s with the sequence 5′ CGACCTCAG 3′ in the 3′ terminal of ITS2 (the sequences of 5.8s and 28s not included). ITS1 goes downstream 18S sub-unit, and ITS2 goes downstream 5.8S sub-unit

| | |
|---|---|
| Clon_DM1 | GTTTCCGTAGGTGAACCTGCGGAAGGATCATTATCGG--TGTT----------------- |
| Clon_DM21 | GTTTCCGTAGGTGAACCTGCGGAAGGATCATTATCGG--TGTT----------------- |
| Clon_DM6 | GTTTCCGTAGGTGAACCTGCGGAAGGATCATTATCGG--TGTT----------------- |
| Clon_DM20 | GTTTCCGTAGGTGAACCTGCGGAAGGATCATTATCGG--TGTT----------------- |
| Clon_DM9 | GTTTCCGTAGGTGAACCTGCGGAAGGATCATTATCGG--TGTT----------------- |
| Clon_DM12 | GTTTCCGTAGGTGAACCTGCGGAAGGATCATTATCGG--TGTT----------------- |
| Clon_DM7 | GTTTCCGTAGGTGAACCTGCGGAAGGATCATTATCGG--TGTT----------------- |
| Clon_DM11 | GTTTCCGTAGGTGAACCTGCGGAAGGATCATTATCGG--TGTT----------------- |
| Clon_DM14 | GTTTCCGTAGGTGAACCTGCGGAAGGATCATTATCGG--TGTT----------------- |
| Clon_DMA | GTTTCCGTAGGTGAACCTGCGGAAGGATCATTATCGG--TGTT----------------- |
| Clon_DF1 | GTTTCCGTAGGTGAACCTGCGGAAGGATCATTATCGG--TGTT----------------- |
| Clon_DF6 | GTTTCCGTAGGTGAACCTGCGGAAGGATCATTATCGG--TGTT----------------- |
| Clon_DF4 | GTTTCCGTAGGTGAACCTGCGGAAGGATCATTATCGG--TGTT----------------- |
| Clon_DF26 | GTTTCCGTAGGTGAACCTGCGGAAGGATCATTATCGG--TGTT----------------- |
| Clon_DF450 | GTTTCCGTAGGTGAACCTGCGGAAGGATCATTATCGG--TGTT----------------- |
| Clon_DF19 | GTTTCCGTAGGTGAACCTGCGGAAGGATCATTATCGG--TGTT----------------- |
| Clon_DF3 | GTTTCCGTAGGTGAACCTGCGGAAGGATCATTATCGG--TGTT----------------- |
| Clon_DF5 | GTTTCCGTAGGTGAACCTGCGGAAGGATCATTATCGG--TGTT----------------- |
| Clon_DF2 | GTTTCCGTAGGTGAACCTGCGGAAGGATCATTATCGG--TGTT----------------- |
| Clon_DF7 | GTTTCCGTAGGTGAACCTGCGGAAGGATCATTATCGG--TGTT----------------- |
| Clon_AS15 | GTTTCCGTAGGTGAACCTGCGGAAGGATCATTAACGGATAGTTGCTTTGCT-TGCA---- |
| Clon_AS14 | GTTTCCGTAGGTGAACCTGCGGAAGGATCATTAACGGATAGTTGCTTTGCT-TGCA---- |
| Clon_AS20 | GTTTCCGTAGGTGAACCTGCGGAAGGATCATTAACGGATAGTTGCTTTGCT-TGCA---- |
| Clon_AS13 | GTTTCCGTAGGTGAACCTGCGGAAGGATCATTAACGGATAGTTGCTTTGCT-TGCA---- |
| Clon_AS10 | GTTTCCGTAGGTGAACCTGCGGAAGGATCATTAACGGATAGTTGCTTTGCT-TGCA---- |
| Clon_AS11 | GTTTCCGTAGGTGAACCTGCGGAAGGATCATTAACGGATAGTTGCTTTGCT-TGCA---- |
| Clon_AS2 | GTTTCCGTAGGTGAACCTGCGGAAGGATCATTAACGGATAGTTGCTTTGCT-TGCA---- |
| Clon_AS12 | GTTTCCGTAGGTGAACCTGCGGAAGGATCATTAACGGATAGTTGCTTTGCT-TGCA---- |
| Clon_AS1 | GTTTCCGTAGGTGAACCTGCGGAAGGATCATTAACGGATAGTTGCTTTGCT-TGCA---- |
| Clon_AS16 | GTTTCCGTAGGTGAACCTGCGGAAGGATCATTAACGGATAGTTGCTTTGCT-TGCA---- |
| Clon_BT8 | GTTTCCGTAGGTGAACCTGCGGAAGGATCATTATTGGATA-TTATTTTGGTGTG------ |
| Clon_BT9 | GTTTCCGTAGGTGAACCTGCGGAAGGATCATTATTGGATA-TTATTTTGGTGTG------ |
| Clon_BT16 | GTTTCCGTAGGTGAACCTGCGGAAGGATCATTATTGGATA-TTATTTTGGTGTG------ |
| Clon_BT3 | GTTTCCGTAGGTGAACCTGCGGAAGGATCATTATTGGATA-TTATTTTGGTGTG------ |
| Clon_BT14 | GTTTCCGTAGGTGAACCTGCGGAAGGATCATTATTGGATA-TTATTTTGGTGTG------ |
| Clon_BT17 | GTTTCCGTAGGTGAACCTGCGGAAGGATCATTATTGGATA-TTATTTTGGTGTG------ |
| Clon_BT13 | GTTTCCGTAGGTGAACCTGCGGAAGGATCATTATTGGATA-TTATTTTGGTGTG------ |
| Clon_BT1 | GTTTCCGTAGGTGAACCTGCGGAAGGATCATTATTGGATA-TTATTTTGGTGTG------ |
| Clon_BT10 | GTTTCCGTAGGTGAACCTGCGGAAGGATCATTATTGGATA-TTATTTTGGTGTG------ |
| Clon_BT15 | GTTTCCGTAGGTGAACCTGCGGAAGGATCATTATTGGATA-TTATTTTGGTGTG------ |
| Clon_TPA1_20 | GTTTCCGTAGGTGAACCTGCGGAAGGATCATTAACGGATCGACAGAA-GAAGCGAAGGA- |
| Clon_TPA1_22 | GTTTCCGTAGGTGAACCTGCGGAAGGATCATTAACGGATCGACAGAA-GAAGCGAAGGA- |
| Clon_TPA1_29 | GTTTCCGTAGGTGAACCTGCGGAAGGATCATTAACGGATCGACAGAA-GAAGCGAAGGA- |
| Clon_TPA1_28 | GTTTCCGTAGGTGAACCTGCGGAAGGATCATTAACGGATCGACAGAA-GAAGCGAAGGA- |
| Clon_TPA1_26 | GTTTCCGTAGGTGAACCTGCGGAAGGATCATTAACGGATCGACAGAA-GAAGCGAAGGA- |
| Clon_TPA1_21 | GTTTCCGTAGGTGAACCTGCGGAAGGATCATTAACGGATCGACAGAA-GAAGCGAAGGA- |
| Clon_TPA1_36 | GTTTCCGTAGGTGAACCTGCGGAAGGATCATTAACGGATCGACAGAA-GAAGCGAAGGA- |
| Clon_TPA1_27 | GTTTCCGTAGGTGAACCTGCGGAAGGATCATTAACGGATCGACAGAA-GAAGCGAAGGA- |
| Clon_TPA1_23 | GTTTCCGTAGGTGAACCTGCGGAAGGATCATTAACGGATCGACAGAA-GAAGCGAAGGA- |
| Clon_TPA1_1 | GTTTCCGTAGGTGAACCTGCGGAAGGATCATTAACGGATCGACAGAA-GAAGCGAAGGA- |
| Clon_TF22 | GTTTCCGTAGGTGAACCTGCGGAAGGATCATTAACGGATCGACAGAA-GC--TGAAAGCC |
| Clon_TF24 | GTTTCCGTAGGTGAACCTGCGGAAGGATCATTAACGGATCGACAGAA-GC--TGAAAGCC |
| Clon_TF3 | GTTTCCGTAGGTGAACCTGCGGAAGGATCATTAACGGATCGACAGAA-GC--TGAAAGCC |
| Clon_TF2 | GTTTCCGTAGGTGAACCTGCGGAAGGATCATTAACGGATCGACAGAA-GC--TGAAAGCC |
| Clon_TF23 | GTTTCCGTAGGTGAACCTGCGGAAGGATCATTAACGGATCGACAGAA-GC--TGAAAGCC |
| Clon_TF1 | GTTTCCGTAGGTGAACCTGCGGAAGGATCATTAACGGATCGACAGAA-GC--TGAAAGCC |
| Clon_TF4 | GTTTCCGTAGGTGAACCTGCGGAAGGATCATTAACGGATCGACAGAA-GC--TGAAAGCC |
| Clon_TF7 | GTTTCCGTAGGTGAACCTGCGGAAGGATCATTAACGGATCGACAGAA-GC--TGAAAGCC |
| Clon_TF15 | GTTTCCGTAGGTGAACCTGCGGAAGGATCATTAACGGATCGACAGAA-GC--TGAAAGCC |
| Clon_TF14 | GTTTCCGTAGGTGAACCTGCGGAAGGATCATTAACGGATCGACAGAA-GC--TGAAAGCC |
| Clon_DP8 | GTTTCCGTAGGTGAACCTGCGGAAGGATCATTATCGGTTTTCTTGAGCA----------- |
| Clon_DP1 | GTTTCCGTAGGTGAACCTGCGGAAGGATCATTATCGGTTTTCTTGAGCA----------- |
| Clon_DP7 | GTTTCCGTAGGTGAACCTGCGGAAGGATCATTATCGGCTTTCTTGAGCA----------- |
| Clon_DP3 | GTTTCCGTAGGTGAACCTGCGGAAGGATCATTATCGGTTTTCTTGAGCAA---------- |
| Clon_DP6 | GTTTCCGTAGGTGAACCTGCGGAAGGATCATTATCGGTTTTCTTGAGCAA---------- |
| Clon_DP9 | GTTTCCGTAGGTGAACCTGCGGAAGGATCATTATCGGTTTTCTTGAGCAA---------- |
| Clon_DP2 | GTTTCCGTAGGTGAACCTGCGGAAGGATCATTATCGGTTTTCTTGAGCA----------- |
| Clon_DP4 | GTTTCCGTAGGTGAACCTGCGGAAGGATCATTATCGGTTTTCTTGAGCA----------- |
| Clon_DP10 | GTTTCCGTAGGTGAACCTGCGGAAGGATCATTATCGATTTTCTTGAGCA----------- |
| Clon_DP5 | GTTTCCGTAGGTGAACCTGCGGAAGGATCATTATCGGTTTTCTTGAGCA----------- |
| Clon_EM4 | GTTTCCGTAGGTGAACCTGCGGAAGGATCATTATCGGTTTTTCGTGGCA----------- |
| Clon_EM21 | GTTTCCGTAGGTGAACCTGCGGAAGGATCATTATCGGTTTTTCGTGGCA----------- |
| Clon_EM2 | GTTTCCGTAGGTGAACCTGCGGAAGGATCATTATCGGTTTTTCGTGGCA----------- |
| Clon_EM23 | GTTTCCGTAGGTGAACCTGCGGAAGGATCATTATCGGTTTTTCGTGGCA----------- |
| Clon_EM3 | GTTTCCGTAGGTGAACCTGCGGAAGGATCATTATCGGTTTTTCGTGGCA----------- |
| Clon_EM24 | GTTTCCGTAGGTGAACCTGCGGAAGGATCATTATCGGTTTTTCGTGGCA----------- |
| Clon_EM22 | GTTTCCGTAGGTGAACCTGCGGAAGGATCATTATCGGTTTTTCGTGGCA----------- |
| Clon_EM1 | GTTTCCGTAGGTGAACCTGCGGAAGGATCATTATCGGTTTTTC--GACA----------- |
| Clon_EM6 | GTTTCCGTAGGTGAACCTGCGGAAGGATCATTATCGGTTTTTCGTGGCA----------- |
| Clon_EM5 | GTTTCCGTAGGTGAACCTGCGGGAGGATCATTATCGGTTATTC--GACA----------- |
| Clon_GD1 | GTTTCCGTAGGTGAACCTGCGGAAGGATCATTAACGGATTGTTTGTC------------- |
| Clon_GD10 | GTTTCCGTAGGTGAACCTGCGGAAGGATCATTAACGGATTGTTTGTC------------- |
| Clon_GD2 | GTTTCCGTAGGTGAACCTGCGGAAGGATCATTAACGGATTGTTTGTC------------- |
| Clon_GD5 | GTTTCCGTAGGTGAACCTGCGGAAGGATCATTAACGGATTGTTTGTT------------- |
| Clon_GD3 | GTTTCCGTAGGTGAACCTGCGGAAGGATCATTAACGGATTGTTTGTC------------- |
| Clon_GD12 | GTTTCCGTAGGTGAACCTGCGGAAGGATCATTAACGGATTGTTTGTC------------- |
| Clon_GD7 | GTTTCCGTAGGTGAACCTGCGGAAGGATCATTAACGGATTGTTTGTT------------- |
| Clon_GD9 | GTTTCCGTAGGTGAACCTGCGGAAGGATCATTAACGGATTGTTTGTC------------- |
| Clon_GD8 | GTTTCCGTAGGTGAACCTGCGGAAGGATCATTAACGGATTGTTTGTC------------- |
| Clon_GD13 | GTTTCCGTAGGTGAACCTGCGGAAGGATCATTAACGGATTGTTTGTC------------- |
| Clon_LD5 | GTTTCCGTAGGTGAACCTGCGGAAGGATCATTAACGGATTGTTTATTCT----------- |
| Clon_LD13 | GTTTCCGTAGGTGAACCTGCGGAAGGATCATTAACGGATTGTTTATTCT----------- |
| Clon_LD14 | GTTTCCGTAGGTGAACCTGCGGAAGGATCATTAACGGATTGTTTATTCT----------- |
| Clon_LD1 | GTTTCCGTAGGTGAACCTGCGGAAGGATCATTAACGGATTGTTTATTCT----------- |
| ClonLD11 | GTTTCCGTAGGTGAACCTGCGGAAGGATCATTAACGGATTGTTTATTCT----------- |
| Clon_LD3 | GTTTCCGTAGGTGAACCTGCGGAAGGATCATTAACGGATTGTTTATTCT----------- |
| Clon_LD2 | GTTTCCGTAGGTGAACCTGCGGAAGGATCATTAACGGATTGTTTATTCT----------- |
| Clon_LD12 | GTTTCCGTAGGTGAACCTGCGGAAGGATCATTAACGGATTGTTTATTCT----------- |
| Clon_LD8 | GTTTCCGTAGGTGAACCTGCGGAAGGATCATTAACGGATTGTTTATTCT----------- |
| Clon_LD15 | GTTTCCGTAGGTGAACCTGCGGAAGGATCATTAACGGATTGTTTATTCT----------- |
| | ********************** ********** * |
| Clon_DM1 | ----------------------------------------TTTTTT-------------- |
| Clon_DM21 | ----------------------------------------TTTTTT-------------- |
| Clon_DM6 | ----------------------------------------TTTTTT-------------- |
| Clon_DM20 | ----------------------------------------TTTTTT-------------- |
| Clon_DM9 | ----------------------------------------TTTTTT-------------- |
| Clon_DM12 | ----------------------------------------TTTTTT-------------- |
| Clon_DM7 | ----------------------------------------TTTTTT-------------- |
| Clon_DM11 | ----------------------------------------TTTTTT-------------- |
| Clon_DM14 | ----------------------------------------TTTTTT-------------- |
| Clon_DMA | ----------------------------------------TTTTTT-------------- |
| Clon_DF1 | ----------------------------------------TTT--T-------------- |
| Clon_DF6 | ----------------------------------------TTT--T-------------- |
| Clon_DF4 | ----------------------------------------TTTG-T-------------- |
| Clon_DF26 | ----------------------------------------TTTG-T-------------- |
| Clon_DF4_50 | ----------------------------------------TTT--T-------------- |
| Clon_DF19 | ----------------------------------------TTT--T-------------- |
| Clon_DF3 | ----------------------------------------TTTGTT-------------- |
| Clon_DF5 | ----------------------------------------TTTGTT-------------- |
| Clon_DF2 | ----------------------------------------TTTGTT-------------- |
| Clon_DF7 | ----------------------------------------TTTGTT-------------- |
| Clon_AS15 | -TTTGT----------------------------------TTTGCT-------------- |
| Clon_AS14 | -TTTGT----------------------------------TTTGCT-------------- |
| Clon_AS20 | -TTTGT----------------------------------TTTGCT-------------- |
| Clon_AS13 | -TTTGT----------------------------------TTTGCT-------------- |
| Clon_AS10 | -TTTGT----------------------------------TTTGCT-------------- |
| Clon_AS11 | -TTTACC---------------------------------TTTGCT-------------- |
| Clon_AS2 | -TTTAC----------------------------------TTTGCT-------------- |
| Clon_AS12 | -TTTACC---------------------------------TTTGCT-------------- |
| Clon_AS1 | -TTTAC----------------------------------TTTGCT-------------- |
| Clon_AS16 | -TTTAC----------------------------------TTTGCT-------------- |
| Clon_BT8 | -----------------------------------------TGATT-------------- |
| Clon_BT9 | -----------------------------------------TGATT-------------- |
| Clon_BT16 | -----------------------------------------TGATT-------------- |
| Clon_BT3 | -----------------------------------------TGATT-------------- |
| Clon_BT14 | -----------------------------------------TGATT-------------- |
| Clon_BT17 | -----------------------------------------TGATT-------------- |
| Clon_BT13 | -----------------------------------------TGATT-------------- |
| Clon_BT1 | -----------------------------------------TGATT-------------- |
| Clon_BT10 | -----------------------------------------TGATT-------------- |
| Clon_BT15 | -----------------------------------------TGATT-------------- |
| Clon_TPA1_20 | GTTCA-----G-------------------------CTCTTTCACT-------------- |
| Clon_TPA1_22 | GTTCA-----G-------------------------CTCTTTCACT-------------- |
| Clon_TPA1_29 | GTTCA-----G-------------------------CTCTTTCACT-------------- |
| Clon_TPA1_28 | GTTCA-----G-------------------------CTCTTTCACT-------------- |
| Clon_TPA1_26 | GTTCA-----G-------------------------CTCTTTCACT-------------- |
| Clon_TPA1_21 | GTTCA-----G-------------------------CTCTTTCACT-------------- |
| Clon_TPA1_36 | GTTCA-----G-------------------------CTCTTTCACT-------------- |
| Clon_TPA1_27 | GTTCA-----G-------------------------CTCTTTCACT-------------- |
| Clon_TPA1_23 | GTTCA-----G-------------------------CTCTTTCACT-------------- |
| Clon_TPA1_1 | GTTCA-----G-------------------------CTCTTTCACT-------------- |
| Clon_TF22 | GTCTGCTGTTGTGCTCTTGCAGTGCATCATCATCATCACTTTCACT-------------- |
| Clon_TF24 | GTCTGCTGTTGTGCTCTTGCAGTGCATCATCATCATCACTTTCACT-------------- |
| Clon_TF3 | GTCTGCTGTTGTGCTCTTGCAGTGCATCATCATCATCACTTTCACT-------------- |
| Clon_TF2 | GTCTGCTGTTGTGCTCTTGCAGTGCATCATCATCATCACTTTCACT-------------- |
| Clon_TF23 | GTCTGCTGTTGTGCTCTTGCAGTGCATCATCATCATCACTTTCACT-------------- |
| Clon_TF1 | GTCTGTTGTTGTGCTCTTGCAGTGCATCATCATTATCACTTTCACT-------------- |
| Clon_TF4 | GTCTGTTGTTGTGCTCTTGCAGTGCATCATCATTATCACTTTCACT-------------- |
| Clon_TF7 | GTCTGTTGTTGTGCTCTTGCAGTGCATCATCATTATCACTTTCACT-------------- |
| Clon_TF15 | GTCTGCTGTTGTGCTCTTGCAGTGCATCATCATCATCACTTTCACT-------------- |
| Clon_TF14 | GTTTGTTGTTGTGCTCTTGCGGTGCATCATCATTATCACTTTCACT-------------- |
| Clon_DP8 | ----------------------------------------TTCATTT------------- |
| Clon_DP1 | ----------------------------------------TTCATTT------------- |
| Clon_DP7 | ----------------------------------------TTCATTTT------------ |
| Clon_DP3 | ----------------------------------------TTTATTT------------- |
| Clon_DP6 | ----------------------------------------TTTATTT------------- |
| Clon_DP9 | ----------------------------------------TTTATTT------------- |
| Clon_DP2 | ----------------------------------------TTTATTT------------- |
| Clon_DP4 | ----------------------------------------TTTATTT------------- |
| Clon_DP10 | ----------------------------------------TTCATTTT------------ |
| Clon_DP5 | ----------------------------------------TTTATTT------------- |
| Clon_EM4 | ----------------------------------------TTCATTT------------- |
| Clon_EM21 | ----------------------------------------TTCATTT------------- |
| Clon_EM2 | ----------------------------------------TTCATTT------------- |
| Clon_EM23 | ----------------------------------------TTCATTT------------- |
| Clon_EM3 | ----------------------------------------TTCATTT------------- |
| Clon_EM24 | ----------------------------------------TTCATTT------------- |
| Clon_EM22 | -----------------------------------------TTCATTT------------ |
| Clon_EM1 | ----------------------------------------GT--TTT------------- |
| Clon_EM6 | ----------------------------------------TTCATTT------------- |
| Clon_EM5 | ----------------------------------------GT--TTT------------- |
| Clon_GD1 | ----------------------------------------TTAATTTTTGCAAT-TTAC- |
| Clon_GD10 | ----------------------------------------TTGATTTT--CAAT-TTATC |
| Clon_GD2 | ----------------------------------------TTGATTTT--CAAT-TTATC |
| Clon_GD5 | ----------------------------------------TTGATTTT--CAAT-TTATC |
| Clon_GD3 | ----------------------------------------TTGATTTT--CAAT-TTATC |
| Clon_GD12 | ----------------------------------------TTGATTTT--CAAT-TTATC |
| Clon_GD7 | ----------------------------------------TTGATTTT--CAAT-TTATC |
| Clon_GD9 | ----------------------------------------TTGATTTT--CAAT-TTATC |
| Clon_GD8 | ----------------------------------------TTGATTTA--CAACGTTACA |
| Clon_GD13 | ----------------------------------------TTGATTTA--CAACGTTACA |
| Clon_LD5 | ----------------------------------------TTCATTGC----ATTTCACA |
| Clon_LD13 | ----------------------------------------TTCATTGC----ATTTCACA |
| Clon_LD14 | ----------------------------------------TTCATTGC----ATTTCACA |
| Clon_LD1 | ----------------------------------------TTCATTGC----ATTTCACA |
| Clon_LD11 | ----------------------------------------TTCATTGC----ATTTCACA |
| Clon_LD3 | ----------------------------------------TTCATTGC----ATTTCACA |
| Clon_LD2 | ----------------------------------------TTCATTGC----ATTTCACA |
| Clon_LD12 | ----------------------------------------TTCATTGC----ATTTCACA |
| Clon_LD8 | ----------------------------------------TTCATTGC----ATTTCA-- |
| Clon_LD15 | ----------------------------------------TTCATTGC----ATTTCA-- |
| | * * |
| Clon_DM1 | ---G--------------------------CACTTGA----TT--CTTAAATCGAACGAA |
| Clon_DM21 | ---G--------------------------CACTTGA----TT--CTTAAATCGAACGAA |
| Clon_DM6 | ---G--------------------------CACTTGA----TT--CTTAAATCGAACGAA |
| Clon_DM20 | ---G--------------------------CACTTGA----TT--CTTAAATCGAACGAA |
| Clon_DM9 | --TG--------------------------CACTTGA----TT--CTTAAATCGAACGAA |
| Clon_DM12 | --TG--------------------------CACTTGA----TT--CTTAAATCGAACGAA |
| Clon_DM7 | ---G--------------------------CACTTGA----TT--CTTAAATCGAACGAA |
| Clon_DM11 | --TG--------------------------CACTTGA----TT--CTTAAATCGAACGAA |
| Clon_DM14 | --TG--------------------------CACTTGA----TT--CTTAAATCGAACGAA |
| Clon_DMA | ---G--------------------------CACTTGA----TT--CTTAAATCGAACGAA |
| Clon_DF1 | --TGAA-----CAATGAA-----------ACACTTGA----TT--CTCAATTCGAACGAT |
| Clon_DF6 | --TGAA-----CAATGAA-----------ACACTTGA----TT--CTCAATTCGAACGAT |
| Clon_DF4 | --TGAA-----CAATGAA-----------ACACTTGA----TT--CTCAATTCGAACGAT |
| Clon_DF26 | --TGAA-----CAATGAA-----------ACACTTGA----TT--CTCAATTCGAACGAT |
| Clon_DF4_50 | --TGAA-----CAATGAA-----------ACACTTGA----TT--CTCAATTCGAACGAT |
| Clon_DF19 | --TGAA-----CAATGAA-----------ACACTTGA----TT--CTCAATTCGAACGAT |
| Clon_DF3 | --TGAA-----CAATGAA-----------ACACTTGA----TT--CTCAATTCGAACGAT |
| Clon_DF5 | --TGAA-----CAATGAA-----------ACACTTGA----TT--CTCAATTCGAACGAT |
| Clon_DF2 | --TGAA-----CAATGAA-----------ACACTTGA----TT--CTCAATTCGAACGAT |
| Clon_DF7 | --TGAA-----CAATGAA-----------ACACTTGA----TT--CTCAATTCGAACGAT |
| Clon_AS15 | GCTAAAGAAACAATCA-------------ATATCCAAACCTTT--ACCA-CTTGAACGC- |
| Clon_AS14 | GCTAAAGAAACAATCA-------------ATATCCAAACCTTT--ACCA-CTTGAACGC- |
| Clon_AS20 | GCTAAAGAAACAATCA-------------ATATCCAAACCTTT--ACCA-CTTGAACGC- |
| Clon_AS13 | GCTAAAGAAACAATCA-------------ATATCCAAACCTTT--ACCA-CTTGAACGC- |
| Clon_AS10 | GCTAAAGAAACAATCA-------------ATATCCAAACCTTT--ACCA-CTTGAACGC- |
| Clon_AS11 | GCTAAAGAAACAATCA-------------ATATCCAAACCTTT--ACCT-CTTGAACGC- |
| Clon_AS2 | GCTAAAGAAACAATCA-------------ATATCCAAACCTTT--ACCT-CTTGAACGC- |
| Clon_AS12 | GCTAAAGAAACAATCA-------------ATATCCAAACCTTT--ACCA-CTTGAACGC- |
| Clon_AS1 | GCTAAAGAAACAATCA-------------ATATCCAAACCTTT--ACCA-CTTGAACGC- |
| Clon_AS16 | GCTAAAGAAACAATCA-------------ATATCCAAACCTTT--ACCT-CTTGAACGC- |
| Clon_B78 | ATTCA--------TCA-------------TCATGCTAAATTTT--ATC----TAAA---- |
| Clon_BT9 | ATTCA--------TCA-------------TCATGCTAAATTTT--ATC----TAAA---- |
| Clon_BT16 | ATTCA--------TCA-------------TCATGCTAAATTTT--ATC----TAAA---- |
| Clon_BT3 | ATTCA--------TCA-------------TCATGCTAAATTTT--ATC----TAAA---- |
| Clon_BT14 | ATTCA--------TCA-------------T---GCTAAATTTT--ATC----TAAA---- |
| Clon_BT17 | ATTCA--------TCA-------------TCATGCTAAATTTT--ATC----TAAA---- |
| Clon_BT13 | ATTCA--------TCA-------------T---GCTAAATTTT--ATC----TAAA---- |
| Clon_BT1 | ATTCA--------TCA-------------TCATGCTAA-TTTT--ATC----TAAA---- |
| Clon_BT10 | ATTCA--------TCA-------------TCATGCTAAATTTT--ATC----TAAA---- |
| Clon_BT15 | ATTCA--------TCA-------------TCATGCTAAATTTT--ATC----TAAA---- |
| Clon_TPA1_20 | GCCAC------TGTCAC------------GTATCCAAACCTTT---TTG-CTTGAACGC- |
| Clon_TPA1_22 | GCCAC------TGTCAC------------GTATCCAAACCTTT---TTG-CTTGAACGC- |
| Clon_TPA1_29 | GCCAC------TGTCAC------------GTATCCAAACCTTT---TTG-CTTGAACGC- |
| Clon_TPA1_28 | GCCAC------TGTCAC------------GTATCCAAACCTTT---TTG-CTTGAACGC- |
| Clon_TPA1_26 | GCCAC------TGTCAC------------GTATCCAAACCTTT---TTG-CTTGAACGC- |
| Clon_TPA1_21 | GCCAC------TGTCAC------------GTATCCAAACCTTT---TTG-CTTGAACGC- |
| Clon_TPA1_36 | GCCAC------TGTCAC------------GTATCCAAACCTTT---TTG-CTTGAACGC- |
| Clon_TPA1_27 | GCCAC------TGTCAC------------GTATCCAAACCTTT---TTG-CTTGAACGC- |
| Clon_TPA1_23 | GCCAC------TGTCAC------------GTATCCAAACCTTT---TTG-CTTGAACGC- |
| Clon_TPA1_1 | GCCAC------TGTCAC------------GTATCCAAACCTTT---TTG-CTTGAACGC- |
| Clon_TF22 | GCCAC------TGTCAC------------GTATCCAAACCTTT---TTG-CTTGAACGC- |
| Clon_TF24 | GCCAC------TGTCAC------------GTATCCAAACCTTT---TTG-CTTGAACGC- |
| Clon_TF3 | GCCAC------TGTCAC------------GTATCCAAACCTTT---TTG-CTTGAACGC- |
| Clon_TF2 | GCCAC------TGTCAC------------GTATCCAAACCTTT---TTG-CTTGAACGC- |
| Clon_TF23 | GCCAC------TGTCAC------------GTATCCAAACCTTT---TTG-CTTGAACGC- |
| Clon_TF1 | GCCAC------TGTCAC------------GTATCCAAACCTTT---TTG-CTTGAACGC- |
| Clon_TF4 | GCCAC------TGTCAC------------GTATCCAAACCTTT---TTG-CTTGAACGC- |
| Clon_TF7 | GCCAC------TGTCAC------------GTATCCAAACCTTT---TTG-CTTGAACGC- |
| Clon_TF15 | GCCAC------TGTCAC------------GTATCCAAACCTTT---TTG-CTTGAACGC- |
| Clon_TF14 | GCCAC------TGTCAC------------GTATCCAAACCTTT---TTG-CTTGAACGC- |
| Clon_DP8 | GCTTAA-----AAGAA-------------ACATTCGA----TT--ATCAATTCGAACGAA |
| Clon_DP1 | GCTTAA-----AAGAA-------------ACATTCGA----TT--ATCAATTCGAACGAA |
| Clon_DP7 | GCTTAA-----AAGAA-------------ACATTCGA----TT--ATCAATTCGAACGAA |
| Clon_DP3 | GCTTAA-----AAGAA-------------ACATTCGA----TT--ATCAATTCGAACGAA |
| Clon_DP6 | GCTTAA-----AAGAA-------------ACATTCGA----TT--ATCAATTCGAACGAA |
| Clon_DP9 | GCTTAA-----AAGAA-------------ACATTCGA----TT--ATCAATTCGAACGAA |
| Clon_DP2 | GCCCAA-----AAGAA-------------ACATTCGA----TT--ATCAATTCGAACGAA |
| Clon_DP4 | GCCCAA-----AAGAA-------------ACATTCGA----TT--ATCAATTCGAACGAA |
| Clon_DP10 | GCCCAA-----AAGAA-------------ACATTCGA----TT--ATCAATTCGAACGAA |
| Clon_DP5 | GCCCAA-----AAGAA-------------ACATTCGA----TT--ATCAATTCGAACGAA |
| Clon_EM4 | GCT-GT-----CGGAA-------------ACATTCGA----TT--ATCAATTCGAACGA- |
| Clon_EM21 | GCT-GT-----CGGAA-------------ACATTCGA----TT--ATCAATTCGAACGA- |
| Clon_EM2 | GCT-GT-----CGGAA-------------ACATTCGA----TT--ATCAATTCGAACGA- |
| Clon_EM23 | GCT-GT-----CGGAA-------------ACATTCGA----TT--ATCAATTCGAACGA- |
| Clon_EM3 | GCT-GT-----CGGAA-------------ACATTCGA----TT--ATCAATTCGAACGA- |
| Clon_EM24 | GCT-GT-----CGGAA-------------ACATTCGA----TT--ATCAATTCGAACGA- |
| Clon_EM22 | GCT-GT-----CGGAA-------------ACATTCGA----TT--ATCAATTCGAACGA- |
| Clon_EM1 | GCT-GA-----CGGAA-------------ACATTCGA----TT--ATCAATTCGAACGA- |
| Clon_EM6 | GCT-GT-----CGGAA-------------ACATTCGA----TT--ATCAATTCGAACGA- |
| Clon_EM5 | GCT-GA-----CGGAA-------------ACATTCGA----TT--ATCAATTCGAACGA- |
| Clon_GD1 | -TTGTAA----TATTAAA-ACGAGCATCAATATCCGAACCTTTCAAAAAAATCGAACGA- |
| Clon_GD10 | ATTGTTT----TATCATACACGAGCATCAATATCCGAACCTTTCAAAAAAATCGAACGA- |
| Clon_GD2 | ATTGTTT----TATCATACACGAGCATCAATATCCGAACCTTTCAAAAAAATCGAACGA- |
| Clon_GD5 | ATTGTTT----TATCATACACGAGCATCAATATCCGAACCTTTCAAAAAAATCGAACGA- |
| Clon_GD3 | ATTGTTT----TATCATACACGAGCATCAATATCCGAACCTTTCAAAAAAATCGAACGA- |
| Clon_GD12 | ATTGTTT----TATCATACACGAGCATCAATATCCGAACCTTTCAAAAAAATCGAACGA- |
| Clon_GD7 | ATTGTTT----TATCATACACGAGCATCAATATCCGAACCTTTCAAAAAAATCGAACGA- |
| Clon_GD9 | ATTGTTT----TATCATACACGAGCATCAATATCCGAACCTTTCAAAAAAATCGAACGA- |
| Clon_GD8 | ATTGTTT----TATCATACACGAGCATCAATATCCGAACCTTTCAAAAAAATCGAACGA- |
| Clon_GD13 | ATTGTTT----TATCATACACGAGCATCAATATCCGAACCTTTCAAAAAAATCGAACGA- |
| Clon_LD5 | --TGCA-----TGTTGGAACAAAGCAAAAATATCCGAACCTTTCAAACAAATCGAACGA- |
| Clon_LD13 | --TGCA-----TGTTGGAACAAAGCAAAAATATCCGAACCTTTCAAACAAATCGAACGA- |
| Clon_LD14 | --TGCA-----TGTTGGAACAAAGCAAAAATATCCGAACCTTTCAAACAAATCGAACGA- |
| Clon_LD1 | --TGCA-----TGTTGGAACAAAGCAAAAATATCCGAACCTTTCAAACAAATCGAACGA- |
| Clon_LD11 | --TGCA-----TGTTGGAACAAAGCAAAAATATCCGAACCTTTCAAACAAATCGAACGA- |
| Clon_LD3 | --TGCA-----TGTTGGAACAAAGCAAAAATATCCGAACCTTTCAAACAAATCGAACGA- |
| Clon_LD2 | --TGCA-----TGTTGGAACAAAGCAAAAATATCCGAACCTTTCAAACAAATCGAACGA- |
| Clon_LD12 | --TGCA-----TGTTGGAACAAAGCAAAAATATCCGAACCTTTCAAACAAATCGAACGA- |
| Clon_LD8 | --TGCA-----TGTTGGAACAAAGCAAAAATATCCGAACCTTTCAAACAAATCGAACGA- |
| Clon_LD15 | --TGCA-----TGTTGGAACAAAGCAAAAATATCCGAACCTTTCAAACAAATCGAACGA- |
| | * * ** |
| Clon_DM1 | AAGTTGCCCGTTATCACAA----------------ACGGATCGAC-----------T--- |
| Clon_DM21 | AAGTTGCCCGTTATCACAA----------------ACGGATCGAC-----------T--- |
| Clon_DM6 | AAGTTGCCCGTTATCACAA----------------ACGGATCGAC-----------T--- |
| Clon_DM20 | AAGTTGCCCGTTATCACAA----------------ACGGATCGAC-----------T--- |
| Clon_DM9 | AAGTTGCCCGTTATCACAA----------------ACGGATCGAC-----------T--- |
| Clon_DM12 | AAGTTGCCCGTTATCACAA----------------ACGGATCGAC-----------T--- |
| Clon_DM7 | AAGTTGCCCGTTATCACAA----------------ACGGATCGAC-----------T--- |
| Clon_DM11 | AAGTTGCCCGTTATCACAA----------------ACGGATCGAC-----------T--- |
| Clon_DM14 | AAGTTGCCCGTTATCACAA----------------ACGGATCGAC-----------T--- |
| Clon_DMA | AAGTTGCCCGTTATCACAA----------------ACGGATCGAC-----------T--- |
| Clon_DF1 | AAGTTGCCCGTTATCACAA----------------ACGGATCGAC-----------T--- |
| Clon_DF6 | AAGTTGCCCGTTATCACAA----------------ACGGATCGAC-----------T--- |
| Clon_DF4 | AAGTTGCCCGTTATCACAA----------------ACGGATCGAC-----------T--- |
| Clon_DF26 | AAGTTGCCCGTTATCACAA----------------ACGGATCGAC-----------T--- |
| Clon_DF4_50 | AAGTTGCCCGTTATCACAA----------------ACGGATCGAC-----------T--- |
| Clon_DF19 | AAGTTGCCCGTTATCACAA----------------ACGGATCGAC-----------T--- |
| Clon_DF3 | AAGTTGCCCGTTATCACAA----------------ACGGATCGAC-----------T--- |
| Clon_DF5 | AAGTTGCCCGTTATCACAA----------------ACGGATCGAC-----------T--- |
| Clon_DF2 | AAGTTGCCCGTTATCACAA----------------ACGGATCGAC-----------T--- |
| Clon_DF7 | AAGTTGCCCGTTATCACAA----------------ACGGATCGAC-----------T--- |
| Clon_AS15 | CTATTGCCCGTTAGCATACCCCC------ATGCTAATGAGCTGATCATGCGTTGGTT-CT |
| Clon_AS14 | CTATTGCCCGTTAGCATACCCCC------ATGCTAATGAGCTGATCATGCGTTGGTT-CT |
| Clon_AS20 | CTATTGCCCGTTAGCATACCCCC------ATGCTAATGAGCTGATCATGCGTTGGTT-CT |
| Clon_AS13 | CTATTGCCCGTTAGCATACCCCC------ATGCTAATGAGCTGATCATGCGTTGGTT-CT |
| Clon_AS10 | CTATTGCCCGTTAGCATACCCCC------ATGCTAATGAGCTGATCATGCGTTGGTTTCT |
| Clon_AS11 | CTATTGCCCGTTAGCATATCC--------ATGCTAATGAGCTGATCATGCGTTGGTT-CT |
| Clon_AS2 | CTATTGCCCGTTAGCATACCCCC------ATGCTAATGAGCTGATCATGCGTTGGTT-CT |
| Clon_AS12 | CTATTGCCCGTTAGCATACCCCC------ATGCTAATGAGCTGATCATGCGTTGGTT-CT |
| Clon_AS1 | CTATTGCCCGTTAGCATATCC--------ATGCTAATGAGCTGACCATGCGTTGGTT-CT |
| Clon_AS16 | CTATTGCCCGTTAGCATATCC--------ATGCTAATGAGCTGATCATGCGTTGGTT-CT |
| Clon_B78 | ---TTTTGTGTTTTCGGAC----------------ACGAAGCCAT----------TT--- |
| Clon_B79 | ---TTTTGTGTTTTCGGAC----------------ACGAAGCCAT----------TT--- |
| Clon_B716 | ---TTTTGTGTTTTCGGAC----------------ACGAAGCCAT----------TT--- |
| Clon_B73 | ---TTTTGTGTTTTCGGAC----------------ACGAAGCCAT----------TT--- |
| Clon_B714 | ---TTTTGTGTTTTCGGAC----------------ACGAA-TATT----------TT--- |
| Clon_B717 | ---TTTTGTGTTTTCGGAC----------------ACGAA-TATT----------TT--- |
| Clon_B713 | ---TTTTGTGTTTTCGGAC----------------ACGAAGCCAT----------TT--- |
| Clon_B71 | ---TTTTGTGTTTTCGGAC----------------ACGAAGCCAT----------TT--- |
| Clon_B710 | ---TTTTGTGTTTTCGGAC----------------ACGAA-TTAT----------TT--- |
| Clon_B715 | ---TTTTGTGTTTTCGGAC----------------ACGAA-TTAT----------TT--- |
| Clon_TPA1_20 | AAATTGCCCGTTACCA-GAAGTA-----ACCAAAA-TGGGCTTATCATG------TT--- |
| Clon_TPA1_22 | AAATTGCCCGTTACCA-GAAGTA-----ACCAAAA-TGGGCTTATCATG------TT--- |
| Clon_TPA1_29 | AAATTGCCCGTTACCA-GAAGTA-----ACCAAAA-TGGGCTTATCATG------TT--- |
| Clon_TPA1_28 | AAATTGCCCGTTACCA-GAAGTA-----ACCAAAA-TGGGCTTATCATG------TT--- |
| Clon_TPA1_26 | AAATTGCCCGTTACCA-AATGTA-----ACCAAAA-TGGGCTTATCATG------TT--- |
| Clon_TPA1_21 | AAATTGCCCGTTACCA-AATGTA-----ACCAAAA-TGGGCTTATCATG------TT--- |
| Clon_TPA1_36 | AAATTGCCCGTTACCA-AATGTA-----ACCAAAA-TGGGCTTATCATG------TT--- |
| Clon_TPA1_27 | AAATTGCCCGTTACCA-AATGTA-----ATCAAAA-TGGGCTTATCATG------TT--- |
| Clon_TPA1_23 | AAATTGCCCGTTACCA-AATGTA-----ACCAAAAATGGGCTTATCATG------TT--- |
| Clon_TPA1_1 | AAATTGCCCGTTACCA-AATGTA-----ACCAAAAATGGGCTTATCATG------TT--- |
| Clon_TF22 | AAATTGCCCGTTACCAAAA---------GCTAAAAATGGGCTTATCATG------TT--- |
| Clon_TF24 | AAATTGCCCGTTACCAAAA---------GCTAAAAATGGGCTTATCATG------TT--- |
| Clon_TF3 | AAATTGCCCGTTACCAAAA---------GCTAAAAATGGGCTTATCATG------TT--- |
| Clon_TF2 | AAATTGCCCGTTACCAAAA---------GCTAAAAATGGGCTTATCATG------TT--- |
| Clon_TF23 | AAATTGCCCGTTACCAAAA---------GCTAAAAATGGGCTTATCATG------TT--- |
| Clon_TF1 | AAATTGCCCGTTACCAAAA---------GCTAAAAATGGGCTTATCATG------TT--- |
| Clon_TF4 | AAATTGCCCGTTACCAAAA---------GCTAAAAATGGGCTTATCATG------TT--- |
| Clon_TF7 | AAATTGCCCGTTACCAAAA---------GCTAAAAATGGGCTTATCATG------TT--- |
| Clon_TF15 | AAATTGCCCGTTACCAAAA---------GCTAAAAATGGGCTTATCATG------TT--- |
| Clon_TF14 | AAATTGCCCGTTACCAAAA---------GCTAAAAATGGGCTTATCATG------TT--- |
| Clon_DP8 | TCGTTGCCCGTTATCACAA----------------ACGGATCGAC-----------T--- |
| Clon_DP1 | TCGTTGCCCGTTATCACAA----------------ACGGATCGAC-----------T--- |
| Clon_DP7 | TCGTTGCCCGTTATCACAA----------------ACGGATCGAC-----------T--- |
| Clon_DP3 | AAGTTGCCCGTTATCACAA----------------ATGGATCGAC-----------T--- |
| Clon_DP6 | AAGTTGCCCGTTATCACAA----------------ATGGATCGAC-----------T--- |
| Clon_DP9 | AAGTTGCCCGTTATCACAA----------------ATGGATCGAC-----------T--- |
| Clon_DP2 | TCGTTGCCCGTTATCACAA----------------ACGGATCGAC-----------T--- |
| Clon_DP4 | TCGTTGCCCGTTATCACAA----------------ACGGATCGAC-----------T--- |
| Clon_DP10 | TCGTTGCCCGTTATCACAA----------------ACGGATCGAC-----------T--- |
| Clon_DP5 | AAGTTGCCCGTTATCACAA----------------ATGGATCGAC-----------T--- |
| Clon_EM4 | CAGTTGCCCGTTATCACAA----------------ATGGAACGAC-----------T--- |
| Clon_EM21 | CAGTTGCCCGTTATCACAA----------------ATGGAACGAC-----------T--- |
| Clon_EM2 | CAGTTGCCCGTTATCACAA----------------ATGGAACGAC-----------T--- |
| Clon_EM23 | CAGTTGCCCGTTATCACAA----------------ATGGAACGAC-----------T--- |
| Clon_EM3 | CAGTTGCCCGTTATCACAA----------------ATGGAACGAC-----------T--- |
| Clon_EM24 | CAGTTGCCCGTTATCACAA----------------ATGGAACGAC-----------T--- |
| Clon_EM22 | CAGTTGCCCGTTATCACAA----------------ATGGAACGAC-----------T--- |
| Clon_EM1 | CAGTTGCCCGTTATCACAA----------------ATGGAACGAC-----------T--- |
| Clon_EM6 | CAGTTGCCCGTTATCACAA----------------ATGGAACGAC-----------T--- |
| Clon_EM5 | CAGTTGCCCGTTATCACAA----------------ATGGAACGAC-----------T--- |
| Clon_GD1 | CAGTTGCCCGTTATCACAA----------------ATGGAACGAC-----------TGGC |
| Clon_GD10 | TAGTTGCCCGTTATCAAAA----------------ATGGGTTGAC-----------TGGC |
| Clon_GD2 | TAGTTGCCCGTTATCAAAA----------------ATGGGTTGAC-----------TGGC |
| Clon_GD5 | TAGTTGCCCGTTATCAAAA----------------ATGGGTTGAC-----------TGGC |
| Clon_GD3 | TAGTTGCCCGTTATCAAAA----------------ATGGGTTGAC-----------TGGC |
| Clon_GD12 | TAGTTGCCCGTTATCAAAA----------------ATGGGTTGAC-----------TGGC |
| Clon_GD7 | TAGTTGCCCGTTATCAAAA----------------ATGGGTTGAC-----------TGGC |
| Clon_GD9 | TAGTTGCCCGTTATCAAAA----------------ATGGGTTGAC-----------TGGC |
| Clon_GD8 | CAGTTGCCCGTTATCAAAA----------------ATGGGTTGAC-----------TGGC |
| Clon_GD13 | CAGTTGCCCGTTATCAAAA----------------ATGGGTTGAC-----------TGGC |
| Clon_LD5 | AAGTTGCCCGTTATCAAAA----------------ATGGGTTGAC-----------TGAT |
| Clon_LD13 | AAGTTGCCCGTTATCAAAA----------------ATGGGTTGAC-----------TGAT |
| Clon_LD14 | AAGTTGCCCGTTATCAAAA----------------ATGGGTTGAC-----------TGAT |
| Clon_LD1 | AAGTTGCCCGTTATCAAAA----------------ATGGGTTGAC-----------TGAT |
| Clon_LD11 | AAGTTGCCCGTTATCAAAA----------------ATGGGTTGAC-----------TGAT |
| Clon_LD3 | AAGTTGCCCGTTATCAAAA----------------ATGGGTTGAC-----------TGAT |
| Clon_LD2 | AAGTTGCCCGTTATCAAAA----------------ATGGGTTGAC-----------TGAT |
| Clon_LD12 | AAGTTGCCCGTTATCAAAA----------------ATGGGTTGAC-----------TGAT |
| Clon_LD8 | AAGTTGCCCGTTATCAAAA----------------ATGGGTTGAC-----------TGAT |
| Clon_LD15 | AAGTTGCCCGTTATCAAAA----------------ATGGGTTGAC-----------TGAT |
| | ** * * * * * |
| Clon_DM1 | -GACTTG----TGTGTTGCG-----------------------------------ATCGG |
| Clon_DM21 | -GACTTG----TGTGTTGCG-----------------------------------ATCGG |
| Clon_DM6 | -GACTTG----TGTGTTGCG-----------------------------------ATCGG |
| Clon_DM20 | -GACTTG----TGTGTTGCG-----------------------------------ATCGG |
| Clon_DM9 | -GACTTG----TGTGTTGCG-----------------------------------ATCGG |
| Clon_DM12 | -GACTTG----TGTGTTGCG-----------------------------------ATCGG |
| Clon_DM7 | -GACTT-----TGTGTTGCG-----------------------------------ATCGG |
| Clon_DM11 | -GACTTG----TGTGTTGCG-----------------------------------ATCGG |
| Clon_DM14 | -GACTTG----TGTGTTGCG-----------------------------------ATCGG |
| Clon_DMA | -GACTTG----TGTGTTGCG-----------------------------------ATCGG |
| Clon_DF1 | -GACTT------GTGTTGCG-----------------------------------ATCGG |
| Clon_DF6 | -GACTT------GTGTTGCG-----------------------------------ATTGG |
| Clon_DF4 | -GACTT------GTGTTGCG-----------------------------------ATCGG |
| Clon_DF26 | -GACTT------GTGTTGCG-----------------------------------ATCGG |
| Clon_DF4_50 | -GACTT------GTGTTGCG-----------------------------------ATTGG |
| Clon_DF19 | -GACTT------GTGTTGCG-----------------------------------ATTGG |
| Clon_DF3 | -GACTT------GTGTTGCG-----------------------------------ATTGG |
| Clon_DF5 | -GACTT------GTGTTGCG-----------------------------------ATCGG |
| Clon_DF2 | -GACTT------GTGTTGCG-----------------------------------ATCGG |
| Clon_DF7 | -GACTT------GTGTTGCG-----------------------------------ATCGG |
| Clon_AS15 | ACATGTC----AGTGTGACCCAGAGAAAGGCTACCAACCCTT-----------TACTCGG |
| Clon_AS14 | ACATGTC----AGTGTGACCCAGAGAAAGGCTACCAACCCTT-----------TACTCGG |
| Clon_AS20 | ACATGTC----AGTGTGACCCAGAGAAAGGCTACCAACCCTT-----------TACTCGG |
| Clon_AS13 | ACATGTC----AGTGTGACCCAGAGAAAGGCTACCAACCCTT-----------TACTCGG |
| Clon_AS10 | ACATGTC----AGTGTGACCCAGAGAAAGGCTACCATCCCTT-----------TACTCGG |
| Clon_AS11 | ACATGTC----AGTGTGACCCAGAGAAAGGCTACCA---CCT-----------TACTCGG |
| Clon_AS2 | ACATGTC----AGTGTGACCCAGAGAAAGGCTACCA---CCT-----------TACTCGG |
| Clon_AS12 | ACATGTC----AGTGTGACCCAGAGAAAGGCTACCAACCCCT-----------TACTCGG |
| Clon_AS1 | ACATGTC----AGTGTGACCCAGAGAAAGGCTACCA-CCCCT-----------TACTCGG |
| Clon_AS16 | ACATGTC----AGTGTGACCCAGAGAAAGGCTACCAACCCCT-----------TACTCGG |
| Clon_BT8 | ---CGTT----AGT-TGATC----------------------------------ATTGAG |
| Clon_BT9 | ---CGTT----AGT-TGATC----------------------------------ATTGAG |
| Clon_BT16 | ---CGTT----TGT-TGATC----------------------------------ATTGAG |
| Clon_BT3 | ---CGTT----AGT-TGATC----------------------------------ATTGAG |
| Clon_BT14 | ---TGCT----AGT-TGATC----------------------------------ATTGAG |
| Clon_BT17 | ---TGCT----AGT-TGATC----------------------------------ATTGAG |
| Clon_BT13 | ---CGTT----AGT-TGATC----------------------------------ATTGAG |
| Clon_BT1 | ---CGTT----AGT-TGATC----------------------------------ATTGAG |
| Clon_BT10 | ---TGCT----AGT-TGATC----------------------------------ATTGAG |
| Clon_BT15 | ---TGCT----AGT-TGATC----------------------------------ATTGGG |
| Clon_TPA1_20 | AGGC--G----AGT-TGTGTCC-------------------------------GTGTGTG |
| Clon_TPA1_22 | AGGC--G----AGT-TGTGTCC-------------------------------GTGTGTG |
| Clon_TPA1_29 | AGGC--G----AGT-TGTGTCC-------------------------------GTGTGTG |
| Clon_TPA1_28 | AGGC--G----AGT-TGTGTCC-------------------------------GTGTGTG |
| Clon_TPA1_26 | AGGCC-G----AGT-TGTGTCC-------------------------------GTGTGTG |
| Clon_TPA1_21 | AGGCC-G----AGT-TGTGTCC-------------------------------GTGTGTG |
| Clon_TPA1_36 | AGGCC-G----AGT-TGTGTCC-------------------------------GTGTGTG |
| Clon_TPA1_27 | AGGC--G----AGA-TGTGTCC-------------------------------GTGTGTG |
| Clon_TPA1_23 | AGGC--G----AGT-TGTGTCC-------------------------------GTGTGTG |
| Clon_TPA1_1 | AGGC--G----AGT-TATGTCC-------------------------------GTGTGTG |
| Clon_TF22 | AGGCGAG----AGT---TGT-----------------------------------GCGTG |
| Clon_TF24 | AGGCGAG----AGT---TGT-----------------------------------GCGTG |
| Clon_TF3 | AGGCGAG----AGT---TGT-----------------------------------GCGTG |
| Clon_TF2 | AGGCGAG----AGT---TGT-----------------------------------GCGTG |
| Clon_TF23 | AGGCGAG----AGT---TGT-----------------------------------GCGTG |
| Clon_TF1 | AGGCGAG----AGT---TGT-----------------------------------GCGTG |
| Clon_TF4 | AGGCGAG----AGT---TGT-----------------------------------GCGTG |
| Clon_TF7 | AGGCGAG----AGT---TGT-----------------------------------GCGTG |
| Clon_TF15 | AGGCGAG----AGT---TGT-----------------------------------GCGTG |
| Clon_TF14 | AGGCGAG----AGT---TGT-----------------------------------GCGTG |
| Clon_DP8 | -GGCAAT----TGT----------------------------------------TGACAG |
| Clon_DP1 | -GGCAAT----TGT----------------------------------------TGACAG |
| Clon_DP7 | -GGCAAT----TGT----------------------------------------TGACAT |
| Clon_DP3 | -GGCAAT----TGT----------------------------------------TGACAC |
| Clon_DP6 | -GGCAAT----TGT----------------------------------------TGACAC |
| Clon_DP9 | -GGCAAT----TGT----------------------------------------TGACAC |
| Clon_DP2 | -GGCAAT----TGT----------------------------------------TGACAT |
| Clon_DP4 | -GGCAAT----TGT----------------------------------------TGACAT |
| Clon_DP10 | -GGCAAT----TGT----------------------------------------TGACAC |
| Clon_DP5 | -GGCAAT----TGT----------------------------------------TGACAT |
| Clon_EM4 | -GGCAAT----TGT----------------------------------------TGTGAT |
| Clon_EM21 | -GGCAAT----TGT----------------------------------------TGTGAT |
| Clon_EM2 | -GGCAAT----TGT----------------------------------------TGTGAT |
| Clon_EM23 | -GGCAAT----TGT----------------------------------------TGTGAT |
| Clon_EM3 | -GGCAAT----TGT----------------------------------------TGTGAT |
| Clon_EM24 | -GGCAAT----TGT----------------------------------------TGTGAT |
| Clon_EM22 | -GGCAAT----TGT----------------------------------------TGTGAT |
| Clon_EM1 | -GGCAAT----TGT----------------------------------------TGTGAT |
| Clon_EM6 | -GGCAAT----TGT----------------------------------------TGTGAT |
| Clon_EM5 | -GGCAAT----TGT----------------------------------------TGTGAT |
| Clon_GD1 | GAGCGAT-TATGAT---------------TGT-A--------------------ATCTAA |
| Clon_GD10 | GAGCGAT-TATGAT---------------TGT-A--------------------ATCTAA |
| Clon_GD2 | GAGCGAT-C--GAT---------------TATGA--------------------ATCGAG |
| Clon_GD5 | GAGCGAT-C--GAT---------------TATGA--------------------ATCGAG |
| Clon_GD3 | GAGCGAT-C--GAT---------------TATGA--------------------ATCGAG |
| Clon_GD12 | GAGCGAT-C--GAT---------------TATGA--------------------ATCGAG |
| Clon_GD7 | GAGCGAT-C--GAT---------------TATGA--------------------ATCGAG |
| Clon_GD9 | GAGCGAT-C--GAT---------------TATGA--------------------ATCGAG |
| Clon_GD8 | GAGCGAT-C--GAT---------------TATGA--------------------ATCGAG |
| Clon_GD13 | GAGCGAT-C--GAT---------------TATGA--------------------ATCGAG |
| Clon_LD5 | GAACGATGTGCGATGTAACCGCGTGCATGCATGGT-------------------ATCGAG |
| Clon_LD13 | GAACGATGTGCGATGTAACCGCGTGCATGCATGGT-------------------ATCGAG |
| Clon_LD14 | GAACGATGTGCGATGTAACCGCGTGCATGCATGGT-------------------ATCGAG |
| Clon_LD1 | GAACGATGTGCGATGTAACCGCGTGCATGCATGGT-------------------ATCGAG |
| Clon_LD11 | GAACGATGTGCGATGTAACCGCGTGCATGCATGGT-------------------ATCGAG |
| Clon_LD3 | GAACGATGTGCGATGTAACCGCGTGCATGCATGGT-------------------ATCGAG |
| Clon_LD2 | GAACGATGTGCGATGTAACCGCGTGCATGCATGGT-------------------ATCGAG |
| Clon_LD12 | GAACGATGTGCGATGTAACCGCATGCATGCATGGT-------------------ATCGAG |
| Clon_LD8 | GAACGATGTGCGATGTAACCGCGTGCATGCATGGT-------------------ATCGAG |
| Clon_LD15 | GAACGATGTGCGATGTAACCGCGTGCATGCATGGT-------------------ATCGAG |
| Clon_DM1 | TGGTGT-CA-------------CATTGAAAGTTCTGAAGT-------TAA---------- |
| Clon_DM21 | TGGTGT-CA---------------TTGAAAGTTCTGAAGTG-----TTAA---------- |
| Clon_DM6 | TGGTGT-CA---------------TTGAAAGTTCTGAAGT-------TAA---------- |
| Clon_DM20 | TGGTGT-CA---------------TTGAAAGTTCTGAAGT-------TAA---------- |
| Clon_DM9 | TGGTGT-CA-------------CATTGAAAGTTCTGAAGT-------TAA---------- |
| Clon_DM12 | TGGTGT-CA-------------CATTGAAAGTTCTGAAGT-------TAA---------- |
| Clon_DM7 | TGGTGT-CA---------------TTGAAAGTTCTGAAGT-------TGAT--------- |
| Clon_DM11 | TGGTGT-CA-------------CATTGAAAGTTCTGAAGT-------TAA---------- |
| Clon_DM14 | TGGTGT-CA---------------TTGAAAGTTCTGAAGTG-----TTAA---------- |
| Clon_DMA | TGGTGT-CA---------------TTGAAAGTTCTGAAGTA-----TTAA---------- |
| Clon_DF1 | TGGTGT-CA---------------TTGAAAGTTCTGAAGT-------TGAA--------- |
| Clon_DF6 | TGGTGT-CA---------------TTGAAAGTTCTGAAGT-------TGAA--------- |
| Clon_DF4 | TGGTGT-CA---------------TTGAAAGTTCTGAAGT-------TGAA--------- |
| Clon_DF26 | TGGTGT-CA---------------TTGAAAGTTCTGAAGT-------TGAA--------- |
| Clon_DF4_50 | TGGTGT-CA---------------TTGAAAGTTCTGAAGT-------TGAA--------- |
| Clon_DF19 | TGGTGT-CA---------------TTGAAAGTTCTGAAGT-------TGAA--------- |
| Clon_DF3 | TGGTGT-CA---------------TTGAAAGTTCTGAAGT-------TGAA--------- |
| Clon_DF5 | TGGTGT-CA---------------TTGAAAGTTCTGAAGT-------TGAA--------- |
| Clon_DF2 | TGGTGT-CA---------------TTGAAAGTTCTGAAGT-------TGAA--------- |
| Clon_DF7 | TGGTGT-CA---------------TTGAAAGTTCTGAAGT-------TGAA--------- |
| Clon_AS15 | TACAAC----------------TTTTGAACGCTTGCCTGT------TTAACT-TGTCTGT |
| Clon_AS14 | TACAAC----------------TTTTGAACGCTTGCCTGT------TTAACT-TGTCTGT |
| Clon_AS20 | TACAAC----------------TTTTGAACGCTTGCCTGT------TTAACT-TGTCTGT |
| Clon_AS13 | TACAAC----------------TTTTGAACGCTTGCCTGT------TTAACT-TGTCTGT |
| Clon_AS10 | TACAAC----------------TTTTGAACGCTTGCCTGT------TTAACT-TGTCTGT |
| Clon_AS11 | TACAACATC-------------CTTTGAACGCTTGCCTGT------TTAACT-TGTCTGT |
| Clon_AS2 | TACAACATC-------------CTTTGAACGCTTGCCTGT------TTAACT-TGTCTGT |
| Clon_AS12 | TACAACATC-------------CTTTGAACGCTTGCCTGT------TTAACT-TGTCTGT |
| Clon_AS1 | TACAACATC-------------CTTTGAACGCTTGCCTGT------TTAACT-TGTCTGT |
| Clon_AS16 | TACAACATC-------------CTTTGAACGCTTGCCTGT------TTAACT-TGTCTGT |
| Clon_BT8 | TAGCAT----------------TCCTG--TGATTGTC-GT------TTGA---------- |
| Clon_BT9 | TAGCAT----------------TCCTG--TGATTGTC-GT------TTGA---------- |
| Clon_BT16 | TAGCAT----------------TCCTG--TGATTGTC-TT------TTGA---------- |
| Clon_BT3 | TAGCAT----------------TCCTG--TGATTGTC-GT------TTGA---------- |
| Clon_BT14 | TAGCAT----------------TCCTG--TGATTGTC-GT------TTGA---------- |
| Clon_BT17 | TAGCAT----------------TCCTG--TGATTGTC-GT------TTGA---------- |
| Clon_BT13 | TAGCAT----------------TCCTG--TGATTGTC-TT------TTGA---------- |
| Clon_BT1 | TAGCAT----------------TCCTG--TGATTGTC-AT------TTGA---------- |
| Clon_BT10 | TAGCAT----------------TCCTG--TGATTGTC-TT------TTGA---------- |
| Clon_BT15 | TAGCAT----------------TCCTG--TGATTGTC-TT------TTGA---------- |
| Clon_TPA1_20 | TGGTACACC-------------CGTTGAACGCTTGAG-AC-------TAA----GC--AG |
| Clon_TPA1_22 | TGGTACACC-------------CGTTGAACGCTTGAG-AC-------TAA----GC--AG |
| Clon_TPA1_29 | TGGTACACC-------------CGTTGAACGCTTGAG-AC-------TAA----GC--AG |
| Clon_TPA1_28 | TGGTACACC-------------CGTTGAACGCTTGAG-AC-------TAA----GC--AG |
| Clon_TPA1_26 | TGGTACACC-------------CGTTGAACGCTTGAG-GC-------TAA----GC--AG |
| Clon_TPA1_21 | TGGTACACC-------------CGTTGAACGCTTGAG-GC-------TAA----GC--AG |
| Clon_TPA1_36 | TGGTACACC-------------CGTTGAACGCTTGAG-GC-------TAA----GC--AG |
| Clon_TPA1_27 | TGGTACACC-------------CGTTGAACGCTTGAG-AC-------TAA----GC--AG |
| Clon_TPA1_23 | TGGTACACC-------------CGTTGAACGCTTGAG-GC-------TAA----GC--AG |
| Clon_TPA1_1 | TGGTACACC-------------CGTTGAACGCTTGAG-GC-------TAA----GC--AG |
| Clon_TF22 | TGGTACACC-------------CGTTGAACGCTTGAG-GC-------TAA----GC--AG |
| Clon_TF24 | TGGTACACC-------------CGTTGAACGCTTGAG-GC-------TAA----GC--AG |
| Clon_TF3 | TGGTACACC-------------CGTTGAACGCTTGAG-GC-------TAA----GC--AG |
| Clon_TF2 | TGGTACACC-------------CGTTGAACGCTTGAG-GC-------TAA----GC--AG |
| Clon_TF23 | TGGTACACC-------------CGTTGAACGCTTGAG-GC-------TAA----GC--AG |
| Clon_TF1 | TGGTACACC-------------CGTTGAACGCTTGAG-GC-------TAA----GC--AG |
| Clon_TF4 | TGGTACACC-------------CGTTGAACGCTTGAG-GC-------TAA----GC--AG |
| Clon_TF7 | TGGTACACC-------------CGTTGAACGCTTGAG-GC-------TAA----GC--AG |
| Clon_TF15 | TGGTACACC-------------CGTTGAACGCTTGAG-GC-------TAA----GC--AG |
| Clon_TF14 | TGGTACACC-------------CGTTGAACGCTTGAG-GC-------TAA----GC--AG |
| Clon_DP8 | TGATGAGGT-------------TATTGAAAGCTC-------------TGA---------- |
| Clon_DP1 | TGATGAGGT-------------TATTGAAAGCTC-------------TGA---------- |
| Clon_DP7 | TGATGAGGT-------------TATTGAAAGCTC-------------TGA---------- |
| Clon_DP3 | TGATGAGGT-------------TATTGAAAGCTC-------------TGA---------- |
| Clon_DP6 | TGATGAGGT-------------TATTGAAAGCTC-------------TGA---------- |
| Clon_DP9 | TGATGAGGT-------------TATTGAAAGCTC-------------TGA---------- |
| Clon_DP2 | TGATGAGGT-------------TATTGAAAGCTC-------------TGA---------- |
| Clon_DP4 | TGATGAGGT-------------TATTGAAAGCTC-------------TGA---------- |
| Clon_DP10 | TGATGAGGT-------------TATTGAAAGCTC-------------TGA---------- |
| Clon_DP5 | TGATGAGGT-------------TATTGAAAGCTC-------------TGA---------- |
| Clon_EM4 | TGAGGTTGT-------------CATTGAAAGCTC-------------TGA---------- |
| Clon_EM21 | TGAGGTTGT-------------CATTGAAAGCTC-------------TGA---------- |
| Clon_EM2 | TGAGGTTGT-------------CATTGAAAGCTC-------------TGA---------- |
| Clon_EM23 | TGAGGTTGT-------------CATTGAAAGCTC-------------TGA---------- |
| Clon_EM3 | TGAGGTTGT-------------CATTGAAAGCTC-------------TGA---------- |
| Clon_EM24 | TGAGGTTGT-------------CATTGAAAGCTC-------------TGA---------- |
| Clon_EM22 | TGAGGTTGT-------------CATTGAAAGCTC-------------TGA---------- |
| Clon_EM1 | TGAGGTTGT-------------CATTGAAAGCTC-------------TGA---------- |
| Clon_EM6 | TGAGGTTGT-------------CATTGAAAGCTC-------------TGA---------- |
| Clon_EM5 | TGAGGTTGT-------------CATTGAAAGCTC-------------TGA---------- |
| Clon_GD1 | GGTGTAAAA-----------TGCATCGAAAGCTTAATGTT------GCAATTGTGCATAA |
| Clon_GD10 | GGTGTAAAA-----------TGCATCGAAAGCTTAATGTT------GCAATTGTGCATAA |
| Clon_GD2 | TGTTGTGGA-----------TGCATCGAAAGCTTAATGTT------GCAATTGTGCATAC |
| Clon_GD5 | TGTTGTGGA-----------TGCATCGAAAGCTTAATGTT------GCAATTGTGCATAC |
| Clon_GD3 | TGTTGTGGA-----------TGCATCGAAAGCTTAATGTT------GCAATTGTGCATAC |
| Clon_GD12 | TGTTGTGGA-----------TGCATCGAAAGCTTAATGTT------GCAATTGTGCATAC |
| Clon_GD7 | TGTTGTGGA-----------TGCATCGAAAGCTTAATGTT------GCAATTGTGCATAC |
| Clon_GD9 | TGTTGTGGA-----------TGCATCGAAAGCTTAATGTT------GCAATTGTGCATAC |
| Clon_GD8 | TGTTGTGGA-----------TGCATCGAAAGCTTAATGTT------GCAATTGTGCATAC |
| Clon_GD13 | TGTTGTGGA-----------TGCATCGAAAGCTTAATGTT------GCAATTGTGCATAC |
| Clon_LD5 | AAATGTGAAAAGATGTGCAATGTATCGAAAGCTTGATGTT------TTGCTTGCTTG-AT |
| Clon_LD13 | AAATGTGAAAAGATGTGCAATGTATCGAAAGCTTGATGTT------TTGCTTGCTTG-AT |
| Clon_LD14 | AAATGTGAAAAGATGTGCAATGTATCGAAAGCTTGATGTT------TTGCTTGCTTG-AT |
| Clon_LD1 | AAATGTGAAAAGATGTGCAATGTATCGAAAGCTTGATGTT------TTGCTTGCTTG-AT |
| Clon_LD11 | AAATGTGAAAAGATGTGCAATGTATCGAAAGCTTGATGTT------TTGCTTGCTTG-AT |
| Clon_LD3 | AAATGTGAAAAGATGTGCAATGTATCGAAAGCTTGATGTT------TTGCTTGCTTG-AT |
| Clon_LD2 | AAATGTGAAAAGATGTGCAATGTATCGAAAGCTTGATGTT------TTGCTTGCTTG-AT |
| Clon_LD12 | AAATGTGAAAAGATGTGCAATGTATCGAAAGCTTGATGTT------TTGCTTGCTTG-AT |
| Clon_LD8 | AAATGTGAAAAGATGTGCAATGTATCGAAAGCTTGATGTT------TTGCTTGCTTG-AT |
| Clon_LD15 | AAATGTGAAAAGATGTGCAATGTATCGAAAGCTTGATGTT------TTGCTTGCTTG-AT |
| | * * * |
| Clon_DM1 | ----ACTTG-------------------GCTT-------------TATA----------T |
| Clon_DM21 | ----ACTTG-------------------GCTT-------------TATA----------T |
| Clon_DM6 | ----ACTTG-------------------GCTT-------------TATA----------T |
| Clon_DM20 | ----ACTTG-------------------GCTT-------------TATA----------T |
| Clon_DM9 | ----ACTTG-------------------GCTT-------------TATA----------T |
| Clon_DM12 | ----ACTTG-------------------GCTT-------------TATA----------T |
| Clon_DM7 | ----ACTTG-------------------GCTT-------------TATA----------T |
| Clon_DM11 | ----ACTTG-------------------GCTT-------------TATA----------T |
| Clon_DM14 | ----ACTTG-------------------GCTT-------------TATA----------T |
| Clon_DMA | ----ACTTG-------------------GCTT-------------TATA----------T |
| Clon_DF1 | ----ACTTG-------------------GCTT-------------TT------------T |
| Clon_DF6 | ----ACTTG-------------------GCTT-------------TA------------T |
| Clon_DF4 | ----ACTTG-------------------GCTT-------------TA------------T |
| Clon_DF26 | ----ACTTG-------------------GCTT-------------TA------------T |
| Clon_DF4_50 | ----ACTTG-------------------GCTT-------------TA------------T |
| Clon_DF19 | ----ACTTG-------------------GCTT-------------TA------------T |
| Clon_DF3 | ----ACTTG-------------------GCTT-------------TA------------T |
| Clon_DF5 | ----ACTTG-------------------GCTT-------------TA------------T |
| Clon_DF2 | ----ACTTG-------------------GCTT-------------TA------------T |
| Clon_DF7 | ----ACTTG-------------------GCTT-------------TA------------T |
| Clon_AS15 | GCACGCTT---TGCATACTGTCTCTACCACGCCAAATAAACCT-TTTAGG---------T |
| Clon_AS14 | GCACGCTT---TGCATACTGTCTCTACCACGCCAAATAAACCT-TTTAGG---------T |
| Clon_AS20 | GCACGCTT---TGCATACTGTCTCTACCACGCCAAATAAACCT-TTTAGG---------T |
| Clon_AS13 | GCACGCTT---TGCATACTGTCTCTACCACGCCAAATAAACCT-TTTAGG---------T |
| Clon_AS10 | GCACGCTT---TGCATACTGTCTCTACCACGCCAAATAAACCT-TTTAGG---------T |
| Clon_AS11 | GCACGCTT---TGCATACTGTCTCTACCACGCCAAATAAACCT-TTTAGG---------T |
| Clon_AS2 | GCACGCTT---TGCATACTGTCTCTACCACGCCAAATAAACCT-TTTAGG---------T |
| Clon_AS12 | GCACGCTT---TGCATACTGTCTCTACCACGCCAAATAAACCT-TTTAGG---------T |
| Clon_AS1 | GCACGCTT---TGCATACTGTCTCTACCACGCCAAATAAACCCGTTTAGG---------T |
| Clon_AS16 | GCACGCTT---TGCATACTGTCTCTACCACGCCAAATAAACCT-TTTAGG---------T |
| Clon_BT8 | --ACGCTT---TGCA-------------ACATTA----------TTTTGA---------T |
| Clon_BT9 | --ACGCTT---TGCA-------------ACATTA----------TTTTGA---------T |
| Clon_BT16 | --ACGCTT---TGCA-------------ACATTA----------TTTTGA---------T |
| Clon_BT3 | --ACGCTT---TGCA-------------ACATTA----------TTTTGA---------T |
| Clon_BT14 | --ACGCTT---TGCA-------------ACATTA----------TTTTGA---------T |
| Clon_BT17 | --ACGCTT---TGCA-------------ACATTA----------TTTTGA---------T |
| Clon_BT13 | --ACGCTT---TGCA-------------ACATTA----------TTTTGA---------T |
| Clon_BT1 | --ACGCTT---TGCA-------------ACATTA----------TTTTGA---------T |
| Clon_BT10 | --ACGTTA---TGCA-------------ACATT-----------TATTGG---------T |
| Clon_BT15 | --ACGCTA---TGCA-------------ACATT-----------TATTGG---------T |
| Clon_TPA1_20 | A---GTT----TGCAA-GAGGCTG----GCGCCAG-----------CAAC---------A |
| Clon_TPA1_22 | A---GTT----TGCAA-GAGGCTG----GCGCCAG-----------CAAC---------A |
| Clon_TPA1_29 | A---GTT----TGCAA-GAGGCTG----GCGCCAG-----------CAGC---------A |
| Clon_TPA1_28 | A---GTT----TGCAA-GAGGCTG----GCGCCAG-----------CAAC---------A |
| Clon_TPA1_26 | A---GTT----TGCAA-GAGGCTG----GCGCCAG-----------CAGC---------A |
| Clon_TPA1_21 | A---GTT----TGCAA-GAGGCTG----GCGC--------------CAGC---------A |
| Clon_TPA1_36 | A---GTT----TGCAA-GAGGCTG----GCGCTAG-----------CAGC---------A |
| Clon_TPA1_27 | A---GTT----TGCAA-GAGGCTG----GCGCCAG-----------CAGC---------A |
| Clon_TPA1_23 | A---GTT----TGCAA-GAGGCTG----GCGC--------------CAGC---------A |
| Clon_TPA1_1 | A---GTT----TGCAA-GAGGCTG----GCGC--------------CAGC---------A |
| Clon_TF22 | A---GTT----TACAA-GAGGCTG----GCGCCAA-----------TAGC---------A |
| Clon_TF24 | A---GTT----TACAA-GAGGCTG----GCGCCAA-----------TAGC---------A |
| Clon_TF3 | A---GTT----TACAA-GAGGCTG----GCGCCAA-----------TAGC---------A |
| Clon_TF2 | A---GTT----TGCAA-GAGGCTG----GCGCCAA-----------TAGC---------A |
| Clon_TF23 | A---GTT----TGCAA-GAGGCTG----GCGCCAA-----------TAGC---------A |
| Clon_TF1 | A---GTT----TGCAA-GAGGCTG----GCGCCAA-----------TAGC---------A |
| Clon_TF4 | A---GTT----TGCAA-GAGGCTG----GCGCCAA-----------TAGC---------A |
| Clon_TF7 | A---GTT----TGCAA-GAGGCTG----GCGCCAA-----------TAGC---------A |
| Clon_TF15 | A---GTT----TGCAA-GAGGCTG----GCGCCAA-----------TAGC---------A |
| Clon_TF14 | A---GTT----TGCAA-GAGGCTG----GCGCCAA-----------TAGC---------A |
| Clon_DP8 | ---AATT---------------------GTATC-------------CTGG---------T |
| Clon_DP1 | ---AATT---------------------GTATC-------------CTGG---------T |
| Clon_DP7 | ---AATT---------------------GTATC-------------CTGG---------T |
| Clon_DP3 | ---AATT---------------------GTATC-------------CTGG---------T |
| Clon_DP6 | ---AATT---------------------GTATC-------------CTGG---------T |
| Clon_DP9 | ---AATT---------------------GTATC-------------CTGG---------T |
| Clon_DP2 | ---AATT---------------------GTATC-------------CTGG---------T |
| Clon_DP4 | ---AATT---------------------GTATC-------------CTGG---------T |
| Clon_DP10 | ---AATT---------------------GTATC-------------CTGG---------T |
| Clon_DP5 | ---AATT---------------------GTATC-------------CTGG---------T |
| Clon_EM4 | ---AGTT---------------------TTGTC-------------CTGG---------T |
| Clon_EM21 | ---AGTT---------------------TTGTC-------------CTGG---------T |
| Clon_EM2 | ---AGTT---------------------GTATC-------------CTGG---------T |
| Clon_EM23 | ---AGTT---------------------GTATC-------------CCGG---------T |
| Clon_EM3 | ---AGTT---------------------GTATC-------------CTGG---------T |
| Clon_EM24 | ---AGTT---------------------GTATC-------------CTGG---------T |
| Clon_EM22 | ---AGTT---------------------GTATC-------------CTGG---------T |
| Clon_EM1 | ---AGTT---------------------TTGTC-------------CTGG---------T |
| Clon_EM6 | ---AGTT---------------------GTATC-------------CTGG---------T |
| Clon_EM5 | ---AGTT---------------------GTATC-------------CTGG---------T |
| Clon_GD1 | TG-TGTTGAAG--CAGGTTAAATG---AACGTTTAT---------ATGA----------T |
| Clon_GD10 | TG-TGTTGAAG--CAGGTTAAATG---AACGTTTAT---------ATGA----------T |
| Clon_GD2 | CG-TGTTGAAG--CAGGTTAAATG---AACGTTTAT---------ATGA----------T |
| Clon_GD5 | CG-TGTTGAAG--CAGGTTAAATG---AACGTTTAT---------ATGA----------T |
| Clon_GD3 | CG-TGTTGAAG--CAGGTTAAATG---AACGTTTAT---------ATGA----------T |
| Clon_GD12 | CG-TGTTGAAG--CAGGTTAAATG---AACGTTTAT---------ATGA----------T |
| Clon_GD7 | CG-TGTTGAAG--CAGGTTAAATG---AACGTTTAT---------ATGA----------T |
| Clon_GD9 | CG-TGTTGAAG--CAGGTTAAATG---AACGTTTAT---------ATGA----------T |
| Clon_GD8 | CG-TGTTGAAG--CAGGTTAAATG---AACGTTTAT---------ATGA----------T |
| Clon_GD13 | CG-TGTTGAAG--CAGGTTAAATG---AACGTTTAT---------ATGA----------T |
| Clon_LD5 | TGCTGATGATGTTCGAATCAATTGC-TAGTGTTTGTGCAGA----ATAAACAAGACATTT |
| Clon_LD13 | TGCTGATGATGTTCGAATCAATTGC-TAGTGTTTGTGCAGA----ATAAACAAGACATTT |
| Clon_LD14 | TGCTGATGATGTTCGAATCAATTGC-TAGTGTTTGTGCAGA----ATAAACAAGACATTT |
| Clon_LD1 | TGCTGATGATGTTCGAATCAATTGC-TAGTGTTTGTGCAGA----ATAAACAAGACATTT |
| Clon_LD11 | TGCTGATGATGTTCGAATCAATTGC-TAGTGTTTGTGCAGA----ATAAACAAGACATTT |
| Clon_LD3 | TGCTGATGATGTTCGAATCAATTGC-TAGTGTTTGTGCAGA----ATAAACAAGACATTT |
| Clon_LD2 | TGCTGATGATGTTCGAATCAATTGC-TAGTGTTTGTGCAGA----ATAAACAAGACATTT |
| Clon_LD12 | TGCTGATGATGTTCGAATCAATTGC-TAGTGTTTGTGCAGA----ATAAACAAGACATTT |
| Clon_LD8 | TGCTGATGATGTTCGAATCAATTGC-TAGTGTTTGTGCAGA----ATAAACAAGACATTT |
| Clon_LD15 | TGCTGATGATGTTCGAATCAATTGC-TAGTGTTTGTGCAGA----ATAAACAAGACATTT |
| Clon_DM1 | T-----ATA-TCTGGGTAA-CAT------GGAAAAAAGCA-------------------- |
| Clon_DM21 | T-----ATA-TCTGGGTAA-CAT------GGAAAAAAGCA-------------------- |
| Clon_DM6 | T-----ATA-TCTGGGTAA-CAT------GGAAAAAAGCA-------------------- |
| Clon_DM20 | T-----ATA-TCTGGGTAA-CAT------GGAAAAAAGCA-------------------- |
| Clon_DM9 | T-----ATA-TCTGGGTAA-CAT------GGAAAAAAGCA-------------------- |
| Clon_DM12 | T-----ATA-TCTGGGTAA-CAT------GGAAAAAAGCA-------------------- |
| Clon_DM7 | T-----A---TCTGGGTAA-CAT------GGAAAAAAGCA-------------------- |
| Clon_DM11 | T-----ATA-TCTGGGTAA-CAT------GGAAAAAAGCA-------------------- |
| Clon_DM14 | T-----ATA-TCTGGGTAA-CAT------GGAAAAAAGCA-------------------- |
| Clon_DMA | T-----A---TCTGGGTAA-CAT------GGAAAAAAGCA-------------------- |
| Clon_DF1 | T-----ATA-TCTGGGTAA-CAT------GGAAAAAAGCA-------------------- |
| Clon_DF6 | T-----ATA-TCTGGGTAA-CAT------GGAAAAAAGCA-------------------- |
| Clon_DF4 | T-----ATA-TCTGGGTAA-CAT------GGAAAAAAGCA-------------------- |
| Clon_DF26 | T-----ATA-TCTGGGTAA-CAT------GGAAAAAAGCA-------------------- |
| Clon_DF4_50 | T-----ATA-TCTGGGTAA-CAT------GGAAAAAAGCA-------------------- |
| Clon_DF19 | T-----ATA-TCTGGGTAA-CAT------GGAAAAAAGCA-------------------- |
| Clon_DF3 | T-----ATA-TCTGGGTAA-CAT------GGAAAAAAGCA-------------------- |
| Clon_DF5 | T-----ATA-TCTGGGTAA-CAT------GGAAAAAAGCA-------------------- |
| Clon_DF2 | T-----ATA-TCTGGGTAA-CAT------GGAAAAAAGCA-------------------- |
| Clon_DF7 | T-----ATA-TCTGGGTAA-CAT------GGAAAAAAGCA-------------------- |
| Clon_AS15 | TGATTCATT-T-TGGGCAATCATAT---GAAATG-AATCATTTCCTAAAA--------AG |
| Clon_AS14 | TGATTCATT-T-TGGGCAATCATAT---GAAATG-AATCATTTCCTAAAA--------AG |
| Clon_AS20 | TGATTCATT-T-TGGGCAATCATAT---GAAATG-AATCATTTCCTAAAA--------AG |
| Clon_AS13 | TGATTCATT-T-TGGGCAATCATAT---GAAATG-AATCATTTCCTAAAA--------AG |
| Clon_AS10 | TGATTCATT-T-TGGGCAATCATAT---GAAATG-AATCATTTCCTAAAA--------AG |
| Clon_AS11 | TGATTCATT-T-TGGGCAATCATAT---GAAATG-AATCATTTCCTAAAA--------AG |
| Clon_AS2 | TGATTCATT-T-TGGGCAATCATAT---GAAATG-AATCATTTCCTAAAA--------AG |
| Clon_AS12 | TGATTCATT-T-TGGGCAATCATAT---GAAATG-AATCATTTCCTAAAA--------AG |
| Clon_AS1 | TGATTCATT-T-TGGGCAATCATAT---GAAATG-AATCATTTCCTAAAA--------AG |
| Clon_AS16 | TGATTCATT-T-TGGGCAATCATAT---GAAATG-AATCATTTCCTAAAA--------AG |
| Clon_BT8 | T--TT-GTG-TGTGGGGGATTT----------TGCACACA-------------------- |
| Clon_BT9 | T--TT-GTG-TGTGGGGGATTT----------TGCACACA-------------------- |
| Clon_BT16 | T--TT-GTG-TGTGGGGGATTT----------TGCACACA-------------------- |
| Clon_BT3 | T--TT-GTG-TGTGGGGGATTT----------TGCACACA-------------------- |
| Clon_BT14 | T--TT-GTG-TGTGGGGGATTT----------TGCACACA-------------------- |
| Clon_BT17 | T--TT-GTG-TGTGGGGGATTT----------TGCACACA-------------------- |
| Clon_BT13 | T--TT-GTG-TGTGGGGGATTT----------TGCACACA-------------------- |
| Clon_BT1 | T--TT-GTG-TGTGGGGGATTT----------TGCACACA-------------------- |
| Clon_BT10 | T--TT-GTG-TGTGGGGGATTT----------TGCACACAA------------------- |
| Clon_BT15 | T--TT-GTG-TGTGGGGGATTT----------TGCACACA-------------------- |
| Clon_TPA1_20 | GCAC-CATT-CATGGGCAATCAT-T---GGAATGTGTGCG-------------------- |
| Clon_TPA1_22 | GCAC-CATT-CATGGGCAATCAT-T---GGAATGTGTGCG-------------------- |
| Clon_TPA1_29 | GCAC-CATT-CATGGGCAATCAT-T---GGAATGTGTGCG-------------------- |
| Clon_TPA1_28 | GCAC-CATT-CATGGGCAATCAT-T---GGAATGTGTGCG-------------------- |
| Clon_TPA1_26 | GCAC-CATT-CATGGGCAATCAT-T---GGAATGTGTGCG-------------------- |
| Clon_TPA1_21 | GCAC-CATT-CATGGGCAATCAA-T---GGAATGTGTGCT-------------------- |
| Clon_TPA1_36 | GCAC-CATT-CATGGGCAATCAT-T---GGAATGTGTGCG-------------------- |
| Clon_TPA1_27 | GCAC-CATT-CATGGGCAATCAT-T---GGAATGTGTGCG-------------------- |
| Clon_TPA1_23 | GCAC-CATT-CATGGGCAATCAT-T---GGAATGTGTGCG-------------------- |
| Clon_TPA1_1 | GCAC-CATT-CATGGGCAATCAT-T---GGAATGTGTGCG-------------------- |
| Clon_TF22 | TCAC-CATT-TGTGGGCAATCAT-T---GGAATGTGTGCA-------------------- |
| Clon_TF24 | TCAC-CATT-TGTGGGCAATCAT-T---GGAATGTGTGCA-------------------- |
| Clon_TF3 | TCAC-CATT-TGTGGGCAATCAT-T---GGAATGTGTGCA-------------------- |
| Clon_TF2 | TCAC-CATT-CATGGGCAATCAT-T---GGAATGTGTGCA-------------------- |
| Clon_TF23 | TCAC-CATT-CATGGGCAATCAT-T---GGAATGTGTGCA-------------------- |
| Clon_TF1 | TCAC-CATT-CATGGGCAATCAT-T---GGAATGTGTGCA-------------------- |
| Clon_TF4 | TCAC-CATT-CATGGGCAATCAT-T---GGAATGTGTGCA-------------------- |
| Clon_TF7 | TCAC-CATT-CATGGGCAATCAT-T---GGAATGTGTGCG-------------------- |
| Clon_TF15 | TCAC-CATT-CATGGGCAATCAT-T---GGAATGTGTGCA-------------------- |
| Clon_TF14 | TCAC-CATT-CATGGGCAATCAT-T---GGAATGTGTGCA-------------------- |
| Clon_DP8 | T--TTCGTG-T-TGGGCGA-CAT------GGATGAAAACA-------------------- |
| Clon_DP1 | T--TTCGTG-T-TGGGCGA-CAT------GGATGAAAACA-------------------- |
| Clon_DP7 | T--TTCGTG-T-TGGGCGG-CAT------GGATGAAAACA-------------------- |
| Clon_DP3 | T--TTCGTG-T-TGGGCAA-CAT------GGATGAAAACA-------------------- |
| Clon_DP6 | T--TTCGTG-T-TGGGCAA-CAT------GGATGAAAACA-------------------- |
| Clon_DP9 | T--TTCGTG-T-TGGGCAA-CAT------GGATGAAAACA-------------------- |
| Clon_DP2 | T--TTCGTG-T-TGGGCGA-CAT------GGATGAAAACA-------------------- |
| Clon_DP4 | T--TTCGTG-T-TGGGCGA-CAT------GGATGAAAACA-------------------- |
| Clon_DP10 | T--TTCGTG-T-TGGGCGA-CAT------GGATGAAAACA-------------------- |
| Clon_DP5 | T--TTCGTG-T-TGGGCGA-CAT------GGATGAAAACA-------------------- |
| Clon_EM4 | C--TTTATG-T-TGGGCAA-CAT------GGATAAAGACG-------------------- |
| Clon_EM21 | C--TTTATG-T-TGGGCAA-CAT------GGATAAAGACG-------------------- |
| Clon_EM2 | C--TTTATG-T-TGGGCAA-CAT------GGATAAAGACG-------------------- |
| Clon_EM23 | C--TTTATG-T-TGGGCAA-CAT------GGATAAAGACA-------------------- |
| Clon_EM3 | C--TTTACG-T-AGGGCAA-CAT------GGATAAAGACG-------------------- |
| Clon_EM24 | C--TTTACG-T-AGGGCAA-CAT------GGATAAAGACG-------------------- |
| Clon_EM22 | C--TTTACG-T-AGGGCAA-CAT------GGATAAAGACG-------------------- |
| Clon_EM1 | C--TTTATG-T-TGGGCAA-CAT------GGATAAAGACA-------------------- |
| Clon_EM6 | C--TTTATG-T-GGGGCAA-CAT------GGATAAAGACA-------------------- |
| Clon_EM5 | C--TTTATG-T-GGGGCAA-CAT------GGATAAAGACA-------------------- |
| Clon_GD1 | CTGGGAATACTTTGGGCAACCAAAT---ACGAAGTATCCAATATTTTAAAGGATCCAAAC |
| Clon_GD10 | CTGGGAATACTTTGGGCAACCAAAT---ACGAAGTATCCAATATTTTAAAGGATCCAAAC |
| Clon_GD2 | CTGGGAATACTTTGGGCAACCAAAT---ACGAAGTATCCAATATTTTAAAGGATCCAAAC |
| Clon_GD5 | CTGGGAATACTTTGGGCAACCAAAT---ACGAAGTATCCAATATTTTAAAGGATCCAAAC |
| Clon_GD3 | CTGGGAATACTTTGGGCAACCAAAT---ACGAAGTATCCAATATTTTAAAGGATCCAAAC |
| Clon_GD12 | CTGGGAATACTTTGGGCAACCAAAT---ACGAAGTATCCAATATTTTAAAGGATCCAAAC |
| Clon_GD7 | CTGGGAATACTTTGGGCAACCAAAT---ACGAAGTATCCAATATTTTAAAGGATCCAAAC |
| Clon_GD9 | CTGGGAATACTTTGGGCAACCAAAT---ACGAAGTATCCAATATTTTAAAGGATCCAAAC |
| Clon_GD8 | CTGGGAATACTTTGGGCAACCAAAT---ACGAAGTATCCAATATTTTAAAGGATCCAAAC |
| Clon_GD13 | CTGGGAATACTTTGGGCAACCAAAT---ACGAAGTATCCAATATTTTAAAGGATCCAAAC |
| Clon_LD5 | TTGAGAATACTTTGGGCAACCGAATTATACGAAGTATTCAAT----CAAA-------AAT |
| Clon_LD13 | TTGAGAATACTTTGGGCAACCGAATTATACGAAGTATTCAAT----CAAA-------AAT |
| Clon_LD14 | TTGAGAATACTTTGGGCAACCGAATTATACGAAGTATTCAAT----CAAA-------AAT |
| Clon_LD1 | TTGAGAATACTTTGGGCAACCGAATTATACGAAGTATTCAAT----CAAA-------AAT |
| Clon_LD11 | TTGAGAATACTTTGGGCAACCGAATTATACGAAGTATTCAAT----CAAA-------AAT |
| Clon_LD3 | TTGAGAATACTTTGGGCAACCGAATTATACGAAGTATTCAAT----CAAA-------AAT |
| Clon_LD2 | TTGAGAATACTTTGGGCAACCGAATTATACGAAGTATTCAAT----CAAA-------AAT |
| Clon_LD12 | TTGAGAATACTTTGGGCAACCGAATTATACGAAGTATTCAAT----CAAA-------AAT |
| Clon_LD8 | TTGAGAATACTTTGGGCAACCGAATTATACGAAGTATTCAAT----CAAA-------AAT |
| Clon_LD15 | TTGAGAATACTTTGGGCAACCGAATTATACGAAGTATTCAAT----CAAA-------AAT |
| | *** |
| Clon_DM1 | -TTAG--------TTAGACTTCAT-------AACAATGG--------------------- |
| Clon_DM21 | -TTAG--------TTAGACTTCAT-------AACAATGG--------------------- |
| Clon_DM6 | -ATAG--------TTAGACTTCAT-------AACAATGG--------------------- |
| Clon_DM20 | -ATAG--------TTAGACTTCAT-------AACAATGG--------------------- |
| Clon_DM9 | -TTAG--------TTTGACTTCAT-------AGCAATGG--------------------- |
| Clon_DM12 | -TTAG--------TTTGACTTCAT-------AGCAATGG--------------------- |
| Clon_DM7 | -TTAG--------TTGGACTTCAT-------AATAATGG--------------------- |
| Clon_DM11 | -TTAG--------TTTGACTTCAT-------AGCAATGG--------------------- |
| Clon_DM14 | -TTAG--------TTGGACTTCAT-------AATAATGG--------------------- |
| Clon_DMA | -GTAG--------TTAGACTTCAT-------AACAATGG--------------------- |
| Clon_DF1 | -TTAG--------TTGGACTTCTTT----AAAGCAATGG--------------------- |
| Clon_DF6 | -TTAG--------TTGGACTTCTTT----AAAGCAATGG--------------------- |
| Clon_DF4 | -TTAG--------TTGGACTTCTTT----AAAGCAATGG--------------------- |
| Clon_DF26 | -TTAG--------TTGGACTTCTTT----AAAGCAATGG--------------------- |
| Clon_DF4_50 | -TTAG--------TTCAACTTCAT-------AATAATGG--------------------- |
| Clon_DF19 | -TTAG--------TTCAACTTCAT-------AATAATGG--------------------- |
| Clon_DF3 | -TTAG--------TTCAACTTCAT-------AATAATGG--------------------- |
| Clon_DF5 | -TTAG--------TTCAACTTCAT-------AATAATGG--------------------- |
| Clon_DF2 | -TTAG--------TTCAACTTCAT-------AATAATGG--------------------- |
| Clon_DF7 | -TTAG--------TTAGACTTCTAT----AAAGCAATGG--------------------- |
| Clon_AS15 | GCGGC--------TGTTGGTTGAGAAAGATAGCATACAGTGCTG------TAAGCAGACT |
| Clon_AS14 | GCGGC--------TGTTGGTTGAGAAAGATAGCATACAGTGCTG------TAAGCAGACT |
| Clon_AS20 | GCGGC--------TGTTGGTTGAGAAAGATAGCATACAGTGCTG------TAAGCAGACT |
| Clon_AS13 | GCGGC--------TGTTGGTTGAGAAAGATAGCATACAGTGCTG------TAAGCAGACT |
| Clon_AS10 | GCGGC--------TGTTGGTTGAGAAAGATAGCATACAGTGCT-------TAAGCAGACT |
| Clon_AS11 | GCGGC--------TGTTGGTTGAGAAAGATAGCATACAGTGCTG------TAAGCAGACT |
| Clon_AS2 | GCGGC--------TGTTGGTTGAGAAAGATAGCATACAGTGCT-------TAAGCAGACT |
| Clon_AS12 | GCGGC--------TGTTGGTTGAGAAAGATAGCATACAGTGCTG------TAAGCAGACT |
| Clon_AS1 | GCGGC--------TGTTGGTTGAGAAAGATAGCATACAGTGCTG------TAAGCAGACT |
| Clon_AS16 | GCGGC--------TGTTGGTTGAGAAAGATAGCATACAGTGCT-------TAAGCAGACT |
| Clon_BT8 | ---GC--------AATCGATCTA-----ATGGTTTGCGGT------------------CC |
| Clon_BT9 | ---GC--------AATCGATCTA-----ATGGTTTGCGGT------------------CC |
| Clon_BT16 | ---GC--------AATCGATCTA-----ATGGTTTGCGGT------------------CC |
| Clon_BT3 | ---GC--------AATCGATCTA-----ATGGTTTGCGGT------------------CC |
| Clon_BT14 | ---GT--------AATCGATCTA-----ATGGTTTGCGGT------------------CC |
| Clon_BT17 | ---GC--------AATCGATTTA-----ATGGTTTGCGGT------------------CC |
| Clon_BT13 | ---GC--------AATCGATCTA-----ATGGTTTGCGGT------------------CC |
| Clon_BT1 | ---GC--------AATCGATTTA-----ATGGTTTGCGGT------------------CC |
| Clon_BT10 | ---GT--------AATCGATCTA-----ATGGTTTGCGGT------------------CC |
| Clon_BT15 | ---GC--------AATCGATTTA-----ATGGTTTGCGGT------------------CC |
| Clon_TPA1_20 | GTTGT--------GCGCAATTTA---AGGCCACTTGCATGAG------------TTGCTT |
| Clon_TPA1_22 | GTTGT--------GCGCAATTTA---AGGCCACTTGCATGAG------------TTGCTT |
| Clon_TPA1_29 | GTTGT--------GCGCAATTTA---AGGCCACTTGCATGAG------------TTGCTT |
| Clon_TPA1_28 | GTTGT--------GTGCAATTTA---AGGCCACTTGCATGAG------------TTGCTT |
| Clon_TPA1_26 | GTTGT--------GCGCAATTTA---AGGCCACTTGCATGAG------------TTGCTT |
| Clon_TPA1_21 | GTTGT--------GCGCAATTTA---AGGCCACTTGCATGAG------------TTGCTT |
| Clon_TPA1_36 | GTTGT--------GCGCAATTTA---AGGCCACTTGCATGAG------------TTGCTT |
| Clon_TPA1_27 | GTTGT--------GCGCAATTTA---AGGCCACTTGCATGAG------------TTGCTT |
| Clon_TPA1_23 | GTTGT--------GCGCAATTTA---AGGCCACTTGCATGAG------------TTGCTT |
| Clon_TPA1_1 | GTTGT--------GCGCAATTTA---AGGCCACTTGCATGAG------------TTGCTT |
| Clon_TF22 | GTTGT--------GCGCAATTTA---AGGCCACTTGCATGAG------------TTGCTT |
| Clon_TF24 | GTTGT--------GCGCAATTTA---AGGCCACTTGCATGAG------------TTGCTT |
| Clon_TF3 | GTTGT--------GCGCAATTTA---AGGCCACTTGCATGAG------------TTGCTT |
| Clon_TF2 | GTTGT--------GCGCAATTTA---AGGCCACTTGCATGAG------------TTGCTT |
| Clon_TF23 | GTTGT--------GCGCAATTTA---AGGCCACTTGCATGAG------------TTGCTT |
| Clon_TF1 | GTTGT--------GCGCAATTTA---AGGCCACTTGCATGAG------------TTGCTT |
| Clon_TF4 | GTTGT--------GCGCAATTTA---AGGCCACTTGCATGAG------------TTGCTT |
| Clon_TF7 | GTTGT--------GCGCAATTTA---AGGCCACTTGCATGAG------------TTGCTT |
| Clon_TF15 | GTTGT--------GCGCAATTTA---AGGCCACTTGCATGAG------------TTGCTT |
| Clon_TF14 | GTTGT--------GCGCAATTTA---AGGCCACTTGCATGAG------------TTGCTT |
| Clon_DP8 | -ATGG--------GAGAATTTC-------TTAATTTAGG--------------------- |
| Clon_DP1 | -ATGG--------GAGAATTTC-------TTAATTTAGG--------------------- |
| Clon_DP7 | -ATGG--------GAGAATTTC-------TTAATTTAGG--------------------- |
| Clon_DP3 | -ATGG--------GAGAATTTC-------TTAATTTAGG--------------------- |
| Clon_DP6 | -ATGG--------GAGAATTTC-------TTAATTTAGG--------------------- |
| Clon_DP9 | -GTGG--------GAGAATTTC-------TTAATTTAGG--------------------- |
| Clon_DP2 | -ATGG--------GAGAATTTC-------TTAATTTAGG--------------------- |
| Clon_DP4 | -ATGG--------GAGAATTTC-------TTAATTTAGG--------------------- |
| Clon_DP10 | -ATGG--------GAGAATTTC-------TGAATTTAGG--------------------- |
| Clon_DP5 | -ATGG--------GACAATTTC-------TTAATTTAGG--------------------- |
| Clon_EM4 | -TCGG--------GTGAACTTC-------TTAATTT-GG--------------------- |
| Clon_EM21 | -TCGG--------GTGAACTTC-------TTAATTT-GG--------------------- |
| Clon_EM2 | -TCGG--------GTGAACTTC-------TCAATT--GG--------------------- |
| Clon_EM23 | -CCGG--------GTGAACTTC-------TCAATTA-GG--------------------- |
| Clon_EM3 | -TCGG--------GTGAACTTC-------TTAATTT-GG--------------------- |
| Clon_EM24 | -TCGG--------GTGAACTTC-------TTAATTT-GG--------------------- |
| Clon_EM22 | -TCGG--------GTGAACTTC-------TTAATTT-GG--------------------- |
| Clon_EM1 | -TCGG--------GTGAACTTC-------TCAATTG-GG--------------------- |
| Clon_EM6 | -TCGG--------GTGAACTTC-------TCAATTA-GG--------------------- |
| Clon_EM5 | -TCGG--------GTGAACTTC-------TCAATTA-GG--------------------- |
| Clon_GD1 | GGTGCTGCTCG--GCC-AATCATGCAAATGTGTAATATTG------------ATTTGTTT |
| Clon_GD10 | GGTGCTGCTCG--GCC-AATCATGCAAATGTGTAATATTG------------ATTTGTTT |
| Clon_GD2 | GGTGCTGCTCG--GCC-AATCATGCAAATGTGTAATATTG------------ATTTGTTT |
| Clon_GD5 | GGTGCTGCTCG--GCC-AATCATGCAAATGTGTAATATTG------------ATTTGTTT |
| Clon_GD3 | GGTGCTGCTCG--GCC-AATCATGCAAATGTGTAATATTG------------ATTTGTTT |
| Clon_GD12 | GGTGCTGCTCG--GCC-AATCATGCAAATGTGTAATATTG------------ATTTGTTT |
| Clon_GD7 | GGTGCTGCTCG--GCC-AATCATGCAAATGTGTAATATTG------------ATTTGTTT |
| Clon_GD9 | GGTGCTGCTCG--GCC-AATCATGCAAATGTGTAATATTG------------ATTTGTTT |
| Clon_GD8 | GGTGCTGCTCG--GCC-AATCATGCAAATGTGTAATATTG------------ATTTGTTT |
| Clon_GD13 | GGTGCTGCTCG--GCC-AATCATGCAAATGTGTAATATTG------------ATTTGTTT |
| Clon_LD5 | GTTGCTGCTTAATGCTTGATTGATGAACGCGCATCACATCACAGAAGTAGACATCGATTC |
| Clon_LD13 | GTTGCTGCTTAATGCTTGATTGATGAACGCGCATCACATCACAGAAGTAGACATCGATTC |
| Clon_LD14 | GTTGCTGCTTAATGCTTGATTGATGAACGCGCATCACATCACAGAAGTAGACATCGATTC |
| Clon_LD1 | GTTGCTGCTTAATGCTTGATTGATGAACGCGCATCACATCACAGAAGTAGACATCGATTC |
| Clon_LD11 | GTTGCTGCTTAATGCTTGATTGATGAACGCGCATCACATCACAGAAGTAGACATCGATTC |
| Clon_LD3 | GTTGCTGCTTAATGCTTGATTGATGAACGCGCATCACATCACAGAAGTAGACATCGATTC |
| Clon_LD2 | GTTGCTGCTTAATGCTTGATTGATGAACGCGCATCACATCACAGAAGTAGACATCGATTC |
| Clon_LD12 | GTTGCTGCTTAATGCTTGATTGATGAACGCGCATCACATCACAGAAGTAGACATCGATTC |
| Clon_LD8 | GTTGCTGCTTAATGCTTGATTGATGAACGC--ATCACATCACAGAAGTAGACATCGATTC |
| Clon_LD15 | GTTGCTGCTTAATGCTTGATTGATGAACGC--ATCACATCACAGAAGTAGACATCGATTC |
| Clon_DM1 | -----AA---------CTTGA------------------CACTTA-----CCGGTGT--- |
| Clon_DM21 | -----AA---------CTTGA------------------CACTTA-----CCGGTGT--- |
| Clon_DM6 | -----AA---------CTTGA------------------CACTTA-----CCGGTGT--- |
| Clon_DM20 | -----AA---------CTTGA------------------CACTTA-----CCGGTGT--- |
| Clon_DM9 | -----AA---------CTTGA------------------CACTTA-----CCGGTGT--- |
| Clon_DM12 | -----AA---------CTTGA------------------CACTTA-----CCGGTGT--- |
| Clon_DM7 | -----AA---------CTTGA------------------CACTTA-----CCGGTGT--- |
| Clon_DM11 | -----AA---------CTTGA------------------CACTTA-----CCGGTGT--- |
| Clon_DM14 | -----AA---------CTTGA------------------CACTTA-----CCGGTGT--- |
| Clon_DMA | -----AA---------CTTGA------------------CACTTA-----CCGGTGT--- |
| Clon_DF1 | -----AA---------CTTGA------------------CACTTA-----CCGATGT--- |
| Clon_DF6 | -----AA---------CTTGA------------------CACTTA-----CCGATGT--- |
| Clon_DF4 | -----AA---------CTTGA------------------CACTTA-----CCGATGT--- |
| Clon_DF26 | -----AA---------CTTGA------------------CACTTA-----CCGATGT--- |
| Clon_DF4_50 | -----AA---------CTTGA------------------CACTTA-----CCGATGT--- |
| Clon_DF19 | -----AA---------CTTGA------------------CACTTA-----CCGATGT--- |
| Clon_DF3 | -----AA---------CTTGA------------------CACTTA-----CCGATGT--- |
| Clon_DF5 | -----AA---------CTTGA------------------CACTTA-----CCGATGT--- |
| Clon_DF2 | -----AA---------CTTGA------------------CACTTA-----CCGATGT--- |
| Clon_DF7 | -----AA---------CTTGA------------------CACTTG-----CCTATGT--- |
| Clon_AS15 | AACCAAACTGAAGCACCCGTACCAG--GTA--TACCAGCCAATTA-----CCGCAC---- |
| Clon_AS14 | AACCAAACTGAAGCACCCGTACCAG--GTA--TACCAGCCAATTA-----CCGCAC---- |
| Clon_AS20 | AACCAAACTGAAGCACCCGTACCAG--GTA--TACCAGCCAATTA-----CCGCAC---- |
| Clon_AS13 | AACCAAACTGAAGCACCCGTACCAG--GTA--TACCAGCCAATTA-----CCGCAC---- |
| Clon_AS10 | AACCAAACTGAAGCACCCGTACCAG--GTA--TACCAGCCAATTA-----TCGCAC---- |
| Clon_AS11 | AACCAAACTGAAGCACCCGTACCAG--GTA--TACCAGCCAATTA-----TCGCAC---- |
| Clon_AS2 | AACCAAACTGAAGCACCCGTACCAG--GTA--TACCAGCCAATTA-----TCGCAC---- |
| Clon_AS12 | AACCAAACTGAAGCACCCGTACCAG--GTA--TACCAGCCAATTA-----CCGCAC---- |
| Clon_AS1 | AACCAAACTGAAGCACCCGTACCAGCAGTA--TACCAGCCAATTA-----CCGCAC---- |
| Clon_AS16 | AACCAAACTGAAGCACCCGTACCAG--GTA--TACCAGCCAATTA-----CCGCAC---- |
| Clon_BT8 | A--CAAGTTAAAA----TGTACT----------------CGATTG-----ACA------- |
| Clon_BT9 | A--CAAGTTAAAA----TGTACT----------------CGATTG-----ACA------- |
| Clon_BT16 | A--CAAGTTAAAA----TGTACT----------------CGATTG-----ACA------- |
| Clon_BT3 | A--CAAGTTAAAA----TGTACT----------------CGATTG-----ACA------- |
| Clon_BT14 | A--CAAGTTAAAA----TGTACT----------------CGATTG-----ACA------- |
| Clon_BT17 | A--CAAGTTAAAA----TGTACT----------------TGATTG-----ACA------- |
| Clon_BT13 | A--CAAGTTAAAA----TGTACT----------------CGATTG-----ACA------- |
| Clon_BT1 | A--CAAGTTAAAA----TGTACT----------------CGATTG-----ACA------- |
| Clon_BT10 | A--CAAGTTAAAA----TGTACT----------------CGATTG-----ACA------- |
| Clon_BT15 | A--CAAGTTAAAA----TGTACT----------------CGATTG-----ACA------- |
| Clon_TPA1_20 | AG---AATT------CTTCCACGAA-------------GCAAGTA-----CCACCATTTG |
| Clon_TPA1_22 | AG---AATT------CTTCCACGAA-------------GCAAGTA-----CCACCATTTG |
| Clon_TPA1_29 | AG---AATT------CTTCCACGAA-------------GCAAGTA-----CCGCCATTTG |
| Clon_TPA1_28 | AG---AATT------CTTCCACGAA-------------GCAAGTA-----CCACCATTTG |
| Clon_TPA1_26 | AG---AATT------TCTTTACAAA-------------GCAAGTA-----CCACCATTTG |
| Clon_TPA1_21 | AG---AATT------CTTCCACGAA-------------GCAAGTA-----CCACCATTTG |
| Clon_TPA1_36 | AG---AATT------CTTCCACGAA-------------GCAAGTA-----CCACCATTTG |
| Clon_TPA1_27 | AG---AATT------CTTCCACGAA-------------GCAAGTA-----CCACCATTTG |
| Clon_TPA1_23 | AG---AATT------CTTCCACGAA-------------GCAAGTA-----CCACCATTTG |
| Clon_TPA1_1 | AG---AATT------CTTCCACGAA-------------GCAAGTA-----CCACCATTTG |
| Clon_TF22 | AG---AA--------CTTCCTTGAAA-----------AGCAGGTG-----T-ACTAACTA |
| Clon_TF24 | AG---AA--------CTTCCTTGAAA-----------AGCAGGTG-----T-ACTAACTA |
| Clon_TF3 | AG---AA--------CTTCCTTGAAA-----------AGCAGGTG-----T-ACTAACTA |
| Clon_TF2 | AG---AA--------CTTCCTTGAAA-----------AGCAGGTG-----T-ACTAACTA |
| Clon_TF23 | AG---AA--------CTTCCTTGAAA-----------AGCAGGTG-----T-ACTAACTA |
| Clon_TF1 | AG---AA--------CTTCCTTGAAA-----------AGCAGGTG-----T-ACTAACTA |
| Clon_TF4 | AG---AA--------CTTCCTTGAAA-----------AGCAGGTG-----T-ACTAACTA |
| Clon_TF7 | AG---AA--------CTTCCTTGAAA-----------AGCAGGTG-----T-ACTAACTA |
| Clon_TF15 | AG---AA--------CTTCCTTGAAA-----------AGCAGGTG-----T-ACTAACTA |
| Clon_TF14 | AG---AA--------CTTCCTTGAAA-----------AGCAGGTG-----T-ACTAACTA |
| Clon_DP8 | -----AG---------CTAAAC-----------------CGA--A-----TCA-TGT--- |
| Clon_DP1 | -----AG---------CTAAAC-----------------CGA--A-----TCA-TGT--- |
| Clon_DP7 | -----AG---------CTGGAC-----------------CGA--A-----TCA-TGT--- |
| Clon_DP3 | -----AG---------CTGGAT-----------------CGA--A-----TCA-TCT--- |
| Clon_DP6 | -----AG---------CTGGAT-----------------CGA--A-----TCA-TCT--- |
| Clon_DP9 | -----AG---------CTGGAT-----------------CGA--A-----TCA-TCT--- |
| Clon_DP2 | -----AG---------CTGGAC-----------------CGA--A-----TCA-TGT--- |
| Clon_DP4 | -----AG---------CTGGAC-----------------CGA--A-----TCA-TGT--- |
| Clon_DP10 | -----GG---------TTGGAC-----------------CGA--A-----TCA-TGT--- |
| Clon_DP5 | -----AG---------TAAGAC-----------------CGA--A-----TCA-TGT--- |
| Clon_EM4 | -----AG---------CCTGA------------------CAATTA-----TCAATGTG-- |
| Clon_EM21 | -----AG---------CCTGA------------------CAATTA-----TCAATGTG-- |
| Clon_EM2 | -----AG---------CCTGA------------------CAATCA-----TCAATGTG-- |
| Clon_EM23 | -----AG---------CCTGA------------------CAATTA-----TCAATGTG-- |
| Clon_EM3 | -----AG---------CCTGA------------------CAATTA-----TCAATGTG-- |
| Clon_EM24 | -----AG---------CCTGA------------------CAATTA-----TCAATGTG-- |
| Clon_EM22 | -----AG---------CCTGA------------------CAATTA-----TCAATGTG-- |
| Clon_EM1 | -----AG---------CCTGA------------------CAATTA-----TCAATGTG-- |
| Clon_EM6 | -----AG---------CCTGA------------------CAATTA-----TCAATGTG-- |
| Clon_EM5 | -----AG---------CCTGA------------------CAATTA-----TCAATGTG-- |
| Clon_GD1 | ATTCAAGTCGG----CTGGCATT---------------TCACTTA---CCTTGATT--A- |
| Clon_GD10 | ATTCAAGTCGG----CTGGCATT---------------TCACTTA---CCTTGATT--A- |
| Clon_GD2 | ATTCAAGTCGG----CTGGCATT---------------TCACTTA---CCTTGATT--A- |
| Clon_GD5 | ATTCAAGTCGG----CTGGCATT---------------TCACTTA---CCTTGATT--A- |
| Clon_GD3 | ATTCAAGTCGG----CTGGCATT---------------TCACTTA---CCTTGATT--A- |
| Clon_GD12 | ATTCAAGTCGG----CTGGCATT---------------TCACTTA---CCTTGATT--A- |
| Clon_GD7 | ATTCAAGTCGG----CTGGCATT---------------TCACTTA---CCTTGATT--A- |
| Clon_GD9 | ATTCAAGTCGG----CTGGCATT---------------TCACTTA---CCTTGATT--A- |
| Clon_GD8 | ATTCAAGTCGG----CTGGCATT---------------TCACTTA---CCTTGATT--A- |
| Clon_GD13 | ATTCAAGTCGG----CTGGCATT---------------TCACTTA---CCTTGATT--A- |
| Clon_LD5 | AATTGAATCGG----CTGGCATTGCTATGAC------ATTCCATAATTCCTCAATTCCAT |
| Clon_LD13 | AATTGAATCGG----CTGGCATTGCTATGAC------ATTCCATAATTCCTCAATTCCAT |
| Clon_LD14 | AATTGAATCGG----CTGGCATTGCTATGAC------ATTCCATAATTCCTCAATTCCAT |
| Clon_LD1 | AATTGAATCGG----CTGGCATTGCTATGAC------ATTCCATAATTCCTCAATTCCAT |
| Clon_LD11 | AATTGAATCGG----CTGGCATTGCTATGAC------ATTCCATAATTCCTCAATTCCAT |
| Clon_LD3 | AATTGAATCGG----CTGGCATTGCTATGAC------ATTCCATAATTCCTCAATTCCAT |
| Clon_LD2 | AATTGAATCGG----CTGGCATTGCTATGAC------ATTCCATAATTCCTCAATTCCAT |
| Clon_LD12 | AATTGAATCGG----CTGGCATTGCTATGAC------ATTCCATAATTCCTCAATTCCAT |
| Clon_LD8 | AATTGAATCGG----CTGGCATTGCTATGAC------ATTCCATAA--CCTCAATTCCAT |
| Clon_LD15 | AATTGAATCGG----CTGGCATTGCTATGAC------ATTCCATAA--CCTCAATTCCAT |
| Clon_DM1 | -----------ACAAC--------------TTTGTCGGCGATTCTC--CTTGCT-ATCTG |
| Clon_DM21 | -----------ACAAC--------------TTTGTCGGCGATTCTC--CTTGCT-ATCTG |
| Clon_DM6 | -----------ACAAC--------------TTTGTCGGCGATTCTC--CTTGCT-ATCTG |
| Clon_DM20 | -----------ACAAC--------------TTTGTCGGCGATTCTC--CTTGCT-ATCTG |
| Clon_DM9 | -----------ACAAC--------------TTTGTCGGCGATTCTC--CTTGCT-ATCTG |
| Clon_DM12 | -----------ACAAC--------------TTTGTCGGCGATTCTC--CTTGCT-ATCTG |
| Clon_DM7 | -----------ACAAC--------------TTTGTCGGCGATTCTC--CTTGCT-ATCTG |
| Clon_DM11 | -----------ACAAC--------------TTTGTCGGCGATTCTC--CTTGCT-ATCTG |
| Clon_DM14 | -----------ACAAC--------------TTTGTCGGCGATTCTC--CTTGCT-ATCTG |
| Clon_DMA | -----------ACAAC--------------TTTGTCGGCGATTCTC--CTTGCT-ATCTG |
| Clon_DF1 | -----------ACAAC--------------TTTGTCGGCGATTCTC--CTTGCT-ATCTG |
| Clon_DF6 | -----------ACAAC--------------TTTGTCGGCGATTCTC--CTTGCT-ATCTG |
| Clon_DF4 | -----------ACAAC--------------TTTGTCGGCGATTCTC--CTTGCT-ATCTG |
| Clon_DF26 | -----------ACAAC--------------TTTGTCGGCGATTCTC--CTTGCT-ATCTG |
| Clon_DF4_50 | -----------ACAAC--------------TTTGTCGGCGATTCTC--CTTGCT-ATCTG |
| Clon_DF19 | -----------ACAAC--------------TTTGTCGGCGATTCTC--CTTGCT-ATCTG |
| Clon_DF3 | -----------ACAAC--------------TTTGTCGGCGATTCTC--CTTGCT-ATCTG |
| Clon_DF5 | -----------ACAAC--------------TTTGTCGGCGATTCTC--CTTGCT-ATCTG |
| Clon_DF2 | -----------ACAAC--------------TTTGTCGGCGATTCTC--CTTGCT-ATCTG |
| Clon_DF7 | -----------ACAAC--------------TTTGTCGGCGATTCTC--CTTGCT-ATCTG |
| Clon_AS15 | -----------ACACCAAATGACATGAACTTTCCATGAAAGCTCTCCTTGTATC-ATTCG |
| Clon_AS14 | -----------ACACCAAATGACATGAACTTTCCATGAAAGCTCTCCTTGTATC-ATTCG |
| Clon_AS20 | -----------ACACCAAATGACATGAACTTTCCATGAAAGCTCTCCTTGTATC-ATTCG |
| Clon_AS13 | -----------ACACCAAATGACATGAACTTTCCATGAAAGCTCTCCTTGTATC-ATTCG |
| Clon_AS10 | -----------ACACCAAATGACATGAACTTTCCATGAAAGCTCTCCTTGTATC-ATTCG |
| Clon_AS11 | -----------ACACCAAATGACATGAACTTTCCATGAAAGCTCTCCTTGTATC-ATTCG |
| Clon_AS2 | -----------ACACCAAATGACATGAACTTTCCATGAAAGCTCTCCTTGTATC-ATTCG |
| Clon_AS12 | -----------ACACCAAATGACATGAACTTTCCATGAAAGCTCTCCTTGTATC-ATTCG |
| Clon_AS1 | -----------ACACCAAATGACATGAACTTTCCATGAAAGCTCTCCTTGTATC-ATTCG |
| Clon_AS16 | -----------ACACCAAATGACATGAACTTTCCATGAAAGCTCTCCTTGTATC-ATTCG |
| Clon_BT8 | -----------ACACCA--------------TTTATG------CTTTGTACATC-GTT-- |
| Clon_BT9 | -----------ACACCA--------------TTTATG------CTTTGTACATC-GTT-- |
| Clon_BT16 | -----------ACACCA--------------TTTATG------CTTTGTACATC-GTT-- |
| Clon_BT3 | -----------ACACCA--------------TTTATG------CTTTGTACATC-GTT-- |
| Clon_BT14 | -----------ACACCA--------------TTTATG------CTTTGTACATC-GTT-- |
| Clon_BT17 | -----------ACACCA--------------TTTATG------CTTTGTACATC-GTT-- |
| Clon_BT13 | -----------ACACCA--------------TTTATG------CTTTGTACATC-GTT-- |
| Clon_BT1 | -----------ACACCA--------------TTTATG------CTTTGTACATC-GTT-- |
| Clon_BT10 | -----------ACACCA--------------TTTATG------CTTTGTACATC-GTT-- |
| Clon_BT15 | -----------ACACCA--------------TTTATG------CTTTGTACATC-GTT-- |
| Clon_TPA1_20 | AC---------ACAGTACCTCG-------TCTATCTGAAAGCTCTC--CTTGTCCATCTG |
| Clon_TPA1_22 | AC---------ACAGTACCTCG-------TCTATCTGAAAGCTCTC--CTTGTCCATCTG |
| Clon_TPA1_29 | AC---------ACAGTACCTCG-------TCTATCTGAAAGCTCTC--CTTGTCCATCTG |
| Clon_TPA1_28 | AC---------ACAGTACCTCG-------TCTATCTGAAAGCTCTC--CTTGTCCATCTG |
| Clon_TPA1_26 | AC---------ACAGTACCTCG-------TCTATCTGAAAGCTCTC--CTTGTCCATCTG |
| Clon_TPA1_21 | AC---------GCAGTACCTCG-------TCTATCTGAAAGCTCTC--CTTGTCCATCTG |
| Clon_TPA1_36 | AC---------GCAGTACCTCG-------TCTATCTGAAAGCTCTC--CTTGTCCATCTG |
| Clon_TPA1_27 | AC---------GCAGTACCTCG-------TCTATCTGAAAGCTCTC--CTTGTCCATCTG |
| Clon_TPA1_23 | AC---------ACAGTACCTCG-------TCTATCTGAAAGCTCTC--CTTGTCCATCTG |
| Clon_TPA1_1 | AC---------ACAGTACCTCG-------TCTATCTGAAAGCTCTC--CTTGTCCATCTG |
| Clon_TF22 | GCCA-------ACAGTGC-TCG-------TCTATCTGAAAGCTCTC--CTTGTCCATCTG |
| Clon_TF24 | GCCA-------ACAGTGC-TCG-------TCTATCTGAAAGCTCTC--CTTGTCCATCTG |
| Clon_TF3 | GCCA-------ACAGTGC-TCG-------TCTATCTGAAAGCTCTC--CTTGTCCATCTG |
| Clon_TF2 | GCCA-------ACAGTGC-TCG-------TCTATCTGAAAGCTCTC--CTTGTCCATCTG |
| Clon_TF23 | GCCA-------ACAGTGC-TCG-------TCTATCTGAAAGCTCTC--CTTGTCCATCTG |
| Clon_TF1 | GCCA-------ACAGTGC-TCG-------TCTATCTGAAAGCTCTC--CTTGTCCATCTG |
| Clon_TF4 | GCCA-------ACAGTGC-TCG-------TCTATCTGAAAGCTCTC--CTTGTCCATCTG |
| Clon_TF7 | GCCA-------ACAGTGC-TCG-------TCTATCTGAAAGCTCTC--CTTGTCCATCTG |
| Clon_TF15 | GCCA-------ACAGTGC-TCG-------TCTATCTGAAAGCTCTC--CTTGTCCATCTG |
| Clon_TF14 | GCCA-------ACAGTGC-TCG-------TCTATCTGAAAGCTCTC--CTTGTCCATCTG |
| Clon_DP8 | ------------CAAC--------------TTTGTCGGCGATTCTC--CTTGAT-GCCTG |
| Clon_DP1 | ------------CAAC--------------TTTGTCGGCGATTCTC--CTTGAT-GCCTG |
| Clon_DP7 | ------------TAAC--------------TTTGTCGGCGATTCTC--CTTGAT-GCCTG |
| Clon_DP3 | ------------CAAC--------------TTTGTCGGCGATTCTC--CTTGAT-GCCTG |
| Clon_DP6 | ------------CAAC--------------TTTGTCGGCGATTCTC--CTTGAT-GCCTG |
| Clon_DP9 | ------------CAAC--------------TTTGTCGGCGATTCTC--CTTGAT-GCCTG |
| Clon_DP2 | ------------CAAC--------------TTTGTCGGCGATTCTC--CTTGAT-GCCTG |
| Clon_DP4 | ------------CAAC--------------TTTGTCGGCGATTCTC--CTTGAT-GCCTG |
| Clon_DP10 | ------------CAAC--------------TTTGTCGGCGATTCTC--CTTGAT-GCCTG |
| Clon_DP5 | ------------CAAC--------------TTTGTCGGCGATTCTC--CTTGAT-GCCTG |
| Clon_EM4 | ------------TAAC--------------TTTGTCGGCGATTCTC--CTTGAT-GCCTG |
| Clon_EM21 | ------------CAAC--------------TTTGTCGGCGATTCTC--CTTGAT-GCCTG |
| Clon_EM2 | ------------CAAC--------------TTTGTCGGCGATTCTC--CTTGAT-GCCTG |
| Clon_EM23 | ------------CAAC--------------TTTGTCGGCGATTCTC--CTTGAT-GCCTG |
| Clon_EM3 | ------------CAAC--------------TTTGTCGGCGATTCTC--CTTGAT-GCCTG |
| Clon_EM24 | ------------CAAC--------------TTTGTCGGCGATTCTC--CTTGAT-GCCTG |
| Clon_EM22 | ------------CAAC--------------TTTGTCGGCGATTCTC--CTTGAT-GCCTG |
| Clon_EM1 | ------------CAAC--------------TTTGTCGGCGATTCTC--CTTGAT-GCCTG |
| Clon_EM6 | ------------CAAC--------------TTTGTCGGCGATTCTC--CTTGAT-GCCTG |
| Clon_EM5 | ------------CAAC--------------TTTGTCGGCGATTCTC--CTTGAT-GCCTG |
| Clon_GD1 | -----------ATAATTG-TG------TCGCTTGTTGGCAACTCTGCTCATATC---TTG |
| Clon_GD10 | -----------ATAATTG-TG------TCGCTTGTTGGCAACTCTGCTCATATC---TTG |
| Clon_GD2 | -----------ATAATTG-TG------TCGCTTGTTGGCAACTCTGCTCATATC---TTG |
| Clon_GD5 | -----------ATAATTG-TG------TCGCTTGTTGGCAACTCTGCTCATATC---TTG |
| Clon_GD3 | -----------ATAATTG-TG------TCGCTTGTTGGCAACTCTGCTCATATC---TTG |
| Clon_GD12 | -----------ATAATTG-TG------TCGCTTGTTGGCAACTCTGCTCATATC---TTG |
| Clon_GD7 | -----------ATAATTG-TG------TCGCTTGTTGGCAACTCTGCTCATATC---TTG |
| Clon_GD9 | -----------ATAATTG-TG------TCGCTTGTTGGCAACTCTGCTCATATC---TTG |
| Clon_GD8 | -----------ATAATTG-TG------TCGCTTGTTGGCAACTCTGCTCATATC---TTG |
| Clon_GD13 | -----------ATAATTG-TG------TCGCTTGTTGGCAACTCTGCTCATATC---TTG |
| Clon_LD5 | CTTCATGCGATATGATTGGTGA-----TCGTTTGTTGGCAACTCTGCTCATATC---TTG |
| Clon_LD13 | CTTCATGCGATATGATTGGTGA-----TCGTTTGTTGGCAACTCTGCTCATATC---TTG |
| Clon_LD14 | CTTCATGCGATATGATTGGTGA-----TCGTTTGTTGGCAACTCTGCTCATATC---TTG |
| Clon_LD1 | CTTCATGCGATATGATTGGTGA-----TCGTTTGTTGGCAACTCTGCTCATATC---TTG |
| Clon_LD11 | CTTCATGCGATATGATTGGTGA-----TCGTTTGTTGGCAACTCTGCTCATATC---TTG |
| Clon_LD3 | CTTCATGCGATATGATTGGTGA-----TCGTTTGTTGGCAACTCTGCTCATATC---TTG |
| Clon_LD2 | CTTCATGCGATATGATTGGTGA-----TCGTTTGTTGGCAACTCTGCTCATATC---TTG |
| Clon_LD12 | CTTCATGCGATATGATTGGTGA-----TCGTTTGTTGGCAACTCTGCTCATATC---TTG |
| Clon_LD8 | CTTCATGCGATATGATTGGTGA-----TCGTTTGTTGGCAACTCTGCTCATATC---TTG |
| Clon_LD15 | CTTCATGCGATATGATTGGTGA-----TCGTTTGTTGGCAACTCTGCTCATATC---TTG |
| | * * |
| Clon_DM1 | -T-TGGTGCCTAGTCTACGGTTCCTGTTTA-------TCCTCGGGATGAACGTGTGGATG |
| Clon_DM21 | -T-TGGTGCCTAGTCTACGGTTCCTGTTTA-------TCCTCGGGATGAACGTGTGGATG |
| Clon_DM6 | -T-TGGTGCCTAGTCTACGGTTCCTGTTTA-------TCCTCGGGATGAACGTGTGGATG |
| Clon_DM20 | -T-TGGTGCCTAGTCTACGGTTCCTGTTTA-------TCCTCGGGATGAACGTGTGGATG |
| Clon_DM9 | -T-TGGTGCCTAGTCTACGGTTCCTGTTTA-------TCCTCGGGATGAACGTGTGGATG |
| Clon_DM12 | -T-TGGTGCCTAGTCTACGGTTCCTGTTTA-------TCCTCGGGATGAACGTGTGGATG |
| Clon_DM7 | -T-TGGTGCCTACTCTACGGTTCCTGTTTA-------TCCTCGGGATGAACGTGTGGATG |
| Clon_DM11 | -T-TGGTGCCTAGTCTACGGTTCCTGTTTA-------TCCTCGGGATGAACGTGTGGATG |
| Clon_DM14 | -T-TGGTGCCTAGTCTACGGTTCCTGTTTA-------TCCTCGGGATGAACGTGTGGATG |
| Clon_DMA | -T-TGGTGCCTACTCTACGGTTCCTGTCTA-------TCCTCGGGATGAACGTGTGGATG |
| Clon_DF1 | -T-TGGTGCCTATTCTACGGTTCCTGTTTG-------TCCTCGGGATAAACGTG--GATG |
| Clon_DF6 | -T-TGGTGCCTATTCTACGGTTCCTGTTTA-------TCCTCGGGATGAACGTG--GATG |
| Clon_DF4 | -T-TGGTGCCTATTCTACGGTTCCTGTTTG-------TCCTCGGGATGAACGTG--GATG |
| Clon_DF26 | -T-TGGTGCCTATTCTACGGTTCCTGTTTG-------TCCTCGGGATGAACGTG--GATG |
| Clon_DF4_50 | -T-TGGTGCCTATTCTACGGTTCCTGTTTG-------TCCTCGGGATAAACGTG--GATG |
| Clon_DF19 | -T-TGGTGCCTATTCTACGGTTCCTGTTTG-------TCCTCGGGATAAACGTG--GATG |
| Clon_DF3 | -T-TGGTGCCTATTCTACGGTTCCTGTTTG-------TCCTCGGGATAAACGTG--GATG |
| Clon_DF5 | -T-TGGTGCCTATTCTACGGTTCCTGTTTA-------TCCTCGGGATGAACGTG--GATG |
| Clon_DF2 | -T-TGGTGCCTATTCTACGGTTCCTGTTTA-------TCCTCGGGATGAACGTG--GATG |
| Clon_DF7 | -T-TGGTGCCTATTCTACTGTTCCTGTTTG-------TCCTCGGGATAAACGTG--GATG |
| Clon_AS15 | AT-TGGTGCCTAGTCTGCGTTTCCTGCCA-----AATTTTTGGCGT------TG--GATG |
| Clon_AS14 | AT-TGGTGCCTAGTCTGCGTTTCCTGCCA-----AATTTTTGGCGT------TG--GATG |
| Clon_AS20 | AT-TGGTGCCTAGTCTGCGTTTCCTGCCA-----AATTTTTGGCGT------TG--GATG |
| Clon_AS13 | AT-TGGTGCCTAGTCTGCGTTTCCTGCCA-----AATTTTTGGCGT------TG--GATG |
| Clon_AS10 | AT-TGGTGCCTAGTCTGCGTTTCCTGCCA-----AATTTTTGGCGT------TG--GATG |
| Clon_AS11 | AT-TGGTGCCTAGTCTGCGTTTCCTGCCA-----AATTTTTGGCGT------TG--GATG |
| Clon_AS2 | AT-TGGTGCCTAGTCTGCGTTTCCTGCCA-----AATTTTTGGCGT------TG--GATG |
| Clon_AS12 | AT-TGGTGCCTAGTCTGCGTTTCCTGCCA-----AATTTTTGGCGT------TG--GATG |
| Clon_AS1 | AT-TGGTGCCTAGTCTGCGTTTCCTGCCA-----AATTTTTGGCGT------TG--GATG |
| Clon_AS16 | AT-TGGTGCCTAGTCTGCGTTTCCTGCCA-----AATTTTTGGCGT------TG--GATG |
| Clon_BT8 | AT-CGGTGCCTAGTCTGCGGTTC-TGTC---------CTTTAGTGA------TG--GATG |
| Clon_BT9 | AT-CGGTGCCTAGTCTGCGGTTC-TGTC---------CTTTAGTGA------TG--GATG |
| Clon_BT16 | AT-CGGTGCCTAGTCTGCGGTTC-TGTC---------CACTAGTGA------TG--GATG |
| Clon_BT3 | AT-CGGTGCCTAGTCTGCGGTTC-TGTC---------CTTTAGTGA------TG--GATG |
| Clon_BT14 | AT-CGGTGCCTAGTCTGCGGTTC-TGTC---------CTTTAGTGA------TG--GATG |
| Clon_BT17 | AC-CGGTGCCTAGTCTGCGGTTC-TGTC---------CTTTAGTGA------TG--GATG |
| Clon_BT13 | AT-CGGTGCCTAGTCTGCGGTTC-TGTC---------CTTTAGTGA------TG--GATG |
| Clon_BT1 | AC-CGGTGCCTAGTCTGCGGTTC-TGTC---------CTTTAGTGA------TG--GATG |
| Clon_BT10 | AC-CGGTGCCTAGTCTGCGGTTC-TGTC---------CACTAGTGA------TG--GATG |
| Clon_BT15 | AC-CGGTGCCTAGTCTGCGGTTC-TGTC---------CTTTAGTGA------TG--GATG |
| Clon_TPA1_20 | AT-TGGTGCCTAGTCTGCGCTTCCTGCC---------TCTTAACGGAGGCT-TG--GATG |
| Clon_TPA1_22 | AT-TGGTGCCTAGTCTGCGCTTCCTGCC---------TCTTAACGGAGGCT-TG--GATG |
| Clon_TPA1_29 | AT-TGGTGCCTAGTCTGCGCTTCCTGCC---------TCTTAACGGAGGCT-TG--GATG |
| Clon_TPA1_28 | AT-TGGTGCCTAGTCTGCGCTTCCTGCC---------TCTTAACGGAGGCT-TG--GATG |
| Clon_TPA1_26 | AT-TGGTGCCTAGTCTGCGCTTCCTGCC---------TCTTAACGGAGGCT-TG--GATG |
| Clon_TPA1_21 | AT-TGGTGCCTAGTCTGCGTTTCCTGCC---------TTCTAACAGAGGCT-TG--GATG |
| Clon_TPA1_36 | AT-TGGTGCCTAGTCTGCGCTTCCTGCT---------TCCTAACGGAGGCT-TG--GATG |
| Clon_TPA1_27 | AT-TGGTGCCTAGTCTGCGCTTCCTGCC---------TTCTAACAGAGGCT-TG--GATG |
| Clon_TPA1_23 | AT-TGGTGCCTAGTCTGCGCTTCCTGCC---------TTCTAACAGAGGCT-TG--GATG |
| Clon_TPA1_1 | AT-TGGTGCCTAGTCTGCGCTTCCTGCC---------TTCTAACAGAGGCT-TG--GATG |
| Clon_TF22 | AT-TGGTGCCTAGTCTGCGCTTCCTGCC---------TTCTAACAGAGGCT-TG--GATG |
| Clon_TF24 | AT-TGGTGCCTAGTCTGCGCTTCCTGCC---------TTCTAACAGAGGCT-TG--GATG |
| Clon_TF3 | AT-TGGTGCCTAGTCTGCGCTTCCTGCC---------TTCTAACAGAGGCT-TG--GATG |
| Clon_TF2 | AT-TGGTGCCTAGTCTGCGCTTCCTGCC---------TTCTAACAGAGGCT-TG--GATG |
| Clon_TF23 | AT-TGGTGCCTAGTCTGCGCTTCCTGCC---------TTCTAACAGAGGCT-TG--GATG |
| Clon_TF1 | AT-TGGTGCCTAGTCTGCGCTTCCTGCC---------TTCTAACAGAGGCT-TG--GATG |
| Clon_TF4 | AT-TGGTGCCTAGTCTGCGCTTCCTGCC---------TTCTAACAGAGGCT-TG--GATG |
| Clon_TF7 | AT-TGGTGCCTAGTCTGCGCTTCCTGCC---------TTCTAACAGAGGCT-TG--GATG |
| Clon_TF15 | AT-TGGTGCCTAGTCTGCGCTTCCTGCC---------TTCTAACAGAGGCT-TG--GATG |
| Clon_TF14 | AT-TGGTGCCTAGTCTGCGCTTCCTGCC---------TTCTAACAGAGGCT-TG--GATG |
| Clon_DP8 | -T-TGGTGCCTAGCCTACAGTTCCTGTTTG-------TCCTCGGGACAGATGTG--GATG |
| Clon_DP1 | -T-TGGTGCCTAGCCTACAGTTCCTGTTTG-------TCCTCGGGACAGATGTG--GATG |
| Clon_DP7 | -T-TGGTGCCTAGCCTACAGTTCCTGTTTG-------TCCTCGGGACAGATGTG--GATG |
| Clon_DP3 | -T-TGGTGCCTAGCCTACAGTTCCTGTTTG-------TCCTCGGGACAGATGTG--GATG |
| Clon_DP6 | -T-TGGTGCCTAGCCTACAGTTCCTGTTTG-------TCCTCGGGACAGATGTG--GATG |
| Clon_DP9 | -T-TGGTGCCTAGCCTACAGTTCCTGTTTG-------TCCTCGGGACAGATGTG--GATG |
| Clon_DP2 | -T-TGGTGCCTAGCCTACAGTTCCTGTTTG-------TCCTCGGGACAGATGTG--GATG |
| Clon_DP4 | -T-TGGTGCCTAGCCTACAGTTCCTGTTTG-------TCCTCGGGACAGATGTG--GATG |
| Clon_DP10 | -T-TGGTGCCTAGCCTACAGTTCCTGTTTG-------TCCTCGGGACAGATGTG--GATG |
| Clon_DP5 | -T-TGGTGCCTAGCCTACAGTTCCTGTTTG-------TCCTCGGGACAGATGTG--GATG |
| Clon_EM4 | -T-TGGTGCCTAGCCTACAGTTCCTGTCCA-------TCCTCGGGACAGATGTG--GATG |
| Clon_EM21 | -T-TGGTGCCTAGCCTACAGTTCCTGTCCA-------TCCTCGGGACAGATGTG--GATG |
| Clon_EM2 | -T-TGGTGCCTAGCCTACAGTTCCTGTCCA-------TCCTCGGGACAGATGTG--GATG |
| Clon_EM23 | -T-TGGTGCCTAGCCTACAGTTCCTGTCCA-------TCCTCGGGACAGATGTG--GATG |
| Clon_EM3 | -T-TGGTGCCTAGCCTACAGTTCCTGTCCA-------TCCTCGGGACAGATGTG--GATG |
| Clon_EM24 | -T-TGGTGCCTAGCCTACAGTTCCTGTCCA-------TCCTCGGGACAGATGTG--GATG |
| Clon_EM22 | -T-TGGTGCCTAGCCTACAGTTCCTGTCCA-------TCCTCGGGACAGATGTG--GATG |
| Clon_EM1 | -T-TGGTGCCTAGCCTACAGTTCCTGTCCA-------TCCTCGGGACAGATGTG--GATG |
| Clon_EM6 | -T-TGGTGCCTAGCCTACAGTTCCTGTCCA-------TCCTCGGGACAGATGTG--GATG |
| Clon_EM5 | -T-TGGTGCCTAGCCTACAGTTCCTGTCCA-------TCCTCGGGACAGATGTG--GATG |
| Clon_GD1 | TCCTGGTGTCTAGTCCGCTATTCCTAACAA-------AT-TAAAAACTTGTATG--GATG |
| Clon_GD10 | TCCTGGTGTCTAGTCCGCTATTCCTAACAA-------AT-TAAAAACTTGTATG--GATG |
| Clon_GD2 | TTCTGGTGTCTAGTCCGCTATTCCTAACAA-------AT-TAAAAACTTGTATG--GATG |
| Clon_GD5 | TTCTGGTGTCTAGTCCGCTATTCCTAACAA-------AT-TAAAAACTTGTATG--GATG |
| Clon_GD3 | TTCTGGTGTCTAGTCCGCTATTCCTAACAA-------AT-TAAAAACTTGTATG--GATG |
| Clon_GD12 | TTCTGGTGTCTAGTCCGCTATTCCTAACAA-------AT-TAAAAACTTGTATG--GATG |
| Clon_GD7 | TTCTGGTGTCTAGTCCGCTATTCCTAACAA-------AT-TAAAAACTTGTATG--GATG |
| Clon_GD9 | TTCTGGTGTCTAGTCCGCTATTCCTAACAA-------AT-TAAAAACTTGTATG--GATG |
| Clon_GD8 | TTCTGGTGTCTAGTCCGCTATTCCTAACAA-------AT-TAAAAACTTGTATG--GATG |
| Clon_GD13 | TCCTGGTGTCTAGTCCGCTATTCCTAACAA-------AT-TAAAAACTTGTATG--GATG |
| Clon_LD5 | CT-TGGTGCCTAGTCCGCTGTTCCTGCTTT-------CCGTAAGGATTGTCGTG--GATG |
| Clon_LD13 | CT-TGGTGCCTAGTCCGCTGTTCCTGCTTT-------CCGTAAGGATTGTCGTG--GATG |
| Clon_LD14 | CT-TGGTGCCTAGTCCGCTGTTCCTGCTTT-------CCGTAAGGATTGTCGTG--GATG |
| Clon_LD1 | CT-TGGTGCCTAGTCCGCTGTTCCTGCTTT-------CCGTAAGGATTGTCGTG--GATG |
| Clon_LD11 | CT-TGGTGCCTAGTCCGCTGTTCCTGCTTT-------CCGTAAGGATTGTCGTG--GATG |
| Clon_LD3 | CT-TGGTGCCTAGTCCGCTGTTCCTGCTTT-------CCGTAAGGATTGTCGTG--GATG |
| Clon_LD2 | CT-TGGTGCCTAGTCCGCTGTTCCTGCTTT-------CCGTAAGGATTGTCGTG--GATG |
| Clon_LD12 | CT-TGGTGCCTAGTCCGCTGTTCCTGCTTT-------CCGTAAGGATTGTCGTG--GATG |
| Clon_LD8 | CT-TGGTGCCTAGTCCGCTGTTCCTGCTTT-------CCGTAAGGATTGTCGTG--GATG |
| Clon_LD15 | CT-TGGTGCCTAGTCCGCTGTTCCTGCTTT-------CCGTAAGGATTGTCGTG--GATG |
| | **** *** * * *** * * ** **** |
| Clon_DM1 | TAG-TGTGTCGCTTG------------TAATGAG----------------TGCCGCTAGG |
| Clon_DM21 | TAG-TGTGTCGCTTG------------TAATGAG----------------TGCCGCTAGG |
| Clon_DM6 | TAG-TGTGTCGCTTG------------TAATGAG----------------TGCCGCTAGG |
| Clon_DM20 | TAG-TGTGTCGCTTG------------TAATGAG----------------TGCCGCTAGG |
| Clon_DM9 | TAG-TGTGTCGCTTG------------TAATGAG----------------TGCCGCTAGG |
| Clon_DM12 | TAG-TGTGTCGCTTG------------TAATGAG----------------TGCCGCTAGG |
| Clon_DM7 | TAG-TGTGTCGCTTG------------TAATGAG----------------TGCCGCTAGG |
| Clon_DM11 | TAG-TGTGTCGCTTG------------TAATGAG----------------TGCCGCTAGG |
| Clon_DM14 | TAG-TGTGTCGCTTG------------TAATGAG----------------TGCCGCTAGG |
| Clon_DMA | TAG-TGTGTCGCTTG------------TAATGAG----------------TGCCGCTAGG |
| Clon_DF1 | TAG-TGTGTCGCTTG------------TAATGAG----------------TGCCGCTAGG |
| Clon_DF6 | TAG-TGTGTCGCTTG------------TAATGAG----------------TGCCGCTAGG |
| Clon_DF4 | TAG-TGTGTCGCTTG------------TAATGAG----------------TGCCGCTAGG |
| Clon_DF26 | TAG-TGTGTCGCTTG------------TAATGAG----------------TGCCGCTAGG |
| Clon_DF4_50 | TAG-TGTGTCGCTTG------------TAATGAG----------------TGCCGCTAGG |
| Clon_DF19 | TAG-TGTGTCGCTTG------------TAATGAG----------------TGCCGCTAGG |
| Clon_DF3 | TAG-TGTGTCGCTTG------------TAATGAG----------------TGCCGCTAGG |
| Clon_DF5 | TAG-TGTGTCGCTTG------------TAATGAG----------------TGCCGCTAGG |
| Clon_DF2 | TAG-TGTGTCGCTTG------------TAATGAG----------------TGCCGCTAGG |
| Clon_DF7 | TAG-TGTGTCGCTTG------------TAATGAG----------------TGCCGCTAGG |
| Clon_AS15 | CAG-GGTGTCAGCCAGTTTTGCGACTCTTTTGGG---TTGCTTCTGCTGCTGCT---AGG |
| Clon_AS14 | CAG-GGTGTCAGCCAGTTTTGCGACTCTTTTGGG---TTGCTTCTGCTGCTGCT---AGG |
| Clon_AS20 | CAG-GGTGTCAGCCAGTTTTGCGACTCTTTTGGG---TTGCTTCTGCTGCTGCT---AGG |
| Clon_AS13 | CAG-GGTGTCAGCCAGTTTTGCGACTCTTTTGGG---TTGCTTCTGCTGCTGCT---AGG |
| Clon_AS10 | CAG-GGTGTCAGCCAGTTTTGCGACTCTTTTGGG---TTGCTTCTGCTGCTGCT---AGG |
| Clon_AS11 | CAG-GGTGTCAGCCAGTTTTGCGACTCTTTTGGG---TTGCTTCTGCTGCTGCT---AGG |
| Clon_AS2 | CAG-GGTGTCAGCCAGTTTTGCGACTCTTTTGGG---TTGCTTCTGCTGCTGCT---AGG |
| Clon_AS12 | CAG-GGTGTCAGCCAGTTTTGCGACTCTTTTGGG---TTGCTTCTGCTGCTGCT---AGG |
| Clon_AS1 | CAG-GGTGTCAGCCAGTTTTGCGACTCTTTTGGG---TTGCTTCTGCTGCTGCT---AGG |
| Clon_AS16 | CAG-GGTGTCAGCCA--TTTGCGACTCTTTTGGG---TTGCTTCTGCTGCTGCT---AGG |
| Clon_BT8 | CAG-GGTATCA-------------TTGTATAAGA---T-------------GCT---AGG |
| Clon_BT9 | CAG-GGTATCA-------------TTGTATAAGA---T-------------GCT---AGG |
| Clon_BT16 | CAG-GGTATCA-------------T--TATTAGA---T-------------GCT---AGG |
| Clon_BT3 | CAG-GGTATCA-------------T--TTATAGA---T-------------GCT---AGG |
| Clon_BT14 | CAG-GGTATCA-------------T--TTATAGA---T-------------GCT---AGG |
| Clon_BT17 | CAG-GGTATCA-------------T--TTATAGA---T-------------GCT---AGG |
| Clon_BT13 | CAG-GGTATCA-------------T--TTATAGA---T-------------GCT---AGG |
| Clon_BT1 | CAG-GGTATCA-------------TT--ATTAGA---T-------------GCT---AGG |
| Clon_BT10 | CAG-GGTATCA-------------TTGTATAAGA---T-------------GCT---AGG |
| Clon_BT15 | CAG-GGTGTCAGTTGTGTGAGTG-----GCTAAA--CCCCTCTCCACGGCTGCTGCTAGG |
| Clon_TPA1_20 | CAG-GGTGTCAGTTGTGTGAGTG-----GCTAAA--CCCCTCTCCACGGCTGCTGCTAGG |
| Clon_TPA1_22 | CAG-GGTGTCAGTTGTGTGAGTG-----GCTAAA--CCCCTCTCCACGGCTGCTGCTAGG |
| Clon_TPA1_29 | CAG-GGTGTCAGTTGTGTGAGTG-----GCTAAA--CCCCTCTCCACGGCTGCTGCTAGG |
| Clon_TPA1_28 | CAG-GGTGTCAGTTGTGTGAGTG-----GCTAAA--CCCCTCTCCACGGCTGCTGCTAGG |
| Clon_TPA1_26 | CAG-GGTGTCAGTTGTGTGAGTG-----GCTAAA--CCCCTCTCCACGGCTGCTGCTAGG |
| Clon_TPA1_21 | CAG-GGTGTCAGTTGTGTGAGTG-----GCTAAA--CCCCTCTCCACGGCTGCTGCTAGG |
| Clon_TPA1_36 | CAG-GGTGTCAGTTGTGTGAGTG-----GCTAAA--CCCCGCTCGGCAGCTGCTGCTAGG |
| Clon_TPA1_27 | CAG-GGTGTCAGTTGTGTGAGTG-----GCTAAA--CCCCTCTCCACGGCTGCTGCTAGG |
| Clon_TPA1_23 | CAG-GGTGTCAGTTGTGTGAGTG-----GCTAAA--CCCCGCTCGGCAGCTGCTGCTAGG |
| Clon_TPA1_1 | CAG-GGTGTCAGTTGTGTGAGTG-----GCTAAA--CCCCTCTCCACGGCTGCTGCTAGG |
| Clon_TF22 | CAG-GGTGTCAGTTGAGTGTGTGTGGGTGTTAAAGCCTCCACTGCTCAATTGCTGCTAGG |
| Clon_TF24 | CAG-GGTGTCAGTTGAGTGTGTGTGGGTGTTAAAGCCTCCACTGCTCAATTGCTGCTAGG |
| Clon_TF3 | CAG-GGTGTCAGTTGAGTGTGTGTGGGTGTTAAAGCCTCCACTGCTCAATTGCTGCTAGG |
| Clon_TF2 | CAG-GGTGTCAGTTGAGTGTGTGTGGGTGTTAAAGCCTCCACTGCTCAATTGCTGCTAGG |
| Clon_TF23 | CAG-GGTGTCAGTTGAGTGTGTGTGGGTGTTAAAGCCTCCACTGCTCAATTGCTGCTAGG |
| Clon_TF1 | CAG-GGTGTCAGTTGAGTGTGTGTGGGTGTTAAAGCCTCCACTGCTCAATTGCTGCTAGG |
| Clon_TF4 | CAG-GGTGTCAGTTGAGTGTGTGTGGGTGTTAAAGCCTCCACTGCTCAATTGCTGCTAGG |
| Clon_TF15 | CAG-GGTGTCAGTTGAGTGTGTGTGGGTGTTAAAGCCTCCACTGCTCAATTGCTGCTAGG |
| Clon_TF14 | CAG-GGTGTCAGTTGAGTGTGTGTGGGTGTTAAAGCCTCCACTGCTCAATTGCTGCTAGG |
| Clon_DP8 | TAG-TGTGTCGCTT-------------TGTT--------CTTGCAACAAGTGCCGCTAGG |
| Clon_DP1 | TAG-TGTGTCGCTT-------------TGTT--------CTTGCAACAAGTGCCGCTAGG |
| Clon_DP7 | TAG-TGTGTCGCTT-------------TGTT--------CTTGAAACAAGTGCCGCTAGG |
| Clon_DP3 | TAG-TGTGTCGCTT-------------TGTT--------CTTGAAACAAGTGCCGCTAGG |
| Clon_DP6 | TAG-TGTGTCGCTT-------------TGTT--------CTTGAAACAAGTGCCGCTAGG |
| Clon_DP9 | TAG-TGTGTCGCTT-------------TGTT--------CTTGAAACAAGTGCCGCTAGG |
| Clon_DP2 | TAG-TGTGTCGCTT-------------TGTTGTT-----TTTGCAACAAGTGCCGCTAGG |
| Clon_DP4 | TAG-TGTGTCGCTT-------------TGTTGTT-----TTTGCAACAAGTGCCGCTAGG |
| Clon_DP10 | TAG-TGTGTCGCTT-------------TGTTGTT-----TTTGCAACAAGTGCCGCTAGG |
| Clon_DP5 | TAG-TGTGTCGCTT-------------TGTTGTT-----TTTGCAACAAGTGCCGCTAGG |
| Clon_EM4 | TAG-TGTGTCGCTT-------------CAACA---------------AAGTGCCGCTAGG |
| Clon_EM21 | TAG-TGTGTCGCTT-------------CAACA---------------AAGTGCCGCTAGG |
| Clon_EM2 | TAG-TGTGTCGCTT-------------TATCA---------------AAGTGCCGCTAGG |
| Clon_EM23 | TAG-TGTGTCGCTT-------------CATCA---------------AAGTGCCGCTAGG |
| Clon_EM3 | TAG-TGTGTCGCTT-------------CAACA---------------AAGTGCCGCTAGG |
| Clon_EM24 | TAG-TGTGTCGCTT-------------CAACA---------------AAGTGCCGCTAGG |
| Clon_EM22 | TAG-TGTGTCGCTT-------------CAACA---------------AAGTGCCGCTAGG |
| Clon_EM1 | TAG-TGTGTCGCTT-------------CAACA---------------AAGTGCCGCTAGG |
| Clon_EM6 | TAG-TGTGTCGCTT-------------TATCA---------------AAGTGCCGCTAGG |
| Clon_EM5 | TAG-TGTGTCGCTT-------------TATCA---------------AAGTGCCGCTAGG |
| Clon_GD1 | CGG-TGTGTCAGTC-------------TATTGTT-------------GACTGTTGCTAGG |
| Clon_GD10 | CGG-TGTGTCAGTC-------------TATTGTT-------------GACTGTTGCTAGG |
| Clon_GD2 | CGG-TGTGTCAGTC-------------TATTGTT-------------GACTGTTGCTAGG |
| Clon_GD5 | CGG-TGTGTCAGTC-------------TATTGTT-------------GACTGTTGCTAGG |
| Clon_GD3 | CGG-TGTGTCAGTC-------------TATTGTT-------------GACTGTTGCTAGG |
| Clon_GD12 | CGG-TGTGTCAGTC-------------TATTGTT-------------GACTGTTGCTAGG |
| Clon_GD7 | CGG-TGTGTCAGTC-------------TATTGTT-------------GACTGTTGCTAGG |
| Clon_GD9 | CGG-TGTGTCAGTC-------------TATTGTT-------------GACTGTTGCTAGG |
| Clon_GD8 | CGG-TGTGTCAGTC-------------TATTGTT-------------GACTGTTGCTAGG |
| Clon_GD13 | CGG-TGTGTCAGTC-------------TATTGTT-------------GACTGTTGCTAGG |
| Clon_LD5 | CGG-TGTGTCAG-C-------------TAT--AT-------------G-CTGTTGCTAGG |
| Clon_LD13 | CGG-TGTGTCAG-C-------------TAT--AT-------------G-CTGTTGCTAGG |
| Clon_LD14 | CGG-TGTGTCAG-C-------------TAT--AT-------------G-CTGTTGCTAGG |
| Clon_LD1 | CGG-TGTGTCAG-C-------------TAT--AT-------------G-CTGTTGCTAGG |
| Clon_LD11 | CGGGTGTGTCAG-C-------------TAT--AT-------------G-CTGTTGCTAGG |
| Clon_LD3 | CGG-TGTGTCAG-C-------------TAT--AT-------------G-CTGTTGCTAGG |
| Clon_LD2 | CGG-TGTGTCAG-C-------------TAT--AT-------------G-CTGTTGCTAGG |
| Clon_LD12 | CGG-TGTGTCAG-C-------------TAT--AT-------------G-CTGTTGCTAGG |
| Clon_LD8 | CGG-TGTGTCAG-C-------------TAT--AT-------------G-CTGTTGCTAGG |
| Clon_LD15 | CGG-TGTGTCAG-C-------------TAT--AT-------------G-CTGTTGCTAGG |
| | * ** ** * *** |
| Clon_DM1 | TTT-AAATACCAGATTT----GAATGTA-------ATTCGATTGCTGGAGCCACAGGTAC |
| Clon_DM21 | TTT-AAATACCAAATTT----GAATGTA-------ATTCGATTGCTGGAGCCACAGGTAC |
| Clon_DM6 | TTT-AAATACCAGATTCCAACAAATATATT-----GTTTGATTGCTGGAGCCACAGGTAC |
| Clon_DM20 | TTT-AAATACCAGATTCCAACAAATATATT-----GTTTGATTGCTGGAGCCACAGGTAC |
| Clon_DM9 | TTT-AAATACCAGATTCCAACAAATATATT-----GTTTGATTGCTGGAGCCACAGGTAC |
| Clon_DM12 | TTT-AAATACCAGATTCCAACAAATATATT-----GTTTGATTGCTGGAGCCACAGGTAC |
| Clon_DM7 | TTT-AAATACCAGATTCCAACAAATATATT-----GTTTGATTGCTGGAGCCACAGGTAC |
| Clon_DM11 | TTT-AAATACCAGATTCCAACAAATATATT-----GTTTGATTGCTGGAGCCACAGGTAC |
| Clon_DM14 | TTT-AAATACCAGATTCCAACAAATATATT-----GTTTGATTGCTGGAGCCACAGGTAC |
| Clon_DMA | TTT-AAATACCAGATTC----GAATGTA-------ATTCGATTGCTGGAGCCACAGGTAC |
| Clon_DF1 | TTT-AAATACCAGATTC----GAGCATA-------ATTCGATTGCTGGAGCCACAGGTAC |
| Clon_DF6 | TTT-AAATACCAGATTC----GAGCATA-------ATTCGATTGCTGGAGCCACAGATAC |
| Clon_DF4 | TTT-AAATACCAGATTC----GAGCATA-------ATTCGATTGCTGGAGCCACAGGTAC |
| Clon_DF26 | TTT-AAATACCAGATTC----GAGCATA-------ATTCGATTGCTGGAGCCACAGGTAC |
| Clon_DF4_50 | TTT-AAATACCAGATTC----GAGCATA-------ATTCGATTGCTGGAGCCACAGGTAC |
| Clon_DF19 | TTT-AAATACCAGATTC----GAGCATA-------ATTCGATTGCTGGAGCCACAGGTAC |
| Clon_DF3 | TTT-AAATACCAGATTC----GAGCATA-------ATTCGATTGCTGGAGCCACAGGTAC |
| Clon_DF5 | TTT-AAATACCAGATTC----GAGCATA-------ATTCGATTGCTGGAGCCACAGGTAC |
| Clon_DF2 | TTT-AAATACCAGATTC----GAGCATA-------ATTCGATTGCTGGAGCCACAGGTAC |
| Clon_DF7 | TTT-AAATACCAGATTC----GAGCATA-------ATTCGATTGCTGGAGCCACAGGTAC |
| Clon_AS15 | CTTTAAATATC-------------TACG---------CCAAGCGCCGAAGCCTCGAATGC |
| Clon_AS14 | CTT--AATATC-------------TACG---------CCAAGCGCCGAAGCCTCGAATGC |
| Clon_AS20 | CTT-AAATATC-------------TACG---------CCAAGCGCCGAAGCCTCGAATGC |
| Clon_AS13 | CTT-AAATATC-------------TACG---------CCAAGCGCCGAAGCCTCGAATGC |
| Clon_AS10 | CTT-AAATATC-------------TACG---------CCAAGCGCCGAAGCCTCGAATGC |
| Clon_AS11 | CTT-AAATATC-------------TACG---------CCAAGCGCCGAAGCCTCGAATGC |
| Clon_AS2 | CTT-AAATATC-------------TACG---------CCAAGCGCCGAAGCCTCGAATGC |
| Clon_AS12 | CTT-AAATATC-------------TACG---------CCAAGCGCCGAAGCCTCGAATGC |
| Clon_AS1 | CTT-AAATATC-------------TACG---------CCAAGCGCCGAAGCCTCGAATGC |
| Clon_AS16 | CTT-AAATATC-------------TACG---------CCAAGCGCCGAAGCCTCGAATGC |
| Clon_BT8 | CTT-AAATATC----------------A---------TC--GT---GAAGCC--AAGTG- |
| Clon_BT9 | CTT-AAATATC----------------A---------TC--GT---GAAGCC--AAGTG- |
| Clon_BT16 | CTT-AAATATC----------------A---------TTTTGT---GAAGCC--AAGTG- |
| Clon_BT3 | CTT-AAATATC----------------A---------TTTTGT---GAAGCC--AAGTG- |
| Clon_BT14 | CTT-AAATATC----------------A---------TC--AT---GAAGCC--AAGTG- |
| Clon_BT17 | CTT-AAATATC----------------A---------TC--AT---GAAGCC--AAGTG- |
| Clon_BT13 | CTT-AAATATC----------------A---------TTTTGT---GAAGCC--AAGTG- |
| Clon_BT1 | CTT-AAATATC----------------A---------TC--AT---GAAGCC--AATTG- |
| Clon_BT10 | CTT-AAATATC----------------A---------TC--GT---GAAGCC--AAGTG- |
| Clon_BT15 | CTT-AAATATC----------------A---------TC--GT---GAAGCC--AAGTG- |
| Clon_TPA1_20 | CTT-AAATATCAGTGCCA-----GTGCG---------CTGAGCGCCGAAGCCTCAGATGC |
| Clon_TPA1_22 | CTT-AAATATCAGTGCCA-----GTGCG---------CTGAGCGCCGAAGCCTCAGATGC |
| Clon_TPA1_29 | CTT-AAATATCAGTGCCA-----GTGCG---------CTGAGCGCCGAAGCCTCAGATGC |
| Clon_TPA1_28 | CTT-AAATATCAGTGCCA-----GTGCG---------CTGAGCGCCGAAGCCTCAGATGC |
| Clon_TPA1_26 | CTT-AAATATCAGTGCCA-----GTGCG---------CTGAGCGCCGAAGCCTCAGATGC |
| Clon_TPA1_21 | CTT-AAATATCAGTGCCA-----GTGCG---------CTGAGCGCCGAAGCCTCAGATGC |
| Clon_TPA1_36 | CTT-AAATATCAGTGCCA-----GTGCG---------CTGAGCGCCGAAGCCTCAGATGC |
| Clon_TPA1_27 | CTT-AAATATCAGTGCCA-----GTGCG---------CTGAGCGCCGAAGCCTCAGATGC |
| Clon_TPA1_23 | CTT-AAATATCAGTGCCA-----GTGCG---------CTGAGCGCCGAAGCCTCAGATGC |
| Clon_TPA1_1 | CTT-AAATATCAGTGCCA-----GTGCG---------CTGAGCGCCGAAGCCTCAGATGC |
| Clon_TF22 | CTT-AAATATCAGTGCCA-----GTGCG---------CTGAGCGCCGAAGCCTCAGATGC |
| Clon_TF24 | CTT-AAATATCAGTGCCA-----GTGCG---------CTGAGCGCCGAAGCCTCAGATGC |
| Clon_TF3 | CTT-AAATATCAGTGCCA-----GTGCG---------CTGAGCGCCGAAGCCTCAGATGC |
| Clon_TF2 | CTT-AAATATCAGTGCCA-----GTGCG---------CTGAGCGCCGAAGCCTCAGATGC |
| Clon_TF23 | CTT-AAATATCAGTGCCA-----GTGCG---------CTGAGCGCCGAAGCCTCAGATGC |
| Clon_TF1 | CTT-AAATATCAGTGCCA-----GTGCG---------CTGAGCGCCGAAGCCTCAGATGC |
| Clon_TF4 | CTT-AAATATCAGTGCCA-----GTGCG---------CTGAGCGCCGAAGCCTCAGATGC |
| Clon_TF15 | CTT-AAATATCAGTGCCA-----GTGCG---------CTGAGCGCCGAAGCCTCAGATGC |
| Clon_TF14 | CTT-AAATATCAGTGCCA-----GTGCG---------CTGAGCGCCGAAGCCTCAGATGC |
| Clon_DP8 | TTT-AAATACTGGATTCGT---CATCGA---------ACGAATGCCGGAGCCACAGGTTC |
| Clon_DP1 | TTT-AAATACTGGATTCGT---CATCGA---------ACGAATGCCGGAGCCACAGGTTC |
| Clon_DP7 | TTT-AAATACTGGATTCGT---CATCGA---------ACGAATGCCGGAGCCACAGGTTC |
| Clon_DP3 | TTT-AAATACTGGATTCGT---AATTGA---------ACGAATGCCGGAGCCACAGGTTC |
| Clon_DP6 | TTT-AAATACTGGATTCGT---AATTGA---------ACGAATGCCGGAGCCACAGGTTC |
| Clon_DP9 | TTT-AAATACTGGATTCGT---AATTGA---------ACGAATGCCGGAGCCACAGGTTC |
| Clon_DP2 | TTT-AAATACTGGATTCGT---CATCGT---------ACGAATGCCGGAGCCACAGGTTC |
| Clon_DP4 | TTT-AAATACTGGATTCGT---CATCGT---------ACGAATGCCGGAGCCACAGGTTC |
| Clon_DP10 | TTT-AAATACTGGATTCGT---CATGGA---------ACGAATGCCGGAGCCACAGGTTC |
| Clon_DP5 | TTT-AAATACTGGATTCGT---CATCGA---------ACGAATGCCGGAGCCACAGGTTC |
| Clon_EM4 | TTT-AAATACTGGATTCG----AATTAT---------TCGAATGTCGGAGCCACAGGTAC |
| Clon_EM21 | TTT-AAATACTGGATTCG----AATTAT---------TCGAATGTCGGAGCCACAGGTAC |
| Clon_EM2 | TTT-AAATACTGGATTCG----AATTAT---------TCGAATGTCGGAGCCACAGGTAC |
| Clon_EM23 | TTT-AAATACTGGATTCG----AATTAT---------TCGAATGTCGGAGCCACAGGTAC |
| Clon_EM3 | TTT-AAATACTGGATTCG----AATTAT---------TCGAATGTCGGAGCCACAGGTAC |
| Clon_EM24 | TTT-AAATACTGGATTCG----AATTAT---------TCGAATGTCGGAGCCACAGGTAC |
| Clon_EM22 | TTT-AAATACTGGATTCG----AATTAT---------TCGAATGTCGGAGCCACAGGTAC |
| Clon_EM1 | TTT-AAATACTGGATTCG----AATTAT---------TCGAATGTCGGAGCCACAGGTAC |
| Clon_EM6 | TTT-AAATACTGGATTCG----AATTAT---------TCGAATGTCGGAGCCACAGGTAC |
| Clon_EM5 | TTT-AAATACTGGATTCG----AATTAT---------TCGAATGTCGGAGCCACAGGTAC |
| Clon_GD1 | CTT-AAATATCGTTAG--------CTTAAC----TGTT-----GACGAAGCCGCAAGTAT |
| Clon_GD10 | CTT-AAATATCGTTAG--------CTTAAC----TGTT-----GACGAAGCCGCAAGTAT |
| Clon_GD2 | CTT-AAATATCGTTAG--------CTTAAC----TGTT-----GACGAAGCCGCAAGTAT |
| Clon_GD5 | CTT-AAATATCGTTAG--------CTTAAC----TGTT-----GACGAAGCCGCAAGTAT |
| Clon_GD3 | CTT-AAATATCGTTAG--------CTTAAC----TGTT-----GACGAAGCCGCAAGTAT |
| Clon_GD12 | CTT-AAATATCGTTAG--------CTTAAC----TGTT-----GACGAAGCCGCAAGTAT |
| Clon_GD7 | CTT-AAATATCGTTAG--------CTTAAC----TGTT-----GACGAAGCCGCAAGTAT |
| Clon_GD9 | CTT-AAATATCGTTAG--------CTTAAC----TGTT-----GACGAAGCCGCAAGTAT |
| Clon_GD8 | CTT-AAATATCGTTAG--------CTTAAC----TGTT-----GACGAAGCCGCAAGTAT |
| Clon_GD13 | CTT-AAATATCGTTAG--------CTTAAC----TGTT-----GACGAAGCCGCAAGTAT |
| Clon_LD5 | CTT-AAATATCGTTT---------CGCAAT----TATTTGGGAAACGAAGCCGCAAGTAT |
| Clon_LD13 | CTT-AAATATCGTTT---------CGCAAT----TATTTGGGAAACGAAGCCGCAAGTAT |
| Clon_LD14 | CTT-AAATATCGTTT---------CGCAAT----TATTTGGGAAACGAAGCCGCAAGTAT |
| Clon_LD1 | CTT-AAATATCGTTT---------CGCAAT----TATTTGGGAAACGAAGCCGCAAGTAT |
| Clon_LD11 | CTT-AAATATCGTTT---------CGCAAT----TATTTGGGAAACGAAGCCGCAAGTAT |
| Clon_LD3 | CTT-AAATATCGTTT---------CGCAAT----TATTTGGGAAACGAAGCCGCAAGTAT |
| Clon_LD2 | CTT-AAATATCGTTT---------CGCAAT----TATTTGGGAAACGAAGCCGCAAGTAT |
| Clon_LD12 | CTT-AAATATCGTTT---------CGCAAT----TATTTGGGAAACGAAGCCGCAAGTAT |
| Clon_LD8 | CTT-AAATATCGTTT---------CGCAATATTACAATTGGGATACGGAGCCGCAAGTAT |
| Clon_LD15 | CTT-AAATATCGTTT---------CGCAATATTACAATTGGGATACGGAGCCGCAAGTAT |
| | ** **** * **** * |
| Clon_DM1 | AAGGC------ATTTTCTTTTT-------------------------CATTTCAAAACAA |
| Clon_DM21 | AAGGC------ATTTTCTTTTT-------------------------CATTTCAAAACAA |
| Clon_DM6 | AAGGC------ATTTTCTTTTT-------------------------CATTTCAAAACAA |
| Clon_DM20 | AAGGC------ATTTTCTTTTT-------------------------CATTTCAAAACAA |
| Clon_DM9 | AAGGC------ATTTTCTTTTT-------------------------CATTTCAAAACAA |
| Clon_DM12 | AAGGC------ATTTTCTTTTT-------------------------CATTTCAAAACAA |
| Clon_DM7 | GAGGC------ATTTTCTTTTT-------------------------CATTTCAAAACAA |
| Clon_DM11 | AAGGC------ATTTTCTTTTT-------------------------CATTTCAAAACAA |
| Clon_DM14 | AAGGC------ATTTTCTTTTT-------------------------CATTTCAAAACAA |
| Clon_DMA | AAGGC------ATTTTCTTTTT-------------------------CATTTCAAAACAA |
| Clon_DF1 | AAGGC------ATTTTCTTTTT-------------------------CATTTCAAAACAA |
| Clon_DF6 | AAGGC------ATTTTCTTTTT-------------------------CATTTCAAAACAA |
| Clon_DF4 | AAGGC------ATTTTCTTTTT-------------------------CATTTCAAAACAA |
| Clon_DF26 | AAGGC------ATTTTCTTTTT-------------------------CATTTCAAAACAA |
| Clon_DF4_50 | AAGGC------ATTTTCTTTTT-------------------------CATTTCAAAACAA |
| Clon_DF19 | AAGGC------ATTTTCTTTTT-------------------------CATTTCAAAACAA |
| Clon_DF3 | AAGGC------ATTTTCTTTTT-------------------------CATTTCAAAACAA |
| Clon_DF5 | AAGGC------ATTTTCTTTTT-------------------------CATTTCAAAACAA |
| Clon_DF2 | AAGGC------ATTTTCTTTTT-------------------------CATTTCAAAACAA |
| Clon_DF7 | AAGGC------ATTTTCTTTTT-------------------------CATTTCAAAACAA |
| Clon_AS15 | AAGGTT--CTCGCGTACAGTATAC--------CTAGTGTG--CAGTACGTTGA-GTGAGA |
| Clon_AS14 | AAGGTT--CTCGCGTACAGTATAC--------CTAGTGTG--CAGTACGTTGA-GTGAGA |
| Clon_AS20 | AAGGTT--CTCGCGTACAGTATAC--------CTAGTGTG--CAGTACGTTGA-GTGAGA |
| Clon_AS13 | AAGGTT--CTCGCGTACAGCACACA-----CCCAGGTGTGAGCAGTACGTTGA-GTGAGA |
| Clon_AS10 | AAGGTT--CTCGCGTACAGCACACA-----CCCAGGTGTGAGCAGTACGTTGA-GTGAGA |
| Clon_AS11 | AAGGTT--CTCGCGTACAGCACACA-----CCCAGGTGTGAGCAGTACGTTGA-GTGAGA |
| Clon_AS2 | AAGGTT--CTCGCGTACAGCACACA-----CCCAGGTGTGAGCAGTACGTTGA-GTGAGA |
| Clon_AS12 | AAGGTT--CTCGCGTACAGCACACA-----CCTAG-TGTGAGCAGTACGTTGA-GTGAGA |
| Clon_AS1 | AAGGTT--CTCGCGTACAGCACACA-----CCCAGGTGTGAGCAGTACGTTGA-GTGAGA |
| Clon_AS16 | AAGGTT--CTCGCGTACAGCACACA-----CCCAGGTGTGAGCAGTACGTTGA-GTGAGA |
| Clon_BT8 | -----------ATGTACATTA---------------------------GCTAA-GGGAA- |
| Clon_BT9 | -----------ATGTACATTA---------------------------GCTAA-GGGAA- |
| Clon_BT16 | -----------ATGTACATTA---------------------------GCTAA-GGGAA- |
| Clon_BT3 | -----------ATGTACATTA---------------------------GCTAA-GGGAA- |
| Clon_BT14 | -----------ATGTACATTA---------------------------GCTAA-GGGAA- |
| Clon_BT17 | -----------ATGTACATTA---------------------------GCTAA-GGGAA- |
| Clon_BT13 | -----------ATGTACATTA---------------------------GCTAA-GGGAA- |
| Clon_BT1 | -----------ATGTACATTA---------------------------GCTAA-GGGAA- |
| Clon_BT10 | -----------ATGTACATTA---------------------------GCTAA-GGGAA- |
| Clon_BT15 | -----------ATGTACATTA---------------------------GCTAA-GGGAA- |
| Clon_TPA1_20 | AAGGTCGAGTAGTGTGCACTGTTGGGTAAC--CAGCTCT-----GCACGCTGCTGTGAAA |
| Clon_TPA1_22 | AAGGTCGAGTAGTGTGCACTGTTGGGTAAC--CAGCTCT-----GCACGCTGCTGTGAAA |
| Clon_TPA1_29 | AAGGTCGAGTAGTGTGCACTGTTGGGTAAC--CAGCTCT-----GCACGCTGCTGTGAAA |
| Clon_TPA1_28 | AAGGTCGAGTAGTGTGCACTGTTGGGTAAC--CAGCTCT-----GCACGCTGCTGTGAAA |
| Clon_TPA1_26 | AAGGTCGAGTAGTGTGCACTGTTGGGTAAC--CAGCTCT-----GCACGCTGCTGTGAAA |
| Clon_TPA1_21 | AAGGTCGAGTAGTGTGCACTGTTGGGTAAC--CAGCTCT-----GCACGCTGCTGTGAAA |
| Clon_TPA1_36 | AAGGTCGAGTAGTGTGCACTGTTGGGTAAC--CAGCTCT-----GCACGCTGCTGTGAAA |
| Clon_TPA1_27 | AAGGTCGAGTAGTGTGCACTGTTGGGTAAC--CAGCTCT-----GCACGCTGCTGTGAAA |
| Clon_TPA1_23 | AAGGTCGAGTAGTGTGCACTGTTGGGTAAC--CAGCTCT-----GCACGCTGCTGTGAAA |
| Clon_TPA1_1 | AAGGTCGAGTAGTGTGCACTGTTGGGTAAC--CAGCTCT-----GCACGCTGCTGTGAAA |
| Clon_TF22 | AAGGTCGAGTAGTGTGCA--GTTGAGCAAT--CAACG-------GTGCACTGCTGTGAAA |
| Clon_TF24 | AAGGTCGAGTAGTGTGCA--GTTGAGCAAT--CAACG-------GTGCACTGCTGTGAAA |
| Clon_TF3 | AAGGTCGAGTAGTGTGCA--GTTGAGCAAT--CAACG-------GTGCACTGCTGTGAAA |
| Clon_TF2 | AAGGTCGAGTAGTGTGCA--GTTGAGCAAT--CAACG-------GTGCACTGCTGTGAAA |
| Clon_TF23 | AAGGTCGAGTAGTGTGCA--GTTGAGCAAT--CAACG-------GTGCACTGCTGTGAAA |
| Clon_TF1 | AAGGTCGAGTAGTGTGCA--GTTGAGCAAT--CAACG-------GCGCACTGCTGTGAAA |
| Clon_TF4 | AAGGTCGAGTAGTGTGCA--GTTGAGCAAT--CAACG-------GCGCACTGCTGTGAAA |
| Clon_TF15 | AAGGTCGAGTAGTGTGCA--GTTGAGCAAT--CAACG-------GCGCACTGCTGTGAAA |
| Clon_TF14 | AAGGTCGAGTAGTGTGCA--GTTGAGCAAT--CAACG-------GCGCACTGCTGTGAAA |
| Clon_DP8 | AAGGT------ATTTTCTTTTT-------------------------CATTTATGAAAAA |
| Clon_DP1 | AAGGT------ATTTTCTTTTT-------------------------CATTTATGAAAAA |
| Clon_DP7 | AAGGT------ATTTTCTTTTT-------------------------CATTTATGAAAAA |
| Clon_DP3 | AAGGT------ATTTTCTTTTT-------------------------CATTTATGAAAAA |
| Clon_DP6 | AAGGT------ATTTTCTTTTT-------------------------CATTTATGAAAAA |
| Clon_DP9 | AAGGT------ATTTTCTTTTT-------------------------CATTTATGAAAAA |
| Clon_DP2 | AAGGT------ATTTTCTTTTT-------------------------CATTTATGAAAAA |
| Clon_DP4 | AAGGT------ATTTTCTTTTT-------------------------CATTTATGAAAAA |
| Clon_DP10 | AAGGT------ATTTTCTTTTT-------------------------CATTTATGAAAAA |
| Clon_DP5 | AAGGT------ATTTT-TTTTT-------------------------CATTTATGAAAAA |
| Clon_EM4 | AAGGT------ATCT-CTTTTT-------------------------CATTTATGAAAAA |
| Clon_EM21 | AAGGT------ATCT-CTTTTT-------------------------CATTTATGAAAAA |
| Clon_EM2 | AAGGT------ATTT-CTTTTT-------------------------CATTTATGAAAAA |
| Clon_EM23 | AAGGT------ATTT-CTTTTT-------------------------CATTTATGAAAAA |
| Clon_EM3 | AAGGT------ATTT-CTTTTT-------------------------CATTTATGAAAAA |
| Clon_EM24 | AAGGT------ATTT-CTTTTT-------------------------CATTTATGAAAAA |
| Clon_EM22 | AAGGT------ATTT-CTTTTT-------------------------CATTTATGAAAAA |
| Clon_EM1 | AAGGT------ATTT-CTTTTT-------------------------CATTTATGAAAAA |
| Clon_EM6 | GAGGT------ATTT-CTTTTT-------------------------CATTTATGAAAAA |
| Clon_EM5 | GAGGT------ATTT-CTTTTT-------------------------CATTTATGAAAAA |
| Clon_GD1 | GGGGAA----A-CCTTTTCTTT--------------------------ACTTAAACCAAA |
| Clon_GD10 | GGGGAA----A-CCTTTTCTTT--------------------------ACTTAAACCAAA |
| Clon_GD2 | GGGGAA----A-CCTTTTCTTT--------------------------ACTTAAACCAAA |
| Clon_GD5 | GGGGAA----A-CCTTTTCTTT--------------------------ACTTAAACCAAA |
| Clon_GD3 | GGGGAA----A-CCTTTTCTTT--------------------------ACTTAAACCAAA |
| Clon_GD12 | GGGGAA----A-CCTTTTCTTT--------------------------ACTTAAACCAAA |
| Clon_GD7 | GGGGAA----A-CCTTTTCTTT--------------------------ACTTAAACCAAA |
| Clon_GD9 | GGGGAA----A-CCTTTTCTTT--------------------------ACTTAAACCAAA |
| Clon_GD8 | GGGGAA----A-CCTTTTCTTT--------------------------ACTTAAACCAAA |
| Clon_GD13 | GGGGAA----A-CCTTTTCTTT--------------------------ACTTAAACCAAA |
| Clon_LD5 | GGGGAA----AACCTTTTCTTT--------------------------ACTTAAACCAAA |
| Clon_LD13 | GGGGAA----AACCTTTTCTTT--------------------------ACTTAAACCAAA |
| Clon_LD14 | GGGGAA----AACCTTTTCTTT--------------------------ACTTAAACCAAA |
| Clon_LD1 | GGGGAA----AACCTTTTCTTT--------------------------ACTTAAACCAAA |
| Clon_LD11 | GGGGAA----AACCTTTTCTTT--------------------------ACTTAAACCAAA |
| Clon_LD3 | GGGGAA----AACCTTTTCTTT--------------------------ACTTAAACCAAA |
| Clon_LD2 | GGGGAA----AACCTTTTCTTT--------------------------ACTTAAACCAAA |
| Clon_LD12 | GGGGAA----AACCTTTTCTTT--------------------------ACTTAAACCAAA |
| Clon_LD8 | GGGGAA----AACCTTTTCTTT--------------------------ACTTAAACCAAA |
| Clon_LD15 | GGGGAA----AACCTTTTCTTT--------------------------ACTTAAACCAAA |
| | * * |
| Clon_DM1 | CATTGAGCCAGT------------------------------ATC-ATTGA--ATCGTTT |
| Clon_DM21 | CATTGAGCCAGT------------------------------ATC-ATTGA--ATCGTTT |
| Clon_DM6 | CATTGAGCCAGT------------------------------ATC-ATTGA--ATCATTA |
| Clon_DM20 | CATTGAGCCAGT------------------------------ATC-ATTGA--ATCATTA |
| Clon_DM9 | CATTGAGCCAGT------------------------------ATC-ATTGA--ATCATTA |
| Clon_DM12 | CATTGAGCCAGT------------------------------ATC-ATTGA--ATCATTA |
| Clon_DM7 | CATTGAGCCAGT------------------------------ATC-ATTGA--ATCATTA |
| Clon_DM11 | CATTGAGCCAGT------------------------------ATC-ATTGA--ATCGTTT |
| Clon_DM14 | CATTGAGCCAGT------------------------------ATC-ATTGA--ATCGTTT |
| Clon_DMA | CATTGAGCCAGT------------------------------ATC-ATTGA--ATCATTA |
| Clon_DF1 | CATTGAGCCAGT------------------------------ATC-ATTGA--ATCAAGT |
| Clon_DF6 | CATTGAGCCAGT------------------------------ATC-ATTGA--AACAATT |
| Clon_DF4 | CATTGAGCCAGT------------------------------ATC-ATTGA--AACAATT |
| Clon_DF26 | CATTGAGCCAGT------------------------------ATC-ATTGA--AACAATT |
| Clon_DF4_50 | CATTGAGCCAGT------------------------------ATC-ATTGA--AACAATT |
| Clon_DF19 | CATTGAGCCAGT------------------------------ATC-ATTGA--AACAATT |
| Clon_DF3 | CATTGAGCCAGT------------------------------ATC-ATTGA--AACAATT |
| Clon_DF5 | CATTGAGCCAGT------------------------------ATC-ATTGA--AACAATT |
| Clon_DF2 | CATTGAGCCAGT------------------------------ATC-ATTGA--AACAATT |
| Clon_DF7 | CATTGAGCCAGT------------------------------ATC-ATTGA--AACAATT |
| Clon_AS15 | CTGTCTCTGGTCGG-------TTACGGTC-----AAAC-GTAGCC-ACCA---GACGTTC |
| Clon_AS14 | CTGTCTCTGGTCGG-------TTACGGTC-----AAAC-GTAGCC-ACCA---GACGTTC |
| Clon_AS20 | CTGTCTCTGGTCGG-------TTACGGTC-----AAAC GTAGCC-ACCA---GACGTTC |
| Clon_AS13 | CTGTCTCTGGTCGG-------TTACGGTC-----ACAC-GTAACC-ACCACCAGACGTTC |
| Clon_AS10 | CTGTCTCTGGTCGG-------TTACGGTC-----ACAC-GTAACC-ACCACCAGACGTTC |
| Clon_AS11 | CTGTCTCTGGTCGG-------TTACGGTC-----ACAC-GTAACC-ACCACCAGACGTTC |
| Clon_AS2 | CTGTCTCTGGTCGG-------TTGCGGTC-----AAAC-GTAGCC-ACCA---GACGTTC |
| Clon_AS12 | CTGTCTCTGGTCGG-------TTACGGTC-----AAAC-GTAGCC-ACCA---GACGTTC |
| Clon_AS1 | CTGTCTCTGGTCGG-------TTACGGTC-----AAAC-GTAGCC-ACCA---GACGTTC |
| Clon_AS16 | CTGTCTCTGGTCGG-------TTACGGTC-----AAAC-GTAGCCCACCA---GACGTTC |
| Clon_BT8 | -----TGTTAAC---------CCCCGGCCCT---AAACCTCAATGCATTT-----CGATG |
| Clon_BT9 | -----TGTTAAC---------CCCCGGCCCT---AAACCTCAATGCATTT-----CGACG |
| Clon_BT16 | -----TGTAAAC---------CCCCGGCCCT---AAACCTCAATGCATTT-----CGGTG |
| Clon_BT3 | -----TGTTAAC---------CCCCGGCCCT---AAACCTCAATGCATTT-----CGATG |
| Clon_BT14 | -----TGTAAAC---------CCCCGGCCCT---AAACCTCAATGCATTT-----CGATG |
| Clon_BT17 | -----TGTAAAC---------CCCCGGCCCT---AAACCTCAATGCATTT-----CGATG |
| Clon_BT13 | -----TGTAAAC---------CCCCGGCCCT---AAACCTCAATGCATTT-----CGGTG |
| Clon_BT1 | -----TGTAAAC---------CCCCGGCCCT---AAACCTCAATGCATTT-----CGGTG |
| Clon_BT10 | -----TGTTAAC---------CCCCGGCCCT---AAACCTCAATGCATTT-----CGATG |
| Clon_BT15 | -----TGTTAAC---------CCCCGGCCCT---AAACCTCAATGCATTT-----CGATG |
| Clon_TPA1_20 | TATCCAGCGGCT-------------------------------CACGCCG-----C-TTG |
| Clon_TPA1_22 | TATCCAGCGGCT-------------------------------CACGCCG-----C-TTG |
| Clon_TPA1_29 | TATCCAGCGGCT-------------------------------CACGCCG-----C-TTG |
| Clon_TPA1_28 | TATCCAGCGGCT-------------------------------CACGCCG-----C-TTG |
| Clon_TPA1_26 | TATCCAGCGGCT-------------------------------CACGCCG-----C-TTG |
| Clon_TPA1_21 | TATCCAGCGGCT-------------------------------CACGTCG-----C-TTG |
| Clon_TPA1_36 | TATCCAGCGGCT-------------------------------CACGCCG-----C-TTG |
| Clon_TPA1_27 | TATCCAGCGGCT-------------------------------CACGCCG-----C-TTG |
| Clon_TPA1_23 | TATCCAGCGGCT-------------------------------CACGTCG-----C-TTG |
| Clon_TPA1_1 | TATCCAGCGGTT-------------------------------CACGCCG-----T-TTG |
| Clon_TF22 | TATCCAGCGGATGCTGCTGCTTCCCAAATTTT--TGGGTGGCGCACGCCG-----TTTTG |
| Clon_TF24 | TATCCAGCGGATGCTGCTGCTTCCCAAATTTT--TGGGTGGCGCACGCCG-----TTTTG |
| Clon_TF3 | TATCCAGCGGATGCTGCTGCTTCCCAAATTTT--TGTGTGGCGCACGCCG-----TTTTG |
| Clon_TF2 | TATCCAGCGGATGCTGCTGCTCCCCAAATTTT--TGGGTTGCGCACGCCG-----TTTTG |
| Clon_TF23 | TATCCAGCGGATGCTGCTGCTCCCCAAATTTT--TGGGTTGCGCACGCCG-----TTTTG |
| Clon_TF1 | TATCCAGCGGATGCTGCTGCTCTCCGAATTTT--CGGGTGGAGCACGCCG-----TTTTG |
| Clon_TF4 | TATCCAGCGGATGCTGCTGCTCTCCGAATTTT--CGGGTGGAGCACGCCG-----TTTTG |
| Clon_TF7 | TATCCAGCGGATGCTGCTGCTCTCCGAATTTT--CGGGTGGAGCACGCCG-----TTTTG |
| Clon_TF15 | TATCCAGCGGATGCTGCTGCTCTCCGAATTTT--CGGGTGGAGCACGCCG-----TTTTG |
| Clon_TF14 | TATCCAGCGGATGCTGCTGCTTCCCGAATTTT--CGGGTGGCGCACGCCG-----TTTTG |
| Clon_DP8 | CTTCAAGCCAG-------------------------------AAACATTGT-TACAAAAC |
| Clon_DP1 | CTTCAAGCCAG-------------------------------AAACATTGT-TACAAAAC |
| Clon_DP7 | CATTGAGCCAG-------------------------------AAACATCGA-TACAAAGC |
| Clon_DP3 | CATTGAGCCAG-------------------------------AAACATTGA-TACAAAGC |
| Clon_DP6 | CATTGAGCCAG-------------------------------AAACATTGA-TACAAAGC |
| Clon_DP9 | CATTGAGCCAG-------------------------------AAACATTGA-TACAAAGC |
| Clon_DP2 | CATTGAGCCAG-------------------------------AAACATTGT-TACAAACC |
| Clon_DP4 | CATTGAGCCAG-------------------------------AAACATTGT-TACAAACC |
| Clon_DP10 | CATTGAGCCAG-------------------------------AAACATTGT-TACAAACC |
| Clon_DP5 | CTTAAAGCCAG-------------------------------AAACATTGT-TACAAAAC |
| Clon_EM4 | CATTGAGCCAG-------------------------------AATCATTG--TATCATTT |
| Clon_EM21 | CATTGAGCCAG-------------------------------AATCATTG--TATCATTT |
| Clon_EM2 | CATTGAGCCAG-------------------------------AATCATTG--TATCATTG |
| Clon_EM23 | CATTGAGCCAG-------------------------------AATCATTG--TATCATTG |
| Clon_EM3 | CATTGAGCCAG-------------------------------AATCATTG--TATCATTG |
| Clon_EM24 | CATTGAGCCAG-------------------------------AATCATTG--TATCATTG |
| Clon_EM22 | CATTGAGCCAG-------------------------------AATCATTG--TATCATTG |
| Clon_EM1 | CATTGAGCCAG-------------------------------AATCATTG--TATCATTT |
| Clon_EM6 | CATTGAGCCAG-------------------------------AATCATTG--TATCATTG |
| Clon_EM5 | CATTGAGCCAG-------------------------------AATCATTG--TATCATTG |
| Clon_GD1 | CTTTAAGCTAAT-------------------------------ACCATTAA-CATTTATG |
| Clon_GD10 | CTTTAAGCTAAT-------------------------------ACCATTAA-CATTTATG |
| Clon_GD2 | CTTTAAGCTAAT-------------------------------ACCATTAA-CATTTATG |
| Clon_GD5 | CTTTAAGCTAAT-------------------------------ACCATTAA-CATTTATG |
| Clon_GD3 | CTTTAAGCTAAT-------------------------------ACCATTAA-CATTTATG |
| Clon_GD12 | CTTTAAGCTAAT-------------------------------ACCATTAA-CATTTATG |
| Clon_GD7 | CTTTAAGCTAAT-------------------------------ACCATTAA-CATTTATG |
| Clon_GD9 | CTTTAAGCTAAT-------------------------------ACCATTAA-CATTTATG |
| Clon_GD8 | CTTTAAGCTAAT-------------------------------ACCATTAA-CATTTATG |
| Clon_GD13 | CTTTAAGCTAAT-------------------------------ACCATTAA-CATTTATG |
| Clon_LD5 | CTTTAAGCTAAT-------------------------------ACCATTAA-CATTTATG |
| Clon_LD13 | CTTTAAGCTAAT-------------------------------ACCATTAA-CATTTATG |
| Clon_LD14 | CTTTAAGCTAAT-------------------------------ACCATTAA-CATTTATG |
| Clon_LD1 | CTTTAAGCTAAT-------------------------------ACCATTAA-CATTTATG |
| Clon_LD11 | CTTTAAGCTAAT-------------------------------ACCATTAA-CATTTATG |
| Clon_LD3 | CTTTAAGCTAAT-------------------------------ACCATTAA-CATTTATG |
| Clon_LD2 | CTTTAAGCTAAT-------------------------------ACCATTAA-CATTTATG |
| Clon_LD12 | CTTTAAGCTAAT-------------------------------ACCATTAA-CATTTATG |
| Clon_LD8 | CTTTAAGCTAAT-------------------------------ACCATTAA-CATTTATG |
| Clon_LD15 | CTTTAAGCTAAT-------------------------------ACCATTAA-CATTTATG |
| Clon_DM1 | GAATTGTGATTCT-----GAAAATGAATC----------GCAAGAA-------------- |
| Clon_DM21 | GAATTGTGATTCT-----GAAAATGAATC----------GCAAGAA-------------- |
| Clon_DM6 | AAATTGTGATTCTCTATAAACGAAGAATC----------GCAAGAA-------------- |
| Clon_DM20 | AAATTGTGATTCTCTATAAACGAAGAATC----------GCAAGAA-------------- |
| Clon_DM9 | AAATTGTGATTCTCTATAAACGAAGAATC----------GCAAGAA-------------- |
| Clon_DM12 | AAATTGTGATTCTCTATAAACGAGGAATC----------GCAAGAA-------------- |
| Clon_DM7 | AAATTGTGATTCTCTATAAACGAAGAATC----------GCAAGAA-------------- |
| Clon_DM11 | CAATTGTGATTAT-----GAAAATAAATC----------GCAAGAA-------------- |
| Clon_DM14 | GAATTGTGATTCT-----GAAAATGAATC----------GCAAGAA-------------- |
| Clon_DMA | AAATTGTGATTCTCTATAAACGAAGAATC----------GCAAGAA-------------- |
| Clon_DF1 | GAATTGTGATTCT-----GAAAATGAATC----------GCAAGAA-------------- |
| Clon_DF6 | GAATTGTGATTCT-----GCAAATGAATC----------GCAAGAA-------------- |
| Clon_DF4 | GAATTGTGATTCT-----GCAAATGAATC----------GCATGAA-------------- |
| Clon_DF26 | GAATTGTGATTCT-----GCAAATGAATC----------GCATGAA-------------- |
| Clon_DF4_50 | GAATTGTGATTCT-----GCAAATGAATC----------GCAAGAA-------------- |
| Clon_DF19 | GAATTGTGATTCT-----GCAAATGAATC----------GCAAGAA-------------- |
| Clon_DF3 | AAATTGTGATTCT-----GCAAATGAATC----------GCAAGAA-------------- |
| Clon_DF5 | GAATTGTGATTCT-----GCAAATGGATC----------GCAAAAA-------------- |
| Clon_DF2 | AAATTGTGATTCT-----GCAAATGAATC----------GCAAGAA-------------- |
| Clon_DF7 | GAATTGTGATTCT-----GCAAATGGATC----------GCAAAAA-------------- |
| Clon_AS15 | AGT-TTTCATTCTCT----AAACTCAACTT-----A--AGCCAAAATCAAAACTGATTAA |
| Clon_AS14 | AGT-TTTCATTCTCT----AAACTCAACTT-----A--AGCCAAAATCAAAACTGATTAA |
| Clon_AS20 | AGT-TTTCATTCTCT----AAACTCAACTT-----A--AGCCAAAATCAAAACTGATTAA |
| Clon_AS13 | AGT-TTTCATTCTCT----AAACTCAACTT-----A--AGCCAAAATCAAAACTGATTAA |
| Clon_AS10 | AGT-TTTCATTCTCT----AAACTCAACTT-----A--AGCCAAAATCAAAACTGATTAA |
| Clon_AS11 | AGT-TTTCATTCTCT----AAACTCAACTT-----A--AGCCAAAATCAAAACTGATTAA |
| Clon_AS2 | AGT-TTTCATTCTCT----AAACTCAACTT-----A--AGCCAAAATCAAAACTGATTAA |
| Clon_AS12 | AGT-TTTCATTCTCT----AAACTCAACTT-----A--AGCCAAAATCAAAACTGATTAA |
| Clon_AS1 | AGT-TTTCATTCTCT----AAACTCAACTT-----A--AGCCAAAATCAAAACTGATTAA |
| Clon_AS16 | AGT-TTTCATTCTCT----AAACTCAACTT-----A--AGCCAAAATCAAAACTGATTAA |
| Clon_BT8 | CAT-TGTTGTATTAT----AACCAAAATTT-----A--AGCTAA----AAAACTAAATAA |
| Clon_BT9 | CAT-TGTTGTATTCT----AACCAAAATTT-----A--AGCTAA----AAAACTAAATAA |
| Clon_BT16 | CAT-TGTTGTACTTT----AACCAAAATTT-----A--AGCTAA----AAAACTAAATAA |
| Clon_BT3 | CAT-TGTTGTATTCT----AACCAAAATTT-----A--AGCTAA----AAAACTAAATAA |
| Clon_BT14 | CAT-TGTTGTATTAT----AACCAAAAAT------A--AGCTAAGA--AAAATAAAACAA |
| Clon_BT17 | CAT-TGTTGTATTAT----AACCAAAAAT------A--AGCTAAGA--AAAATAAAACAA |
| Clon_BT13 | CAT-TGTTGTACTTT----AACCAAAAAT------A--AGCTAAAA--CAAATAAAACAA |
| Clon_BT1 | CAT-TGTTGTACTTT----AACCAAAATTT-----A--AGCTAA----AAAACTAAATAA |
| Clon_BT10 | CAT-TGTTGTATTCT----AACCAAAATTT-----A--AGCTAA----AAAACTAAATAA |
| Clon_BT15 | CAT-TGTTGTATTCT----AACCAAAATTT-----A--AGCTAA----AAAACTAAATAA |
| Clon_TPA1_20 | GGTACTTCATTCTCT----AACCCCAACTT-----ACAAGCCAAAATCACGA-TGATTTA |
| Clon_TPA1_22 | GGTACTTCATTCTCT----AACCCCAACTT-----ACAAGCCAAAATCACGA-TGATTTA |
| Clon_TPA1_29 | GGTACTTCATTCTCT----AACCCCAACTT-----ACAAGCCAAAATCACGA-TGATTTA |
| Clon_TPA1_28 | GGTACTTCATTCTCT----AACCCCAACTT-----ACAAGCCAAAATCACGA-TGATTTA |
| Clon_TPA1_26 | GGTACTTCATTCTCT----AACCTCAACTT-----ACAAGCCAAAATCACGA-TGATTTA |
| Clon_TPA1_21 | GGTACTTCATTCTCT----AACCCCAACTT-----ACAAGCCAAAATCACGA-TGATTTA |
| Clon_TPA1_36 | GGTACTTCATTCTCT----AACCCCAACTT-----ACAAGCCAAAATCACGA-TGATTTA |
| Clon_TPA1_27 | GGTACTTCATTCTCT----AAACCCAACTT-----ACAAGCCAAAATCACGA-TGATTTA |
| Clon_TPA1_23 | GGTACTTCATTCTCT----AACCCCAACTT-----ACAAGCCAAAATCACGA-TGATTTA |
| Clon_TPA1_1 | GGTACTTCATTCTCT----AACCCCAACTT-----ACAAGCCAAAATCACGA-TGATTTA |
| Clon_TF22 | GGTACTTCATTCTCT----AACCCCAACTT-----ACAAGCCAAAATCATGA-TGATTTA |
| Clon_TF24 | GGTACTTCATTCTCT----AACCCCAACTT-----ACAAGCCAAAATCATGA-TGATTTA |
| Clon_TF3 | GGTACTTCATTCTCT----AACCCCAACTT-----ACAAGCCAAAATCATGA-TGATTTA |
| Clon_TF2 | GGTACTTCATTCTCT----AACCCCAACTT-----ACAAGCCAAAATCATGA-TGATTTA |
| Clon_TF23 | GGTACTTCATTCTCT----AACCCCAACTT-----ACAAGCCGAAATCATGA-TGATTTA |
| Clon_TF1 | GGTACTTCATTCTCT----AACCCCAACTT-----ACAAGCCAAAATCATGA-TGATTTA |
| Clon_TF4 | GGTACTTCATTCTCT----AACCCCAACTT-----ACAAGCCAAAATCATGA-TGATTTA |
| Clon_TF7 | GGTACTTCATTCTCT----AACCCCAACTT-----ACAAGCCAAAATCATGA-TGATTTA |
| Clon_TF15 | GGTACTTCATTCTCT----AACCCCAACTT-----ACAAGCCAAAATCATGA-TGATTTA |
| Clon_TF14 | GGTACTTCATTCTCT----AACCCCAACTT-----ACAAGCCAAAATCATGA-TGATTTA |
| Clon_DP8 | AAATTGTGATTCTCG----AA--AGAATT----------GCAAAAA-------------- |
| Clon_DP1 | AAATTGTGATTCTCG----AA--AGAATT----------GCAAAAA-------------- |
| Clon_DP7 | AAATTGTGATTCTGC----AA--AGAATT----------GCAAAAA-------------- |
| Clon_DP3 | AAATTGTGATTCTGC----AA--AGAATT----------GCAAAAA-------------- |
| Clon_DP6 | AAATTGTGATTCTGC----AA--AGAATT----------GCAAAAA-------------- |
| Clon_DP9 | AAATTGTGATTCTGC----AA--AGAATT----------GCAAAAA-------------- |
| Clon_DP2 | AAATTGTGATTCTGC----AA--AGAATT----------GCAAAAA-------------- |
| Clon_DP4 | AAATTGTGGTTCTGC----AA--AGAATT----------GCAAAAA-------------- |
| Clon_DP10 | AAATTGTGATTCTAC----AA--AGAATT----------GCAAAAA-------------- |
| Clon_DP5 | AAGTTGTGATTCTCG----AA--AGAATT----------GCAATAA-------------- |
| Clon_EM4 | AATTTGTGATTCTCT----ACGTAGAATT----------GCAAACA-------------- |
| Clon_EM21 | AATTTGTGATTCTCT----ACGTAGAATT----------GCAAACA-------------- |
| Clon_EM2 | AATTTGTGATTCTCT----ACGTAGAATT----------GCAAACA-------------- |
| Clon_EM23 | AATTTGTGATTCTCT----ACGCAGAATT----------GCAAACA-------------- |
| Clon_EM3 | AATTTGTGATTCTCT----ACGTAGAATT----------GCAAACA-------------- |
| Clon_EM24 | AATTTGTGATTCTCT----ACGTAGAATT----------GCAAACA-------------- |
| Clon_EM22 | AATTTGTGATTCTCT----ACGTAGAATT----------GCAAACA-------------- |
| Clon_EM1 | AATTTGTGATTCTCT----ACGTAGAATT----------GCAAACA-------------- |
| Clon_EM6 | AATTTGTGATTCTCT----ACGTAGAATT----------GCAAACA-------------- |
| Clon_EM5 | AATTTGTGATTCTCT----ACGTAGAATT----------GCAAACA-------------- |
| Clon_GD1 | GAATTGT-ATTACCTAT-TGGATGCACTT----GTGCTGATGATAGAGATAC-------- |
| Clon_GD10 | GAATTGT-ATTACCTAT-TGGATGCACTT----GTGCTGATGATAGAGATAC-------- |
| Clon_GD2 | GAATTGT-ATTACCTAT-TGGATGCACATTCGTGTGCTGATAATAGAGATAC-------- |
| Clon_GD5 | GAATTGT-ATTACCTAT-TGGATGCACATTCGTGTGCTGATAATAGAGATAC-------- |
| Clon_GD3 | GAATTGT-ATTACCTAT-TGGATGCACTT----GTGCTGATAATAGAGATAC-------- |
| Clon_GD12 | GAATTGT-ATTACCTAT-TGGATGCACTT----GTGCTGATAATAGAGATAC-------- |
| Clon_GD7 | GAATTGT-ATTACCTAT-TGGATGCACTT----GTGCTGATAATAGAGATAC-------- |
| Clon_GD9 | GAATTGT-ATTACCTAT-TGGATGCACTT----GTGCTGATAATAGAGATAC-------- |
| Clon_GD8 | GAATTGT-ATTACCTAT-TGGATGCACTT----GTGCTGATAATAGAGATAC-------- |
| Clon_GD13 | GAATTGT-ATTACCTAT-TGGATGCACTT----GTGCTGATGATAGAGATAC-------- |
| Clon_LD5 | GAATTGT-ATTGTCT---TGAAATANITTT----TAATTATTAAAGCGATGC-------- |
| Clon_LD13 | GAATTGT-ATTGTCT---TGAAATANITTT----TAATTATTAAAGCGATGC-------- |
| Clon_LD14 | GAATTGT-ATTGTCT---TGAAATANITTT----TAATTATTAAAGCGATGC-------- |
| Clon_LD1 | GAATTGT-ATTGTCT---TGAAATANITTT----TAATTATTAAAGCGATGC-------- |
| Clon_LD11 | GAATTGT-ATTGTCT---TGAAATANITTT----TAATTATTAAAGCGATGC-------- |
| Clon_LD3 | GAATTGT-ATTGTCT---TGAAATANITTT----TAATTATTAAAGAGATGC-------- |
| Clon_LD2 | GAATTGT-ATTGTCT---TGAAATANITTT----TAATTATTAAAGCGATGC-------- |
| Clon_LD12 | GAATTGT-ATTGTCT---TGAAATANITTT----TAATTATTAAAGAGATGC-------- |
| Clon_LD8 | GAATTGT-ATTGTCT---TGAAATANITTT----TAATTATTAAAGCGATGC-------- |
| Clon_LD15 | GAATTGT-ATTGTCT---TGAAATANITTT----TAATTATTAAAGCGATGC-------- |
| | * * |
| Clon_DM1 | ---------------------------------------------AGCAAAAA-----TT |
| Clon_DM21 | ---------------------------------------------AGCAAAAA-----TT |
| Clon_DM6 | ---------------------------------------------AGCAAAAA-----TT |
| Clon_DM20 | ---------------------------------------------AGCAAAAA-----TT |
| Clon_DM9 | ---------------------------------------------AGCAAAAA-----TT |
| Clon_DM12 | ---------------------------------------------AGCAAAAA-----TT |
| Clon_DM7 | ---------------------------------------------AGCAAAAA-----TT |
| Clon_DM11 | ---------------------------------------------AGCAAAAA-----TT |
| Clon_DM14 | ---------------------------------------------AGCAAAAA-----TT |
| Clon_DMA | ---------------------------------------------AGCAAAAA-----TT |
| Clon_DF1 | ---------------------------------------------AGCAAAAA-----TT |
| Clon_DF6 | ---------------------------------------------AGCAAAAA-----TT |
| Clon_DF4 | ---------------------------------------------AGCAAAAA-----TT |
| Clon_DF26 | ---------------------------------------------AGCAAAAA-----TT |
| Clon_DF4_50 | ---------------------------------------------AGCAAAAA-----TT |
| Clon_DF19 | ---------------------------------------------AGCAAAAA-----TT |
| Clon_DF3 | ---------------------------------------------AGCAAAAA-----TT |
| Clon_DF5 | ---------------------------------------------AGCAAAAA-----TT |
| Clon_DF2 | ---------------------------------------------AGCAAAAA-----TT |
| Clon_DF7 | ---------------------------------------------AGCAAAAA-----TT |
| Clon_AS15 | CC--TTAAATTGTACCCTTA-TGGGTCAA------------TTTATAAAAAGAC--AATT |
| Clon_AS14 | CC--TTAAATTGTACCCTTA-TGGGTCAA------------TTTATAAAAAGAC--AATT |
| Clon_AS20 | CC--TTAAATTGTACCCTTA-TGGGTCAA------------TTTATAAAAAGAC--AATT |
| Clon_AS13 | CC--TTAAATTGTACCCTTA-TGGGTCAA------------TTTATAAAAAGAC--AATT |
| Clon_AS10 | CC--TTAAATTGTACCCTTA-TGGGTCAA------------TTTATAAAAAGAC--AATT |
| Clon_AS11 | CC--TTAAATTGTACCCTTA-TGGGTCAA------------TTTATAAAAAGAC--AATT |
| Clon_AS2 | CC--TTAAATTGTACCCTTA-TGGGTCAA------------TTTATAAAAAGAC--AATT |
| Clon_AS12 | CC--TTAAATTGTACCCTTA-TGGGTCAA------------TTTATAAAAAGAC--AATT |
| Clon_AS1 | CC--TTAAATTGTACCCTTA-TGGGTCAA------------TTTATAAAAAGAC--AATT |
| Clon_AS16 | CC--TTAAATTGT--CCTTA-TCG-TCAA------------TTTATAAAAAGAC--AATT |
| Clon_BT8 | -----CAAA-----------------------------------ACAAAAAAA------T |
| Clon_BT9 | -----CAAA-----------------------------------ACAAAAAAA------T |
| Clon_BT16 | -----CAAA-----------------------------------ACAAAAAAAA--A--T |
| Clon_BT3 | -----CAAA-----------------------------------ACAAAAAAA------T |
| Clon_BT14 | A---TCAAA-----------------------------------ATAAAAAA-------T |
| Clon_BT17 | A---TCAAA-----------------------------------ATAAAAAA-------T |
| Clon_BT13 | A---TCAAA-----------------------------------ATATAAAA-------T |
| Clon_BT1 | -----CAAA-----------------------------------ACAAAAAAA------T |
| Clon_BT10 | -----CAAA-----------------------------------ACAAAAAA-------T |
| Clon_BT15 | -----AACA-----------------------------------A--AAAAA-------T |
| Clon_TPA1_20 | CCCTTTAAGT-GTTACCTAG--TGT--AAG-----------CTTAAACAATGACAGAATT |
| Clon_TPA1_22 | CCCTTTAAGT-GTTACCTAG--TGT--AAG-----------CTTAAACAATGACAGAATT |
| Clon_TPA1_29 | CCCTTTAAGT-GTTACCTAG--TGT--AAG-----------CTTAAACAATGACAGAATT |
| Clon_TPA1_28 | CCCTTTAAGT-GTTACCTAG--TGT--AAG-----------CTTAAACAATGACAGAATT |
| Clon_TPA1_26 | CCCTTTAAGT-GTTACCTCG--TGT--AAG-----------CTTAAACAATGACAGAATT |
| Clon_TPA1_21 | CCCTTTAAGT-GTTACCTCG--TGT--AAG-----------CTTAAACAATGACAGAATT |
| Clon_TPA1_36 | CCCTTTAAGT-GTTACCTAG--TGT--AAG-----------CTTAAACAATGACAGAATT |
| Clon_TPA1_27 | CCCTTTAAGT-GTTACCTCG--TGT--AAG-----------CTTAAACAATGACAGAATT |
| Clon_TPA1_23 | CCCTTTAAGT-GTTACCTCG--TGT--AAA-----------CTTAAACAATGACAGAATT |
| Clon_TPA1_1 | CCCTTTAAGT-GTTACCTCG--TGT--AAG-----------CTTAAACAATGACAGAATT |
| Clon_TF22 | CCCTTTAAGT-TTTACCTTG--TGT--AAG-----------CTTAAACAATGACAAAATT |
| Clon_TF24 | CCCTTTAAGT-TTTACCTTG--TGT--AAG-----------CTTAAACAATGACAAAATT |
| Clon_TF3 | CCCTTTAAGT-TTTACCTTG--TGT--AAG-----------CTTAAACAATGACAAAATT |
| Clon_TF2 | CCCTTTAAGT-TTTACCTTG--TGT--AAG-----------CTTAAACAATGACAGAATT |
| Clon_TF23 | CCCTTTAAGT-TTTACCTTG--TGT--AAG-----------CTTAAACAATGACAGAATT |
| Clon_TF1 | CCCTTTAAGT-TTTACCTTG--TGT--AAG-----------CTTAAACAATGACAGAATT |
| Clon_TF4 | CCCTTTAAGT-TTTACCTTG--TGT--AAG-----------CTTAAACAATGACAGAATT |
| Clon_TF7 | CCCTTTAAGT-TTTACCTTG--TGT--AAG-----------CTTAAACAATGACAGAATT |
| Clon_TF15 | CCCTTTAAGT-TTTACCTTG--TGT--AAG-----------CTTAAACAATGACAGAATT |
| Clon_TF14 | CCCTTTAAGT-TTTACCTTG--TGT--AAG-----------CTTAAACAATGACAGAATT |
| Clon_DP8 | ----------------------------------------------ACAAAAA-----TA |
| Clon_DP1 | ----------------------------------------------ACAAAAA-----TA |
| Clon_DP7 | ----------------------------------------------ACAAAAA-----TA |
| Clon_DP3 | ----------------------------------------------ACAAAAA-----TA |
| Clon_DP6 | ----------------------------------------------ACAAAAA-----TA |
| Clon_DP9 | ----------------------------------------------ACAAAAA-----TA |
| Clon_DP2 | ----------------------------------------------ACAAAT------TA |
| Clon_DP4 | ----------------------------------------------ACAAAAA-----TA |
| Clon_DP10 | ----------------------------------------------ACAAGAA-----TA |
| Clon_DP5 | ----------------------------------------------ACAAATA-----TA |
| Clon_EM4 | ----------------------------------------------AAAAAAA-----TA |
| Clon_EM21 | ----------------------------------------------AAAAAAA-----TA |
| Clon_EM2 | ----------------------------------------------AAAAAAA-----TA |
| Clon_EM23 | ----------------------------------------------AAAAAA------TA |
| Clon_EM3 | ----------------------------------------------AAAAAAA-----TA |
| Clon_EM24 | ----------------------------------------------AAAAAAA-----TA |
| Clon_EM22 | ----------------------------------------------AAAAAAAA----TA |
| Clon_EM1 | ----------------------------------------------AAAAAAA-----TA |
| Clon_EM6 | ----------------------------------------------AAAAAAA-----TA |
| Clon_EM5 | ----------------------------------------------AAAAAAA-----TA |
| Clon_GD1 | ---------------------------------------------AAAAGAGAT--AAAT |
| Clon_GD10 | ---------------------------------------------AAAAGAGAT--AAAT |
| Clon_GD2 | ---------------------------------------------AAAAGAGAT--AAAT |
| Clon_GD5 | ---------------------------------------------AAAAGAGAT--AAAT |
| Clon_GD3 | ---------------------------------------------AAAAGAGAT--AAAT |
| Clon_GD12 | ---------------------------------------------AAAAGAGAT--AAAT |
| Clon_GD7 | ---------------------------------------------AAAAGAGAT--AAAT |
| Clon_GD9 | ---------------------------------------------AAAAGAGAT--AAAT |
| Clon_GD8 | ---------------------------------------------AAAAGAGAT--AAAT |
| Clon_GD13 | ---------------------------------------------AAAAGAGAT--AAAT |
| Clon_LD5 | ---------------------------------------------AAAAGAGAT--AAAT |
| Clon_LD13 | ---------------------------------------------AAAAGAGAT--AAAT |
| Clon_LD14 | ---------------------------------------------AAAAGAGAT--AAAT |
| Clon_LD1 | ---------------------------------------------AAAAGAGAT--AAAT |
| Clon_LD11 | ---------------------------------------------AAAAGAGAT--AAAT |
| Clon_LD3 | ---------------------------------------------AAAAGAGAT--AAAT |
| Clon_LD2 | ---------------------------------------------AAAAGAGAT--AAAT |
| Clon_LD12 | ---------------------------------------------AAAAGAGAT--AAAT |
| Clon_LD8 | ---------------------------------------------AAAAGAGAT--AAAT |
| Clon_LD15 | ---------------------------------------------AAAAGAGAT--AAAT |
| | * |
| Clon_DM1 | ACTACTGCCAGTGGTGGATCACTCGGCTCGCTGGTCGAGGAAGAACGTAGCTAGCTACGT |
| Clon_DM21 | ACTACTGCCAGTGGTGGATCACTCGGCTCGCTGGTCGAGGAAGAACGTAGCTAGCTACGT |
| Clon_DM6 | ACTACTGCCAGTGGTGGATCACTCGGCTCGCTGGTCGAGGAAGAACGTAGCTAGCTACGT |
| Clon_DM20 | ACTACTGCCAGTGGTGGATCACTCGGCTCGCTGGTCGAGGAAGAACGTAGCTAGCTACGT |
| Clon_DM9 | ACTACTGCCAGTGGTGGATCACTCGGCTCGCTGGTCGAGGAAGAACGTAGGTAGCTACGT |
| Clon_DM12 | ACTACTGCCAGTGGTGGATCACTCGGCTCGCTGGTCGAGGAAGAACGTAGCTAGCTACGT |
| Clon_DM7 | ACTACTGCCAGTGGTGGATCACTCGGCTCGCTGGTCGAGGAAGAACGTAGCTAGCTACGT |
| Clon_DM11 | ACTACTGCCAGTGGTGGATCACTCGGCTCGCTGGTCGAGGAAGAACGTAGCTAGCTACGT |
| Clon_DM14 | ACTACTGCCAGTGGTGGATCACTCGGCTCGCTGGTCGAGGAAGAACGTAGCTAGCTACGT |
| Clon_DMA | ACTACTGCCAGTGGTGGATCACTCGGCCCGCTGGTCGAGGAAGAACGTAGCTAGCTACGT |
| Clon_DF1 | ACTACTGCCAGTGGTGGATCACTCGGCTCGCTGGTCGAGGAAGAACGTAGCTAGCTACGT |
| Clon_DF6 | ACTACTGCCAGTGGTGGATCACTCGGCTCGCTGGTCGAGGAAGAACGTAGCTAGCTACGT |
| Clon_DF4 | ACTACTGCCAGTGGTGGATCACTCGGCTCGCTGGTCGAGGAAGAACGTAGCTAGCTACGT |
| Clon_DF26 | ACTACTGCCAGTGGTGGATCACTCGGCTCGCTGGTCGAGGAAGAACGTAGCTAGCTACGT |
| Clon_DF450 | ACTACTGCCAGTGGTGGATCACTCGGCTCGCTGGTCGAGGAAGAACGTAGCTAGCTACGT |
| Clon_DF19 | ACTACTGCCAGTGGTGGATCACTCGGCTCGCTGGTCGAGGAAGAACGTAGCTAGCTACGT |
| Clon_DF3 | ACTACTGCCAGTGGTGGATCACTCGGCTCGCTGGTCGAGGAAGAACGTAGCTAGCTACGT |
| Clon_DF5 | ACTACTGCCAGTGGTGGATCACTCGGCTCGCTGGTCGAGGAAGAACGTAGCTAGCTACGT |
| Clon_DF2 | ACTACTGCCAGTGGTGGATCACTCGGCTCGCTGGTCGAGGAAGAACGTAGCTAGCTACGT |
| Clon_DF7 | ACTACTGCCAGTGGTGGATCACTCGGCTCGCTGGTCGAGGAAGAACGTAGCTAGCTACGT |
| Clon_AS15 | ACAACTGTTAGTGGTGGATCACTCGGCACGCTGATCGAGGAAGAACGCAGCTAGCTGCGT |
| Clon_AS14 | ACAACTGTTAGTGGTGGATCACTCGGCACGCTGATCGAGGAAGAACGCAGCTAGCTGCGT |
| Clon_AS20 | ACAACTGTTAGTGGTGGATCACTCGGCACGCTGATCGAGGAAGAACGCAGCTAGCTGCGT |
| Clon_AS13 | ACAACTGTTAGTGGTGGATCACTCGGCACGCTGATCGAGGAAGAACGCAGCTAGCTGCGT |
| Clon_AS10 | ACAACTGTTAGTGGTGGATCACTCGGCACGCTGATCGAGGAAGAACGCAGCTAGCTGCGT |
| Clon_AS11 | ACAACTGTTAGTGGTGGATCACTCGGCACGCTGATCGAGGAAGAACGCAGCTAGCTGCGT |
| Clon_AS2 | ACAACTGTTAGTGGTGGATCACTCGGCACGCTGATCGAGGAAGAACGCAGCTAGCTGCGT |
| Clon_AS12 | ACAACTGTTAGTGGTGGATCACTCGGCACGCTGATCGAGGAAGAACGCAGCTAGCTGCGT |
| Clon_AS1 | ACAACTGTTAGTGGTGGATCACTCGGCACGCTGATCGAGGAAGAACGCAGCTAGCTGCGT |
| Clon_AS16 | ACAACTGTTAGTGGTGGATCACTCGGCACGCTGATCGAGGAAGAACGCAGCTAGCTGCGT |
| Clon_BT8 | ATAGCTGTTAGTGGTGGATCACTCGGCTCGCTGGTCGAGGAAGAACGCAGCTAGCTGCGT |
| Clon_BT9 | ACAGCTGTTAGTGGTGGATCACTCGGCTCGCTGGTCGAGGAAGAACGCAGCTAGCTGCGT |
| Clon_BT16 | ACAGCTGTTAGTGGTGGATCACTCGGCTCGCTGGTCGAGGAAGAACGCAGCTAGCTGCGT |
| Clon_BT3 | ATAGCTGTTAGTGGTGGATCACTCGGCTCGCTGGTCGAGGAAGAACGCAGCTAGCTGCGT |
| Clon_BT14 | ATAGCTGTTAGTGGTGGATCACTCGGCTCGCTGGTCGAGGAAGAACGCAGCTAGCTGCGT |
| Clon_BT17 | ACAGCTGTTAGTGGTGGATCACTCGGCTCGCTGGTCGAGGAAGAACGCAGCTAGCTGCGT |
| Clon_BT13 | ACAGCTGTTAGTGGTGGATCACTCGGCTCGCTGGTCGAGGAAGAACGCAGCTAGCTGCGT |
| Clon_BT1 | ACAGCTGTTAGTGGTGGATCACTCGGCTCGCTGGTCGAGGAAGAACGCAGCTAGCTGCGT |
| Clon_BT10 | ACAGCTGTTAGTGGTGGATCACTCGGCTCGCTGGTCGAGGAAGAACGCAGCTAGCTGCGT |
| Clon_BT15 | ACAGCTGTTAGTGGTGGATCACTCGGCTCGCTGGTCGAGGAAGAACGCAGCTAGCTGCGT |
| Clon_TPA1_20 | ACAACTGTTAGTGGTGGATCACTCGGCACGCTGATCGAGGAAGAACGCAGCTAGCTGCGT |
| Clon_TPA_22 | ACAACTGTTAGTGGTGGATCACTCGGCACGCTGATCGAGGAAGAACGCAGCTAGCTGCGT |
| Clon_TPA1_29 | ACAACTGTTAGTGGTGGATCACTCGGCACGCTGATCGAGGAAGAACGCAGCTAGCTGCGT |
| Clon_TPA1_28 | ACAACTGTTAGTGGTGGATCACTCGGCACGCTGATCGAGGAAGAACGCAGCTAGCTGCGT |
| Clon_TPA1_26 | ACAACTGTTAGTGGTGGATCACTCGGCACGCTGATCGAGGAAGAACGCAGCTAGCTGCGT |
| Clon_TPA1_21 | ACAACTGTTAGTGGTGGATCACTCGGCACGCTGATCGAGGAAGAACGCAGCTAGCTGCGT |
| Clon_TPA1_36 | ACAACTGTTAGTGGTGGATCACTCGGCACGCTGATCGAGGAAGAACGCAGCTAGCTGCGT |
| Clon_TPA1_27 | ACAACTGTTAGTGGTGGATCACTCGGCACGCTGATCGAGGAAGAACGCAGCTAGCTGCGT |
| Clon_TPA1_23 | ACAACTGTTAGTGGTGGATCACTCGGCACGCTGATCGAGGAAGAACGCAGCTAGCTGCGT |
| Clon_TPA1_1 | ACAACTGTTAGTGGTGGATCACTCGGCACGCTGATCGAGGAAGAACGCAGCTAGCTGCGT |
| Clon_TF22 | ACAACTGTTAGTGGTGGATCACTCGGCACGCTGATCGAGGAAGAACGCAGCTAGCTGCGT |
| Clon_TF24 | ACAACTGTTAGTGGTGGATCACTCGGCACGCTGATCGAGGAAGAACGCAGCTAGCTGCGT |
| Clon_TF3 | ACAACTGTTAGTGGTGGATCACTCGGCACGCTGATCGAGGAAGGACGCAGCTAGCTGCGT |
| Clon_TF2 | ACAACTGTTAGTGGTGGATCACTCGGCACGCTGATCGAGGAAGAACGCAGCTAGCTGCGT |
| Clon_TF23 | ACAACTGTTAGTGGTGGATCACTCGGCACGCTGATCGAGGAAGAACGCAGCTAGCTGCGT |
| Clon_TF1 | ACAACTGTTAGTGGTGGATCACTCGGCACGCTGATCGAGGAAGAACGCAGCTAGCTGCGT |
| Clon_TF4 | ACAACTGTTAGTGGTGGATCACTCGGCACGCTGATCGAGGAAGAACGCAGCTAGCTGCGT |
| Clon_TF7 | ACAACTGTTAGTGGTGGATCACTCGGCACGCTGATCGAGGAAGAACGCAGCTAGCTGCGT |
| Clon_TF15 | ACAACTGTTAGTGGTGGATCACTCGGCACGCTGATCGAGGAAGAACGCAGCTAGCTGCGT |
| Clon_TF14 | ACAACTGTTAGTGGTGGATCACTCGGCACGCTGATCGAGGAAGAACGCAGCTAGCTGCGT |
| Clon_DP8 | ACTACTGCCAGTGGTGGATCACTCGGCTCGCTGGTCGAGGAAGAACGCAGCTAGCTGCGT |
| Clon_DP1 | ACTACTGCCAGTGGTGGATCACTCGGCTCGCTGGTCGAGGAAGAACGCAGCTAGCTGCGT |
| Clon_DP7 | ACTACTGCCAGTGGTGGATCACTCGGCTCGCTGGTCGAGGAAGAACGCAGCTAGCTGCGT |
| Clon_DP3 | ACTACTGCCAGTGGTGGATCACTCGGCTCGCTGGTCGAGGAAGAACGCAGCTAGCTGCGT |
| Clon_DP6 | ACTACTGCCAGTGGTGGATCACTCGGCTCGCTGGTCGAGGAAGAACGCAGCTAGCTGCGT |
| Clon_DP9 | ACTACTGCCAGTGGTGGATCACTCGGCTCGCTGGTCGAGGAAGAACGCAGCTAGCTGCGT |
| Clon_DP2 | ACTACTGCCAGTGGTGGATCACTCGGCTCGCTGGTCGAGGAAGAACGCAGCTAGCTGCGT |
| Clon_DP4 | ACTACTGCCAGTGGTGGATCACTCGGCTCGCTGGTCGAGGAAGAACGCAGCTAGCTGCGT |
| Clon_DP10 | ACTACTGCCAGTGGTGGATCACTCGGCTCGCTGGTCGAGGAAGAACGCAGCTAGCTGCGT |
| Clon_DP5 | ACTACTGCCAGTGGTGGATCACTCGGCTCGCTGGTCGAGGAAGAACGCAGCTAGCTGCGT |
| Clon_EM4 | ACTACTGCCAGTGGTGGATCACTCGGCTCGCTGGTCGAGGAAGAACGCAGCTAGCTGCGT |
| Clon_EM21 | ACTACTGCCAGTGGTGGATCACTCGGCTCGCTGGTCGAGGAAGAACGCAGCTAGCTGCGT |
| Clon_EM2 | ACTACTGCCAGTGGTGGATCACTCGGCTCGCTGGTCGAGGAAGAACGCAGCTAGCTGCGT |
| Clon_EM23 | ACTACTGCCAGTGGTGGATCACTCGGCTCGCTGGTCGAGGAAGAACGCAGCTAGCTGCGT |
| Clon_EM3 | ACTACTGCCAGTGGTGGATCACTCGGCTCGCTGGTCGAGGAAGAACGCAGCTAGCTGCGT |
| Clon_EM24 | ACTACTGCCAGTGGTGGATCACTCGGCTCGCTGGTCGAGGAAGAACGCAGCTAGCTGCGT |
| Clon_EM22 | ACTACTGCCAGTGGTGGATCACTCGGCTCGCTGGTCGAGGAAGAACGCAGCTAGCTGCGT |
| Clon_EM1 | ACTACTGCCAGTGGTGGATCACTCGGCTCGCTGGTCGAGGAAGAACGCAGCTAGCTGCGT |
| Clon_EM6 | ACTACTGCCAGTGGTGGATCACTCGGCTCGCTGGTCGAGGAAGAACGCAGCTAGCTGCGT |
| Clon_EM5 | ACTACTGCCAGTGGTGGATCACTCGGCTCGCTGGTCGAGGAAGAACGCAGCTAGCTGCGT |
| Clon_GD1 | ACTACTGCCAGTGGTGGATCACTCGGCTCGCTGGTCGAGGAAGAACGCAGCTAGCTGCGT |
| Clon_GD10 | ACTACTGCCAGTGGTGGATCACTCGGCTCGCTGGTCGAGGAAGAACGCAGCTAGCTGCGT |
| Clon_GD2 | ACTACTGCCAGTGGTGGATCACTCGGCTCGCTGGTCGAGGAAGAACGCAGCTAGCTGCGT |
| Clon_GD5 | ACTACTGCCAGTGGTGGATCACTCGGCTCGCTGGTCGAGGAAGAACGCAGCTAGCTGCGT |
| Clon_GD3 | ACTACTGCCAGTGGTGGATCACTCGGCTCGCTGGTCGAGGAAGAACGCAGCTAGCTGCGT |
| Clon_GD12 | ACTACTGCCAGTGGTGGATCACTCGGCTCGCTGGTCGAGGAAGAACGCAGCTAGCTGCGT |
| Clon_GD7 | ACTACTGCCAGTGGTGGATCACTCGGCTCGCTGGTCGAGGAAGAACGCAGCTAGCTGCGT |
| Clon_GD9 | ACTACTGCCAGTGGTGGATCACTCGGCTCGCTGGTCGAGGAAGAACGCAGCTAGCTGCGT |
| Clon_GD8 | ACTACTGCCAGTGGTGGATCACTCGGCTCGCTGGTCGAGGAAGAACGCAGCTAGCTGCGT |
| Clon_GD13 | ACTACTGCCAGTGGTGGATCACTCGGCTCGCTGGTCGAGGAAGAACGCAGCTAGCTGCGT |
| Clon_LD5 | ACTACTGCCAGTGGTGGATCACTCGGCTCGCTGGTCGAGGAAGAACGCAGCTAGCTGCGT |
| Clon_LD13 | ACTACTGCCAGTGGTGGATCACTCGGCTCGCTGGTCGAGGAAGAACGCAGCTAGCTGCGT |
| Clon_LD14 | ACTACTGCCAGTGGTGGATCACTCGGCTCGCTGGTCGAGGAAGAACGCAGCTAGCTGCGT |
| Clon_LD1 | ACTACTGCCAGTGGTGGATCACTCGGCTCGCTGGTCGAGGAAGAACGCAGCTAGCTGCGT |
| Clon_LD11 | ACTACTGCCAGTGGTGGATCACTCGGCTCGCTGGTCGAGGAAGAACGCAGCTAGCTGCGT |
| Clon_LD3 | ACTACTGCCAGTGGTGGATCACTCGGCTCGCTGGTCGAGGAAGAACGCAGCTAGCTGCGT |
| Clon_LD2 | ACTACTGCCAGTGGTGGATCACTCGGCTCGCTGGTCGAGGAAGAACGCAGCTAGCTGCGT |
| Clon_LD12 | ACTACTGCCAGTGGTGGATCACTCGGCTCGCTGGTCGAGGAAGAACGCAGCTAGCTGCGT |
| Clon_LD8 | ACTACTGCCAGTGGTGGATCACTCGGCTCGCTGGTCGAGGAAGAACGCAGCTAGCTGCGT |
| Clon_LD15 | ACTACTGCCAGTGGTGGATCACTCGGCTCGCTGGTCGAGGAAGAACGCAGCTAGCTGCGT |
| | * *** ****************** ***** ********* *** ** ***** *** |
| Clon_DM1 | TAATCGGTGTGAAATGCAGGACACTCTGATCACTCGACATTCGAACGCACATTGCAGCCA |
| Clon_DM21 | TAATCGGTGTGAAATGCAGGACACTCTGATCACTCGACATTCGAACGCACATTGCAGCCA |
| Clon_DM6 | TAATCGGTGTGAAATGCAGGACACTCTGATCACTCGACATTCGAACGCACATTGCAGCCA |
| Clon_DM20 | TAATCGGTGTGAAATGCAGGACACTCTGATCACTCGACATTCGAACGCACATTGCAGCCA |
| Clon_DM9 | TAATCGGTGTGAAATGCAGGACACTCTGATCACTCGACATTCGAACGCACATTGCAGCCA |
| Clon_DM12 | TAATCGGTGTGAAATGCAGGACACTCTGATCACTCGACATTCGAACGCACATTGCAGCCA |
| Clon_DM7 | TAATCGGTGTGAAATGCAGGACACTCTGATCACTCGACATTCGAACGCACATTGCAGCCA |
| Clon_DM11 | TAATCGGTGTGAAATGCAGGACACTCTGATCACTCGACATTCGAACGCACATTGCAGCCA |
| Clon_DM14 | TAATCGGTGTGAAATGCAGGACACTCTGATCACTCGACATTCGAACGCACATTGCAGCCA |
| Clon_DMA | TAATCGGTGTGAAATGCAGGACACTCTGATCACTCGACATTCGAACGCACATTGCAGCCA |
| Clon_DF1 | TAATCGGTGTGAAATGCAGGACACTCTGATCACTCGACATTCGAACGCACATTGCAGCCA |
| Clon_DF6 | TAATCGGTGTGAAATGCAGGACACTCTGATCACTCGACATTCGAACGCACATTGCAGCCA |
| Clon_DF4 | TAATCGGTGTGAAATGCAGGACACTCTGATCACTCGACATTCGAACGCACATTGCAGCCA |
| Clon_DF26 | TAATCGGTGTGAAATGCAGGACACTCTGATCACTCGACATTCGAACGCACATTGCAGCCA |
| Clon_DF4_50 | TAATCGGTGTGAAATGCAGGACACTCTGATCACTCGACATTCGAACGCACATTGCAGCCA |
| Clon_DF19 | TAATCGGTGTGAAATGCAGGACACTCTGATCACTCGACATTCGAACGCACATTGCAGCCA |
| Clon_DF3 | TAATCGGTGTGAAATGCAGGACACTCTGATCACTCGACATTCGAACGCACATTGCAGCCA |
| Clon_DF5 | TAATCGGTGTGAAATGCAGGACACTCTGATCACTCGACATTCGAACGCACATTGCAGCCA |
| Clon_DF2 | TAATCGGTGTGAAATGCAGGACACTCTGATCACTCGACATTCGAACGCACATTGCAGCCA |
| Clon_DF7 | TAATCGGTGTGAAATGCAGGACACTCTGATCACTCGACATTCGAACGCACATTGCAGCCA |
| Clon_AS15 | TAATCGGTATAAAATGCAGGACATGCCGAATACTCGACTTTCGAACGCATATTGCAGCC- |
| Clon_AS14 | TAATCGGTATAAAATGCAGGACATGCCGAATACTCGACTTTCGAACGCATATTGCAGCC- |
| Clon_AS20 | TAATCGGTATAAAATGCAGGACATGCCGAATACTCGACTTTCGAACGCATATTGCAGCC- |
| Clon_AS13 | TAATCGGTATAAAATGCAGGACATGCCGAATACTCGACTTTCGAACGCATATTGCAGCC- |
| Clon_AS10 | TAATCGGTATAAAATGCAGGACATGCCGAATACTCGACTTTCGAACGCATATTGCAGCC- |
| Clon_AS11 | TAATCGGTATAAAATGCAGGACATGCCGAATACTCGACTTTCGAACGCATATTGCAGCC- |
| Clon_AS2 | TAATCGGTATAAAATGCAGGACATGCCGAATACTCGACTTTCGAACGCATATTGCAGCC- |
| Clon_AS12 | TAATCGGTATAAAATGCAGGACATGCCGAATACTCGACTTTCGAACGCATATTGCAGCC- |
| Clon_AS1 | TAATCGGTATAAAATGCAGGACATGCCGAATACTCGACTTTCGAACGCATATTGCAGCC- |
| Clon_AS16 | TAATCGGTATAAAATGCAGGACATGCCGAATACTCGACTTTCGAACGCATATTGCAGCC- |
| Clon_BT8 | TAATCGGTGTAAAATGCAGGACACGCCGAGCACTCGACATTCGAACGCACATTGCAGCC- |
| Clon_BT9 | TAATCGGTGTAAAATGCAGGACACGCCGAGCACTCGACATTCGAACGCACATTGCAGCC- |
| Clon_BT16 | TAATCGGTGTAAAATGCAGGACACGCCGAGCACTCGACATTCGAACGCACATTGCAGCC- |
| Clon_BT3 | TAATCGGTGTAAAATGCAGGACACGCCGAGCACTCGACATTCGAACGCACATTGCAGCC- |
| Clon_BT14 | TAATCGGTGTAAAATGCAGGACACGCCGAGCACTCGACATTCGAACGCACATTGCAGCC- |
| Clon_BT17 | TAATCGGTGTAAAATGCAGGACACGCCGAGCACTCGACATTCGAACGCACATTGCAGCC- |
| Clon_BT13 | TAATCGGTGTAAAATGCAGGACACGCCGAGCACTCGACATTCGAACGCACATTGCAGCC- |
| Clon_BT1 | TAATCGGTGTAAAATGCAGGACACGCCGAGCACTCGACATTCGAACGCACATTGCAGCC- |
| Clon_BT10 | TAATCGGTGTAAAATGCAGGACACGCCGAGCACTCGACATTCGAACGCACATTGCAGCC- |
| Clon_BT15 | TAATCGGTGTAAAATGCAGGACACGCCGAGCACTCGACATTCGAACGCACATTGCAGCC- |
| Clon_TPA1_20 | TAATCGGTATAAAATGCAGGACATGCCGAATACTCGACTTTCGAACGCATATTGCAGCC- |
| Clon_TPA1_22 | TAATCGGTATAAAATGCAGGACATGCCGAATACTCGACTTTCGAACGCATATTGCAGCC- |
| Clon_TPA1_29 | TAATCGGTATAAAATGCAGGACATGCCGAATACTCGACTTTCGAACGCATATTGCAGCC- |
| Clon_TPA1_28 | TAATCGGTATAAAATGCAGGACATGCCGAATACTCGACTTTCGAACGCATATTGCAGCC- |
| Clon_TPA1_26 | TAATCGGTATAAAATGCAGGACATGCCGAATACTCGACTTTCGAACGCATATTGCAGCC- |
| Clon_TPA1_21 | TAATCGGTATAAAATGCAGGACATGCCGAATACTCGACTTTCGAACGCATATTGCAGCC- |
| Clon_TPA1_36 | TAATCGGTATAAAATGCAGGACATGCCGAATACTCGACTTTCGAACGCATATTGCAGCC- |
| Clon_TPA1_27 | TAATCGGTATAAAATGCAGGACATGCCGAATACTCGACTTTCGAACGCATATTGCAGCC- |
| Clon_TPA1_23 | TAATCGGTATAAAATGCAGGACATGCCGAATACTCGACTTTCGAACGCATATTGCAGCC- |
| Clon_TPA1_1 | TAATCGGTATAAAATGCAGGACATGCCGAATACTCGACTTTCGAACGCATATTGCAGCC- |
| Clon_TF22 | TAATCGGTATAAAATGCAGGACATGCCGAATACTCGACTTTCGAACGCATATTGCAGCC- |
| Clon_TF24 | TAATCGGTATAAAATGCAGGACATGCCGAATACTCGACTTTCGAACGCATATTGCAGCC- |
| Clon_TF3 | TAATCGGTATAAAATGCAGGACATGCCGAATACTCGACTTTCGAACGCATATTGCAGCC- |
| Clon_TF2 | TAATCGGTATAAAATGCAGGACATGCCGAATACTCGACTTTCGAACGCATATTGCAGCC- |
| Clon_TF23 | TAATCGGTATAAAATGCAGGACATGCCGAATACTCGACTTTCGAACGCATATTGCAGCC- |
| Clon_TF1 | TAATCGGTATAAAATGCAGGACATGCCGAATACTCGACTTTCGAACGCATATTGCAGCC- |
| Clon_TF4 | TAATCGGTATAAAATGCAGGACATGCCGAATACTCGACTTTCGAACGCATATTGCAGCC- |
| Clon_TF7 | TAATCGGTATAAAATGCAGGACATGCCGAATACTCGACTTTCGAACGCATATTGCAGCC- |
| Clon_TF15 | TAATCGGTATAAAATGCAGGACATGCCGAATACTCGACTTTCGAACGCATATTGCAGCC- |
| Clon_TF14 | TAATCGGTATAAAATGCAGGACATGCCGAATACTCGACTTTCGAACGCATATTGCAGCC- |
| Clon_DP8 | TAATCGGTGTAAAATGCAGGACACGCCGAGCACTCGACATTCGAACGCACATTGCAGCCA |
| Clon_DP1 | TAATCGGTGTAAAATGCAGGACACGCCGAGCACTCGACATTCGAACGCACATTGCAGCCA |
| Clon_DP7 | TAATCGGTGTAAAATGCAGGACACGCCGAGCACTCGACATTCGAACGCACATTGCAGCCA |
| Clon_DP3 | TAATCGGTGTAAAATGCAGGACACGCCGAGCACTCGACATTCGAACGCACATTGCAGCCA |
| Clon_DP6 | TAATCGGTGTAAAATGCAGGACACGCCGAGCACTCGACATTCGAACGCACATTGCAGCCA |
| Clon_DP9 | TAATCGGTGTAAAATGCAGGACACGCCGAGCACTCGACATTCGAACGCACATTGCAGCCA |
| Clon_DP2 | TAATCGGTGTAAAATGCAGGACACGCCGAGCACTCGACATTCGAACGCACATTGCAGCCA |
| Clon_DP4 | TAATCGGTGTAAAATGCAGGACACGCCGAGCACTCGACATTCGAACGCACATTGCAGCCA |
| Clon_DP10 | TAATCGGTGTAAAATGCAGGACACGCCGAGCACTCGACATTCGAACGCACATTGCAGCCA |
| Clon_DP5 | TAATCGGTGTAAAATGCAGGACACGCCGAGCACTCGACATTCGAACGCACATTGCAGCCA |
| Clon_EM4 | TAATCGGTGTAAAATGCAGGACACGCCGAGCACTCGACATTCGAACGCACATTGCAGCCA |
| Clon_EM21 | TAATCGGTGTAAAATGCAGGACACGCCGAGCACTCGACATTCGAACGCACATTGCAGCCA |
| Clon_EM2 | TAATCGGTGTAAAATGCAGGACACGCCGAGCACTCGACATTCGAACGCACATTGCAGCCA |
| Clon_EM23 | TAATCGGTGTAAAATGCAGGACACGCCGAGCACTCGACATTCGAACGCACATTGCAGCCA |
| Clon_EM3 | TAATCGGTGTAAAATGCAGGACACGCCGAGCACTCGACATTCGAACGCACATTGCAGCCA |
| Clon_EM24 | TAATCGGTGTAAAATGCAGGACACGCCGAGCACTCGACATTCGAACGCACATTGCAGCCA |
| Clon_EM22 | TAATCGGTGTAAAATGCAGGACACGCCGAGCACTCGACATTCGAACGCACATTGCAGCCA |
| Clon_EM1 | TAATCGGTGTAAAATGCAGGACACGCCGAGCACTCGACATTCGAACGCACATTGCAGCCA |
| Clon_EM6 | TAATCGGTGTAAAATGCAGGACACGCCGAGCACTCGACATTCGAACGCACATTGCAGCCA |
| Clon_EM5 | TAATCGGTGTAAAATGCAGGACACGCCGAGCACTCGACATTCGAACGCACATTGCAGCCA |
| Clon_GD1 | TAATCGGTGTAAAATGCAGGACACGCCGAGCACTCGACATTCGAACGCACATTGCAGCCA |
| Clon_GD10 | TAATCGGTGTAAAATGCAGGACACGCCGAGCACTCGACATTCGAACGCACATTGCAGCCA |
| Clon_GD2 | TAATCGGTGTAAAATGCAGGACACGCCGAGCACTCGACATTCGAACGCACATTGCAGCCA |
| Clon_GD5 | TAATCGGTGTAAAATGCAGGACACGCCGAGCACTCGACATTCGAACGCACATTGCAGCCA |
| Clon_GD3 | TAATCGGTGTAAAATGCAGGACACGCCGAGCACTCGACATTCGAACGCACATTGCAGCCA |
| Clon_GD12 | TAATCGGTGTAAAATGCAGGACACGCCGAGCACTCGACATTCGAACGCACATTGCAGCCA |
| Clon_GD7 | TAATCGGTGTAAAATGCAGGACACGCCGAGCACTCGACATTCGAACGCACATTGCAGCCA |
| Clon_GD9 | TAATCGGTGTAAAATGCAGGACACGCCGAGCACTCGACATTCGAACGCACATTGCAGCCA |
| Clon_GD8 | TAATCGGTGTAAAATGCAGGACACGCCGAGCACTCGACATTCGAACGCACATTGCAGCCA |
| Clon_GD13 | TAATCGGTGTAAAATGCAGGACACGCCGAGCACTCGACATTCGAACGCACATTGCAGCCA |
| Clon_LD5 | TAATCGGTGTAAAATGCAGGACACGCCGAGCACTCGACATTCGAACGCACATTGCAGCCA |
| Clon_LD13 | TAATCGGTGTAAAATGCAGGACACGCCGAGCACTCGACATTCGAACGCACATTGCAGCCA |
| Clon_LD5 | TAATCGGTGTAAAATGCAGGACACGCCGAGCACTCGACATTCGAACGCACATTGCAGCCA |
| Clon_LD13 | TAATCGGTGTAAAATGCAGGACACGCCGAGCACTCGACATTCGAACGCACATTGCAGCCA |
| Clon_LD14 | TAATCGGTGTAAAATGCAGGACACGCCGAGCACTCGACATTCGAACGCACATTGCAGCCA |
| Clon_LD1 | TAATCGGTGTAAAATGCAGGACACGCCGAGCACTCGACATTCGAACGCACATTGCAGCCA |
| Clon_LD11 | TAATCGGTGTAAAATGCAGGACACGCCGAGCACTCGACATTCGAACGCACATTGCAGCCA |
| Clon_LD3 | TAATCGGTGTAAAATGCAGGACACGCCGAGCACTCGACATTCGAACGCACATTGCAGCCA |
| Clon_LD2 | TAATCGGTGTAAAATGCAGGACACGCCGAGCACTCGACATTCGAACGCACATTGCAGCCA |
| Clon_LD12 | TAATCGGTGTAAAATGCAGGACACGCCGAGCACTCGACATTCGAACGCACATTGCAGCCA |
| Clon_LD8 | TAATCGGTGTAAAATGCAGGACACGCCGAGCACTCGACATTCGAACGCACATTGCAGCCA |
| Clon_LD15 | TAATCGGTGTAAAATGCAGGACACGCCGAGCACTCGACATTCGAACGCACATTGCAGCCA |
| | *** **** * ************ * ** ******* ********** ******* * |
| Clon_DM1 | TTGGATATCCGATGGCTTCGTTTGTCTGAGCGTCGTTATCGAAATTTGA-CAAACCA--- |
| Clon_DM21 | TTGGATATCCGATGGCTTCGTTTGTCTGAGCGTCGTTATCGAAATTTGA-CAAACCA--- |
| Clon_DM6 | TTGGATATCCGATGGCTTCGTTTGTCTGAGCGTCGTTATCGAAATTTGA-CAAACCA--- |
| Clon_DM20 | TTGGATATCCGATGGCTTCGTTTGTCTGAGCGTCGTTATCGAAATTTGA-CAAACCA--- |
| Clon_DM9 | TTGGATATCCGATGGCTTCGTTTGTCTGAGCGTCGTTATCGAAATTTGA-CAAACCA--- |
| Clon_DM12 | TTGGATATCCGATGGCTTCGTTTGTCTGAGCGTCGTTATCGAAATTTGA-CAAACCA--- |
| Clon_DM7 | TTGGATATCCGATGGCTTCGTTTGTCTGAGCGTCGTTATCGAAATTTGA-CAAACCA--- |
| Clon_DM11 | TTGGATATCCGATGGCTTCGTTTGTCTGAGCGTCGTTATCGAAATTTGA-CAAACCA--- |
| Clon_DM14 | TTGGATATCCGATGGCTTCGTTTGTCTGAGCGTCGTTATCGAAATTTGA-CAAACCA--- |
| Clon_DMA | TTGGATATCCGATGGCTTCGTTTGTCTGAGCGTCGTTATCGAAATTTGA-CAAACCA--- |
| Clon_DF1 | TTGGATATCCGATGGCTTCGTTTGTCTGAGCGTCGTTATCGAAATTTGA-CAAACCA--A |
| Clon_DF6 | TTGGATATCCGATGGCTTCGTTTGTCTGAGCGTCGTTATCGAAATTTGA-CAAACCA--A |
| Clon_DF4 | TTGGATATCCGATGGCTTCGTTTGTCTGAGCGTCGTTATCGAAATTTGA-CAAACCA--A |
| Clon_DF26 | TTGGATATCCGATGGCTTCGTTTGTCTGAGCGTCGTTATCGAAATTTGA-CAAACCA--A |
| Clon_DF4_50 | TTGGATATCCGATGGCTTCGTTTGTCTGAGCGTCGTTATCGAAATTTGA-CAAACCA--A |
| Clon_DF19 | TTGGATATCCGATGGCTTCGTTTGTCTGAGCGTCGTTATCGAAATTTGA-CAAACCA--A |
| Clon_DF3 | TTGGATATCCGATGGCTTCGTTTGTCTGAGCGTCGTTATCGAAATTTGA-CAAACCA--A |
| Clon_DF5 | TTGGATATCCGATGGCTTCGTTTGTCTGAGCGTCGTTATCGAAATTTGA-CAAACCA--A |
| Clon_DF2 | TTGGATATCCGATGGCTTCGTTTGTCTGAGCGTCGTTATCGAAATTTGA-CAAACCA--A |
| Clon_DF7 | TTGGATATCCGATGGCTTCGTTTGTCTGAGCGTCGTTATCGAAATTTGA-CAAACCACAA |
| Clon_AS15 | TTGGTCATACCTTGGCTTCGTTTGTCTGAGCGTCGTT---AAC--AAG--CCAAAAA--C |
| Clon_AS14 | TTGGTCATACCTTGGCTTCGTTTGTCTGAGCGTCGTT---AAC--AAG--CCAAAAA--C |
| Clon_AS20 | TTGGTCATACCTTGGCTTCGTTTGTCTGAGCGTCGTT---AAC--AAG--CCAAAAA--C |
| Clon_AS13 | TTGGTCATACCTTGGCTTCGTTTGTCTGAGCGTCGTT---AAC--AAG--CCAAAAA--C |
| Clon_AS10 | TTGGTCATACCTTGGCTTCGTTTGTCTGAGCGTCGTT---AAC--AAG--CCAAAAA--C |
| Clon_AS11 | TTGGTCATACCTTGGCTTCGTTTGTCTGAGCGTCGTT---AAC--AAG--CCAAAAA--C |
| Clon_AS2 | TTGGTCATACCTTGGCTTCGTTTGTCTGAGCGTCGTT---AAC--AAG--CCAAAAA--C |
| Clon_AS12 | TTGGTCATACCTTGGCTTCGTTTGTCTGAGCGTCGTT---AAC--AAG--CCAAAAA--C |
| Clon_AS1 | TTGGTCATACCTTGGCTTCGTTTGTCTGAGCGTCGTT---AAC--AAG--CCAAAAA--C |
| Clon_AS16 | TTGGTCATACCTTGGCTTCGTTTGTCTGAGCGTCGTT---AAC--AAG--CCAAAAA--C |
| Clon_BT8 | TTGGTCATACCTTGGCTTCGTTTGTCTGAGCGTCGTTTGAAATGAAAG--CCACAAA-CC |
| Clon_BT9 | TTGGTCATACCTTGGCTTCGTTTGTCTGAGCGTCGTTTGAAATGAAAG--CCACAAA-CC |
| Clon_BT16 | TTGGTCATACCTTGGCTTCGTTTGTCTGAGCGTCGTTTGAAATGAAAG--CCACAAA-CC |
| Clon_BT3 | TTGGTCATACCTTGGCTTCGTTTGTCTGAGCGTCGTTTGAAATGAAAG--CCACAAA-CC |
| Clon_BT14 | TTGGTCATACCTTGGCTTCGTTTGTCTGAGCGTCGTTTGAAATGAAAG--CCACAAA-CC |
| Clon_BT17 | TTGGTCATACCTTGGCTTCGTTTGTCTGAGCGTCGTTTGAAATGAAAG--CCACAAA-CC |
| Clon_BT13 | TTGGTCATACCTTGGCTTCGTTTGTCTGAGCGTCGTTTGAAATGAAAG--CCACAAA-CC |
| Clon_BT1 | TTGGTCATACCTTGGCTTCGTTTGTCTGAGCGTCGTTTGAAATGAAAG--CCACAAA-CC |
| Clon_BT10 | TTGGTCATACCTTGGCTTCGTTTGTCTGAGCGTCGTTTGAAATGAAAG--CCACAAA-CC |
| Clon_BT15 | TTGGTCATACCTTGGCTTCGTTTGTCTGAGCGTCGTTTGAAATGAAAG--CCACAAA-CC |
| Clon_TPA1_20 | TTGGTCATACCTTGGCTTCGTTTGTCTGAGCGTCGTT---AAT--ATG--CCAAACA--C |
| Clon_TPA1_22 | TTGGTCATACCTTGGCTTCGTTTGTCTGAGCGTCGTT---AAT--ATG--CCAAACA--C |
| Clon_TPA1_29 | TTGGTCATACCTTGGCTTCGTTTGTCTGAGCGTCGTT---AAT--ATG--CCAAACA--C |
| Clon_TPA1_28 | TTGGTCATACCTTGGCTTCGTTTGTCTGAGCGTCGTT---AAT--ATG--CCAAACA--C |
| Clon_TPA1_26 | TTGGTCATACCTTGGCTTCGTTTGTCTGAGCGTCGTT---AAT--ATG--CCAAACA--C |
| Clon_TPA1_21 | TTGGTCATACCTTGGCTTCGTTTGTCTGAGCGTCGTT---AAT--ATG--CCAAACA--C |
| Clon_TPA1_36 | TTGGTCATACCTTGGCTTCGTTTGTCTGAGCGTCGTT---AAT--ATG--CCAAACA--C |
| Clon_TPA1_27 | TTGGTCATACCTTGGCTTCGTTTGTCTGAGCGTCGTT---AAT--ATG--CCAAACA--C |
| Clon_TPA1_23 | TTGGTCATACCTTGGCTTCGTTTGTCTGAGCGTCGTT---AAT--ATG--CCAAACA--C |
| Clon_TPA1_1 | TTGGTCATACCTTGGCTTCGTTTGTCTGAGCGTCGTT---AAT--ATG--CCAAACA--C |
| Clon_TF22 | TTGGTCATACCTTGGCTTCGTTTGTCTGAGCGTCGTT---AAT--ATG--CCAAACA--C |
| Clon_TF24 | TTGGTCATACCTTGGCTTCGTTTGTCTGAGCGTCGTT---AAT--ATG--CCAAACA--C |
| Clon_TF3 | TTGGTCATACCTTGGCTTCGTTTGTCTGAGCGTCGTT---AAT--ATG--CCAAACA--C |
| Clon_TF2 | TTGGTCATACCTTGGCTTCGTTTGTCTGAGCGTCGTT---AAT--ATG--CCAAACA--C |
| Clon_TF23 | TTGGTCATACCTTGGCTTCGTTTGTCTGAGCGTCGTT---AAT--ATG--CCAAACA--C |
| Clon_TF1 | TTGGTCATACCTTGGCTTCGTTTGTCTGAGCGTCGTT---AAT--ATG--CCAAACA--C |
| Clon_TF4 | TTGGTCATACCTTGGCTTCGTTTGTCTGAGCGTCGTT---AAT--ATG--CCAAACA--C |
| Clon_TF7 | TTGGTCATACCTTGGCTTCGTTTGTCTGAGCGTCGTT---AAT--ATG--CCAAACA--C |
| Clon_TF15 | TTGGTCATACCTTGGCTTCGTTTGTCTGAGCGTCGTT---AAT--ATG--CCAAACA--C |
| Clon_TF14 | TTGGTCATACCTTGGCTTCGTTTGTCTGAGCGTCGTT---AAT--ATG--CCAAACA--C |
| Clon_DP8 | TTGGTCATACCTTGGCTTCGTTTGTCTGAGCGTCGTTATCAAATTATGA-CCAAACA--A |
| Clon_DP1 | TTGGTCATACCTTGGCTTCGTTTGTCTGAGCGTCGTTATCAAATTATGA-CCAAACA--A |
| Clon_DP7 | TTGGTCATACCTTGGCTTCGTTTGTCTGAGCGTCGTTATCAAATTATGA-CCAAACA--A |
| Clon_DP3 | TTGGTCATACCTTGGCTTCGTTTGTCTGAGCGTCGTTATCAAATTATGA-CCAAACA--A |
| Clon_DP6 | TTGGTCATACCTTGGCTTCGTTTGTCTGAGCGTCGTTATCAAATTATGA-CCAAACA--A |
| Clon_DP9 | TTGGTCATACCTTGGCTTCGTTTGTCTGAGCGTCGTTATCAAATTATGA-CCAAACA--A |
| Clon_DP2 | TTGGTCATACCTTGGCTTCGTTTGTCTGAGCGTCGTTATCAAATTATGA-CCAAACA--A |
| Clon_DP4 | TTGGTCATACCTTGGCTTCGTTTGTCTGAGCGTCGTTATCAAATTATGA-CCAAACA--A |
| Clon_DP10 | TTGGTCATACCTTGGCTTCGTTTGTCTGAGCGTCGTTATCAAATTATGA-CCAAACA--A |
| Clon_DP5 | TTGGTCATACCTTGGCTTCGTTTGTCTGAGCGTCGTTATCAAATTATGA-CCAAACA--A |
| Clon_EM4 | TTGGTCATACCTTGGCTTCGTTTGTCTGAGCGTCGTTATCAAATTATGA-C-AAATC--A |
| Clon_EM21 | TTGGTCATACCTTGGCTTCGTTTGTCTGAGCGTCGTTATCAAATTATGA-C-AAATC--A |
| Clon_EM2 | TTGGTCATACCTTGGCTTCGTTTGTCTGAGCGTCGTTATCAAATTATGA-C-AAATC--A |
| Clon_EM23 | TTGGTCATACCTTGGCTTCGTTTGTCTGAGCGTCGTTATCAAATTATGA-C-AAATC--A |
| Clon_EM3 | TTGGTCATACCTTGGCTTCGTTTGTCTGAGCGTCGTTATCAAATTATGA-C-AAATC--A |
| Clon_EM24 | TTGGTCATACCTTGGCTTCGTTTGTCTGAGCGTCGTTATCAAATTATGA-C-AAATC--A |
| Clon_EM22 | TTGGTCATACCTTGGCTTCGTTTGTCTGAGCGTCGTTATCAAATTATGA-C-AAATC--A |
| Clon_EM1 | TTGGTCATACCTTGGCTTCGTTTGTCTGAGCGTCGTTATCAAATTATGA-C-AAATC--A |
| Clon_EM6 | TTGGTCATACCTTGGCTTCGTTTGTCTGAGCGTCGTTATCAAATTATGA-C-AAATC--A |
| Clon_EM5 | TTGGTCATACCTTGGCTTCGTTTGTCTGAGCGTCGTTATCAAATTATGA-C-AAATC--A |
| Clon_GD1 | TTGGTCATACCTTGGCTTCGTTTGTCTGAGCGTCGTT---AATTAATAA-TCAAACA--- |
| Clon_GD10 | TTGGTCATACCTTGGCTTCGTTTGTCTGAGCGTCGTT---AATTAATAA-TCAAACA--- |
| Clon_GD2 | TTGGTCATACCTTGGCTTCGTTTGTCTGAGCGTCGTT---AATTAATAA-TCAAACA--- |
| Clon_GD5 | TTGGTCATACCTTGGCTTCGTTTGTCTGAGCGTCGTT---AATTAATAA-TCAAACA--- |
| Clon_GD3 | TTGGTCATACCTTGGCTTCGTTTGTCTGAGCGTCGTT---AATTAATAA-TCAAACA--- |
| Clon_GD12 | TTGGTCATACCTTGGCTTCGTTTGTCTGAGCGTCGTT---AATTAATAA-TCAAACA--- |
| Clon_GD7 | TTGGTCATACCTTGGCTTCGTTTGTCTGAGCGTCGTT---AATTAATAA-TCAAACA--- |
| Clon_GD9 | TTGGTCATACCTTGGCTTCGTTTGTCTGAGCGTCGTT---AATTAATAA-TCAAACA--- |
| Clon_GD8 | TTGGTCATACCTTGGCTTCGTTTGTCTGAGCGTCGTT---AATTAATAA-TCAAACA--- |
| Clon_GD13 | TTGGTCATACCTTGGCTTCGTTTGTCTGAGCGTCGTT---AATTAATAA-TCAAACA--- |
| Clon_LD5 | TTGGTCATACCTTGGCTTCGTTTGTCTGAGCGTCGTT---AATTAATAAATCAAACA--- |
| Clon_LD13 | TTGGTCATACCTTGGCTTCGTTTGTCTGAGCGTCGTT---AATTAATAAATCAAACA--- |
| Clon_LD5 | TTGGTCATACCTTGGCTTCGTTTGTCTGAGCGTCGTT---AATTAATAAATCAAACA--- |
| Clon_LD13 | TTGGTCATACCTTGGCTTCGTTTGTCTGAGCGTCGTT---AATTAATAAATCAAACA--- |
| Clon_LD14 | TTGGTCATACCTTGGCTTCGTTTGTCTGAGCGTCGTT---AATTAATAAATCAAACA--- |
| Clon_LD1 | TTGGTCATACCTTGGCTTCGTTTGTCTGAGCGTCGTT---AATTAATAAATCAAACA--- |
| Clon_LD11 | TTGGTCATACCTTGGCTTCGTTTGTCTGAGCGTCGTT---AATTAATAAATCAAACA--- |
| Clon_LD3 | TTGGTCATACCTTGGCTTCGTTTGTCTGAGCGTCGTT---AATTAATAAATCAAACA--- |
| Clon_LD2 | TTGGTCATACCTTGGCTTCGTTTGTCTGAGCGTCGTT---AATTAATAAATCAAACA--- |
| Clon_LD12 | TTGGTCATACCTTGGCTTCGTTTGTCTGAGCGTCGTT---AATTAATAAATCAAACA--- |
| Clon_LD8 | TTGGTCATACCTTGGCTTCGTTTGTCTGAGCGTCGTT---AATTAATAAATCAAACA--- |
| Clon_LD15 | TTGGTCATACCTTGGCTTCGTTTGTCTGAGCGTCGTT---AATTAATAAATCAAACA--- |
| | ** * * * **** ********** ****** * * |
| Clon_DM1 | AATGGAATAGATCTGTGTC-------------GTCGCG--------ATTCATTTCGGCG- |
| Clon_DM21 | AATGGAATAGATCTGTGTC-------------GTCGCG--------ATTCATTTCGGCG- |
| Clon_DM6 | AATGGAATAGATCTGTGTC-------------GTCGCG--------ATTCATTTCGGCG- |
| Clon_DM20 | AATGGAATAGATCTGTGTC-------------GTCGCG--------ATTCATTTCGGCG- |
| Clon_DM9 | AATGGAATAGATCTGTGTC-------------GTCGCG--------ATTCATTTCGGCG- |
| Clon_DM12 | AATGGAATAGATCTGTGTC-------------GTCGCG--------ATTCATTTCGGCG- |
| Clon_DM7 | AATGGAATAGATCTGTGTC-------------GTCGCG--------ATTCATTTCGGCG- |
| Clon_DM11 | AATGGAATAGATCTGTGTC-------------GTCGCG--------ATTCATTTCGGCG- |
| Clon_DM14 | AATGGAATAGATCTGTGTC-------------GTCGCG--------ATTCATTTCGGCG- |
| Clon_DMA | AATGGAATAGATCTGTGTC-------------GTCGCG--------ATTCATTTCGGCG- |
| Clon_DF1 | AATGGAATAGATCTGTGTC-------------GTCGCG--------ATTCATTTCGGCG- |
| Clon_DF6 | AATGGAATAGATCTGTGTC-------------GTCGCG--------ATTCATTTCGGCG- |
| Clon_DF4 | AATGGAATAGATCTGTGTC-------------GTCGTT--------ATTCATTTCGGCG- |
| Clon_DF26 | AATGGAATAGATCTGTGTC-------------GTCGTT--------ATTCATTTCGGCG- |
| Clon_DF4_50 | AATGGAATAGATCTGTGTC-------------GTCGCG--------ATTCATTTCGGCG- |
| Clon_DF19 | AATGGAATAGATCTGTGTC-------------GTCGCG--------ATTCATTTCGGCG- |
| Clon_DF3 | AATGGAATAGATCTGTGTC-------------GTCGCG--------ATTCATTTCGGCG- |
| Clon_DF5 | AATGGAATAGATCTGTGTC-------------GTCGCG--------ATTCATTTCGGCG- |
| Clon_DF2 | AATGGAATAGATCTGTGTC-------------GTCGCG--------ATTCATTTCGGCG- |
| Clon_DF7 | AATGGAATAGATCTGTGTC-------------GTCGCG--------ATTCATTTCGGCG- |
| Clon_AS15 | TAT-CAAAAGGTCCGTTCAC-----------CTTCGTATCCTAACCTCTTGTCGGGTTGA |
| Clon_AS14 | TAT-CAAAAGGTCCGTTCAC-----------CTTCGTATCCTAACCTCTTGTCGGGTTGA |
| Clon_AS20 | TAT-CAAAAGGTCCGTTCAC-----------CTTCGTATCCTAACCTCTTGTCGGGTTGA |
| Clon_AS13 | TAT-CAAAAGGTCCGTTCAC-----------CTTCGTATCCTAACCTCTTGTCGGGTTGA |
| Clon_AS10 | TAT-CAAAAGGTCCGTTCAC-----------CTTCGTATCCTAACCTCTTGTCGGGTTGA |
| Clon_AS11 | TAT-CAAAAGGTCCGTTCAC-----------CTTCGTATCCTAACCTCTTGTCGGGTTGA |
| Clon_AS2 | TAT-CAAAAGGTCCGTTCAC-----------CTTCGTATCCTAACCTCTTGTCGGGTTGA |
| Clon_AS12 | TAT-CAAAAGGTCCGTTCAC-----------CTTCGTATCCTAACCTCTTGTCGGGTTGA |
| Clon_AS1 | TAT-CAAAAGGTCCGTTCAC-----------CTTCGTATCCTAACCTCTTGTCGGGTTGA |
| Clon_AS16 | TAT-CAAAAGGTCCGTTCAC-----------CTTCGTATCCTAACCTCTTGTCGGGTTGA |
| Clon_BT8 | TATATGCAAGACTCGTTCA--------------TCGTGT--TGAA--TTTGTTT--TTAA |
| Clon_BT9 | TATATGCAAGACTCGTTCA--------------TCGTGT--TGAA--TTTGTTT--TTAA |
| Clon_BT16 | TATATGCAAGACTCGTTCA--------------TCGTGT--TGAA--TTTGTTT--TTAA |
| Clon_BT3 | TATATGCAAGACTCGTTCA--------------TCGTGT--TGAA--TTTGTTT--TTAA |
| Clon_BT14 | TATATGCAAGACTCGTTCA--------------TCGTGT--TGAA--TTTGTTT--TTAA |
| Clon_BT17 | TATATGCAAGACTCGTTCA--------------TCGTGT--TGAA--TTTGTTT--TCAA |
| Clon_BT13 | TATATGCAAGACTCGTTCA--------------TCGTGT--TAAACTTTTGTTT--TTAA |
| Clon_BT1 | TATATGCAAGACTCGTTCA--------------TCGTGT--TAAACTTTTGTTT--TTAA |
| Clon_BT10 | TATATGCAAGACTCGTTCA--------------TCGTGT--TGAA--TTTGTTT--TTAA |
| Clon_BT15 | TATATGCAAGACTCGTTCA--------------TCGTGT--CAAA--TTTGTTT--TTAA |
| Clon_TPA1_20 | CATGCA-AAGGCCTGTTCC---------CCATGTCGTGTC--GATCATTCAGTCA-TCGA |
| Clon_TPA1_22 | CATGCA-AAGGCCTGTTCC---------CCATGTCGTGTC--GATCATTCAGTCA-TCGA |
| Clon_TPA1_29 | TATGCA-AAGGCCTGTTCC---------CCATGTCGTGTC--GATCATTCAGTCA-TCGA |
| Clon_TPA1_28 | CATGCA-AAGGCCTGTTCC---------CCATGTCGTGTC--GATCATTCAGTCA-TCGA |
| Clon_TPA1_26 | CATGCA-AAGGCCTGTTCC---------CCATGTCGTGTC--GATCATTCAGTCA-TCGA |
| Clon_TPA1_21 | CATGCA-AAGGCCTGTTCC---------CCATGTCGTGTC--GATCATTCAGTCA-TCGA |
| Clon_TPA1_36 | TATGCA-AAGGCCTGTTCC---------CCATGTCGTGTC--GATCATTCAGTCA-TCGA |
| Clon_TPA1_27 | CATGCA-AAGGCCTGTTCC---------CCATGTCGTGTC--GATCATTCAGTCA-TCGA |
| Clon_TPA1_23 | CATGCA-AAGGCCTGTTCC---------CCATGTCGTGTC--GATCATTCAGTCA-TCGA |
| Clon_TPA1_1 | CATGCA-AAGGCCTGTTCC---------CCATGTCGTGTC--GATCATTCAGTCA-TCGA |
| Clon_TF22 | CATGCA-AAGGCCTGCTGCACTTGCGTGCAGTGTCGTGTC--GATCATTCAGTCA-TCGA |
| Clon_TF24 | CATGCA-AAGGCCTGCTGCACTTGCGTGCAGTGTCGTGTC--GATCATTCAGTCA-TCGA |
| Clon_TF3 | CATGCA-AAGGCCTGCTGCACTTGCGTGCAGTGTCGTGTC--GATCATTCAGTCA-TCGA |
| Clon_TF2 | CATGCA-AAGGCCTGCTGCACTTGCGTGCAGTGTCGTGTC--GATCATTCAGTCA-TCGA |
| Clon_TF23 | CATGCA-AAGGCCTGCTGCACTTGCGTGCAGTGTCGTGTC--GATCATTCAGTCA-TCGA |
| Clon_TF1 | CATGCA-AAGGCCTGCTGCACTTGCGTGCAGTGTCGTGTC--GATCATTCAGTCA-TCGA |
| Clon_TF4 | CATGCA-AAGGCCTGCTGCACTTGCGTGCAGTGTCGTGTC--GATCATTCAGTCA-TCGA |
| Clon_TF7 | CATGCA-AAGGCCTGCTGCACTTGCGTGCAGTGTCGTGTC--GATCATTCAGTCA-TCGA |
| Clon_TF15 | CATGCA-AAGGCCTGCTGCACTTGCGTGCAGTGTCGTGTC--GATCATTCAGTCA-TCGA |
| Clon_TF14 | CATGCA-AAGGCCTGCTGCACTTGCGTGCAGTGTCGTGTC--GATCATTCAGTCA-TCGA |
| Clon_DP8 | AATGGGATAGATCTGTTTC-------------GTCGTG----------CATA-----CG- |
| Clon_DP1 | AATGGGATAGATCTGTTTC-------------GTCGTG----------CATA-----CG- |
| Clon_DP7 | AATGGGATAGATCTGTGTC-------------GTCGTG----------CATA-----CG- |
| Clon_DP3 | AATGGGATAGATCTGTTTC-------------GTCGTG----------CAAA-----CG- |
| Clon_DP6 | AATGGGATAGATCTGTTTC-------------GTCGTG----------CAAA-----CG- |
| Clon_DP9 | AATGGGATAGATCTGTTTC-------------GTCGTG----------CAAA-----CG- |
| Clon_DP2 | AATGGGATAGATCTGTTTC-------------GTCGTG----------CATA-----CG- |
| Clon_DP4 | AATGGGATAGATCTGTTTC-------------GTCGTG----------CATA-----CG- |
| Clon_DP10 | AATGGGATAGATCTGTTTC-------------GTCGTG----------TAAA-----CG- |
| Clon_DP5 | AATGGGATAGATCTGTTTC-------------GTCGTG----------CATA-----CG- |
| Clon_EM4 | AATGGGATAGATCTGTTTC-------------GTCGTGAT----GTGTTAAATTG--CG- |
| Clon_EM21 | AATGGGATAGATCTGTTTC-------------GTCGTGAT----GTGTTAAATTG--CG- |
| Clon_EM2 | AATGGGATAGATCTGTTTC-------------GTCGTGAT----A--TTAAATTG--CG- |
| Clon_EM23 | AATGGGATAGATCTGTTTC-------------GTCGTGAT----GTGTTAAATTG--CG- |
| Clon_EM3 | AATGGGATAGATCTGTTTC-------------GTCGTGAT----G--TTAAATTG--CG- |
| Clon_EM24 | AATGGGATAGATCTGTTTC-------------GTCGTGAT----G--TTAAATTG--CG- |
| Clon_EM22 | AATGGGATAGATCTGTTTC-------------GTCGTGAT----G--TCAAATTG--CG- |
| Clon_EM1 | AATGGGATAGATCTGTTTC-------------GTCGTGAT----GTGTTAAATTG--CG- |
| Clon_EM6 | AATGGGATAGATCTGTTTC-------------GTCGTGAT--------TGAATTG--CG- |
| Clon_EM5 | AATGGGATAGATCTGTTTC-------------GTCGTGAT--------TGAATTG--CG- |
| Clon_GD1 | --T-CAAAAGACCTGTTTG--------------TCGTATTCC--TCGATTA-CTTGAGAA |
| Clon_GD10 | --T-CAAAAGACCTGTTTG--------------TCGTATTCC--TCGATTA-CTTGAGAA |
| Clon_GD2 | --T-CAAAAGACCTGTTTG--------------TCGTATTCC--TCGATTA-CTTGAGAA |
| Clon_GD5 | --T-CAAAAGACCTGTTTG--------------TCGTATTCC--TCGATTA-CTTGAGAA |
| Clon_GD3 | --T-CAAAAGACCTGTTTG--------------TCGTATTCC--TCGATTA-CTTGAGAA |
| Clon_GD12 | --T-CAAAAGACCTGTTTG--------------TCGTATTCC--TCGATTA-CTTGAGAA |
| Clon_GD7 | --T-CAAAAGACCTGTTTG--------------TCGTATTCC--TCGATTA-CTTGAGAA |
| Clon_GD9 | --T-CAAAAGACCTGTTTG--------------TCGTATTCC--TCGATTA-CTTGAGAA |
| Clon_GD8 | --T-CAAAAGACCTGTTTG--------------TCGTATTCC--TCGATTA-CTTGAGAA |
| Clon_GD13 | --T-CAAAAGACCTGTTTG--------------TCGTATTCC--TCGATTA-CTTGAGAA |
| Clon_LD5 | --T-CAAAAGACCTGTT----------------TCGTATCTTG-TCGTTTAATTCGATGA |
| Clon_LD13 | --T-CAAAAGACCTGTT----------------TCGTATCTTG-TCGTTTAATTCGATGA |
| Clon_LD5 | --T-CAAAAGACCTGTT----------------TCGTATCTTG-TCGTTTAATTCGATGA |
| Clon_LD13 | --T-CAAAAGACCTGTT----------------TCGTATCTTG-TCGTTTAATTCGATGA |
| Clon_LD14 | --T-CAAAAGACCTGTT----------------TCGTATCTTG-TCGTTTAATTCGATGA |
| Clon_LD1 | --T-CAAAAGACCTGTT----------------TCGTATCTTG-TCGTTTAATTCGATGA |
| Clon_LD11 | --T-CAAAAGACCTGTT----------------TCGTATCTTG-TCGTTTAATTCGATGA |
| Clon_LD3 | --T-CAAAAGACCTGTT----------------TCGTATCTTG-TCGTTTAATTCGATGA |
| Clon_LD2 | --T-CAAAAGACCTGTT----------------TCGTATCTTG-TCGTTTAATTCGATGA |
| Clon_LD12 | --T-CAAAAGACCTGTT----------------TCGTATCTTG-TCGTTTAATTCGATGA |
| Clon_LD8 | --T-CAAAAGACCTGTT----------------TCGTATCTTG-TCGTTTAATTCGATGA |
| Clon_LD15 | --T-CAAAAGACCTGTT----------------TCGTATCTTG-TCGTTTAATTCGATGA |
| | * * *** |
| Clon_DM1 | -TCGTGAGATTATTTCTAAACAT-----------------TTCGAA--TGCTGA------ |
| Clon_DM21 | -TCGTGAGATTATTTCTAAACAT-----------------TTCGAA--TGCTGA------ |
| Clon_DM6 | -TCGTGAGATTATTTCTAAACAT-----------------TTCGAA--TGCTGA------ |
| Clon_DM20 | -TCGTGAGATTATTTCTAAACAT-----------------TTCGAA--TGCTGA------ |
| Clon_DM9 | -TCGTGAGATTATTTCTAAACAT-----------------TTCGAA--TGCTGA------ |
| Clon_DM12 | -TCGTGAGATTATTTCTAAACAT-----------------TTCGAA--TGCTGA------ |
| Clon_DM7 | -TCGTGAGATTATTTCTAAACAT-----------------TTCGAA--TGCTGA------ |
| Clon_DM11 | -TCGTGAGATTATTTCTAAACAT-----------------TTCGAA--TGCTGA------ |
| Clon_DM14 | -TCGTGAGATTATTTCTAAACAT-----------------TTCGAA--TGCTGA------ |
| Clon_DMA | -TCGTGAGATTATTTCTAAACAT-----------------TTCGAA--TGCTGA------ |
| Clon_DF1 | -TCGTGAGATTATTTCTAAACAT-----------------TTCGAA--TGCTGA------ |
| Clon_DF6 | -TCGTGAGATTATTTCTAAACAT-----------------TTCGAA--TGCTGA------ |
| Clon_DF4 | -TCGTGAGATTATTTCTAAACAT-----------------TTTGAA--TGCTGA------ |
| Clon_DF26 | -TCGTGAGATTATTTCTAAACAT-----------------TTTGAA--TGCTGA------ |
| Clon_DF4_50 | -TCGTGAGATTATTTCTAAACAT-----------------TTCGAA--TGCTGA------ |
| Clon_DF19 | -TCGTGAGATTATTTCTAAACAT-----------------TTCGAA--TGCTGA------ |
| Clon_DF3 | -TCGTGAGATTATTTCTAAACAT-----------------TTCGAA--TGCTGA------ |
| Clon_DF5 | -TCGTGAGATTATTTCTAAACAT-----------------TTCGAA--TGCTGA------ |
| Clon_DF2 | -TCGTGAGATTATTTCTAAACAT-----------------TTCGAA--TGCTGA------ |
| Clon_DF7 | -TCGTGAGATTATTTCTAAACAT-----------------TTCGAA--TGCTGA------ |
| Clon_AS15 | TTCGTCGGGCCACCTTTAAACACTACTTTAAAATGTACTCTTGTCA--TTTTAA------ |
| Clon_AS14 | TTCGTCGGGCCACCTTTAAACACTACTTTAAAATGTACTCTTGTCA--TTTTAA------ |
| Clon_AS20 | TTCGTCGGGCCACCTTTAAACACTACTTTAAAATGTACTCTTGTCA--TTTTAA------ |
| Clon_AS13 | TTCGTCGGGCCACCTTTAAACACTACTTTAAAATGTACTCTTGTCA--TTTTAA------ |
| Clon_AS10 | TTCGTCGGGCCACCTTTAAACACTACTTTAAAATGTACTCTTGTCA--TTTTAAA----- |
| Clon_AS11 | TTCGTCGGGCCACCTTTAAACACTACTTTAAAATGTACTCTTGTCA--TTTTAA------ |
| Clon_AS2 | TTCGTCGGGCCACCTTTAAACACTACTTTAAAATGTACTCTTGTCA--TTTTAA------ |
| Clon_AS12 | TTCGTCGGGCCACCTTTAAACACTACTTTAAAATGTACTCTTGTCA--TTTTAAA----- |
| Clon_AS1 | TTCGTCGGGCCACCTTTAAACACTACTTTAAAATGTACTCTTGTCA--TTTTAA------ |
| Clon_AS16 | TTCGTCGGGCCACCTTTAAACACTACTTTAAAATGTACTCTTGTCA--TTTTAAA----- |
| Clon_BT8 | CTCGTCGAGTCGCATT---------------AATGT----TTGCCA------AA------ |
| Clon_BT9 | CTCGTCGAGTCGCATT---------------AATGT----TTGCCA------AA------ |
| Clon_BT16 | CTCGTCGAGTCGCATT---------------AATGT----TTGCCA------AA------ |
| Clon_BT3 | CTCGTCGAGTCGCATT---------------AATGT----TTGCCA------AA------ |
| Clon_BT14 | CTCGTCGAGTCGCATT---------------AATGT----TTGCCA------AA------ |
| Clon_BT17 | CTCGTCGAGTCGCATT---------------AATGT----TTGCCA------AA------ |
| Clon_BT13 | CTCGTCGAGTCGCATT---------------AATGT----TTGCCA------AA------ |
| Clon_BT1 | CTCGTCGAGTCGCATT---------------AATGT----TTGCCA------AA------ |
| Clon_BT10 | CTCGTCGAGTCGCATT---------------AATGT----TTGCCA------AA------ |
| Clon_BT15 | CTCGTCGAGTCGCATT---------------AATGT----TTGCCA------AA------ |
| Clon_TPA1_20 | CTCGTCAGGCCAC-TTGTAACACTTCTTTCAACT------TTGTTA---TTGAA------ |
| Clon_TPA1_22 | CTCGTCAGGCCAC-TTGTAACACTTCTTTCAACT------TTGTTA---TTGAA------ |
| Clon_TPA1_29 | CTCGTCAGGCCAC-TTGTAACACTACTTTCAACT------TTGTTA---TTGAA------ |
| Clon_TPA1_28 | CTCGTCAGGCCAC-TTGTAACACTACTTTCAACT------TTGTTA---TTGAA------ |
| Clon_TPA1_26 | CTCGTCAGGCCAC-TTGTAACACTACTTTCAACT------TTGTTA---TTGAA------ |
| Clon_TPA1_21 | CTCGTCAGGCCAC-TTGTAACACTACTTTCAACT------TTGTTA---TTGAA------ |
| Clon_TPA1_36 | CTCGTCAGGCCAC-TTGTAACACTACTTTCAACT------TTGTTA---TTGAA------ |
| Clon_TPA1_27 | CTCGTCAGGCCAC-TTGTAACACTACTTTCAACT------TTGTTA---TTGAA------ |
| Clon_TPA1_23 | CTCGTCAGGCCAC-TTGTAACACTACTTTCAACT------TTGTTA---TTGAA------ |
| Clon_TPA1_1 | CTCGTCAGGCCAC-TTGTAACACTTCTTTCAACT------TTGTTA---TTGAA------ |
| Clon_TF22 | CTCGTCAGGCCAC-TTGTAACACTACTTTCAACT------TTGTTA---TTGAA------ |
| Clon_TF24 | CTCGTCAGGCCAC-TTGTAACACTACTTTCAACT------TTGTTA---TTGAA------ |
| Clon_TF3 | CTCGTCAGGCCAC-TTGTAACACTACTTTCAACT------TTGTTA---TTGAA------ |
| Clon_TF2 | CTCGTCAGGCCAC-TTGTAACACTACTTTCAACT------TTGTTA---TTGAA------ |
| Clon_TF23 | CTCGTCAGGCCAC-TTGTAACACTACTTTCAACT------TTGTTA---TTGAA------ |
| Clon_TF1 | CTCGTCAGGCCAC-TTGTAACACTACTTTCAACT------TTGTTA---TTGAA------ |
| Clon_TF4 | CTCGTCAGGCCAC-TTGTAACACTACTTTCAACT------TTGTTA---TTGAA------ |
| Clon_TF7 | CTCGTCAGGCCAC-TTGTAACACTACTTTCAACT------TTGTTA---TTGAA------ |
| Clon_TF15 | CTCGTCAGGCCAC-TTGTAACACTACTTTCAACT------TTGTTA---TTGAA------ |
| Clon_TF14 | CTCGTCAGGCCAC-TTGTAACACTACTTTCAACT------TTGTTA---TTGAA------ |
| Clon_DP8 | -TCGTCAGGTCATTTCCAAACAT-----------------TTGTTA--TGCTGA------ |
| Clon_DP1 | -TCGTCAGGTCATTTCCAAACAT-----------------TTGTTA--TGCTGA------ |
| Clon_DP7 | -TCGTCAGGTCATTTCCAAACAT-----------------TTGTTA--TGCTGA------ |
| Clon_DP3 | -TCGTCAGATCATTTCCAAACAT-----------------TTGTTA--TGCTGA------ |
| Clon_DP6 | -TCGTCAGATCATTTCCAAACAT-----------------TTGTTA--TGCTGA------ |
| Clon_DP9 | -TCGTCAGATCATTTCCAAACAT-----------------TTGTTA--TGCTGA------ |
| Clon_DP2 | -TCGTCAGGTCATTTCCAAACAT-----------------TTGTTA--TGCTGA------ |
| Clon_DP4 | -TCGTCAGGTCATTTCCAAACAT-----------------TTGTTA--TGCTGA------ |
| Clon_DP10 | -TCGTCAGATCATTTCCAAACAT-----------------TTGTTA--TGCTGA------ |
| Clon_DP5 | -TCGTCAGGTCATTTCCAAACAT-----------------TTGTTA--TGCTGA------ |
| Clon_EM4 | -TCGTCAGATCATTTCCAAACAT-----------------TTGTTA--TGCTGA------ |
| Clon_EM21 | -TCGTCAGATCATTTCCAAACAT-----------------TTGTTA--TGCTGA------ |
| Clon_EM2 | -TCGTCAGATCATTTCCAAACAT-----------------TTGTTA--TGCTGA------ |
| Clon_EM23 | -TCGTCAGATCATTTCCAAACAT-----------------TTGTTA--TGCTGA------ |
| Clon_EM3 | -TCGTCAGATCATTTCCAAACAT-----------------TTGTTA--TGCTGA------ |
| Clon_EM24 | -TCGTCAGATCATTTCCAAACAT-----------------TTGTTA--TGCTGA------ |
| Clon_EM22 | -TCGTCAGATCATTTCCAAACAT-----------------TTGTTA--TGCTGA------ |
| Clon_EM1 | -TCGTCAGATCATTTCCAAACAT-----------------TTGTTA--TGCTGA------ |
| Clon_EM6 | -TCGTCAGATCATTTCCAAACAT-----------------TTGTTA--TGCTGA------ |
| Clon_EM5 | -TCGTCAGATCATTTCCAAACAT-----------------TTGTTA--TGCTGA------ |
| Clon_GD1 | TTCGTCAGGTTCAATTGATACAC--------------TGATTGTCA-ATATTA------T |
| Clon_GD10 | TTCGTCAGGTTCAATTGATACAC--------------TGATTGTCA-ATATTA------T |
| Clon_GD2 | TTCGTCAGGTTCAATTGATACAC--------------TGATTGTCA-ATATTA------T |
| Clon_GD5 | TTCGTCAGGTTCAATTGATACAC--------------TGATTGTCA-ATATTA------T |
| Clon_GD3 | TTCGTCAGGTTCAATTGATACAC--------------TGATTGTCA-ATATTA------T |
| Clon_GD12 | TTCGTCAGGTTCAATTGATACAC--------------TGATTGTCA-ATATTA------T |
| Clon_GD7 | TTCGTCAGGTTCAATTGATACAC--------------TGATTGTCA-ATATTA------T |
| Clon_GD9 | TTCGTCAGGTTCAATTGATACAC--------------TGATTGTCA-ATATTA------T |
| Clon_GD8 | TTCGTCAGGTTCAATTGATACAC--------------TGATTGTCA-ATATTA------T |
| Clon_GD13 | TTCGTCAGGTTCAATTGATACAC--------------TGATTGTCA-ATATTA------T |
| Clon_LD5 | TTCGTCGGGTTTAATTGATACACGC------------TGATTGTCACACACTACGAATGT |
| Clon_LD13 | TTCGTCGGGTTTAATTGATACACGC------------TGATTGTCACACACTACGAATGT |
| Clon_LD5 | TTCGTCGGGTTTAATTGATACACGC------------TGATTGTCACACACTACGAATGT |
| Clon_LD13 | TTCGTCGGGTTTAATTGATACACGC------------TGATTGTCACACACTACGAATGT |
| Clon_LD14 | TTCGTCGGGTTTAATTGATACACGC------------TGATTGTCACACACTACGAATGT |
| Clon_LD1 | TTCGTCGGGTTTAATTGATACACGC------------TGATTGTCACACACTACGAATGT |
| Clon_LD11 | TTCGTCGGGTTTAATTGATACACGC------------TGATTGTCACACACTACGAATGT |
| Clon_LD3 | TTCGTCGGGTTTAATTGATACACGC------------TGATTGTCACACACTACGAATGT |
| Clon_LD2 | TTCGTCGGGTTTAATTGATACACGC------------TGATTGTCACACACTACGAATGT |
| Clon_LD12 | TTCGTCGGGTTTAATTGATACACGC------------TGATTGTCACACACTACGAATGT |
| Clon_LD8 | TTCGTCGGGTTTAATTGATACACGC------------TGATTGTCACACACTACGAATGT |
| Clon_LD15 | TTCGTCGGGTTTAATTGATACACGC------------TGATTGTCACACACTACGAATGT |
| | *** * * |
| Clon_DM1 | --CTCTTTT--TTGGTGTGTGAAGG----------------------------------- |
| Clon_DM21 | --CTCTTTT--TTGGTGTGTGAAGG----------------------------------- |
| Clon_DM6 | --CTCTTTT--TTGGTGTGTGAAGG----------------------------------- |
| Clon_DM20 | --CTCTTTT--TTGGTGTGTGAAGG----------------------------------- |
| Clon_DM9 | --CTCTTTT--TTGGTGTGTGAAGG----------------------------------- |
| Clon_DM12 | --CTCTTTT--TTGGTGTGTGAAGG----------------------------------- |
| Clon_DM7 | --CTCTTTT--TTGGTGTGTGAAGG----------------------------------- |
| Clon_DM11 | --CTCTTTT--TTGGTGTGTGAAGG----------------------------------- |
| Clon_DM14 | --CTCTTTT--TTGGTGTGTGAAGG----------------------------------- |
| Clon_DMA | --CTCTTTT--TTGGTGTGTGAAGG----------------------------------- |
| Clon_DF1 | --CTCTTTTGGTTGGTGTGTGAAGG----------------------------------- |
| Clon_DF6 | --CTCTTTTGGTTGGTGTGTGAAGG----------------------------------- |
| Clon_DF4 | --CTCTTTTGGTTGGTGTGTGAAGG----------------------------------- |
| Clon_DF26 | --CTCTTTTGGTTGGTGTGTGAAGG----------------------------------- |
| Clon_DF4_50 | --CTCTTTTGGTTGGTGTGTGAAGG----------------------------------- |
| Clon_DF19 | --CTCTTTTGGTTGGTGTGTGAAGG----------------------------------- |
| Clon_DF3 | --CTCTTTTGGTTGGTGTGTGAAGG----------------------------------- |
| Clon_DF5 | --CTCTTTTGGTTGGTGTGTGAAGG----------------------------------- |
| Clon_DF2 | --CTCTTTTGGTTGGTGTGTGAAGG----------------------------------- |
| Clon_DF7 | --CTCTTTTGGTTGGTGTGTGAAGG----------------------------------- |
| Clon_AS15 | -TGTGCCCATACGAGCGTAAAGACAG---------------TTAACCA------------ |
| Clon_AS14 | -TGTGCCCATACGAGCGTAAAGACAG---------------TTAACCA------------ |
| Clon_AS20 | -TGTGCCCATACGAGCGTAAAGACAG---------------TTAACCA------------ |
| Clon_AS13 | -TGTGCCCATACGAGCGTAAAGACAG---------------TTAACCA------------ |
| Clon_AS10 | -TGTGCCCATACGAGCGTAAAGACAG---------------TTAACCA------------ |
| Clon_AS11 | -TGTGCCCATACGAGCGTAAAGACAG---------------TTAACCA------------ |
| Clon_AS2 | -TGTGCCCATACGAGCGTAAAGACAG---------------TTAACCA------------ |
| Clon_AS12 | -TGTGCCCATACGAGCGTAAAGACAG---------------TTAACCA------------ |
| Clon_AS1 | -TGTGCCCATACGAGCGTAAAGACAG---------------TTAACCA------------ |
| Clon_AS16 | -TGTGCCCATACGAGCGTAAAGACAG---------------TTAACCA------------ |
| Clon_BT8 | ------------AAG----GAGACTTTT-------------TTAAGAA------------ |
| Clon_BT9 | ------------AAG----GAGACTTTT-------------TTA--AA------------ |
| Clon_BT16 | ------------AAG----GAGAC-----------------TTA--AA------------ |
| Clon_BT3 | ------------AAG----GAGAC-----------------TTA--AA------------ |
| Clon_BT14 | ------------AAG----GAGAC-----------------TTA--AA------------ |
| Clon_BT17 | ------------AAG----GAGAC-----------------TTA--AA------------ |
| Clon_BT13 | ------------AAG----GAGAC-----------------TTA--AA------------ |
| Clon_BT1 | ------------AAG----GAGACTTT--------------TTAGAAA------------ |
| Clon_BT10 | ------------AAG----GAGAC-----------------TTA--AA------------ |
| Clon_BT15 | ------------AAG----GAGAC-----------------TTA--AA------------ |
| Clon_TPA1_20 | --CTGCCCATACGAGCGTAGGGAGAGAG---CCTA-CCAGTTTGCTGG------------- |
| Clon_TPA1_22 | --CTGCCCATACGAGCGTAGGGAGAGAG---CCTA-CCAGTTTGCTGG------------- |
| Clon_TPA1_29 | --CTGCCCATACGAGCGTAGGGAGAGAG---CCTA-CCAGTTTGCTGG------------- |
| Clon_TPA1_28 | --CTGCCCATACGAGCGTAGGGAGAGAG---CCTA-CCAGTTTGCTGG------------- |
| Clon_TPA1_26 | --CTGCCCATACGAGCGTAGGGAGAGAG---CCTA-CCAGTTTGCTGG------------- |
| Clon_TPA1_21 | --CTGCCCATACGAGCGTAGGGAGAGAG---CCTA-CCAGTTTGCTGG------------- |
| Clon_TPA1_36 | --CTGCCCATACGAGCGTAGGGAGAGAG---CCTA-CCAGTTTGCTGG------------- |
| Clon_TPA1_27 | --CTGCCCATACGAGCGTAGGGAGAGAG---CCTA-CCAGTTTGCTGG------------- |
| Clon_TPA1_23 | --CTGCCCATACGAGCGTAGGGAGAGAG---CCTA-CCAGTTTGCTGG------------- |
| Clon_TPA1_1 | --CTGCCCATACGAGCGTAGGGAGAGAG---CCTA-CCAGTTTGCTGG------------- |
| Clon_TF22 | --ATGCCCATACGAGCGTAAGAAGAGAGTTGCTTA-CCAGTTTGCCGG------------- |
| Clon_TF24 | --ATGCCCATACGAGCGTAAGAAGAGAGTTGCTTA-CCAGTTTGCCGG------------- |
| Clon_TF3 | --ATGCCCATACGAGCGTAAGAAGAGAGTTGCTTA-CCAGTTTGCCGG------------- |
| Clon_TF2 | --ATGCCCATACGAGCGTAAGAAGAGAGTTGCTTA-CCAGTTTGCCGG------------- |
| Clon_TF23 | --ATGCCCATACGAGCGTAAGAAGAGAGTTGCTTA-CCAGTTTGCCGG------------- |
| Clon_TF1 | --ATGCCCATACGAGCGTAAGAAGAGAGTTGCTTA-CCAGTTTGCCGG------------- |
| Clon_TF4 | --ATGCCCATACGAGCGTAAGAAGAGAGTTGCTTA-CCAGTTTGCCGG------------- |
| Clon_TF7 | --ATGCCCATACGAGCGTAAGAAGAGAGTTGCTTA-CCAGTTTGCCGG------------- |
| Clon_TF15 | --ATGCCCATACGAGCGTAAGAAGAGAGTTGCTTA-CCAGTTTGCCGG------------- |
| Clon_TF14 | --ATGCCCATACGAGCGTAAGAAGAGAGTTGCTTA-CCAGTTTGCCGG------------- |
| Clon_DP8 | -----CTTTTGTG---GTGAAGAAGG----------------------------------- |
| Clon_DP1 | -----CTTTTGTG---GTGAAGAAGG----------------------------------- |
| Clon_DP7 | -----CTTTTGTG---GTGAAGAAGG----------------------------------- |
| Clon_DP3 | -----CTTTTGTG---GTGAAGAAGG----------------------------------- |
| Clon_DP6 | -----CTTTTGTG---GTGAAGAAGG----------------------------------- |
| Clon_DP9 | -----CTTTTGTG---GTGAAGAAGG----------------------------------- |
| Clon_DP2 | -----CTTTTGTG---GTGAAGAAGG----------------------------------- |
| Clon_DP4 | -----CTTTTGTG---GTGAAGAAGG----------------------------------- |
| Clon_DP10 | -----CTTTTGTG---GTGAAGAAGG----------------------------------- |
| Clon_DP5 | -----CTTTTGTG---GTGAAGAAGG----------------------------------- |
| Clon_EM4 | -----CTCTTGTG---GTGAAGAAGG----------------------------------- |
| Clon_EM21 | -----CTCTTGTG---GTGAAGAAGG----------------------------------- |
| Clon_EM2 | -----CTCTTGTG---GTGAAGAAGG----------------------------------- |
| Clon_EM23 | -----CTCTTGTG---GTGAAGAAGG----------------------------------- |
| Clon_EM3 | -----CTCTTGTG---GTGAAGAAGG----------------------------------- |
| Clon_EM24 | -----CTCTTGTG---GTGAAGAAGG----------------------------------- |
| Clon_EM22 | -----CTCTTGTG---GTGAAGAAGG----------------------------------- |
| Clon_EM1 | -----CTCTTGTG---GTGAAGAAGG----------------------------------- |
| Clon_EM6 | -----CTCTTGTG---GTGAAGAAGG----------------------------------- |
| Clon_EM5 | -----CTCTTGTG---GTGAAGAAGG----------------------------------- |
| Clon_GD1 | GTACGCCCAAAAATGCGTATTGAAGCTGTTTC----GCATATTGCAACAAACATTAATTT |
| Clon_GD10 | GTACGCCCAAAAATGCGTATTGAAGCTGTTTC----GCATATTGCAACAAACATTAATTT |
| Clon_GD2 | GTACGCCCAAAAATGCGTATTGAAGCTGTTTC----GCATATTGCAACAA---TTAATTT |
| Clon_GD5 | GTACGCCCAAAAATGCGTATTGAAGCTGTTTC----GCATATTGCAACAA---TTAATTT |
| Clon_GD3 | GTACGCCCAAAAATGCGTATTGAAGCTGTTTC----GCATATTGCAACAAACATTAATTT |
| Clon_GD12 | GTACGCCCAAAAATGCGTATTGAAGCTGTTTC----GCATATTGCAACAA---TTAATTT |
| Clon_GD7 | GTACGCCCAAAAATGCGTATTGAAGCTGTTTC----GCATATTGCAACAAACATTAATTT |
| Clon_GD9 | GTACGCCCAAGAATGCGTATTGAAGCTGTTTC----GCATATTGCAACAAACATTAATTT |
| Clon_GD8 | GTACGCCCAAAAATGCGTATTGAAGCTGTTTC----GCATATTGCAACAAACATTAATTT |
| Clon_GD13 | GTACGCCCAAAAATGCGTATTGAAGCTGTTTC----GCATATTGCAACAAACATTAATTT |
| Clon_LD5 | GTATGCCCCAAAATTCGTATCGAAGCTTTATCAA--GCATATTGCATC------------ |
| Clon_LD13 | GTATGCCCCAAAATTCGTATCGAAGCTTTATCAA--GCATATTGCATC------------ |
| Clon_LD14 | GTATGCCCCAAAATTCGTATCGAAGCTTTATCAA--GCATATTGCATC------------ |
| Clon_LD1 | GTATGCCCCAAAATTCGTATCGAAGCTTTATCAA--GCATATTGCATC------------ |
| Clon_LD11 | GTATGCCCCAAAATTCGTATCGAAGCTTTATCAA--GCATATTGCATC------------ |
| Clon_LD3 | GTATGCCCCAAAATTCGTATCGAAGCTTTATCAA--GCATATTGCATC------------ |
| Clon_LD2 | GTATGCCCCAAAATTCGTATCGAAGCTTTATCAA--GCATATTGCATC------------ |
| Clon_LD12 | GTATGCCCCAAAATTCGTATCGAAGCTTTATCAA--GCATATTGCATC------------ |
| Clon_LD8 | GTATGCCCCAAAATTCGTATCGAAGCTTTATCAATTGCATATTGCATC------------ |
| Clon_LD15 | GTATGCCCCAAAATTCGTATCGAAGCTTTATCAATTGCATATTGCATC------------ |
| Clon_DM1 | -----------------------CTTTGTA-GC--ACATTCATCA--------------- |
| Clon_DM21 | -----------------------CTTTGTA-GC--ACATTCATCA--------------- |
| Clon_DM6 | -----------------------CTTTGTA-GC--ACATTCATCA--------------- |
| Clon_DM20 | -----------------------CTTTGTA-GC--ACATTCATCA--------------- |
| Clon_DM9 | -----------------------CTTTGTA-GC--ACATTCATCA--------------- |
| Clon_DM12 | -----------------------CTTTGTA-GC--ACATTCATCA--------------- |
| Clon_DM7 | -----------------------CTTTGTA-GC--ACATTCATCA--------------- |
| Clon_DM11 | -----------------------CTTTGTA-GC--ACATTCATCA--------------- |
| Clon_DM14 | -----------------------CTTTGTA-GC--ACATTCATCA--------------- |
| Clon_DMA | -----------------------CTTTGTA-GC--ACATTCATCA--------------- |
| Clon_DF1 | -----------------------CTTTGTA-GC--ACATTCATCAC-------------- |
| Clon_DF6 | -----------------------CTTTGTA-GC--ACATTCATCAC-------------- |
| Clon_DF4 | -----------------------CTTTGTA-GC--ACATTCATCAC-------------- |
| Clon_DF26 | -----------------------CTTTGTA-GC--ACATTCATCAC-------------- |
| Clon_DF4_50 | -----------------------CTTTGTA-GC--ACATTCATCAC-------------- |
| Clon_DF19 | -----------------------CTTTGTA-GC--ACATTCATCAC-------------- |
| Clon_DF3 | -----------------------CTTTGTA-GC--ACATTCATCAC-------------- |
| Clon_DF5 | -----------------------CTTTGTA-GC--ACATTCATCAC-------------- |
| Clon_DF2 | -----------------------CTTTGTA-GC--ACATTCATCAC-------------- |
| Clon_DF7 | -----------------------CTTTGTA-GC--ACATTCATCAC-------------- |
| Clon_AS15 | ----------TACTGATCTT---TTTTGCGTGCCAATACATGCCT-TCCCCTCACGGAGA |
| Clon_AS14 | ----------TACTGATCTT---TTTTGCGTGCCAATACATGCCT-TCCCCTCACGGAGA |
| Clon_AS20 | ----------TACTGATCTT---TTTTGCGTGCCAATACATGCCT-TCCCCTCACGGAGA |
| Clon_AS13 | ----------TACTGATCTT---TTTTGCGTGCCAATACATGCCT-TCCCCTCACGGAGA |
| Clon_AS10 | ----------TACTGATCTT---TTTTGCGTGCCAATACATGCCT-TCCCCTCACGGAGA |
| Clon_AS11 | ----------TACTGATCTT---TTTTGCGTGCCAATACATGCCT-TCCCCTCACGGAGA |
| Clon_AS2 | ----------TACTGATCTT---TTTTGCGTGCCAATACATGCCT-TCCCCTCACGGAGA |
| Clon_AS12 | ----------TACTGATCTT---TTTTGCGTGCCAATACATGCCT-TCCCCTCACGGAGA |
| Clon_AS1 | ----------TACTGATCTT---TTTTGCGTGCCAATACATGCCT-TCCCCTCACGGAGA |
| Clon_AS16 | ----------TACTGATCTT---TTTTGCGTGCCAATACATGCCT-TCCCCTCACGGAGA |
| Clon_BT8 | ----------TAAG---------TTGTCCGTG---GTATGTAC----------------A |
| Clon_BT9 | ----------TAAG---------TTGTCCGTG---GTATGTAC----------------A |
| Clon_BT16 | ----------TAAG---------TTGTCCGTG---GTATGTAC----------------A |
| Clon_BT3 | ----------TAAG---------TTGTCCGTG---GTATGTAC----------------A |
| Clon_BT14 | ----------TAAG---------TTGTCCGTG---GTATGTAC----------------A |
| Clon_BT17 | ----------TAAG---------TTGTCCGTG---GTATGTAC----------------A |
| Clon_BT13 | ----------TAAG---------TTGTCCGTG---GTATGTAC----------------A |
| Clon_BT1 | ----------TAAG---------TTGTCCGTG---GTATGTAC----------------A |
| Clon_BT10 | ----------TAAG---------TTGTCCGTG---GTATGTAC----------------A |
| Clon_BT15 | ----------TAAG---------TTGTCCGTG---GTATGTAC----------------A |
| Clon_TPA1_20 | ----------TACTCGATT--CACTTTGCGTGT--AGATTTGCCGCA--CT-----TGCT |
| Clon_TPA1_22 | ----------TACTCGATT--CACTTTGCGTGT--AGATTTGCCGCA--CT-----TGCT |
| Clon_TPA1_29 | ----------TACTCGATT--CACTTTGCGTGT--AGATTTGCCGCA--CT-----TGCT |
| Clon_TPA1_28 | ----------TACTCGATT--CACTTTGCGTGT--AGATTTGCCGCA--CT-----TGCT |
| Clon_TPA1_26 | ----------TACTCGATT--CACTTTGCGTGT--AGATTTGCCGCA--CT-----TGCT |
| Clon_TPA1_21 | ----------TACTCGATT--CACTTTGCGTGT--AGATTTGCCGCA--CT-----TGCT |
| Clon_TPA1_36 | ----------TACTCGATT--CACTTTGCGTGT--AGATTTGCCGCA--CT-----TGCT |
| Clon_TPA1_27 | ----------TACTCGATT--CACTTTGCGTGT--AGATTTGCCGCA--CT-----TGCT |
| Clon_TPA1_23 | ----------TACTCGATT--CACTTTGCGTGT--AGATTTGCCGCA--CT-----TGCT |
| Clon_TPA1_1 | ----------TACTCGATT--CACTTTGCGTGT--AGATTTGCCGCA--CT-----TGCT |
| Clon_TF22 | ----------TACTCGATT--CACTTTGCGTGT--AGATTTGCCGCAGACCA---ATTCT |
| Clon_TF24 | ----------TACTCGATT--CACTTTGCGTGT--AGATTTGCCGCAGACCA---ATTCT |
| Clon_TF3 | ----------TACTCGATT--CACTTTGCGTGT--AGATTTGCCGCAGACCA---ATTCT |
| Clon_TF2 | ----------TACTCGATT--CACTTTGCGTGT--AGATTTGCCGCAGACCA---ATTCT |
| Clon_TF23 | ----------TACTCGATT--CACTTTGCGTGT--AGATTTGCCGCAGACCA---ATTCT |
| Clon_TF1 | ----------TACTCGATT--CACTTTGCGTGT--AGATTTGCCGCAGACCA---ATTCT |
| Clon_TF4 | ----------TACTCGATT--CACTTTGCGTGT--AGATTTGCCGCAGACCA---ATTCT |
| Clon_TF7 | ----------TACTCGATT--CACTTTGCGTGT--AGATTTGCCGCAGACCA---ATTCT |
| Clon_TF15 | ----------TACTCGATT--CACTTTGCGTGT--AGATTTGCCGCAGACCA---ATTCT |
| Clon_TF14 | ----------TACTCGATT--CACTTTGCGTGT--AGATTTGCCGCAGACCA---ATTCT |
| Clon_DP8 | -----------------------CTTTGTA-GC--ACATTCACT---------------- |
| Clon_DP1 | -----------------------CTTTGTA-GC--ACATTCACT---------------- |
| Clon_DP7 | -----------------------CTTTGTA-GC--ACATTCACT---------------- |
| Clon_DP3 | -----------------------CTTTGTA-GC--ACATTCACT---------------- |
| Clon_DP6 | -----------------------CTTTGTA-GC--ACATTCACT---------------- |
| Clon_DP9 | -----------------------CTTTGTT-GC--ACATTCACT---------------- |
| Clon_DP2 | -----------------------CTTTGTA-GC--ACATTCACT---------------- |
| Clon_DP4 | -----------------------CTTTGTA-GC--ACATTCACT---------------- |
| Clon_DP10 | -----------------------CTTTGTA-GC--ACATTCACT---------------- |
| Clon_DP5 | -----------------------CTTTGTA-GC--ACATTCACT---------------- |
| Clon_EM4 | -----------------------CTTTGTC-GC--ACATTCACT---------------- |
| Clon_EM21 | -----------------------CTTTGTC-GC--ACATTCACT---------------- |
| Clon_EM2 | -----------------------CTTTGTC-GC--ACATTCACT---------------- |
| Clon_EM23 | -----------------------CTTTGTC-GC--ACATTCACT---------------- |
| Clon_EM3 | -----------------------CTTTGTC-GC--ACATTCACT---------------- |
| Clon_EM24 | -----------------------CTTTGTC-GC--ACATTCACT---------------- |
| Clon_EM22 | -----------------------CTTTGTC-GC--ACATTCACT---------------- |
| Clon_EM1 | -----------------------CTTTGTC-GC--ACATTCACT---------------- |
| Clon_EM6 | -----------------------CTTTGTC-GC--ACATTCACT---------------- |
| Clon_EM5 | -----------------------CTTTGTC-GC--ACATTCACT---------------- |
| Clon_GD1 | GCTTGTTCAATGTTGTGATCGGGTTCTGTGCAC--GCGTTTAATT--------------- |
| Clon_GD10 | GTTTGTTCAATGTTGTGATCGGGTTCTGTGCAC--GCGTTTAATT--------------- |
| Clon_GD2 | ---TGTTCAATGTTGTGATCGGGTTCTGTGCAC--GCGTTTAATT--------------- |
| Clon_GD5 | ---TGTTCAATGTTGTGATCGGGTTCTGTGCAC--GCGTTTAATT--------------- |
| Clon_GD3 | GTTTGTTCAATGTTGTGATCGGGTTCTGTGCAC--GCGTTTAATT--------------- |
| Clon_GD12 | ---TGTTCAATGTTGTGATCGGGTTCTGTGCAC--GCGTTTAATT--------------- |
| Clon_GD7 | GTTTGTTCAATGTTGTGATCGGGTTCTGTGCAC--GCGTTTAATT--------------- |
| Clon_GD9 | GTTTGTTCAATGTTGTGATCGGGTTCTGTGCAC--GCGTTTAATT--------------- |
| Clon_GD8 | GTTTGTTCAATGTTGTGATCGGGTTCTGTGCAC--GCGTTTAATT--------------- |
| Clon_GD13 | GTTTGTTCAATGTTGTGATCGGGTTCTGTGCAC--GCGTTTAATT--------------- |
| Clon_LD5 | ----------------GATCGGGTTCTGTGCAC--GCGTTTAATT--------------- |
| Clon_LD13 | ----------------GATCGGGTTCTGTGCAC--GCGTTTAATT--------------- |
| Clon_LD14 | ----------------GATCGGGTTCTGTGCAC--GCGTTTAATT--------------- |
| Clon_LD1 | ----------------GATCGGGTTCTGTGCAC--GCGTTTAATT--------------- |
| Clon_LD11 | ----------------GATCGGGTTCTGTGCAC--GCGTTTAATT--------------- |
| Clon_LD3 | ----------------GATCGGGTTCTGTGCAC--GCGTTTAATT--------------- |
| Clon_LD2 | ----------------GATCGGGTTCTGTGCAC--GCGTTTAATT--------------- |
| Clon_LD12 | ----------------GATCGGGTTCTGTGCAC--GCGTTTAATT--------------- |
| Clon_LD8 | ----------------GATCGGGTTCTGTGCAC--GCGTTTAATT--------------- |
| Clon_LD15 | ----------------GATCGGGTTCTGTGCAC--GCGTTTAATT--------------- |
| | * * |
| Clon_DM1 | ------------------GGGTATTTAGCGTTTC---------------------CAGCT |
| Clon_DM21 | ------------------GGGTATTTAGCGTTTC---------------------CAGCT |
| Clon_DM6 | ------------------GGGTATTTAGCGTTTC---------------------CAGCT |
| Clon_DM20 | ------------------GGGTATTTAGCGTTTC---------------------CAGCT |
| Clon_DM9 | ------------------GGGTATTTAGCGTTTC---------------------CAGCT |
| Clon_DM12 | ------------------GGGTATTTAGCGTTTC---------------------CAGCT |
| Clon_DM7 | ------------------GGGTATTTAGCGTTTC---------------------CAGCT |
| Clon_DM11 | ------------------TGGTATTTAGCGTTTC---------------------CAGCT |
| Clon_DM14 | ------------------GGGTATTTAGCGTTTC---------------------CAGCT |
| Clon_DMA | ------------------GGGTATTTAGCGTTTC---------------------CAGCT |
| Clon_DF1 | ------------TAGAA-GGGTATTTAGCGTTTC---------------------CAGCT |
| Clon_DF6 | ------------TAGAA-GGGTATTTAGCGTTTC---------------------CAGCT |
| Clon_DF4 | ------------TAGAA-GGGTATTTAGCGTTTC---------------------CAGCT |
| Clon_DF26 | ------------TAGAA-GGGTATTTAGCGTTTC---------------------CAGCT |
| Clon_DF4_50 | ------------TAGAA-GGGTATTTAGCGTTTC---------------------CAGCT |
| Clon_DF19 | ------------TAGAA-GGGTATTTAGCGTTTC---------------------CAGCT |
| Clon_DF3 | ------------TAAAAAGGGTATTTAGCGTTTC---------------------CAGCT |
| Clon_DF5 | ------------TAGAA-GGGTATTTAGCGTTTC---------------------CAGCT |
| Clon_DF2 | ------------TAGAA-GGGTATTTAGCGTTTC---------------------CAGCT |
| Clon_DF7 | ------------TAGAA-GGGTATTTAGCGTTTC---------------------CAGCT |
| Clon_AS15 | A-GGTATTTGGATGTAGG--GCTTTTGACACTACATGTCAA------------AATGCTT |
| Clon_AS14 | A-GGTATTTGGATGTAGG--GCTTTTGACACTACATGTCAA------------AATGCTT |
| Clon_AS20 | A-GGTATTTGGATGTAGG--GCTTTTGACACTACATGTCAA------------AATGCTT |
| Clon_AS13 | A-GGTATTTGGATGTAGG--GCTTTTGACACTACATGTCAA------------AATGCTT |
| Clon_AS10 | A-GGTATTTGGATGTAGG--GCTTTTGACACTACATGTCAA------------AATGCTT |
| Clon_AS11 | A-GGTATTTGGATGTAGG--GCTTTTGACACTACATGTCAA------------AATGCTT |
| Clon_AS2 | A-GGTATTTGGATGTAGG--GCTTTTGACACTACATGTCAA------------AATGCTT |
| Clon_AS12 | A-GGTATTTGGATGTAGG--GCTTTTGACACTACATGTCAA------------AATGCTT |
| Clon_AS1 | A-GGTATTTGGATGTAGG--GCTTTTGACACTACATGTCAA------------AATGCTT |
| Clon_AS16 | A-GGTATTTGGATGTAGG--GCTTTTGACACTACATGTCAA------------AATGCTT |
| Clon_BT8 | A----------ACGTG------------CATTACA---------------------GATC |
| Clon_BT9 | A----------ACGTG------------CATTACA---------------------GATC |
| Clon_BT16 | A----------ACGTG------------CATTACA---------------------GATC |
| Clon_BT3 | A----------ACGTG------------CATTACA---------------------GATC |
| Clon_BT14 | A----------ACGTG------------CATTACA---------------------GATC |
| Clon_BT17 | A----------ACGTG------------CATTACA---------------------GATC |
| Clon_BT13 | A----------ACGTG------------CATTACA---------------------GATA |
| Clon_BT1 | A----------ACGTG------------CATTACA---------------------GATC |
| Clon_BT10 | A----------ACGTG------------CATTACA---------------------GATC |
| Clon_BT15 | A----------ACGTG------------CATTACA---------------------GATC |
| Clon_TPA1_20 | GTGGTAGTCTAATGTAGGGGGCTTCTGACACTGCCTGTCAGT---------CAAGCACCT |
| Clon_TPA1_22 | GTGGTAGTCTAATGTAGGGGGCTTCTGACACTGCCTGTCAGT---------CAAGCACCT |
| Clon_TPA1_29 | GTGGTAGTCTAATGTAGGGGGCTTCTGACACTGCCTGTCAGT---------CAAGCACCT |
| Clon_TPA1_28 | GTGGTAGTCTAATGTAGGGGGCTTCTGACACTGCCTGTCAGT---------CAAGCACCT |
| Clon_TPA1_26 | GTGGTAGTCTAATGTAGGGGGCTTCTGACACTGCCTGTCAGT---------CAAGCACCT |
| Clon_TPA1_21 | GTGGTAGTCTAATGTAGGGGGCTTCTGACACTACCTGTCAGT---------CAAGCACCT |
| Clon_TPA1_36 | GTGGTAGTCTAATGTAGGGGGCTTCTGACACTACCTGTCAGT---------CAAGCACCT |
| Clon_TPA1_27 | GTGGTAGTCTAATGTAGGGGGCTTCTGACACTGCCTGTCAGT---------CAAGCACCT |
| Clon_TPA1_23 | GTGGTAGTCTAATGTAGGGGGCTTCTGACACTGCCTGTCAGT---------CAAGCACCT |
| Clon_TPA1_1 | GTGGTAGTCTAATGTAGGGGGCTTCTGACACTGCCTGTCAGT---------CAAGCACCT |
| Clon_TF22 | GCAGTAGTCTAATGTAGGGGGCTTCTGACACTACCTGTCAGT---------TTAGCACCT |
| Clon_TF24 | GCAGTAGTCTAATGTAGGGGGCTTCTGACACTATCTGTCAGT---------TTAGCACCT |
| Clon_TF3 | GCGGTAGTCTAATGTAGGGGGCTTCTGACACTACCTGTCAGT---------TTAGCACCT |
| Clon_TF2 | GCGGTAGTCTAATGTAGGGGGCTTCTGACACTACCTGTCAGT---------TTAGCACCT |
| Clon_TF23 | GCGGTAGTCTAATGTAGGGGGCTTCTGACACTACCTGTCAGT---------TTAGCACCT |
| Clon_TF1 | GCGATAGTCTAATGTAGGGGGCTTCTGACACTACCTGTCAGT---------TTAGCGCCT |
| Clon_TF4 | GCGGTAGTCTAATGTAGGGGGCTTCTGACACTACCTGTCAGT---------TTAGCACCT |
| Clon_7F7 | GCGGTAGTCTAATGTAGGGGGCTTCTGACACTACCTGTCAGT---------TTAGCACCT |
| Clon_7F15 | GCGGTAGTCTAATGTAGGGGGCTTCTGACACTACCTGTCAGT---------TTAGCACCT |
| Clon_7F14 | GCGGTAGTCTAATGTAGGGGGCTTCTGACACTACCTGTCAGT---------TTAGCACCT |
| Clon_DP8 | ------------CACAAGAGGTATTTAGCGTTTC---------------------CAGCT |
| Clon_DP1 | ------------CACAAGAGGTATTTAGCGTTTC---------------------CAGCT |
| Clon_DP7 | ------------CACAAGAGGTATTTAGCGTTTC---------------------CAGCT |
| Clon_DP3 | ------------CACAAAAGGTATTTAGCGTTTC---------------------CAGCT |
| Clon_DP6 | ------------CACAAGAGGTATTTAGCGTTTC---------------------CAGCT |
| Clon_DP9 | ------------CACAAGAGGTATTTAGCGTTTC---------------------CAGCT |
| Clon_DP2 | ------------CACAAAAGGTATTTAGCGTTTC---------------------CAGCT |
| Clon_DP4 | ------------CACAAGAGGTATTTAGCGTTTC---------------------CAGCT |
| Clon_DP10 | ------------CACAAAAGGTATTTAGCGTTTC---------------------CAGCT |
| Clon_DP5 | ------------CACAAGAGGTATTTAGCGTTTC---------------------CAGCT |
| Clon_EM4 | ------------C--AGGAGGTATTTAGCGTTTC---------------------CAGCT |
| Clon_EM21 | ------------C--AGGAGGTATTTAGCGTTTC---------------------CAGCT |
| Clon_EM2 | ------------C--AGGAGGTATTTAGCGTTTC---------------------CAGCT |
| Clon_EM23 | ------------C--AGGAGGTATTTAGCGTTTC---------------------CAGCT |
| Clon_EM3 | ------------C--AGGAGGTATTTAGCGTTTC---------------------CAGCT |
| Clon_EM24 | ------------C--AGGAGGTATTTAGCGTTTC---------------------CAGCT |
| Clon_EM22 | ------------C--AGGAGGTATTTAGCGTTTC---------------------CAGCT |
| Clon_EM1 | ------------C--AGGAGGTATTTAGCGTTTC---------------------CAGCT |
| Clon_EM6 | ------------C--AGGAGGTATTTAGCGTTTC---------------------CAGCT |
| Clon_EM5 | ------------C--AGGAGGTATTTAGCGTTTC---------------------CAGCT |
| Clon_GD1 | -----------ACGC--AGGGCTTTTGGCACATCATGTCAAT-TGCTTGAAATTGCACTA |
| Clon_GD10 | -----------ACGC--AGGGCTTTTGGCACATCATGTCAAT-TGCTTGAAATTGCACTA |
| Clon_GD2 | -----------ACGC--AGGGCTTTTGGCACATCATGTCAAT-TGCTTGAAATTGCACTA |
| Clon_GD5 | -----------ACGC--AGGGCTTTTGGCACATCATGTCAAT-TGCTTGAAATTGCACTA |
| Clon_GD3 | -----------ACGC--AGGGCTTTTGGCACATCATGTCAAT-TGCTTGAAATTGCACTA |
| Clon_GD12 | -----------ACGC--AGGGCTTTTGGCACATCATGTCAAT-TGCTTGAAATTGCACTA |
| Clon_GD7 | -----------ACGC--AGGGCTTTTGGCACATCATGTCAAT-TGCTTGAAATTGCACTA |
| Clon_GD9 | -----------ACGC--AGGGCTTTTGGCACATCATGTCAAT-TGCTTGAAATTGCACTA |
| Clon_GD8 | -----------ACGC--AGGGCTTTTGGCACATCATGTCAAT-TGCTTGAAATTGCACTA |
| Clon_GD13 | -----------ACGC--AGGGCTTTTGGCACATCATGTCAAT-TGCTTGAAATTGCACTA |
| Clon_LD5 | -----------ACGCGCAGGGCTTATGGCACAACATGCCATTATGCCTGGA-TTGCAACA |
| Clon_LD13 | -----------ACGCGCAGGGCTTATGGCACAACATGCCATTATGCCTGGA-TTGCAACA |
| Clon_LD14 | -----------ACGCGCAGGGCTTATGGCACAACATGCCATTATGCCTGGA-TTGCAACA |
| Clon_LD1 | -----------ACGCGCAGGGCTTATGGCACAACATGCCATTATGCCTGGA-TTGCAACA |
| Clon_LD11 | -----------ACGCGCAGGGCTTATGGCACAACATGCCATTATGCCTGGA-TTGCAACA |
| Clon_LD3 | -----------ACGCGCAGGGCTTATGGCACAACATGCCATTATGCCTGGA-TTGCAACA |
| Clon_LD2 | -----------ACGCGCAGGGCTTATGGCACAACATGCCATTATGCCTGGA-TTGCAACA |
| Clon_LD12 | -----------ACGCGCAGGGCTTATGGCACAACATGCCATTATGCCTGGA-TTGCAACA |
| Clon_LD8 | -----------ACGCGCAGGGCTTATGGCACAACATGCCATTATGCCTGGA-TTGCAACA |
| Clon_LD15 | -----------ACGCGCAGGGCTTATGGCACAACATGCCATTATGCCTGGA-TTGCAACA |
| | * |
| Clon_DM1 | AAACAACCCAG----------------------------AATGTGTGC--------CTTG |
| Clon_DM21 | AAACAACCCAG----------------------------AATGTGTGC--------CTTG |
| Clon_DM6 | AAACAACCCAG----------------------------AATGTGTGC--------CTTG |
| Clon_DM20 | AAACAACCCAG----------------------------AATGTGTGC--------CTTG |
| Clon_DM9 | AAACAACCCAG----------------------------AATGTGTGC--------CTTG |
| Clon_DM12 | AAACAACCCAG----------------------------AATGTGTGC--------CTTG |
| Clon_DM7 | AAACAACCCAG----------------------------AATGTGTGC--------CTTG |
| Clon_DM11 | AAACAACCCAG----------------------------AATGTGTGC--------CTTG |
| Clon_DM14 | AAACAACCCAG----------------------------AATGTGTGC--------CTTG |
| Clon_DMA | AAACAACCCAG----------------------------AATGTGTGC--------CTTG |
| Clon_DF1 | AAACAACCCAG----------------------------AATGTGTGC--------CATG |
| Clon_DF6 | AAACAACCCAG----------------------------AATGTGTGC--------CATG |
| Clon_DF4 | AAACAACCCAG----------------------------AATGTGTGC--------CATG |
| Clon_DF26 | AAACAACCCAG----------------------------AATGTGTGC--------CATG |
| Clon_DF4_50 | AAACAACCCAG----------------------------AATGTGTGC--------CATG |
| Clon_DF19 | AAACAACCCAG----------------------------AATGTGTGC--------CATG |
| Clon_DF3 | AAACAACCCAG----------------------------AATGTGTGC--------CATG |
| Clon_DF5 | AAACAACCCAG----------------------------AATGTGTGC--------CATG |
| Clon_DF2 | AAACAACCCAG----------------------------AATGTGTGC--------CATG |
| Clon_DF7 | AAACAACCCAG----------------------------AATGTGTGC--------CATG |
| Clon_AS15 | GAGCAATTAGATCGGGTACCATCTTCT-AGGTG-GAACCAATGTATGT-----TTGCCTT |
| Clon_AS14 | GAGCAATTAGATCGGGTACCATCTTCT-AGGTG-GAACCAATGTATGT-----TTGCCTT |
| Clon_AS20 | GAGCAATTAGATCGGGTACCATCTTCTTAGGTG-GAACCAATGTATGT-----TTGCCTT |
| Clon_AS13 | GAGCAATTAGATCGGGTACCATCTTCTTAGGTG-GAACCAATGTATGT-----TTGCCTT |
| Clon_AS10 | GAGCAATTAGATCGGGTACCATCTTCTTAGGTG-GAACCAATGTATGT-----TTGCCTT |
| Clon_AS11 | GAGCAATTAGATCGGGTACCATCTTCTTAGGTG-GAACCAATGTATGT-----TTGCCTT |
| Clon_AS2 | GAGCAATTAGATCGGGTACCATCTTCT-AGGTG-GAACCAATGTATGT-----TTGCCTT |
| Clon_AS12 | GAGCAATTAGATCGGGTACCATCTTCTTAGGTG-GAACCAATGTATGT-----TTGCCTT |
| Clon_AS1 | GAGCAATTAGATCGGGTACCATCTTCT-AGGTG-GAACCAATGTATGT-----TTGCCTT |
| Clon_AS16 | GAGCAATTAGATCGGGTACCATCTTCTTAGGTG-GAACCAATGTATGT-----TTGCCTT |
| Clon_BT8 | GGACAATTGAA--------CATT-----------------ATGTATGT-----TCACTT- |
| Clon_BT9 | GGACAATTGAA--------CATT-----------------ATGTATGT-----TCACTT- |
| Clon_BT16 | GGACAATTGAA--------CATT-----------------ATGTATGT-----TCACTT- |
| Clon_BT3 | GGACAATTGAA--------CATT-----------------ATGTATGT-----TCACTT- |
| Clon_BT14 | GGACAATTGAA--------CATT-----------------ATGTATGT-----TCACTT- |
| Clon_BT17 | GGACAATTGAA--------CATT-----------------ATGTATGT-----TCACTT- |
| Clon_BT13 | GGACT-TTGAA--------CATT-----------------A---ATGT-----TCACTT- |
| Clon_BT1 | GGACAATTGAA--------CATT-----------------ATGTATGT-----TCACTT- |
| Clon_BT10 | GGACT-TTGAA--------CATT-----------------A---ATGT-----TCACTT- |
| Clon_BT15 | GGACAATTGAA--------CATT-----------------ATGTATAT-----TCACTT- |
| Clon_TPA1_20 | GAGCACTGTGT-CG--CATCGATGC--CAAGCGCTTAAAGCTGTGCGC--GTCTC-CA-T |
| Clon_TPA1_22 | GAGCACTGTGT-CA--CATCGATGCACCAGGCGCTTAAAGCTGTGCGC--GTCTC-CA-T |
| Clon_TPA1_29 | GAGCACTGTGT-CA--CATCGATGCACCAGGCGCTTAAAGCTGTGCGC--GTCTC-CA-T |
| Clon_TPA1_28 | GAGCACTGTGT-CA--CATCGATGCACCAAGCGCTTAAAGCTGTGCGC--GTCTC-CA-T |
| Clon_TPA1_26 | GAGCACTGTGT-CA--CATCGATGCACCAAGCGCTTAAAGCTGTGCGC--GTCTC-CA-T |
| Clon_TPA1_21 | GAGCACTGTGT-CA--CATCGATGC--CAAGCGCTTAAAGCTGTGCGC--GTCTC-CA-T |
| Clon_TPA1_36 | GAGCACTGTGT-CG--CATCGATGCACCAAGCGCTTAAAGCTGTGCGC--GTCTC-CAAT |
| Clon_TPA1_27 | GAGCACTGTGT-CA--CATCGATGCACCAAGCGCTTAAAGCTGTGCGC--GTCTC-CA-T |
| Clon_TPA1_23 | GAGCACTGTGT-CA--CATCGATGCACCAAGCGCTTAAAGCTGTGCGC--GTCTC-CA-T |
| Clon_TPA1_1 | GAGCACTGTGT-CG--CATCGATGC--CAAGCGCTTAAAGCTGTGCGC--GTCTC-CA-T |
| Clon_TF22 | GAGCACTGTGT-CA--CATCGCTGC--TGCGCA-TCAAAACTGTGCAAATTTCTCACTAT |
| Clon_TF24 | GAGCACTGTGT-CA--CATCGCTGC--TGCGCA-TCAAAACTGTGCAAATTTCTCACTAT |
| Clon_TF3 | GAGCACTGTGT-CA--CATCGTTGC--TGCGCA-TCAAAACTGTGCAAATTTCTCACTAT |
| Clon_TF2 | GAGCACTGTGT-CA--CATCGCTGC--TGCGCA-TCAAAACTGTGCAAATTTCTCACTAT |
| Clon_TF23 | GAGCACTGTGT-CA--CATCGCTGC--TGCGCA-TCAAAACTGTGCAAATTTCTCACTAT |
| Clon_TF1 | GAGCACTGTGT-CA--CATCGCTGC--TGCGCA-TCAAAACTGTGCAAATTTCTCACTAT |
| Clon_TF4 | GAGCACTGTGT-CA--CATCGCTGC--TGCGCA-TCAAAACTGTGCAAATTTCTCACTAT |
| Clon_TF7 | GAGCACTGTGT-CA--CATCGTTGC--TGCGCA-TCAAAACTGTGCAAATTTCTCACTAT |
| Clon_TF15 | GAGCACTGTGT-CA--CATCGCTGC--TGCGCA-TCAAAACTGTGCAAATTTCTCACTAT |
| Clon_TF14 | GAGCACTGTGT-CA--CATCGCTGC--TGCGCA-TCAAAACTGTGCAAATTTCTCACTAT |
| Clon_DP8 | AAACACCTCGA-----------------------------ATGTGTGC--------CTTG |
| Clon_DP1 | AAACACCTCGA-----------------------------ATGTGTGC--------CTTG |
| Clon_DP7 | AAACACCTCGA-----------------------------ATGTGTGC--------CTTG |
| Clon_DP3 | AAACACCTCGA-----------------------------ATGTGTGC--------CTTG |
| Clon_DP6 | AAACACCTCGA-----------------------------ATGTGTGC--------CTTG |
| Clon_DP9 | AAACACCTCGA-----------------------------ATGTGTGC--------CTTG |
| Clon_DP2 | AAACACCTCGA-----------------------------ATGTGTGC--------CTTG |
| Clon_DP4 | AAACACCTCGA-----------------------------ATGTGTGC--------CTTG |
| Clon_DP10 | TAACACCTTGA-----------------------------ATGTGTGC--------CTTG |
| Clon_DP5 | AAACACCTTGA-----------------------------ATGTGTGC--------CTTG |
| Clon_EM4 | AAACGCCCGGA-----------------------------ATGTGTGC--------CTTT |
| Clon_EM21 | AAACGCCCGGA-----------------------------ATGTGTGC--------CTTT |
| Clon_EM2 | AAACGCCCGGA-----------------------------ATGTGTGC--------CTTT |
| Clon_EM23 | AAACGCCCGGA-----------------------------ATGTGTGC--------CTTT |
| Clon_EM3 | AAACGCCCGGA-----------------------------ATGTGTGC--------CTTT |
| Clon_EM24 | AAACGCCCGGA-----------------------------ATGTGTGC--------CTTT |
| Clon_EM22 | AAACGCCCGGA-----------------------------ATGTGTGC--------CTTT |
| Clon_EM1 | AAACGCCCGGA-----------------------------ATGTGTGC--------CTTT |
| Clon_EM6 | AAACGCCCTGA-----------------------------ATGTGTGC--------CTTT |
| Clon_EM5 | AAACGCCCGGA-----------------------------ATGTGTGC--------CTTT |
| Clon_GD1 | GAATATTCCAAACTG--ATCATACAACTGATGA-----CAATATGTGC------TTCTGA |
| Clon_GD10 | GAATATTCCAAACTG--ATCATACAACTGATGA-----CAATATGTGC------TTCTGA |
| Clon_GD2 | GAATATTCCAAACTG--ATCATACAACTGATGA-----CAATATGTGC------TTCTGA |
| Clon_GD5 | GAATATTCCAAACTG--ATCATACAACTGATGA-----CAATATGTGC------TTCTGA |
| Clon_GD3 | GAATATTCCAAACTG--ATCATACAACTGATGA-----CAATATGTGC------TTCTGA |
| Clon_GD12 | GAATATTCCAAACTG--ATCATACAACTGATGA-----CAATATGTGC------TTCTGA |
| Clon_GD7 | GAATATTCCAAACTG--ATCATACAACTGATGA-----CAATATGTGC------TTCTGA |
| Clon_GD9 | GAATATTCCAAACTG--ATCATACAACTGATGA-----CAATATGTGC------TTCTGA |
| Clon_GD8 | GAATATTCCAAACTG--ATCATACAACTGATGA-----CAATATGTGC------TTCTGA |
| Clon_GD13 | GAATATTCCAAACTG--ATCATACAACTGATGA-----CAATATGTGC------TTCTGA |
| Clon_LD5 | GAATATTCCAAACCG--ATCAT--AACTGA--------CAATATGTGC------TTCTGA |
| Clon_LD13 | GAATATTCCAAACCG--ATCAT--AACTGA--------CAATATGTGC------TTCTGA |
| Clon_LD14 | GAATATTCCAAACCG--ATCAT--AACTGA--------CAATATGTGC------TTCTGA |
| Clon_LD1 | GAATATTCCAAACCG--ATCAT--AACTGA--------CAATATGTGC------TTCTGA |
| Clon_LD11 | GAATATTCCAAACCG--ATCAT--AACTGA--------CAATATGTGC------TTCTGA |
| Clon_LD3 | GAATATTCCAAACCG--ATCAT--AACTGA--------CAATATGTGC------TTCTGA |
| Clon_LD2 | GAATATTCCAAACCG--ATCAT--AACTGA--------CAATATGTGC------TTCTGA |
| Clon_LD12 | GAATATTCCAAACCG--ATCAT--AACTGA--------CAATATGTGC------TTCTGA |
| Clon_LD8 | GAATATTCCAAACCG--ATCAT--AACTGA--------CAATATGTGC------TTCTGA |
| Clon_LD15 | GAATATTCCAAACCG--ATCAT--AACTGA--------CAATATGTGC------TTCTGA |
| | * |
| Clon_DM1 | TAT-A---------------------------------TTGC------------------ |
| Clon_DM21 | TAT-A---------------------------------TTGC------------------ |
| Clon_DM6 | TAT-A---------------------------------TCGC------------------ |
| Clon_DM20 | TAT-A---------------------------------TTGC------------------ |
| Clon_DM9 | TAT-A---------------------------------TTGC------------------ |
| Clon_DM12 | TAT-A---------------------------------TTGC------------------ |
| Clon_DM7 | TAT-A---------------------------------TTGC------------------ |
| Clon_DM11 | TAT-A---------------------------------TTGC------------------ |
| Clon_DM14 | TAT-A---------------------------------TTGC------------------ |
| Clon_DMA | TAT-A---------------------------------TTGC------------------ |
| Clon_DF1 | TATGA---------------------------------TTAC------------------ |
| Clon_DF6 | TATGA---------------------------------TTAC------------------ |
| Clon_DF4 | TATGA---------------------------------TAAC------------------ |
| Clon_DF26 | TATGA---------------------------------TAAC------------------ |
| Clon_DF4_50 | TATGA---------------------------------TTAC------------------ |
| Clon_DF19 | TATGA---------------------------------TTAC------------------ |
| Clon_DF3 | TATGA---------------------------------TTAC------------------ |
| Clon_DF5 | TATGA---------------------------------TTAC------------------ |
| Clon_DF2 | TATGA---------------------------------TTAC------------------ |
| Clon_DF7 | TATGA---------------------------------TTAC------------------ |
| Clon_AS15 | TACATTT--CAGTCTCGAATGGTTAATGCAACA----TTTAA-TGCTTGTACC-TTTACT |
| Clon_AS14 | TACATTT--CAGTCTCGAATGGTTAATGCAACA----TTTAA-TGCTTGTACC-TTTACT |
| Clon_AS20 | TACATTT--CAGTCTCGAATGGTTAATGCAACA----TTTAA-TGCTTGTACC-TTTACT |
| Clon_AS13 | TACATTT--CAGTCTCGAATGGTTAATGCAACA----TTTAA-TGCTTGTACC-TTTACT |
| Clon_AS10 | TACATTT--CAGTCTCGAATGGTTAATGCAACA----TTTAA-TGCTTGTACC-TTTACT |
| Clon_AS11 | TACATTT--CAGTCTCGAATGGTTAATGCAACA----TTTAA-TGCTTGTACC-TTTACT |
| Clon_AS2 | TACATTT--CAGTCTCGAATGGTTAATGCAACA----TTTAA-TGCTTGTACC-TTTACT |
| Clon_AS12 | TACATTT--CAGTCTCGAATGGTTAATGCAACA----TTTAA-TGCTTGTACC-TTTACT |
| Clon_AS1 | TACATTT--CAGTCTCGAATGGTTAATGCAACA----TTTAA-TGCTTGTACC-TTTACT |
| Clon_AS16 | TACATTT--CAGTCTCGAATGGTTAATGCAACA----TTTAA-TGCTTGTACC-TTTACT |
| Clon_BT8 | --------------------------------A----TTGA------------------- |
| Clon_BT9 | --------------------------------A----TTGA------------------- |
| Clon_BT16 | --------------------------------A----TTGA------------------- |
| Clon_BT3 | --------------------------------A----TTGA------------------- |
| Clon_BT14 | --------------------------------A----TTGA------------------- |
| Clon_BT17 | --------------------------------A----TTGA------------------- |
| Clon_BT13 | --------------------------------A----TTGAG------------------ |
| Clon_BT1 | --------------------------------A----TTGA------------------- |
| Clon_BT10 | --------------------------------A----TTGAG------------------ |
| Clon_BT15 | --------------------------------A----TTGAG------------------ |
| Clon_TPA1_20 | TACAAT---CAGACTCGGATGA-------AGCC----CTCG-----TAACGA-------- |
| Clon_TPA1_22 | TACAAT---CAGACTCGGATGA-------AGCC----CTCG-----TAACGA-------- |
| Clon_TPA1_29 | TACAAT---CAGACTCGGATGA-------AGCC----CTCG-----TAACGA-------- |
| Clon_TPA1_28 | TACAAT---CAGACTCGGATGA-------AGCA----CTCG-----TAACGA-------- |
| Clon_TPA1_26 | TACAAT---CAGACTCGGATGA-------AGCA----CTCG-----TAACGA-------- |
| Clon_TPA1_21 | TACAAT---CAGACTCGGATGA-------AGCA----CTCG-----TAACGA-------- |
| Clon_TPA1_36 | TACAAT---CAGACTCGGATGA-------AGTA----CTCG-----TAACGA-------- |
| Clon_TPA1_27 | TACAAT---CAGACTCGGATGA-------AGCA----CTCG-----TAACGA-------- |
| Clon_TPA1_23 | TACAAT---CAGACTCGGATGA-------AGCC----CTCG-----TAACGA-------- |
| Clon_TPA1_1 | TACAAT---CAGACTCGGATGA-------AGCA----CTCG-----TAACGA-------- |
| Clon_TF22 | TACAAT---CAGACTCGGATGA-------AGCA----CTCG-----TAACGA-------- |
| Clon_TF24 | TACAAT---CAGACTCGGATGA-------AGCA----CTCG-----TAACGA-------- |
| Clon_TF3 | TACAAT---CAGACTCGGATGA-------AGCA----CTCG-----TAACGA-------- |
| Clon_TF2 | TACAAT---CAGACTCGGATGA-------AGCA----CTCG-----TAACGA-------- |
| Clon_TF23 | TACAAT---CAGACTCGGATGA-------AGCA----CTCG-----TAACGA-------- |
| Clon_TF1 | TACAAT---CAGACTCGGATGA-------AGCA----CTCG-----TAACGA-------- |
| Clon_TF4 | TACAAT---CAGACTCGGATGA-------AGCA----CTCG-----TAACGA-------- |
| Clon_TF7 | TACAAT---CAGACTCGGATGA-------AGCA----CTCG-----TAACGA-------- |
| Clon_TF15 | TACAAT---CAGACTCGGATGA-------AGCA----CTCG-----TAACGA-------- |
| Clon_TF14 | TACAAT---CAGACTCGGATGA-------AGCA----CTCG-----TAACGA-------- |
| Clon_DP8 | CTTAA--------------------------------CCAA------------------- |
| Clon_DP1 | CTTAA--------------------------------CCAA------------------- |
| Clon_DP7 | CATAA--------------------------------CCAA------------------- |
| Clon_DP3 | TATAA--------------------------------CCAA------------------- |
| Clon_DP6 | CATAA--------------------------------CCAA------------------- |
| Clon_DP9 | TATAA--------------------------------CCAGT------------------ |
| Clon_DP2 | TATAA--------------------------------CCAAT------------------ |
| Clon_DP4 | TATAA--------------------------------CCGGT------------------ |
| Clon_DP10 | TATAA--------------------------------CCAA------------------- |
| Clon_DP5 | CATAA--------------------------------CCAA------------------- |
| Clon_EM4 | TGCTA--------------------------------CTT-------------------- |
| Clon_EM21 | TGCTA--------------------------------CTT-------------------- |
| Clon_EM2 | TGCTA--------------------------------CTT-------------------- |
| Clon_EM23 | TGCTA--------------------------------CTT-------------------- |
| Clon_EM3 | TGCTA--------------------------------CTT-------------------- |
| Clon_EM24 | TGCTA--------------------------------CTT-------------------- |
| Clon_EM22 | TGCTA--------------------------------CTT-------------------- |
| Clon_EM1 | TGCTA--------------------------------CTT-------------------- |
| Clon_EM6 | TGCTA--------------------------------CTT-------------------- |
| Clon_EM5 | TGCTA--------------------------------CTT-------------------- |
| Clon_GD1 | TACGAT------ACTTTGGTGAAA-------------CATA--------ATTCTCA-ATT |
| Clon_GD10 | TACGAT------ACTTTGGTGAAA-------------CATA--------ATTCTCA-ATT |
| Clon_GD2 | TACGAT------ACTTTGGTGAAA-------------CATA--------GCTAAAACAAT |
| Clon_GD5 | TACGAT------ACTTTGGTGAAA-------------CATA--------GCTAAAACAAT |
| Clon_GD3 | TACGAT------ACTTTGGTGAAA-------------CATA--------GCTAAAACAAT |
| Clon_GD12 | TACGAT------ACTTTGGTGAAA-------------CATA--------GCTAAAACAAT |
| Clon_GD7 | TACGAT------ACTTTGGTGAAA-------------CATA--------GCTAAAACAAT |
| Clon_GD9 | TACGAT------ACTTTGGTGAAA-------------CATA--------GCTAAAACAAT |
| Clon_GD8 | TACGAT------ACTTTGGTGAAA-------------CATA--------ATTCTCA-ATT |
| Clon_GD13 | TACGAT------ACTTTGGTGAAA-------------CATA--------GCTAAAACAAT |
| Clon_LD5 | TACATTT---GAAATTTGGTGGTA-------------TTTGTTCTGTATGTTGTGTTCTT |
| Clon_LD13 | TACATTT---GAAATTTGGTGGTA-------------TTTGTTCTGTATGTTGTGTTCTT |
| Clon_LD14 | TACATTT---GAAATTTGGTGGTA-------------TTTGTTCTGTATGTTGTGTTCTT |
| Clon_LD1 | TACATTT---GAAATTTGGTGGTA-------------TTTGTTCTGTATGTTGTGTTCTT |
| Clon_LD11 | TACATTT---GAAATTTGGTGGTA-------------TTTGTTCTGTATGTTGTGTTCTT |
| Clon_LD3 | TACATTT---GAAATTTGGTGGTA-------------TTTGTTCTGTATGTTGTGTTCTT |
| Clon_LD2 | TACATTT---GAAATTTGGTGGTA-------------TTTGTTCTGTATGTTGTGTTCTT |
| Clon_LD12 | TACATTT---GAAATTTGGTGGTA-------------TTTGTTCTGTATGTTGTGTTCTT |
| Clon_LD8 | TACATTT---GAAATTTGGTGGTA-------------TTTGTTCTGTATGTTGTGTTCTT |
| Clon_LD15 | TACATTT---GAAATTTGGTGGTA-------------TTTGTTCTGTATGTTGTGTTCTT |
| Clon_DM1 | CAACAT---GGATG------------------AGCATTGTAGAATGTATAACTTTAAATT |
| Clon_DM21 | CAACAT---GGATG------------------AGCATTGTAGAATGTATAACTTTAAATT |
| Clon_DM6 | CAACAT---GGATT------------------AGTATTGTAGAATTTGTAACTTTAAATT |
| Clon_DM20 | CAACAT---GGATG------------------AGCATTGTAGAATGTATAACTTTAAATT |
| Clon_DM9 | CAACAT---GGATT------------------AGTATTGCG-----TGTAAATTGCAATT |
| Clon_DM12 | CAACAT---GGATT------------------AGTATTGCG-----TGTAAATTGCAATT |
| Clon_DM7 | CAACAT---GGATT------------------AGTATTGCG-----TGTAAATTGCAATT |
| Clon_DM11 | CAACAT---GGATG------------------AGCATTGTAGAATGTATAACTTTAAATT |
| Clon_DM14 | CAACAT---GGATT------------------AGCATTGTAGAATGTATAACTTTAAATT |
| Clon_DMA | CAACAT---GGATT------------------AGTATTGCG-----TGTAAATTGCAATT |
| Clon_DF1 | CAACAT---GGATT------------------AACATT----------CAAATTTCAATT |
| Clon_DF6 | CAACAT---GGATT------------------AACATT----------CAAATTTCAATT |
| Clon_DF4 | CAACAT---GGATT------------------AATATT----------CAAATTTCAATT |
| Clon_DF26 | CAACAT---GGATT------------------AATATT----------CAAATTTCAATT |
| Clon_DF4_50 | CAACAT---GGATT------------------AACATT----------CAAATTTCAATT |
| Clon_DF19 | CAACAT---GGATT------------------AACATT----------CAAATTTCAATT |
| Clon_DF3 | CAACAT---GGATT------------------AATATT----------CAAATTTCAATT |
| Clon_DF5 | CAACAT---GGATT------------------AATATT----------CAAATTTCAATT |
| Clon_DF2 | CAACAT---GGATT------------------AACATT----------CAAATTTCAATT |
| Clon_DF7 | CAACAT---GGATT------------------AATATT----------CAAATTTCAATT |
| Clon_AS15 | TGAGAA---GTGTCA-CAATAGTTTAC-CCTTACTAGTAGAGA---CTTGACCGTAAAAC |
| Clon_AS14 | TGAGAA---GTGTCA-CAATAGTTTAC-CCTTACTAGTAGAGA---CTTGACCGTAAAAC |
| Clon_AS20 | TGAGAA---GTGTCA-CAATAGTTTAC-CCTTACTAGTAGAGA---CTTGACCGTAAAAC |
| Clon_AS13 | TGAGAA---GTGTCA-CAATAGTTTAC-CCTTACTAGTAGAGA---CTTGACCGTAAAAC |
| Clon_AS10 | TGAGAA---GTGTCA-CAATAGTTTAC-CCTTACTAGTAGAGA---CTTGACCGTAAAAC |
| Clon_AS11 | TGAGAA---GTGTCA-CAATAGTTTAC-CCTTACTAGTAGAGA---CTTGACCGTAAAAC |
| Clon_AS2 | TGAGAA---GTGTCA-CAATAGTTTAC-CCTTACTAGTAGAGA---CTTGACCGTAAAAC |
| Clon_AS12 | TGAGAA---GTGTCA-CAATAGTTTAC-CCTTACTAGTAGAGA---CTTGACCGTAAAAC |
| Clon_AS1 | TGAGAA---GTGTCA-CAATAGTTTAC-CCTTACTAGTAGAGA---CTTGACCGTAAAAC |
| Clon_AS16 | TGAGAA---GTGTCA-CAATAGTTTAC-CCTTACTAGTAGAGA---CTTGACCGTAAAAC |
| Clon_BT8 | -AAAAA---AC-TC--CAATA-----------ACAA--------------ACAACAAAA- |
| Clon_BT9 | -AAAAA---AC-TC--CAATA-----------ACAA--------------ACAACAAAA- |
| Clon_BT16 | -AAAAA---AC-TC--CAATA-----------ACAA--------------ACAACAAAA- |
| Clon_BT3 | -AAAAA---AC-TC--CAATA-----------ACAA--------------ACAACAAAA- |
| Clon_BT14 | -AAAAA---AC-TC--CAATA-----------ACAA--------------ACAACAAAA- |
| Clon_BT17 | -AAAAA---AC-TC--CAATA-----------ACAA--------------ACAACAAAA- |
| Clon_BT13 | -AAAAA---AC-TC--CAATA-----------ACAA--------------ACAACAAAA- |
| Clon_BT1 | -AAAAA---AC-TC--CAATA-----------ACAA--------------ACAACAAAA- |
| Clon_BT10 | -AAAAA---AC-TC--CAATA-----------ACAA--------------ACAACAAAA- |
| Clon_BT15 | -AAAAA----C-TC--CAATA-----------ACAA--------------ACAACAAAA- |
| Clon_TPA1_20 | TGAACT---GTGTT--TA------CA----CTAAAACTTTG------CATACTGCACATT |
| Clon_TPA1_22 | TGAACT---GTGTT--TA------CA----CTAAAACTTTG------CATACTGCACATT |
| Clon_TPA1_29 | TGAACT---GTGTT--TA------CA----CTAAAACTTTG------CATACTGCACATT |
| Clon_TPA1_28 | TGAACT---GTGTT--TA------CA----CTAAAACTTTG------CATACTGCACATT |
| Clon_TPA1_26 | TGAACT---GTGTT--TA------CA----CTAAAACTTTG------CATACTGCACATT |
| Clon_TPA1_21 | TGAACT---GTGTT--TA------CA----CTAAAACTTTG------CATACTGCGCAC- |
| Clon_TPA1_36 | TGAACT---GTGTT--TA------CA----CTAAAACTTTG------CATACTGCGCAC- |
| Clon_TPA1_27 | TGAACT---GTGTT--TA------CA----CTAAAACTTTG------CATACTGCACATT |
| Clon_TPA1_23 | TGAACT---GTGTT--TA------CA----CTAAAACTTTG------CATACTGCACATT |
| Clon_TPA1_1 | TGAACT---GTGTT--TA------CA----CTAAAACTTTG------CATACTGCACATT |
| Clon_TF22 | TGAATT---GTGTT--TA------CAC--GCTAAAACTTTG------CATACT----TTG |
| Clon_TF24 | TGAATT---GTGTT--TA------CAC--GCTAAAACTTTG------CATACT----TTG |
| Clon_TF3 | TGAATT---GTGTT--TA------CAC--GCTAAAACTTTG------CATACT----TTG |
| Clon_TF2 | TGAATT---GTGTT--TA------CA----CTAAAACTTTG------CATACT----TTG |
| Clon_TF23 | TGAATT---GTGTT--TA------CG----CTAAAACTTTG------CATACTAAAACTG |
| Clon_TF1 | TGAATT---GTGTT--TA------CA----CTAAAACTTTG------CATACT----TTG |
| Clon_TF4 | TGAATT---GTGTT--TA------CA----CTAAAACTTTG------CATACT----TTG |
| Clon_TF7 | TGAATT---GTGTT--TA------CA----CTAAAACTTTG------CATACT----TTG |
| Clon_TF15 | TGAATT---GTGTT--TA------CA----CTAAAACTTTG------CATACT----TTG |
| Clon_TF14 | TGAATT---GTGTT--TA------CG----CTAAAACTTTG------CATACTAAAACTG |
| Clon_DP8 | --AATA---TAGTCG-CAA-------------ATCATTGTC-------CAAAACAAAAC- |
| Clon_DP1 | --AATA---TAGTCG-CAA-------------ATCATTGTC-------CAAAACAAAAC- |
| Clon_DP7 | --AATA---TAGTTG-CAA-------------ATCATTGTA-------CAAAATAAAAC- |
| Clon_DP3 | --AGTA---TAGTCG-CAA-------------ATCATTGTC-------CAA--TAAAA-- |
| Clon_DP6 | --AGTA---TAGTCG-CAA-------------ATCATTGTA-------CAAATCAAAAC- |
| Clon_DP9 | -CAAAA---TGGTTGACAA-------------ATAAT-GTA-------CCA-----ATC- |
| Clon_DP2 | -CAAAA---TGGTTGACAA-------------ATCAATGT--------CCAAT--AAAA- |
| Clon_DP4 | -CAAAA---TGGTTGACAA-------------ATCAATGTA-------CCAAT-----C- |
| Clon_DP10 | --AGTA---TAGTCG-CAA-------------ATCATTGT--------CCAAT--AAAA- |
| Clon_DP5 | --AGTA---TAGTCG-CAA-------------ATCATTGTA-------CAAATCAAAAC- |
| Clon_EM4 | ----TC---AAGTCAACAG-------------ATCATTGTT-------CCAAAACAAAT- |
| Clon_EM21 | ----TC---AAGTCAACAG-------------ATCATTGTT-------CCAAAACAAAT- |
| Clon_EM2 | ----TC---AAGTCAACAG-------------ATCATTGTT-------CCAAAACAAAT- |
| Clon_EM23 | ----TC---AAGTCAACAG-------------ATCATTGTT-------CCAAAACAAAT- |
| Clon_EM3 | ----TC---AAGTCAACAG-------------ATCATTGTT-------GCAAAACAAAT- |
| Clon_EM24 | ----TC---AAGTCAACAG-------------ATCATTGTT-------GCAAAACAAAT- |
| Clon_EM22 | ----TC---AAGTCAACAG-------------ATCATTGTT-------CCAAAACAAAT- |
| Clon_EM1 | ----TC---AAGTCAACAG-------------ATCATTGTT-------CCAAAACAAAT- |
| Clon_EM6 | ----TC---AAGTCAACAG-------------ATCATTGTT-------CCAAAA-AAAT- |
| Clon_EM5 | ----TC---AAGTCAACAG-------------ATCATTGTT-------CCAAAA-AAAT- |
| Clon_GD1 | CCAAGT---GTGT-ATGGG-----------CT--AATTTTT------TAG--CGCATATG |
| Clon_GD10 | CCAAGT---GTGT-ATGGG-----------CT--AATTTTT------TAG--CGCATATG |
| Clon_GD2 | CCAAGT---GTGT-ATGGG-----------CT--AATTTTT------TAG--CGCATATG |
| Clon_GD5 | CCAAGT---GTGT-ATGGG-----------CT--AATTTTT------TAG--CGCATATG |
| Clon_GD3 | CCAAGT---GTGT-ATGGG-----------CT--AATTTTT------TAG--CGCATATG |
| Clon_GD12 | CCAAGT---GTGT-ATGGG-----------CT--AATTTTT------TAG--CGCATATG |
| Clon_GD7 | CCAAGT---GTGT-ATGGG-----------CT--AATTTTT------TAG--CGCATATG |
| Clon_GD9 | CCAAGT---GTGT-ATGGG-----------CT--AATTTTT------TAG--CGCATATG |
| Clon_GD8 | CCAAGT---GTGT-ATGGG-----------CT--AATTTTT------TAG--CGCATATG |
| Clon_GD13 | CCAAGT---GTGT-ATGGG-----------CT--AATTTTT------TAG--CGCATATG |
| Clon_LD5 | TCATTT--TGTGTGATTGA-----------TTGTAATTATT------GTGATTACAATTG |
| Clon_LD13 | TCATTT--TGTGTGATTGA-----------TTGTAATTATT------GTGATTACAATTG |
| Clon_LD14 | TCATTT--TGTGTGATTGA-----------TTGTAATTATT------GTGATTACAATTG |
| Clon_LD1 | TCATTT--TGTGTGATTGA-----------TTGTAATTATT------GTGATTACAATTG |
| Clon_LD11 | TCATTT--TGTGTGATTGA-----------TTGTAATTATT------GTGATTACAATTG |
| Clon_LD3 | TCATTT--TGTGTGATTGA-----------TTGTAATTATT------GTGATTACAATTG |
| Clon_LD2 | TCATTTGTTGTGTAATTGA-----------TTGTAATTATT------GTGATTACAATTG |
| Clon_LD12 | TCATCT--TGTGTGATTGA-----------TTGTAATTATT------GTGATTACAATTG |
| Clon_LD8 | TCATTTGTTGTGTAATTGA-----------TTGTAATTTTT-----------TACAATTG |
| Clon_LD15 | TCATTTGTTGTGTAATTGA-----------TTGTAATTTTT-----------TACAATTG |
| | * * |
| Clon_DM1 | --------------------GTCTATATTCGACCTCAGATCAAGCGAGACTA--- |
| Clon_DM21 | --------------------GTCTATATTCGACCTCAGATCAAGCGAGACTA--- |
| Clon_DM6 | --------------------GTCTATATTCGACCTCAGATCAAGCGAGACTA--- |
| Clon_DM20 | --------------------GTCTATATTCGACCTCAGATCAAGCGAGACTA--- |
| Clon_DM9 | --------------------GTCTATATTCGACCTCAGATCAAGCGAGACTA--- |
| Clon_DM12 | --------------------GTCTATATTCGACCTCAGATCAAGCGAGACTA--- |
| Clon_DM7 | --------------------GTCTATATTCGACCTCAGATCGAGCGAGACTA--- |
| Clon_DM11 | --------------------GTCTATATTCGACCTCAGATCAAGCGAGACTA--- |
| Clon_DM14 | --------------------GTCTATATTCGACCTCAGATCAAGCGAGACTA--- |
| Clon_DMA | --------------------GTCTATATTCGACCTCAGATCAAGCGAGACTA--- |
| Clon_DF1 | --------------------GTCTATATTCGACCTCAGATCAAGCGAGACTA--- |
| Clon_DF6 | --------------------GTCTATATTCGACCTCAGATCAAGCGAGACTA--- |
| Clon_DF4 | --------------------GTCTATATTCGACCTCAGATCAAGCGAGACTA--- |
| Clon_DF26 | --------------------GTCTATATTCGACCTCAGATCAAGCGAGACTA--- |
| Clon_DF4_50 | --------------------GTCTATATTCGACCTCAGATCAAGCGAGACTA--- |
| Clon_DF19 | --------------------GTCTATATTCGACCTCAGATCAAGCGAGACTA--- |
| Clon_DF3 | --------------------GTCTATATTCGATCTCAGATCAAGCGAGACTA--- |
| Clon_DF5 | --------------------GTCTATATTCGACCTCAGATCAAGCGAGACTA--- |
| Clon_DF2 | --------------------GTCTATATTCGACCTCAGATCAAGCGAGACTA--- |
| Clon_DF7 | --------------------GTCTATATTCGACCTCAGATCAAGCGAGACTA--- |
| Clon_AS15 | AG-TCACTGATCTTCCGGT-GTTAACCTTCGACCTCAGGTCAAGCGAGATTA--- |
| Clon_AS14 | AG-TCACTGATCTTCTGGT-GTTAACCTTCGACCTCAGGTCAAGCGAGATTA--- |
| Clon_AS20 | AG-TCACTGATCTTCTGGT-GTTAACCTTCGACCTCAGGTCAAGCGAGATTA--- |
| Clon_AS13 | AG-TCACTGATCTTCTGGT-GTTAACCTTCGACCTCAGGTCAAGCGAGATTA--- |
| Clon_AS10 | AG-TCACTGATCTTCTGGT-GTTAACCTTCGACCTCAGGTCAAGCGAGATTA--- |
| Clon_AS11 | AG-TCACTGATCTTCTGGT-GTTAACCTTCGACCTCAGGTCAAGCGAGATTA--- |
| Clon_AS2 | AG-TCACTGATCTTCTGGT-GTTAACCTTCGACCTCAGGTCAAGCGAGATTA--- |
| Clon_AS12 | AG-TCACTGATCTTCTGGT-GTTAACCTTCGACCTCAGGTCAAGCGAGATTA--- |
| Clon_AS1 | AG-TCACTGATCTTCTGGT-GTTAACCTTCGACCTCAGGTCAAGCGAGATTA--- |
| Clon_AS16 | AG-TCACTGATCTTCTGGT-GTTAACCTTCGACCTCAGGTCAAGCGAGATTA--- |
| Clon_BT8 | ---TCA---AAGTTTT----GTCAAA-TTCGACCTCAGATCAAGCGAGATTA--- |
| Clon_BT9 | ---TCA---AAGTTTT----GTCAAA-TTCGACCTCAGATCAAGCGAGATTA--- |
| Clon_BT16 | ---TCA---AAATTTT----GTCAAA-TTCGACCTCAGATCAAGCGAGATTA--- |
| Clon_BT3 | ---TCA---AATTTTT----GTCAAA-TTCGACCTCAGATCAAGCGAGATTA--- |
| Clon_BT14 | ---TCA---AATTTTT----GTCAAA-TTCGACCTCAGATCAAGCGAGATTA--- |
| Clon_BT17 | ---TCA---AAGTTTT----GTCAAA-TTCGACCTCAGATCAAGCGAGATTA--- |
| Clon_BT13 | ---TCA---AAGTTTT----GTCAAA-TTCGACCTCAGATCAAGCGAGATTA--- |
| Clon_BT1 | ---TCA---AAGTTTT----GTCAAA-TTCGACCTCAGATCAAGCGAGATTA--- |
| Clon_BT10 | ---TCA------TTTT----GTCAAA-TTCGACCTCAGATCAAGCGAGATTA--- |
| Clon_BT15 | ---TCA---AAATTTT----GTCAAA-TTCGACCTCAGATCAAGCGAGATTA--- |
| Clon_TPA1_20 | GATTTGT----GCAGTTGTTGTTAACTTTCGACCTCAGATCAAGCGAGATTA--- |
| Clon_TPA1_22 | GATTTGT----GCAGTTGTTGTTAACTTTCGACCTCAGATCAAGCGAGATTA--- |
| Clon_TPA1_29 | GATTTGT----GCAGTTGTTGTTAACTTTCGACCTCAGATCAAGCGAGATTA--- |
| Clon_TPA1_28 | GATTTGT----GCAGTTGTTGTTAACTTTCGACCTCAGATCAAGCGAGATTA--- |
| Clon_TPA1_26 | GATTTGT----GCAGTTGTTGTTAACTTTCGACCTCAGATCAAGCGAGATTA--- |
| Clon_TPA1_21 | -ATTTGT----GCAGTTGTTGTTAACTTTCGACCTCAGATCAAGCGAGATTA--- |
| Clon_TPA1_36 | -ATTTGT----GCAGTTGTTGTTAACTTTCGACCTCAGATCAAGCGAGATTA--- |
| Clon_TPA1_27 | GATTTGT----GCAGTTGTTGTTAACTTTCGACCTCAGATCAAGCGAGATTA--- |
| Clon_TPA1_23 | GATTTGT----GCAGTTGTTGTTAACTTTCGACCTCAGATCAAGCGAGATTA--- |
| Clon_TPA1_1 | GATTTGT----GCAGTTGTTGTTAACTITCGACCTCAGATCAAGCGAGATTA--- |
| Clon_TF22 | TG--CA-----GATGT GTTGTTAACTITCGACCTCAGATCAAGCGAGATTA--- |
| Clon_TF24 | TG--CA-----GATGT-GTTGTTAACTTTCGACCTCAGATCAAGCGAGATTA--- |
| Clon_TF3 | TGTGCA-----GTTGT--TTGTTAACTTTCGACCTCAGATCAAGCGAGATTA--- |
| Clon_TF2 | TG--CA-----GATGT GTTGTTAACTITCGACCTCAGATCAAGCGAGATTA--- |
| Clon_TF23 | TG--CA-----GTTGT--TTGTTAACTTTCGACCTCAGATCAAGCGAGATTA--- |
| Clon_TF1 | TG--CA-----GATGT-GTTGTTAACTITCGACCTCAGATCAAGCGAGATTA--- |
| Clon_TF4 | TG--CA-----GATGT-GTTGTTAACTITCGACCTCAGGTCAAGCGAGATTA--- |
| Clon_TF7 | TG--CA-----GATGT-GTTGTTAACTITCGACCTCAGATCAAGCGAGATTA--- |
| Clon_TF15 | TG--CA-----GATGT-GTTGTTAACTITCGACCTCAGATCAAGCGAGATTA--- |
| Clon_TF14 | TG--CA-----GTTGT--TTGTTAACTTTCGACCTCAGATCAAGCGAGATTA--- |
| Clon_DP8 | --------------------GTCTAATTTCGACCTCAGATCAAGCGAGAATA--- |
| Clon_DP1 | --------------------GTCTAATTTCGACCTCAGATCAAGCGAGAATA--- |
| Clon_DP7 | --------------------GTCTAATTTCGACCTCAGATCAAGCGAGAATA--- |
| Clon_DP3 | --------------------GTCTAATTTCGACCTCAGATCAAGCGAGAATA--- |
| Clon_DP6 | --------------------GTCTAATTTCGACCTCAGATCAAGCGAGAATA--- |
| Clon_DP9 | --------------------GTCTAATTTCGACCTCAGATCAAGCGAGAATA--- |
| Clon_DP2 | --------------------GTCTAATTTCGACCTCAGATCAAGCGAGAATA--- |
| Clon_DP4 | --------------------GTCTAATTTCGACCTCAGATCAAGCGAGAATA--- |
| Clon_DP10 | --------------------GTCTAATTTCGACCTCAGATCAAGCGAGAATA--- |
| Clon_DP5 | --------------------GTCTAATTTCGACCTCAGATCAAGCGAGAATA--- |
| Clon EM4 | --------------------GTCTAATTTCGACCTCAGATCAAGCGAGACTA--- |
| Clon EM21 | --------------------GTCTAATTTCGACCTCAGATCAAGCGAGACTA--- |
| Clon_EM2 | --------------------GTCTAATTTCGACCTCAGATCAAGCGAGACTA--- |
| Clon EM23 | --------------------GTCTAATTTCGACCTCAGATCAAGCGAGACTA--- |
| Clon EM3 | --------------------GTCTAATTTCGACCTCAGATCAAGCGAGACTA--- |
| Clon EM24 | --------------------GTCTAATTTCGACCTCAGATCAAGCGAGACTA--- |
| Clon_EM22 | --------------------GTCTAATTTCGACCTCAGATCAAGCGAGACTA--- |
| Clon EM1 | --------------------GTCTAATTTCGACCTCAGATCAAGCGAGACTA--- |
| Clon EM6 | --------------------GTCTAATTTCGACCTCAGATCAAGCGAGACTA--- |
| Clon EM5 | --------------------GTCTAATTTCGACCTCAGATCAAGCGAGACTA--- |
| Clon GD1 | TTTTT------------TTTGTAAACTTTCGACCTCAGATCAAGCGAGATTA--- |
| Clon_GD10 | TTTTT------------TTTGTAAACTTTCGACCTCAGATCAAGCGAGATTA--- |
| Clon_GD2 | TTTTT------------TTTGTAAACTTTCGACCTCAGATCAAGCGAGATTA--- |
| Clon_GD5 | TTTTT------------TTTGTAAACTTTCGACCTCAGATCAAGCGAGATTA--- |
| Clon_GD3 | TTTTT------------TTTGTAAACTTTCGACCTCAGATCAAGCGAGATTA--- |
| Clon_GD12 | TTTTT------------TTTGTAAACTTTCGACCTCAGATCAAGCGAGATTA--- |
| Clon_GD7 | TTTTT------------TTTGTAAACTTTCGACCTCAGATCAAGCGAGATTA--- |
| Clon_GD9 | TTTTT------------TTTGTAAACTTTCGACCTCAGATCAAGCGAGATTA--- |
| Clon_GD8 | TTTTT------------TTTGTAAACTTTCGACCTCAGATCAAGCGAGATTA--- |
| Clon_GD13 | TTTTT------------TTTGTAAACTTTCGACCTCAGATCAAGCGAGATTA--- |
| Clon_LD5 | ATATTG-----------TTTGTAAACTTTCGACCTCAGATCAAGCGAGATTA--- |
| Clon_LD13 | ATATTG-----------TTTGTAAACTTTCGACCTCAGATCAAGCGAGATTA--- |
| Clon_LD14 | ATATTG-----------TTTGTAAACTTTCGACCTCAGATCAAGCGAGATTA--- |
| Clon_LD1 | ATATTG-----------TTTGTAAACTTTCGACCTCAGATCAAGCGAGATTA--- |
| Clon_LD11 | ATATTG-----------TTTGTAAACTTTCGACCTCAGATCAAGCGAGATTA--- |
| Clon_LD3 | ATATTG-----------TTTGTAAACTTTCGACCTCAGATCAAGCGAGATTA--- |
| Clon_LD2 | ATATTG-----------TTTGTAAACTTTCGACCTCAGATCAAGCGAGATTA--- |
| Clon_LD12 | ATATTG-----------TTTGTAAACTTTCGACCTCAGATCAAGCGAGATTA--- |
| Clon_LD8 | ATATTG-----------TTTGTAAACTTTCGACCTCAGATCAAGCGAGATTA--- |
| Clon_LD15 | ATATTG-----------TTTGTAAACTTTCGACCTCAGATCAAGCGAGATTA--- |
| | ** * ***** ***** ** ******* ** |

### Example 1

DNA extraction from single individuals: Individuals are carefully isolated under a stereoscopic microscope by the aid of entomological needles. If they came from a lot pools preserved in ethanol, wash once in a clean ethanol 70% solution. Each individual is deposited on a 1.5 ml micro-tube place on ice.

This protocol has been also successfully applied to extract DNA from 10-50 individuals in 70% ethanol. In this case, ethanol is removed after centrifugation (14000 g, 5 seconds).

1. Add 100 μl of Extraction Buffer (Tris-HCl 10 mM pH 8.0, EDTA 25 mM pH 8.0 y NaCl 100 mM) and homogenise by the aid of a Pellet pestle adapted to the micro-tube (e.g. Pellet pestle, Sigma). A good homogenisation is crucial.
2. Add 10 μl of SDS al 10% (1% final) and mix gently.
3. Add 10 μl of proteinase K (stock: 10 mg/ml) (Final 1 mg/ml), mix gently and incubate 37° C., 2 hours.
4. Add 20 μl of NaCl 5M (final≈0.5 M NaCl) and mix gently.
5. Add 15 μl of cetyltrimethylammonium bromide (CTAB) (Stock: CTAB 10% in NaCl 0.5M, 62° C.) and incubate for 10 minutes at 62° C. Mix gently several times during incubation.
6. Add 0.5 volumes of equilibrated phenol and 0.5 volumes of chloroform: isoamyl alcohol (24:1). Mix gently for two minutes and centrifuge at 11000 g for 3 minutes.
7. Carefully remove approximately 110 μl of the aqueous phase and transfer to a clean micro-tube. Add 0.5 volumes of distilled water (˜55 μl), add 1 volume of isopropanol (˜170 μl), mix gently, wait two minutes and centrifuge at 18000 g for 15 minutes.
8. Remove isopropanol immediately after centrifugation by inverting the tube. Add 700 μl of 70% ethanol to wash the pellet (generally, it cannot be seen) and centrifuge at 18000 g for 15 minutes.
9. Remove ethanol immediately after centrifugation by inverting the tube and place the tube containing the pellet to let it dry on the bench or in a laminar flow cabinet (drying should be completed before 1 hour) (do not let dry more than necessary).

### 10. Add 20 μl of TE 0.1× (Stock TE 1×: Tris-HCl 10 mM pH 8.0 y EDTA 10 mM pH 8.0).

### 11. Quantify

DNA extraction from mite cultures: The same protocol, but adding 4× volumes in points 1, 2, 3, 4, 5, 7, 8 and adding RNAase (0.1 μg/μl) in point 10, has also been applied for DNA extraction from 20 mg of frozen mite cultures.

DNA extraction from environmental samples or purified mite fractions: Use DNeasy Blood and Tissue Kit (Qiagen) from purified fractions (bodies or faeces, 20 mg) and environmental samples (50 mg) and follow manufacturer instructions for purification of total DNA.

### Example 2

### One-Step Multiplex-PCR Using One or More First (Forward) Primers and a Single Second (Reverse) Primer Hybridising to 5.8S

Tested for species identification in cultures of all species, and in purified fractions of Dermatophagoides pteronyssinus and D. farinae.

Identifies the following species DNA: Tyrophagus fanetzhangorum, Lepidoglyphus destructor, Glycyphagus domesticus, Dermatophagoides pteronyssinus, Tyrophagus putrescentiae, Blomia tropicalis, Euroglyphus maynei, Dermatophagoides microceras, Acarus siro, Dermatophagoides farinae.

1. DNA extraction. See Example 1

### 3. Multiplex-PCR.

Primers: The reaction may be performed

a) using the combination of the set of ten forward (first) primers (see Table 1) with the reverse (second) primer RAst5.8S (5′-TGCGTTCGAAWGTCGAGT-3′), W=A or T
b) using any combination of two or more primers, the reverse (second) primer being one of them.

PCR reaction: a final volume of 25 μL contains

50-150 ng of DNA template

1×PCR Buffer II

200 μM dNTP mix

0.6 μM forward primers mix [0.06 μM each in this case where all ten are used]1 (Table 1)

0.6 μM reverse primer (RAst5.8S)

1.5 mM MgCl2

0.6 mg/mL purified BSA (New England Biolabs, ref. B9001S)

1 U de AmpliTaq Gold DNA Polymerase (Applied Biosystems)

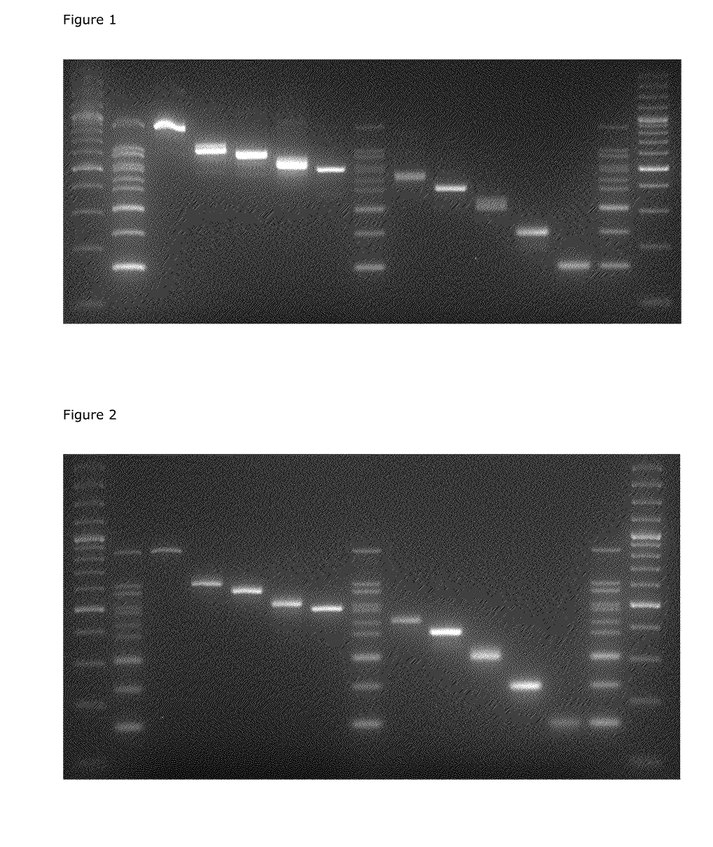

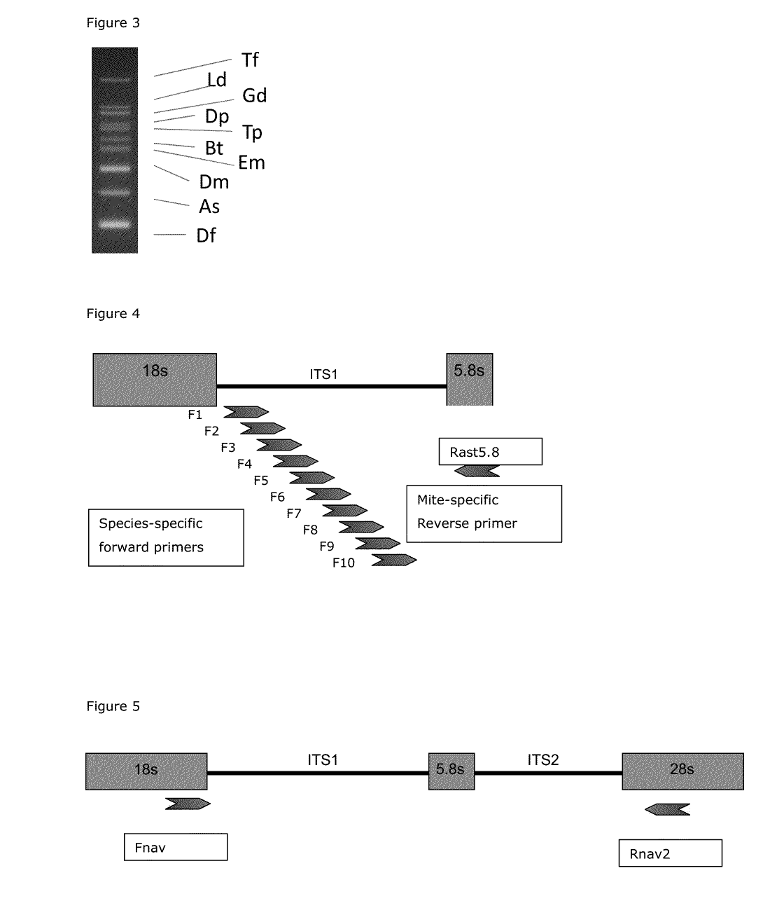

PCR Cycle: PCR Cycle: One hold 10 min 95° C., 40 cycles [30s 95° C., 30s 58° C., 2 min 72° C.], 1 hold 10 min 72° C. PCR products are visualised in agarose gel at 3% [NuSieve low melting agarose (Lonza): D-2 Agarose (Pronadisa), 1:1 proportion]. Results obtained are shown in FIG. 1.

TABLE 1
Forward primers for the Multiplex-PCR. The
approximate size of the amplicon produced by each
forward primer in combination with the reverse
primer RAst5.8S is indicated in the name of the
forward primers.
Primer Sequences (Species)

| | |
|---|---|
| F1Tf_824 | GACAGAAGCTGAAAGCCGT |
| | (Tyrophagus fanetzhangorum) |
| F1Ld_608 | GATGTTCGAATCAATTGCTAGTG |
| | (Lepidoglyphus destructor) |
| F1Gd_567 | GCATACCGTGTTGAAGCAGG |
| | (Glycyphagus domesticus) |
| F1Dp_501 | GATCGACTGGCAATTGTTGAC |
| | (Dermatophagoides pteronyssinus) |
| F1Tp_474 | CGCCATTTGACACAGTACC |
| | (Tyrophagus putrescentiae) |
| F1Bt_419 | TGTGTGTGGGGGATTTTGC (Blomia tropicalis) |
| F1Em_384 | GAGCCTGACAATTATCAATGTGC |
| | (Euroglyphus maynei) |
| F1Dm_304 | CGGGATGAACGTGTGGATG |
| | (Dermatophagoides micro ceras) |
| F1As_234 | GTCGGTTACGGTCAAACG (Acarus siro) |
| F1Df_159 | GAAACAATTGAATTGTGATTCTGC |
| | (Dermatophagoides farinae) |

### Example 3

### Two-Step Multiplex-PCR

Required for the analysis of environmental samples, samples showing a low efficiency in the PCR after performing Example 2, or analysis of contaminations in cultures. Identifies the following species DNA: Tyrophagus fanetzhangorum, Lepidoglyphus destructor, Glycyphagus domesticus, Dermatophagoides pteronyssinus, Tyrophagus putrescentiae, Blomia tropicalis, Euroglyphus maynei, Dermatophagoides micro ceras, Acarus siro, Dermatophagoides farinae.

1. DNA extraction. See Example 1
2. ITS1-ITS2 amplification.

Primers:
FNav
(5′-AGAGGAAGTAAAAGTCGTAACAAG-3′)
and
RNav2
(5-ATATGCTTAAATTCAGCGGG-3′)

PCR reaction: a final volume of 25 μL contains

50-150 ng of DNA template

1×PCR Buffer II

200 μM dNTP mix

0.4 μM each primer (FNav and RNav2)

1.5 mM MgCl2

1 U de AmpliTaq Gold DNA Polymerase (Applied Biosystems)

PCR Cycle: One hold 10 min 95° C., 40 cycles [30s 95° C., 30s 58° C., 2 min 72° C.], 1 hold 10 min 72° C. PCR products may not be visualised after gel electrophoresis.

3. Multiplex-PCR amplification.

Primers: The reaction may be performed
a) using the combination of the set of 10 forward primers (see Table 2) with the reverse primer RAst5.8S (5′-TGCGTTCGAAWGTCGAGT-3′), W=T or A
b) using any combination of two or more primers, the reverse primer being one of them.

PCR reaction: a final volume of 25 μL contains

5 μl of the PCR products obtained in step 2, dilution 1/500 in MQ water

1×PCR Buffer II

200 μM dNTP mix

0.6 μM forward primers mix [0.06 μM each] (Table 2)

0.6 μM reverse primer (RAst5.8S)

1.5 mM MgCl2

0.6 mg/mL purified BSA (New England Biolabs, ref. B9001S)

1 U de AmpliTaq Gold DNA Polymerase (Applied Biosystems)

PCR Cycle: One hold 10 min 95° C., 35 cycles [30s 95° C., 60s 62° C.], 1 hold 10 min 72° C. PCR products are visualised in agarose gel at 3% [NuSieve low melting agarose (Lonza): D-2 Agarose (Pronadisa), 1:1 proportion]., Results obtained are shown in FIG. 2.

TABLE 2
Forward primers for the Multiplex-PCR. The size
of the amplicon produced by each forward primer
in combination with the reverse primer RAst5.8S
is indicated in the name ofthe forward primers.
Primer Sequence (species in parenthesis)

| | |
|---|---|
| F1Tf_824 | GACAGAAGCTGAAAGCCGT |
| | (Tyrophagus fanetzhangorum) |
| F1Ld_608 | GATGTTCGAATCAATTGCTAGTG |
| | (Lepidoglyphus destructor) |
| F1Gd_567 | GCATACCGTGTTGAAGCAGG |
| | (Glycyphagus domesticus) |
| F1Dp_501 | GATCGACTGGCAATTGTTGAC |
| | (Dermatophagoides pteronyssinus) |
| F1Tp_474 | CGCCATTTGACACAGTACC |
| | (Tyrophagus putrescentiae) |
| F1Bt_419 | TGTGTGTGGGGGATTTTGC |
| | (Blomia tropicalis) |
| F1Em_384 | GAGCCTGACAATTATCAATGTGC |
| | (Euroglyphus maynei) |
| F1Dm_304 | CGGGATGAACGTGTGGATG |
| | (Dermatophagoides micro ceras) |
| F1As_234 | GTCGGTTACGGTCAAACG (Acarus siro) |
| F1Df_159 | GAAACAATTGAATTGTGATTCTGC |
| | (Dermatophagoides farinae) |

### Example 4

### Marker Adapted for Identification of Allergy-Causing Mites

### (Ma Marker)

1. PCR amplification of marker bands.

ITS1 marker bands for each species are obtained by PCR amplification following Example 3, and increasing the total volume of the PCRs to 100 μL (increase the template and the units of polymerase proportionally).

Perform a gel in order to verify the correct size of the PCR products.

2. Marker bands mix
a. Purify PCR products using a standard commercial kit.
b. Quantify DNA by a standard method to obtain the concentration (ng/μL). A minimum concentration of 100 ng/μL should be obtained
c. In base to the concentration, calculate the volume (μL) of each PCR product that would contain 5 μg of DNA.
d. Multiply the volumes calculated in step “c” by their corresponding correction factors shown Table 3 (volumes are corrected in base to the size of the amplicons), and introduce the resulting volumes in clean micro-tubes (one micro-tube for each PCR product).
e. Add MQ water to each micro-tube till a total volume of 50 μL and mix by vortex.
f. To verify that all calculations are correct, run an agarose gel, charging in different lanes 1 ul of each PCR product prepared in step “e”. Net bands of similar intensity should be seen for all PCR products.
g. If all bands show the same intensity, continue in step h.
h. If the intensity of some bands is low, add 1-10 μL of the purified PCR products to the corresponding micro-tubes in order increase the DNA contents. Continue again in “step f”.
i. Mix the content of the ten micro-tubes prepared in step “e” in a single vial, adding 50 μL of a standard 10× blue sample buffer.
j. To use the marker, charge 5-10 μL in agarose gels.

TABLE 3
Approximate size of PCR products obtained by the amplification
of DNA from different species by Two-step Multiplex-PCR
(Example 3) and correction factors to prepare the Ma Marker.
Species Approx. Size of PCR product (bp) Correction factor in bold

Tyrophagus fanetzhangorum (Tf) 824; 0.19
Lepidoglyphus destructor (Ld) 608; 0.26
Glycyphagus domesticus (Gd) 567; 0.28
Dermatophagoides pteronyssinus (Dp) 501; 0.32
Tyrophagus putrescentiae (Tp) 474; 0.34
Blomia tropicalis (Bt) 419; 0.38
Euroglyphus maynei (Em) 384; 0.41
Dermatophagoides microceras (Dm) 304; 0.52
Acarus siro (As) 234; 0.68
Dermatophagoides farinae (Df) 159; 1.00

### Example 5

### One-Step Multiplex-PCR Using One or More First (Reverse) Primers and a Single Second (Forward) Primer Hybridising to 18S

Tested for species identification of D. pteronyssinus, D. farinae and/or B. tropicalis in cultures of the ten species: Tyrophagus fanetzhangorum, Lepidoglyphus destructor, Glycyphagus domesticus, Dermatophagoides pteronyssinus, Tyrophagus putrescentiae, Blomia tropicalis, Euroglyphus maynei, Dermatophagoides micro ceras, Acarus siro, Dermatophagoides farinae.

1. DNA extraction. See Example 1

### 3. Multiplex-PCR.

Primers: The reaction may be performed

a) using the combination of the set of three reverse (first) primers (see Table 4 below) with the forward (second) primer FRibNav (5′-AGAGGAAGTAAAAGTCGTAACAAG-3′)
b) using any combination of two or more primers, the forward (second) primer being one of them.

PCR reaction: a final volume of 25 μL contains

50-150 ng of DNA template

1×PCR Buffer II

200 μM dNTP mix

0.6 μM reverse primers mix [0.2 μM of each in this case where all three are used] (Table 4)

0.6 μM forward primer (FRibNav)

1.5 mM MgCl2

0.6 mg/mL purified BSA (New England Biolabs, ref. B9001S)

1.5 U AmpliTaq Gold DNA Polymerase (Applied Biosystems)

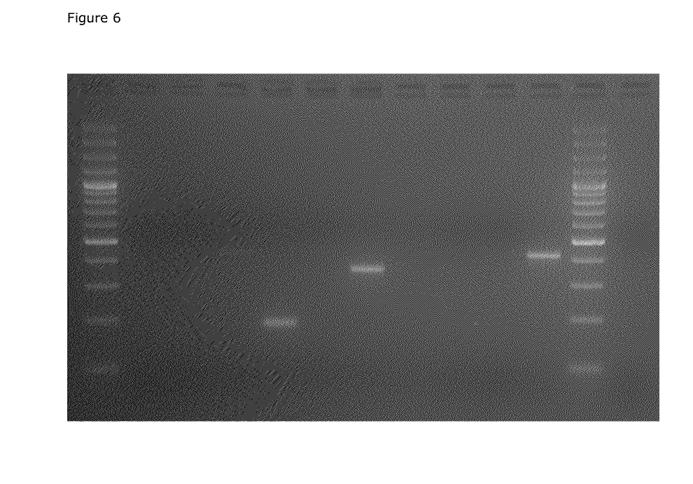

PCR Cycle: One hold 10 min 95° C., 40 cycles [30s 95° C., 30s 58° C., 2 min 72° C.], 1 hold 7 min 72° C. PCR products are visualised in agarose gel at 3% [NuSieve low melting agarose (Lonza): D-2 Agarose (Pronadisa), 1:1 proportion]. Results obtained are shown in FIG. 6.

TABLE 4
Reverse (first) primers for the Multiplex-PCR. The
approximate size of the amplicon produced by each
reverse primer in combination with the forward
primer FRibNav is indicated in the name of the
reverse primers.
Primer Sequences (Species)

| | |
|---|---|
| R1Dp_181 | GCTTTCAATAACCTCATCAGTGTC |
| | (Dermatophagoides pteronyssinus) |
| R1Bt_347 | CCATCACTAAAGGACAGAACCGC |
| | (Blomia tropicalis) |
| R1Df_419 | CTCCAGCAATCGAATTATGCTC |
| | (Dermatophagoides farinae) |

### REFERENCES

Cruickshank R H (2002) “Molecular markers for the phylogenetics of mites and ticks”. System Appl Acarol 7:3-14.
Lava Kumar, P., Fenton, B., Jones, A. T. (1999) Identi®cation of Cecidophyopsis mites (Acari: Eriophyidae) based on variable simple sequence repeats of ribosomal DNA internal transcribed spacer-1 sequences via multiplex PCR. Insect Molecular Biology. 1999; 8(3); 347-357
Navajas, M., Lagnel, J., Fauvel, G. & de Moraes, G. (1999) Sequence variation of ribosomal internal transcribed spacers (ITS) in commercially important Phytoseiidae mite. Experimental and Applied Acarology, 23, 851-859.
Noge K, Mori N, Tanaka C, Nishida R, Tsuda M, Kuwahara Y. Identification of astigmatid mites using the second internal transcribed spacer (ITS2) region and its application for phylogenetic study. Exp Appl Acarol. 2005; 35:29-46.
Spieksma F T M, “Identification of house-dust mites”, Aerobiologia 1990; 187-192.
Suarez-Martinez E B, Montealegre F, Sierra-Montes J M. Molecular identification of pathogenic house dust mites using 12S rRNA sequences. Electrophoresis. 2005; 26:2927-34.
Thet-Em T, Tungtrongchitr A, Tiewcharoen S, Malainual N., “Multiplex PCR for identifying common dust mites species (D. pteronyssinus, D. farinae and B. tropicalis)”, Asian Pac J Allergy Immunol 2012; 30:224-30.
Wong S F, Chong A L, Mak J W, Tan J, Ling S J, Ho T M. “Molecular identification of house dust mites and storage mites”. Exp Appl Acarol. 2011; 55:123-33.

1. A method for preparing a mite allergen extract of one or more different Astigmata mite species in a sample, the method comprising the steps of:
a) extracting mite DNA from the sample, wherein the mite DNA comprises rDNA;
b) amplifying, a region of the rDNA of each of the mite species to be identified using
i. one or more, first primers each first primer specifically hybridising to the ITS1 sequence of the rDNA of each of the mite species to be identified, or the complementary sequence thereof, and
ii. one or more, second primers specifically hybridising to a sequence selected from any of the 18S, 58S or 28S sequences of the rDNA of the mite species to be identified, or the complementary sequence thereof,
to produce an amplicon specific to each mite species to be identified, and;
c) identifying the mite species by evaluating a characteristic of the amplicon,
wherein mite allergens are extracted from said sample based on identified mite species.

2. The method according to claim 1, wherein under step b) the amplicon produced has a molecular size which is characteristic of the specific mite species to be identified.

3. The method according to claim 1, wherein under step c) the mite species is identified by evaluating the molecular size of the amplicon which is characteristic of the mite species to be identified.

4. (canceled)

5. The method according to claim 1, wherein under step b) two or more amplicons specific to the mite species to be identified are produced, which amplicons differ in length by at least 15 bp.

6. The method according to claim 1, wherein the second primer is at least 90% identical to at least 15 consecutive nucleotides of said sequence of any of the Astigmata mite species to be identified.

7. The method according to claim 1, wherein the one or more first primers used in step b) i. contains at least 3, such as 4, 5 or 6 consecutive nucleotides at the 3′ end with exact complementarity to any ITS1 sequence of the mite species to be identified.

8. The method according to claim 1, wherein the one or more first primers used in step b) i. is at least about 70% identical to the sequence of any corresponding part of the ITS1 sequence or a complementary part thereof of the mite species to be identified.

9. (canceled)

10. The method according to claim 1, wherein step c) is performed by comparing the molecular size(s) of the amplicon(s) to the molecular sizes of reference nucleotides of a molecular marker composition, the sizes of the reference nucleotides spanning the relevant base pair interval.

11. The method according to claim 10, wherein the sizes of the reference nucleotides correspond to the sizes of the amplicons characteristic of the mite species to be identified.

12. The method according to claim 1, wherein step b) is preceded by a preamplification step, such as by PCR, wherein rDNA containing the ITS1 region of any Astigmata mite species in the sample is amplified using a first primer specifically hybridising to the 18S sequence of the rDNA and a second primer specifically hybridising to a sequence selected from the 5.8S and 28S sequences of the rDNA.

13-15. (canceled)

16. The method according to claim 1, wherein two or more first primers are used, each primer specifically hybridising to the ITS1 sequences of one mite species to be identified and not cross-hybridising to other mite species to be identified.

17. (canceled)

18. The method according to claim 1, wherein said first primer referred to in b) i. comprises a sequence at least about 70% identical to the ITS1 of a sequence selected from any one of SEQ ID NOs:1-100, or the complementary sequence thereof, or a fragment thereof.

19. (canceled)

20. (canceled)

21. The method according to claim 1, wherein said first primer comprises a sequence at least about 70%, identical to a nucleic acid sequence selected from the list consisting of SEQ ID NO:101, 102, 103, 104, 105, 106, 107, 108, 109, 110, 122, 123, and 124, or the complementary sequence thereof, or fragment thereof, or complementary sequence thereof.

22. (canceled)

23. The method according to claim 1, wherein said second primer comprises a nucleic acid sequence at least about 70% identical to a fragment of 5.8S in a sequence selected from any one of SEQ ID NOs:1-100, or the complementary sequence thereof.

24. The method according claim 1, wherein said second primer comprises a nucleic acid sequence at least about 70% identical to a fragment of 18S in a sequence selected from any one of SEQ ID NOs:1-100, or the complementary sequence thereof.

25. The method according to claim 1, wherein said one or more different species in the Astigmata suborder is/are selected from the group consisting of: Tyrophagus fanetzhangorum, Lepidoglyphus destructor, Glycyphagus domesticus, Dermatophagoides pteronyssinus, Tyrophagus putrescentiae, Blomia tropicalis, Euroglyphus maynei, Dermatophagoides microceras, Acarus siro, and Dermatophagoides farinae.

26. An isolated nucleic acid molecule at least about 80% identical to a nucleic acid sequence selected from the list consisting of SEQ ID NO:1-100 or fragment thereof, or complementary sequence thereof, which nucleic acid molecule further comprises a detectable label.

27-33. (canceled)

34. A composition comprising at least 2 different nucleic acid molecules of different species in the Astigmata suborder identified in claim 17 with or without a detectable label.

35-41.

42. A molecular size marker composition comprising one or more polynucleotides, such as a DNA of a size corresponding to one or more amplicons unique to specific mite species in the Astigmata suborder.

43. Kit of parts comprising:
a) A composition as defined in claim 18; and
b) A molecular size marker, comprising one or more polynucleotides, such as a DNA of a size corresponding to one or more amplicons unique to specific species in the Astigmata suborder,
wherein said kit identifies at least one mite species in the Astigmata suborder.

44-57. (canceled)


SEQUENCE LISTING (124 sequences)

SEQ ID NO: 1 - DNA, 907 bp, Dermatophagoides microceras
gtttccgtaggtgaacctgcggaaggatcattatcggtgttttttttgcacttgattcttaaatcgaacgaaaagttgcccgttatcacaaacggatcgactgacttgtgtgttgcgatcggtggtgtcacattgaaagttctgaagttaaacttggcttttatattatatctgggtaacatggaaaaaagcattagttagacttcataacaatggaacttgacacttaccggtgtacaactttgtcggcgattctccttgctatctgttggtgcctagtctacggttcctgtttatcctcgggatgaacgtgtggatgtagtgtgtcgcttgtaatgagtgccgctaggtttaaataccagatttgaatgtaattcgattgctggagccacaggtacaaggcattttctttttcatttcaaaacaacattgagccagtatcattgaatcgtttgaattgtgattctgaaaatgaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctaaacaacccagaatgtgtgccttgtatattgccaacatggatgagcattgtagaatgtataactttaaattgtctatattcgacctcagatcaagcgagacta

SEQ ID NO: 2 - DNA, 907 bp, Dermatophagoides microceras
gtttccgtaggtgaacctgcggaaggatcattatcggtgttttttttgcacttgattcttaaatcgaacgaaaagttgcccgttatcacaaacggatcgactgacttgtgtgttgcgatcggtggtgtcattgaaagttctgaagtgttaaacttggcttttatattatatctgggtaacatggaaaaaggcattagttagacttcataacaatggaacttgacacttaccggtgtacaactttgtcggcgattctccttgctatctgttggtgcctagtctacggttcctgtttatcctcgggatgaacgtgtggatgtagtgtgtcgcttgtaatgagtgccgctaggtttaaataccaaatttgaatgtaattcgattgctggagccacaggtacaaggcattttctttttcatttcaaaacaacattgagccagtatcattgaatcgtttgaattgtgattctgaaaatgaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctaaacaacccagaatgtgtgccttgtatattgccaacatggatgagcattgtagaatgtataactttaaattgtctatattcgacctcagatcaagcgagacta

SEQ ID NO: 3 - DNA, 916 bp, Dermatophagoides microceras
gtttccgtaggtgaacctgcggaaggatcattatcggtgtttttgttgcacttgattcttaaatcgaacgaaaagttgcccgttatcacaaacggatcgactgacttgtgtgttgcgatcggtggtgtcattgaaagttctgaagttaaacttggcttttatattatatctgggtgacatggaaaaaagcaatagttagacttcataacaatggaacttgacacttaccggtgtacaactttgtcggcgattctccttgctatctgttggtgcctagtctacggttcctgtttatcctcgggatgaacgtgtggatgtagtgtgtcgcttgtaatgagtgccgctaggtttaaataccagattccaacaaatatattgtttgattgctggagccacaggtacaaggcattttctttttcatttagaaacaacattgagccagtatcattgaatcattaaaattgtgattctctataaacgaagaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctaaacaacccagaatgtgtgccttgtatatcgccaacatggattagtattgtagaatttgtaactttaaattgtctatattcgacctcagatcaagcgagacta

SEQ ID NO: 4 - DNA, 916 bp, Dermatophagoides microceras
gtttccgtaggtgaacctgcggaaggatcattatcggtgtttttgttgcacttgattcttaaatcgaacgaaaagttgcccgttatcacaaacggatcgactgacttgtgtgttgcgatcggtggtgtcattgaaagttctgaagttaaacttggcttttatattatatctgggtgacatggaaaaaagcaatagttagacttcataacaatggaacttgacacttaccggtgtacaactttgtcggcgattctccttgctatctgttggtgcctagtctacggttcctgtttatcctcgggatgaacgtgtggatgtagtgtgtcgcttgtaatgagtgccgctaggtttaaataccagattccaacaaatatattgtttgattgctggagccacaggtacaaggcattttctttttcatttaaaaacaacattgagccagtatcattgaatcattaaaattgtgattctctataaacgaagaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcctttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctaaacaacccagaatgtgtgccttgtatattgccaacatggatgagcattgtagaatgtataactttaaattgtctatattcgacctcagatcaagcgagacta

SEQ ID NO: 5 - DNA, 914 bp, Dermatophagoides microceras
gtttccgtaggtgaacctgcggaaggatcattatcggtgtttttttttgcacttgattcttaaatcgaacgaaaagttgcccgttatcacaaacggatcgactgacttgtgtgttgcgatcggtggtgtcacattgaaagttctgaagttaaacttggcttttatattatatctgggtgacatggaaaaaagtattagtttgacttcatagcaatggaacttgacacttaccggtgtacaactttgtcggcgattctccttgctatctgttggtgcctagtctacggttcctgtttatcctcgggatgaacgtgtggatgtagtgtgtcgcttgtaatgagtgccgctaggtttaaataccagattccaacaaatatattgtttgattgctggagccacaggtacaaggcattttctttttcatttaaaaacaacattgagccagtatcattgaatcattaaaattgtgattctctataaacgaagaatcgcaagaaagcaaaaattactactgccagtggtggatcactcggctcgctggtcgaggaagaacgtaggtagctacgttaatcggtgtgaaatgcaggacactctgatcactcgacattcgaacgcacattgcagccattggatatccgatggcttcgtttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctagacaacccagaatgtgtgccttgtatattgccaacatggattagtattgcgtgtaaattgcaattgtctatattcgacctcagatcaagcgagacta

SEQ ID NO: 6 - DNA, 914 bp, Dermatophagoides microceras
gtttccgtaggtgaacctgcggaaggatcattatcggtgtttttttttgcacttgattcttaaatcgaacgaaaagttgcccgttatcacaaacggatcgactgacttgtgtgttgcgatcggtggtgtcacattgaaagttctgaagttaaacttggcttttatattatatctgggtgacatggaaaaaagcattagtttgacttcatagcaatggaacttgacacttaccggtgtacaactttgtcggcgattctccttgctatctgttggtgcctagtctacggttcctgtttatcctcgggatgaacgtgtggatgtagtgtgtcgcttgtaatgagtgccgctaggtttaaataccagattccaacaaatatattgtttgattgctggagccacaggtacaaggcattttctttttcatttaaaaacaacattgagccagtatcattgaatcattaaaattgtgattctctataaacgaggaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctaaacaacccagaatgtgtgccttgtatattgccaacatggattagtattgcgtgtaaattgcaattgtctatattcgacctcagatcaagcgagacta

SEQ ID NO: 7 - DNA, 909 bp, Dermatophagoides microceras
gtttccgtaggtgaacctgcggaaggatcattatcggtgtttttgttgcacttgattcttaaatcgaacgaaaagttgcccgttatcacaaacggatcgactgactttgtgttgcgatcggtggtgtcattgaaagttctgaagttgatgcttggcttttatattatctgggtgacatggaaaaaagcattagttggacttcataataatggaacttgacacttaccggtgtacaactttgtcggcgattctccttgctatctgttggtgcctactctacggttcctgtttatcctcgggatgaacgtgtggatgtagtgtgtcgcttgtaatgagtgccgctaggtttaaataccagattccaacaaatatattgtttgattgctggagccacaggtacgaggcattttctttttcatttaaaaacaacattgagccagtatcattgaatcattaaaattgtgattctctataaacgaagaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctaaacaacccagaatgtgtgccttgtatattgccaacatggattagtattgcgtgtaaattgcaattgtctatattcgacctcagatcgagcgagacta

SEQ ID NO: 8 - DNA, 914 bp, Dermatophagoides microceras
gtttccgtaggtgaacctgcggaaggatcattatcggtgtttttttttgcacttgattcttaaatcgaacgaaaagttgcccgttatcacaaacggatcgactgacttgtgtgttgcgatcggtggtgtcacattgaaagttctgaagttaaacttggcttttatattatatctgggtgacatggaaaaaagcattagtttgacttcatagcaatggaacttgacacttaccggtgtacaactttgtcggcgattctccttgctatctgttggtgcctagtctacggttcctgtttatcctcgggatgaacgtgtggatgtagtgtgtcgcttgtaatgagtgccgctaggtttaaataccagattccaacaaatatattgtttgattgctggagccacaggtacaaggcattttctttttcatttcaaaacaacattgagccagtatcattgaatcgtttcaattgtgattatgaaaataaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcatggtatttagcgtttccagctaaacaacccagaatgtgtgccttgtatattgccaacatggatgagcattgtagaatgtataactttaaattgtctatattcgacctcagatcaagcgagacta

SEQ ID NO: 9 - DNA, 914 bp, Dermatophagoides microceras
gtttccgtaggtgaacctgcggaaggatcattatcggtgtttttttttgcacttgattcttaaatcgaacgaaaagttgcccgttatcacaaacggatcgactgacttgtgtgttgcgatcggtggtgtcattgaaagttctgaagtgttaaacttggcttttatattatatctgggtgacatggaaaaaagcattagttggacttcataataatggaacttgacacttaccggtgtacaactttgtcggcgattctccttgctatctgttggtgcctagtctacggttcctgtttatcctcgggatgaacgtgtggatgtagtgtgtcgcttgtaatgagtgccgctaggtttaaataccagattccaacaaatatattgtttgattgctggagccacaggtacaaggcattttctttttcatttcagaacaacattgagccagtatcattgaatcgtttgaattgtgattctgaaaatgaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcctttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctaaacaacccagaatgtgtgccttgtatattgccaacatggattagcattgtagaatgtataactttaaattgtctatattcgacctcagatcaagcgagacta

SEQ ID NO: 10 - DNA, 905 bp, Dermatophagoides microceras
gtttccgtaggtgaacctgcggaaggatcattatcggtgttttttttgcacttgattcttaaatcgaacgaaaagttgcccgttatcacaaacggatcgactgacttgtgtgttgcgatcggtggtgtcattgaaagttctgaagtattaaacttggcttttatattatctgggtgacatggaaaagagcagtagttagacttcataacaatggaacttgacacttaccggtgtacaactttgtcggcgattctccttgctatctgttggtgcctactctacggttcctgtctatcctcgggatgaacgtgtggatgtagtgtgtcgcttgtaatgagtgccgctaggtttaaataccagattcgaatgtaattcgactgctggagccacaggtacaaggcattttctttttcatttcaaaacaacattgagccagtatcattgaatcattaaaattgtgattctctataaacgaagaatcgcaagaaagcaaaaattactactgccagtggtggatcactcggcccgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccaaatggaatagatctgtgtcgtcgcgattcatttcggcgtcgtgagattatttctaaacatttcgaatgctgactcttttttggtgtgtgaaggctttgtagcacattcatcagggtatttagcgtttccagctaaacaacccagaatgtgtgccttgtatattgccaacatggattagtattgcgtgtaaatatcaattgtctatattcgacctcagatcaagcgagacta

SEQ ID NO: 11 - DNA, 912 bp, Dermatophagoides farinae
gtttccgtaggtgaacctgcggaaggatcattatcggtgtttttttgaacaatgaaacacttgattctcaattcgaacgatgtgttgcccgttatcacaaacggatcgactgacttgtgttgcgatcggtggtgtcattgaaagttctgaagttgaaacttggctttttttatatctgggtgacatggaaaaaagcattagttggacttctttaaagcaatggaacttgacacttaccgatgtacaactttgttggcgattctccttgctatctgttggtgcctattctacggttcctgtttgtcctcgggataaacgtggatgtagtgtgtcgcttgtaatgagtgccgctaggtttaaataccagattcgagcataattcgattgctggagccacaggtacaaggcattttctttttcatttcaaaacaacattgagccagtatcattgaaacaagtgaattgtgattctgaaaatgaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcaaaatttgacaaaccaaaatgaaataaatctgtatcgtcgcgattcatttcggcgtcgtgagattatttctaaacatttcgaatgctgactcctttggtgattatttaatggctttgtagcacattcatcactagaagggtatttagcgtttccagctaaacaacccagaatgtgtgccatgtatgattaccatcatggatcaacattcaaatttcaattgtctatattcgacctcagatcaagcgagacta

SEQ ID NO: 12 - DNA, 912 bp, Dermatophagoides farinae
gtttccgtaggtgaacctgcggaaggatcattatcggtgtttttttgaacaataaaacacttgattctcaatttgaacgatgtgttgcccgttatcacaaacggatcgactgacttgtgttgcgattggtggtgtcattgaaagttctgaagttgaaacttggcttttattatatctgggtgacatggaaaaaagcattagttggacttctttaaagcaatggaacttgacacttaccgatgtacaactttgttggcgattctccttgctatctgttggtgcctattctacggttcctgtttatcctcgggatgaacgtggatgtagtgtgtcgcttgtaatgagtgccgctaggtttaaataccagattcgagcataattcgattgctggagccacagatacaaggcattttctttttcatttcaaaacaacattgagccagtatcattgaaacaattgaattgtgattctgcaaatgaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcaaaatttgacaaaccaaaatgaaataaatctgtatcgtcgcgattcatttcggcgtcgtgagattatttctaaacatttcgaatgctgactcctttggtgattatttaatggctttgtagcacattcatcactagaagggtatttagcgtttccagctaaacaacccagaatgtgtgccatgtatgattaccatcatggatcaacattcaaatttcaattgtctatattcgacctcagatcaagcgagacta

SEQ ID NO: 13 - DNA, 915 bp, Dermatophagoides farinae
gtttccgtaggtgaacctgcggaaggatcattatcggtgtttttgtttgaacaatgaaacacttgattatcaattcgaacgatgtgttgcccgttatcacaaacggatcgactgacttgtgttgcgatcggtggtgtcattgaaagttctgaagttgaaacttggtttttattatatctgggtgacatggaaaaaaacattagttggacttctttaaagcaatggaacttgacacttaccgatgtacaactttgttggcgattctccttgctatctgttggtgcctattctacggttcctgtttgtcctcgggatgaacgtggatgtagtgtgtcgcttgtaatgagtgccgctaggtttaaataccagattcgagcataattcgattgctggagccacaggtacaaggcattttctttttcatttcaaaacaacattgagccagtatccattgaaacaattgaattgtgattctgcaaatgaatcgcatgaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcaaaatttgacaaaccaaaatggaataaatctgtatcgtcgttattcatttcggcgtcgtgagattatttctaaacattttgaatgctgactcctttggtgattatttaatggctttgtagcacgttcatcactaaaagggtatttagcgtttccagctaaacaacccagaatgtgtgccatgtatgataaccatcatggatcaatattcaaatttcaattgtctatattcgacctcagatcaagcgagacta

SEQ ID NO: 14 - DNA, 915 bp, Dermatophagoides farinae; identical to SEQ ID NO: 13

SEQ ID NO: 15 - DNA, 909 bp, Dermatophagoides farinae
gtttccgtaggtgaacctgcggaaggatcattatcggtgtttttttgaacaatgaaacacttgattctcaattcgaacgatgtgttgcccgttatcacaaacggatcgactgacttgtgttgcgattggtggtgtcattgaaagttctgaagttgaaacttggcttttattatatcagggtgacatggaaaaaagcattagttcaacttcataataatggaacttgacacttaccgatgtacaactttgtcggcgattctccttgctatctgttggtgcctattctacggttcctgtttgtcctcgggataaacgtggatgtagtgtgtcgcttgtaatgagtgccgctaggtttaaataccagattcgagcataattcgattgctggagccacaggtacaaggcattttctttttcatttcaaagcaacattgagccagtatcattgaaacaattgaattgtgattctgcaaatgaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcaaaatttgacaaaccaaaatggaataaatccgtatcgtcgcgattcatttcggtgtcgtgagattatttctaaacatttcgaatgctgactcctttggtgattatttgatggctttgtagcacattcatcactagaagggtatttagcgtttccagctaaacaacccagaatgtgtgccatgtatgattaccatgatggattaacattcaaattagaattgtctatattcgacctcagatcaagcgagacta

SEQ ID NO: 16 - DNA, 909 bp, Dermatophagoides farinae; identical to SEQ ID NO: 15

SEQ ID NO: 17 - DNA, 912 bp, Dermatophagoides farinae
gtttccgtaggtgaacctgcggaaggatcattatcggtgtttttgtttgaacaatgaaacacttgattctcaattcgaacgatgtgttgcccgttatcacaaacggatcgactgacttgtgttgcgattggtggtgtcattgaaagttctgaagttgaaacttggcttttattatatctgggtaacatggaaaaaagcattagttcaacttcataataatggaacttgacacttaccgatgtacaactttgtcggcgattctccttgctatctgttggtgcctattctacggttcctgtttgtcctcgggataaacgtggatgtagtgtgtcgcttgtaatgagtgccgctaggtttaaataccagattcgagcataattcgattgctggagccacaggtacaaggcattttctttttcatttcaaaacaacattgagccagtatcattgaaacaattaaattgtgattctgcaaatgaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcaaaatttgacaaaccaaaatggaataaatccgtatcgtcgcgattcatttcggtgtcgtgagattatttctaaacatttcgaatgctgactcctttggtgattatttgatggctttgtagcacattcatcactaaaaagggtatttagcgtttccagctaaacaacccagaatgtgtgccatgtatgattaccatcatggatcaatattcaaattacaattgtctatattcgatctcagatcaagcgagacta

SEQ ID NO: 18 - DNA, 911 bp, Dermatophagoides farinae
gtttccgtaggtgaacctgcggaaggatcattatcggtgtttttgtttgaacaatgaaacacttgattctcaattcgaacgatgtgttgcccgttatcacaaacggatcgactgacttgtgttgcgatcggtggtgtcattgaaagttctgaaattgaaacttggcttttattatatctgggtaacatggaaaaaagcattagttcaacttcataataatggaacttgacacttaccgatgtacaactttgttggcgattctccttgctatctgttggtgcctattctacggttcctgtttatcctcgggatgaacgtggatgtagtgtgtcgcttgtaatgagtgccgctaggtttaaataccagattcgagcataattcgattgctggagccacaggtacaaggcattttctttttcatttcaaaacaacattgagccagtatcattgaaacaattgaattgtgattctgcaaatggatcgcaaaaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgataaaccaaaatgaaataaatctgtatcgtcgcgattcatttcggtgtcgtgagattatttctaaacatttcgaatgctgactcctttggtgattatttgatggctttgtagcacattcatcactagaagggtatttagcatttccagctaaacaacccagaatgtgtgccatgtatgattaccataatggatcaatattcaaatttcaattgtctatattcgacctcagatcaagcgagacta

SEQ ID NO: 19 - DNA, 911 bp, Dermatophagoides farinae
gtttccgtaggtgaacctgcggaaggatcattatcggtgtttttgtttgaacaatgaaacacttgattctcaattcgaacgatgtgttgcccgttatcacaaacggatcgactgacttgtgttgcgatcggtggtgtcattgaaagttctgaaattgaaacttggcttttattatatctgggtaacatggaaaaaagcattagttcaacttcataataatggaacttgacacttaccgatgtacaactttgtcggcgattctccttgctatctgttggtgcctattctacggttcctgtttatcctcgggatgaacgtggatgtagtgtgtcgcttgtaatgagtgccgctaggtttaaataccagattcgagcataattcgattgctggagccacaggtacaaggcattttctttttcatttcaaaacaacattgagccagtatcattgaaacaattaaattgtgattctgcaaatgaatcgcaagaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccaaaatgaaatagatctttatcgtcgcgattcatttcggtgtcgtgagattatttctaaacatttcgaatgctgactcctttggtgattatttgatggctttgtagcacattcatcactagaagggtatttagcgtttccagctaaacaacccagaatgtgtgccatgtatgattaccatgatggattaacattcaaattagaattgtctatattcgacctcagatcaagcgagacta

SEQ ID NO: 20 - DNA, 915 bp, Dermatophagoides farinae
gtttccgtaggtgaacctgcggaaggatcattatcggtgtttttgtttgaacaatgaaacacttgattctcaattcgaacgatgtgttgcccgttatcacaaacggatcgactgacttgtgttgcgatcggtggtgtcattgaaagttctgaagttgaaacttggcttttattatatctgggtgacatggaaaaagcattagttagacttctataaagcaatggaacttgacacttgcctatgtacaactttgtcggcgattctccttgctatctgttggtgcctattctactgttcctgtttgtcctcgggataaacgtggatgtagtgtgtcgcttgtaatgagtgccgctaggtttaaataccagattcgagcataattcgattgctggagccacaggtacaaggcattttctttttcatttcaaaacaacattgagccagtatcattgaaacaattgaattgtgattctgcaaatggatcgcaaaaaagcaaaaattactactgccagtggtggatcactcggctcgctggtcgaggaagaacgtagctagctacgttaatcggtgtgaaatgcaggacactctgatcactcgacattcgaacgcacattgcagccattggatatccgatggcttcgtttgtctgagcgtcgttatcgaaatttgacaaaccacaaaatgaaatagatctgtatcgtcgcgattcatttccgcgtcgtgagattatttctaaacatttcgaatgctgattcctttggtgattatttgatggctttgtagcacatttatcactagaagggtatttagcgtttccagctaaacaacccagaatgtgtgccatgtatgattaccatgatggatcaatattcaaattactattgtctatattcgacctcagatcaagcgagacta

SEQ ID NO: 21 - DNA, 1396 bp, Acarus siro
gtttccgtaggtgaacctgcggaaggatcattaacggatagttgctttgcttgcatttgtttgcttgctaaagaaacaatcaatatccaaacctttaccacttgaacgcctattgcccgttagcatacccccatgctaatgagctgatcatgcgttggttctacatgtcagtgtgacccagagaaaggctaccaaccctttactcggtacaacttttgaacgcttgcctgtttaacttgtctgtgcacgctttgcatactgtctctaccacgccaaataaaccttttaggttgattcattttgggcaatcatatgaaatgaatcatttcctaaaaaggcggctgttggttgagaaagatagcatacagtgctgtaagcagactaaccaaactgaagcacccgtaccaggtataccagccaattaccgcacacaccaaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggctttaaatatctacgccaagcgccgaagcctcgaatgcaaggttctcgcgtacagtatacctagtgtgcagtacgttgagtgagactgtctctggtcggttacggtcaaacgtagccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttctaggtggaaccaatgtatgtttgcctttacatttcagtctcgaatggttaatgcaacatttaatgcttgtacctttacttgagaagtgtcacaatagtttacccttactagtagagacttgaccgtaaaacagtcactgatcttccggtgttaaccttcgacctcaggtcaagcgagatta

SEQ ID NO: 22 - DNA, 1394 bp, Acarus siro
gtttccgtaggtgaacctgcggaaggatcattaacggatagttgctttgcttgcatttgtttgcttgctaaagaaacaatcaatatccaaacctttaccacttgaacgcctattgcccgttagcatacccccatgctaatgagctgatcatgcgttggttctacatgtcagtgtgacccagagaaaggctaccaaccctttactcggtacaacttttgaacgcttgcctgtttaacttgtctgtgcacgctttgcatactgtctctaccacgccaaataaaccttttaggttgattcattttgggcaatcatatgaaatgaatcatttcctaaaaaggcggctgttggttgagaaagatagcatacagtgctgtaagcagactaaccaaactgaagcacccgtaccaggtataccagccaattaccgcacacaccaaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggcttaatatctacgccaagcgccgaagcctcgaatgcaaggttctcgcgtacagtatacctagtgtgcagtacgttgagtgagactgtctctggtcggttacggtcaaacgtagccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttctaggtggaaccaatgtatgtttgcctttacatttcagtctcgaatggttaatgcaacatttaatgcttgtacctttacttgagaagtgtcacaatagtttacccttactagtagagacttgaccgtaaaacagtcactgatcttctggtgttaaccttcgacctcaggtcaagcgagatta

SEQ ID NO: 23 - DNA, 1396 bp, Acarus siro
gtttccgtaggtgaacctgcggaaggatcattaacggatagttgctttgcttgcatttgtttgcttgctaaagaaacaatcaatatccaaacctttaccacttgaacgcctattgcccgttagcatacccccatgctaatgagctgatcatgcgttggttctacatgtcagtgtgacccagagaaaggctaccaaccctttactcggtacaacttttgaacgcttgcctgtttaacttgtctgtgcacgctttgcatactgtctctaccacgccaaataaaccttttaggttgattcattttgggcaatcatatgaaatgaatcatttcctaaaaaggcggctgttggttgagaaagatagcatacagtgctgtaagcagactaaccaaactgaagcacccgtaccaggtataccagccaattaccgcacacaccaaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggcttaaatatctacgccaagcgccgaagcctcgaatgcaaggttctcgcgtacagtatacctagtgtgcagtacgttgagtgagactgtctctggtcggttacggtcaaacgtagccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttcttaggtggaaccaatgtatgtttgcctttacatttcagtctcgaatggttaatgcaacatttaatgcttgtacatttacttgagaagtgtcacaatagtttacccttactagtagagacttgaccgtaaaacagtcactgatcttctggtgttaaccttcgacctcaggtcaagcgagatta

SEQ ID NO: 24 - DNA, 1404 bp, Acarus siro
gtttccgtaggtgaacctgcggaaggatcattaacggatagttgctttgcttgcatttgtttgcttgctaaagaaacaatcaatatccaaacctttaccacttgaacgcctattgcccgttagcatacccccatgctaatgagctgatcatgcgttggttctacatgtcagtgtgacccagagaaaggctaccaaccctttactcggtacaacttttgaacgcttgcctgtttaacttgtctgtgcacgctttgcatactgtctctaccacgccaaataaaccttttaggttgattcattttgggcaatcatatgaaatgaatcatttcctaaaaaggcggctgttggttgagaaagatagcatacagtgctgtaagcagactaaccaaactgaagcacctgtaccaggtataccagccaattaccgcacacaccaaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggcttaaatatctacgccaagcgccgaagcctcgaatgcaaggttctcgcgtacagcacacacccaggtgtgagcagtacgttgagtgagactgtctctggtcggttacggtcacacgtaaccaccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttcttaggtggaaccaatgtatgtttgcctttacatttcagtctcgaatggttaatgcaacatttaatgcttgtacatttacttgagaagtgtcacaatagtttacccttactagtagagacttgaccgtaaaacagtcactgatcttctggtgttaaccttcgacctcaggtcaagcgagatta

SEQ ID NO: 25 - DNA, 1405 bp, Acarus siro
gtttccgtaggtgaacctgcggaaggatcattaacggatagttgctttgcttgcatttgtttgcttgctaaagaaacaatcaatatccaaacctttaccacttgaacgcctattgcccgttagcatacccccatgctaatgagctgatcatgcgttggtttctacatgtcagtgtgacccagagaaaggctaccatccctttactcggtacaacttttgaacgcttgcctgtttaacttgtctgtgcacgctttgcatactgtctctaccacgccaaataaaccttttaggttgattcattttgggcaatcacatgaaatgaatcatttcctagaaaggcggctgttggttgagagagatagcatacagtgcttaagcagactaaccaaactgaagcacccgtaccaggtataccagccaattatcacacacacctaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggcttaaatatctacgccaagcgccgaagcctcgaatgcaaggttctcgcgtacagcacacacccaggtgtgagcagtacgttgagtgagactgtctctggtcggttacggtcacacgtaaccaccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttcttaggtggaaccaatgtatgtttgcctttacatttcagtctcgaatggttaatgcaacatttaatgcttgtacatttacttgagaagtgtcacaatagtttacccttactagtagagacttgaccgtaaaacagtcactgatcttctggtgttaaccttcgacctcaggtcaagcgagatta

SEQ ID NO: 26 - DNA, 1403 bp, Acarus siro
gtttccgtaggtgaacctgcggaaggatcattaacggatagttgctttgcttgcatttaccttgcttgctaaagaaacaatcaatatccaaacctttacctcttgaacgcctattgcccgttagcatatccatgctaatgagctgatcatgcgttggttctacatgtcagtgtgacccagagaaaggctaccaccttactcggtacaacatcctttgaacgcttgcctgtttaacttgtctgtgcacgccttgcatactgtctctaccacgccaaataaaccttttaggttgattcattttgggcaatcacatgaaatgaatcatttcctagaaaggcggctgttggttgagagagatagcatacagtgcagtaagcagactaaccaaactgaagcacccgtaccaggtataccagccaattatcacacacacctaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggcttaaatatctacgccaagcgccgaagcctcgaatgcaaggttctcgcgtacagcacacacccaggtgtgagcagtacgttgagtgagactgtctctggtcggttacggtcacacgtaaccaccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttcttaggtggaaccaatgtatgtttgcctttacatttcagtctcgaatggttaatgcaacatttaatgcttgtacctttacttgagaagtgtcacaatagtttacccttactagtagagacttgaccgtaaaacagtcactgatcttctggtgttaaccttcgacctcaggtcaagcgagatta

SEQ ID NO: 27 - DNA, 1399 bp, Acarus siro
gtttccgtaggtgaacctgcggaaggatcattaacggatagttgctttgcttgcatttacttgcttgctaaagaaacaatcaatatccaaacctttacctcttgaacgcctattgcccgttagcatacccccatgctaatgagctgatcatgcgttggttctacatgtcagtgtgacccagagaaaggctaccaccttactcggtacaacatcctttgaacgcttgcctgtttaacttgtctgtgcacgccttgcatactgtctctaccacgccaaataaaccttttaggttgattcattttgggcaatcacatgaaatgaatcatttcctagaaaggcggctgttggttgagagagatagcatacagtgcttaagcagactaaccaaactgaagcacccgtaccaggtataccagccaattatcacacacacctaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggcttaaatatctacgccaagcgccgaagcctcgaatgcaaggttctcgcgtacagcacacacccaggtgtgagcagtacgttgagtgagactgtctctggtcggttgcggtcaaacgtagccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttctaggtggaaccaatgtatgtttgcctttacatttcagtctcgaatggttaatgcaacatttaatgcttgtacctttacttgagaagtgtcacaatagtttacccttactagtagagacttgaccgtaaaacagtcactgatcttctggtgttaaccttcgacctcaggtcaagcgagatta

SEQ ID NO: 28 - DNA, 1405 bp, Acarus siro
gtttccgtaggtgaacctgcggaaggatcattaacggatagttgctttgcttgcatttaccttgcttgctaaagaaacaatcaatatccaaacctttaccacttgaacgcctattgcccgttagcatacccccatgctaatgagctgatcatgcgttggttctacatgtcagtgtgacccagagaaaggctaccaaccccttactcggtacaacatcctttgaacgcttgcctgtttaacttgtctgtgcacgccttgcatactgtctctaccacgccaaataaaccttttaggttgattcattttgggcaatcacatgaaatgaatcatttcctagaaaggcggctgttggttgagaaagatagcatacagtgctgtaagcagactaaccaaactgaagcacctgtaccaggtataccagccaattaccgcacacaccaaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggcttaaatatctacgccaagcgccgaagcctcgaatgcaaggttctcgcgtacagcacacacctagtgtgagcagtacgttgagtgagactgtctctggtcggttacggtcaaacgtagccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttcttaggtggaaccaatgtatgtttgcctttacatttcagtctcgaatggttaatgcaacatttaatgcttgtacatttacttgagaagtgtcacaatagtttacccttactagtagagacttgaccgtaaaacagtcactgatcttctggtgttaaccttcgacctcaggtcaagcgagatta

SEQ ID NO: 29 - DNA, 1398 bp, Acarus siro
gtttccgtaggtgaacctgcggaaggatcattaacggatagttgctttgcttgcatttacttgcttgctaaagaaacaatcaatatccaaacctttaccacttgaacgcctattgcccgttagcatatccatgctaatgagctgaccatgcgttggttctacatgtcagtgtgacccagagaaaggctaccaccccttactcggtacaacatcctttgaacgcttgcctgtttaacttgtctgtgcacgctttgcatactgtctctaccacgccaaataaacccgtttaggttgattcattttgggcaatcatatgaaatgaatcatttcctaaataggtggctgttggttgagaaagatagcatacagtgcagtaagcagactaaccaaactgaagcacccgtaccagcagtataccagccaattaccgcacacaccaaatgacatgaactttccatgaaagctctccttgtatcattcgattggtgcctagtctgcgtttcctgccaaatttttggcgttggatgcagggtgtcagccagttttgcgactcttttgggttgcttctgctgctgctaggcttaaatatctacgccaagcgccgaagcctcgaatgcaaggttctcgcgtacagtatacctagtgtgcagtacgttgagtgagactgtctctggtcggttacggtcaaacgtagccaccagacgttcagttttcattctctaaactcaacttaagccaaaatcaaaactgattaaccttaaattgtacccttatgggtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttctaggtggaaccaatgtatgtttgcctttacatttcagtctcgaatggttaatgcaacatttaatgcttgtacctttacttgagaagtgtcacaatagtttacccttactagtagagacttgaccgtaaaacagtcactgatcttctggtgttaaccttcgacctcaggtcaagcgagatta

SEQ ID NO: 30 - DNA, 1393 bp, Acarus siro
gtttccgtaggtgaacctgcggaaggatcattaacggatagttgctttgcttgcatttacttgcttgctaaagaaacaatcaatatccaaacctttacctcttgaacgcctattgcccgttagcatatccatgctaatgagctgatcatgcgttggttctacatgtcagtgtgacccagagaaaggctaccaaccccttactcggtacaacatcctttgaacgcttgcctgtttaacttgtctgtgcacgccttgcatactgtctctaccacgccaaataaaccttttaggttgattcattttgggcaatcatatgaaatgaatcatttcctagaaaggcggctgttggttgagagagatagcatacagtgcttaagcagactaaccaaactgaagcacccgtaccaggtataccagccaattaccgcacacaccaaatgacatgaactttccatgaaagctctccttgtatcattcgattggtgcctagtctgcgtttcctgccaaatttttggcgttggatgcagggtgtcagcaactagcaactcttttgggtagcttctgctgctgctaggcttaaatatctacgccaagcgccgaagcctcgaatgcaaggttctcgcgtacagtatacctagtgtgcagtacgttgagtgagactgtctctggtcggttacggtcaaacgtagcccaccagacgttcagttttcattctctaaactcaacttaagccaaaatcaaaactgattaaccttaaattgacttcatcgtcaatttataaaaagacaattacaactgttagtggtggatcactcggcacgctgatcgaggaagaacgcagctagctgcgttaatcggtataaaatgcaggacatgccgaatactcgactttcgaacgcatattgcagccttggtcataccttggcttcgtttgtctgagcgtcgttaacaagccaaaaactatcaaaaggtccgttcaccttcgtatcctaacctcttgtcgggttgattcgtcgggccacctttaaacactactttaaaatgtactcttgtcattttaaatgtgcccatacgagcgtaaagacagttaaccatactgatcttttttgcgtgccaatacatgccttcccctcacggagaaggtatttggatgtagggcttttgacactacatgtcaaaatgcttgagcaattagatcgggtaccatcttcttaggtggaaccaatgtatgtttgcctttacatttcagtctcgaatggttaatgcaacatttaatgcttgtacatttacttgagaagtgtcacaatagtttacccttactagtagagacttgaccgtaaaacagtcactgatcttctggtgttaaccttcgacctcaggtcaagcgagatta

SEQ ID NO: 31 - DNA, 899 bp, Blomia tropicalis
gtttccgtaggtgaacctgcggaaggatcattattggatattattttggtgtgtgattattcatcatcatgctaaattttatctaaattttgtgttttcggacacgaagccatttcgttagttgatcattgagtagcattcctgtgattgtcgtttgaacgctttgcaacattattttgattttgtgtgtgggggattttgcacacagcaatcgatctaatggtttgcggtccacaagttaaaatgtactcgattgacaacaccatttatgctttgtacatcgttatcggtgcctagtctgcggttctgtcctttagtgatggatgcagggtatcattgtataagatgctaggcttaaatatcatcgtgaagccaagtgatgtacattagctaagggaatgttaacccccggccctaaacctcaatgcatttcgatgcattgttgtattataaccaaaatttaagctaaaaaactaaataacaaaacaaaaaaatatagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgttgaatttgtttttaactcgtcgagtcgcattaatgtttgccaaaaaggagacttttttaagaataagttgtccgtggtatgtacaaacgtgcattacagatcggacaattgaacattatgtatgttcacttattgaaaaaaactccaataacaaacaacaaaatcaaagttttgtcaaattcgacctcagatcaagcgagatta

SEQ ID NO: 32 - DNA, 897 bp, Blomia tropicalis
gtttccgtaggtgaacctgcggaaggatcattattggatattattttggtgtgtgattattcatcctcatgctaaattttatctaaattttgtgttttcggacacgaagccatttcgttagttgatcattgagtagcattcttgtgattgtcgtttgaacgctttgcaacattattttgattttgtgtgtgggggattttgcacacagcaatcgatctaatggtttgcggtccacaagttaaaatgtactcgattgacaacaccatttatgctttgtacatcgttatcggtgcctagtctgcggttctgtcctttagtgatggatgcagggtatcattgtataagatgctaggcttaaatatcatcgtgaagccaagtgatgtacattagctaagggaatgttaacccccggccctaaacctcaatgcatttcgacgcattgttgtattctaaccaaaatttaagctaaaaaactaaataacaaaacaaaaaaatacagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgttgaatttgtttttaactcgtcgagtcgcattaatgtttgccaaaaaggagacttttttaaataagttgtccgtggtatgtacaaacgtgcattacagatcggacaattgaacattatgtatgttcacttattgaaaaaaactccaataacaaacaacaaaatcaaagttttgtcaaattcgacctcagatcaagcgagatta

SEQ ID NO: 33 - DNA, 895 bp, Blomia tropicalis
gtttccgtaggtgaacctgcggaaggatcattattggatattattttggtgtgtgattattcatcatcatgctaaattttatctaaattttgtgttttcggacacgaagcaatttcgtttgttgatcattgagtagcattcttgtgattgttttttgaacgctttgcaacattattttgattttgtgtgtgggggattttgcacacagcaatcgatctaatggtttgcggtccacaagttaaaatgtactcgattgacaacaccatttatgctttgtacatcgttatcggtgcctagtctgcggttctgtccactagtgatggatgcagggtatcattattagatgctaggcttaaatatcattttgtgaagccaagtgatgtacattagctaagggaatgtaaacccccggccctaaacctcaatgcatttcggtgcattgttgtactttaaccaaaatttaagctaaaaaactaaataacaaaacaaaaaaaaatacagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgttgaatttgtttttaactcgtcgagtcgcattaatgtttgccaaaaaggagacttaaataagttgtccgtggtatgtacaaacgtgcattacagatcggacaattgaacattatgtatgttcacttattgaaaggaactccaataacaaacaacaaaatcaaaattttgtcaaattcgacctcagatcaagcgagatta

SEQ ID NO: 34 - DNA, 893 bp, Blomia tropicalis
gtttccgtaggtgaacctgcggaaggatcattattggatattattttggtgtgtgattattcatcatcatgctaaattttatctaaattttgtgttttcggacacgaagccatttcgttagttgatcattgagtagcattcttgtgattgtcgtttgaacgctttgcaacattattttgattttgtgtgtgggggattttgcacacagcaatcgatctaatggtttgcggtccacaagttaaaatgtactcgattgacaacaccatttatgctttgtacatcgttatcggtgcctagtctgcggttctgtcctttagtgatggatgcagggtatcattattagatgctaggcttaaatatcattttgtgaagccaagtgatgtacattagctaagggaatgttaacccccggccctaaacctcaatgcatttcgatgcattgttgtattctaaccaaaatttaagctaaaaaactaaataacaaaacaaaaaaatatagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgttgaatttgtttttaactcgtcgagtcgcattaatgtttgccaaaaaggagacttaaataagttgtccgtggtatgtacaaacgtgcattacagatcggacaattgaacattatgtatgttcacttattgagaaaaactccaataacaaacaaccaaatcaaatttttgtcaaattcgacctcagatcaagcgagatta

SEQ ID NO: 35 - DNA, 889 bp, Blomia tropicalis
gtttccgtaggtgaacctgcggaaggatcattattggatattattttggtgtgtgattattcatcatgctaaattttatctaaattttgtgttttcggacacgaatatttttgctagttgatcattgagtagcattcttgtgattgttgtttgaacgctttgcaacattattttgattttgtgtgtgggggattttgcacacagtaatcgatctaatggtttgcggtccacaagttaaaatgtactcgattgacaacaccatttatgctttgtacatcgttatcggtgcctagtctgcggttctgtcctttagtgatggatgcagggtatcattattagatgctaggcttaaatatcatcatgaagccaagtgatgtacattagctaagggaatgtaaacccccggccctaaacctcaatgcatttcgatgcattgttgtattataaccaaaaataagctaagaaaaataaaacaaatcaaaataaaaaatatagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgttgaatttgtttttaactcgtcgagtcgcattaatgtttgccaaaaaggagacttaaataagttgtccgtggtatgtacaaacgtgcattacagatcggacaattgaacattatgtatgttcacttattgagaaaaactccaataacaaacaaccaaatcaaatttttgtcaaattcgacctcagatcaagcgagatta

SEQ ID NO: 36 - DNA, 892 bp, Blomia tropicalis
gtttccgtaggtgaacctgcggaaggatcattattggatattattttggtgtgtgattattcatcatcatgctaaattttatctaaattttgtgttttcggacacgaatatttttgctagttgatcattgagtagcattcttgtgattgttgtttgaacgctttgcaacattattttgattttgtgtgtgggggattttgcacacagcaatcgatttaatggtttgcggtccacaagttaaaatgtacttgattgacaacactattcatgctttgtacatcgttaccggtgcctagtctgcggttctgtcctttagtgatggatgcagggtatcattattagatgctaggcttaaatatcatcatgaagccaagtgatgtacattagctaagggaatgtaaacccccggccctaaacctcaatgcatttcgatgcattgttgtattataaccaaaaataagctaagaaaaataaaacaaatcaaaataaaaaatacagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgttgaatttgttttcaactcgtcgagtcgcattaatgtttgccaaaaaggagacttaaataagttgtccgtggtatgtacaaacgtgcattacagatcggacaattgaacattatgtatgttcacttattgaaaaaaactccaataacaaacaacaaaatcaaagttttgtcaaattcgacctcagatcaagcgagatta

SEQ ID NO: 37 - DNA, 891 bp, Blomia tropicalis
gtttccgtaggtgaacctgcggaaggatcattattggatattattttggtgtgtgattattcatcatgctaaattttatctaaattttgtgttttcggacacgaagcaatttcgttagttgatcattgagtagcattcttgtgattgttttttgaacgctttgcaacattattttgattttgtgtgtgggggattttgcacacagcaatcgatctaatggtttgcggtccacaagttaaaatgtactcgattgacaacaccatttatgctttgtacatcgttatcggtgcctagtctgcggttctgtcctttagtgatggatgcagggtatcattattagatgctaggcttaaatatcattttgtgaagccaagtgatgtacattagctaagagaatgtaaacccccggccctaaacctcaatgcatttcggtgcattgttgtactttaaccaaaaataagctaaaacaaataaaacaaatcaaaatataaaatacagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgttaaacttttgtttttaactcgtcgagtcgcattaatgtttgccaaaaaggagacttaaataagttgtccgtggtatgtacaaacgtgcattacagataggactttgaacattaatgttcacttattgagaaaaaactccaataacaaacaacaaaatcaaagttttgtcaaattcgacctcagatcaagcgagatta

SEQ ID NO: 38 - DNA, 898 bp, Blomia tropicalis
gtttccgtaggtgaacctgcggaaggatcattattggatattattttggtgtgtgattattcatcatcatgctaatcatatctaaattttgtgttttcggacacgaagcaatttcgttagttgatcattgagtagcattcttgtgattgttatttgaacgctttgcaacattattttgatttttgtgtgtgggggattttgcacacagcaatcgatttaatggattgcggttcacaagttaaaatgtactcgattgacaacaccatttatgcattgtacatcgttaccggtgcctagtctgcggttctgtcctttagtgatggatgcagggtatcattataagatgctaggcttaaatatcatcatgaagccaattgatgtacattagctaagggaatgtaaacccccggccctaaacctcaatgcatttcggtgcattgttgtactttaaccaaaatttaagctaaaaaactaaataacaaaacaaaaaaatacagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagacttgttcatcgtgttaaacttttattgttaactcgtcgagtcgcattaatgtttgccaaaaaggagactttttagaaataagttgtccgtggtatgtacaaacgtgcattacagatcggacaattgaacattatgtatgttcacttattgagaaaaactccaataacaaacaacaaaatcaaagttttgtcaaattcgacctcagatcaagcgagatta

SEQ ID NO: 39 - DNA, 883 bp, Blomia tropicalis
gtttccgtaggtgaacctgcggaaggatcattattggatattattttggtgtgtgattattcatcatcatgctaaattttatctaaattttgtgttttcggacacgaatatttttgctagttgatcattgagtagcattcttgtgattgttttttgaacgttatgcaacatttattggttttgtttgtgggggattttgcacacaagtaattagtacaatggtttgcggttcacaagtttaaatgtactcgattgacaacaccatttatgctttgtacatcgttaccggtgcctagtctgcggttctgtccactagtgatggatgcagggtatcattattagatgctaggcttaaatatcatcgtgaagccaagtgatgtacattagctaagggaatgttaacccccggccctaaacctcaatgcatttcgatgcattgttgtattctaaccaaaatttaagctaaaaaactaaataacaaaacaaaaaatacagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgttgaatttgtttttaactcgtcgagtcgcgttaatgtttgccaaaaaggagacttaaataagttgtccgtggtatgtacaaacgtgcattacagatcggactttgaacattaatgttcacttattgagaaaaaactccaataacaaacaacaaaatcattttgtcaaattcgacctcagatcaagcgagatta

SEQ ID NO: 40 - DNA, 888 bp, Blomia tropicalis
gtttccgtaggtgaacctgcggaaggatcattattggatattattttggtgtgtgattattcatcatcatgctaaattttatctaaattttgtgttttcggacacgaatatttttgctagttgatcattgggtagcattcttgtgattgttttttgaacgctatgcaacatttattggttttgtgtgtgggggaatttgcacacagcaatcgatttaatggattgcggttcacaagtttaaatgtactcgattgacaacaccatttatgcattgtacatcgttaccggtgcctagtctgcggttctgtcctttagtgatggatgcagggtatcattgtataagatgctaggcttaaatatcatcgtgaagccaagtgatgtacattagctaagggaatgttaacccccggccctaaacctcaatgcatttcgatgcattgttgtattctaaccaaaatttaagctaaaaaactaaataaaacaaaaaaatacagctgttagtggtggatcactcggctcgctggtcgaggaagaacgcagctagctgcgttaatcggtgtaaaatgcaggacacgccgagcactcgacattcgaacgcacattgcagccttggtcataccttggcttcgtttgtctgagcgtcgtttgaaatgaaagccacaaactatatgcaagactcgttcatcgtgtcaaatttgtttttaactcgtcgagtcgcattaatgtttgccaaaaaggagacttaaataagttgtccgtggtatgtacaaacgtgcattacagatcggacaattgaacattatgtatattcacttattgagaaaaactccaataacaaacaacaaaatcaaaattttgtcaaattcgacctcagatcaagcgagatta

SEQ ID NO: 41 - DNA, 1263 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccagaagtaaccaaaatgggcttatcatgttaggcgagttgtgtccgtgtgtgtggtacacccgttgaacgcttgagactaagcagagtttgcaagaggctggcgccagcaacagcaccattcatgggcaatcattggaatgtgtgcggttgtgcgcaatttaaggccacttgcatgagttgcttagaattcttccacgaagcaagtaccaccatttgacacagtacctcgtctatctgaaagctctccttgtccatctgattggtgcctagtctgcgcttcctgcctcttaacggaggcttggatgcagggtgtcagttgtgtgagtggctaaacccctctccacggctgctgctaggcttaaatatcagtgccagtgcgctgagcgccgaagcctcagatgcaaggtcgagtagtgtgcactgttgggtaaccagctctgcacgctgctgtgaaatatccagcggctcacgccgcttgggtacttcattctctaaccccaacttacaagccaaaatcacgatgatttaccctttaagtgttacctagtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacaccatgcaaaggcctgttccccatgtcgtgtcgatcattcagtcatcgactcgtcaggccacttgtaacacttctttcaactttgttattgaactgcccatacgagcgtagggagagagcctaccagtttgctggtactcgattcactttgcgtgtagatttgccgcacttgctgtggtagtctaatgtagggggcttctgacactgcctgtcagtcaagcacctgagcactgtgtcgcatcgatgccaagcgcttaaagctgtgcgcgtctccattacaatcagactcggatgaagccctcgtaacgatgaactgtgtttacactaaaactttgcatactgcacattgatttgtgcagttgttgttaactttcgacctcagatcaagcgagatta

SEQ ID NO: 42 - DNA, 1265 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccagaagtaaccaaaatgggcttatcatgttaggcgagttgtgtccgtgtgtgtggtacacccgttgaacgcttgagactaagcagagtttgcaagaggctggcgccagcaacagcaccattcatgggcaatcattggaatgtgtgcggttgtgcgcaatttaaggccacttgcatgagttgcttagaattcttccacgaagcaagtaccaccatttgacacagtacctcgtctatctgaaagctctccttgtccatctgattggtgcctagtctgcgcttcctgcctcttaacggaggcttggatgcagggtgtcagttgtgtgagtggctaaacccctctccacggctgctgctaggcttaaatatcagtgccagtgcgctgagcgccgaagcctcagatgcaaggtcgagtagtgtgcactgttgggtaaccagctctgcacgctgctgtgaaatatccagcggctcacgccgcttgggtacttcattctctaaccccaacttacaagccaaaatcacgatgatttaccctttaagtgttacctagtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacaccatgcaaaggcctgttccccatgtcgtgtcgatcattcagtcatcgactcgtcaggccacttgtaacacttctttcaactttgttattgaactgcccatacgagcgtagggagagagcctaccagtttgctggtactcgattcactttgcgtgtagatttgccgcacttgctgtggtagtctaatgtagggggcttctgacactgcctgtcagtcaagcacctgagcactgtgtcacatcgatgcaccaggcgcttaaagctgtgcgcgtctccattacaatcagactcggatgaagccctcgtaacgatgaactgtgtttacactaaaactttgcatactgcacattgatttgtgcagttgttgttaactttcgacctcagatcaagcgagatta

SEQ ID NO: 43 - DNA, 1265 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccagaagtaaccaaaatgggcttatcatgttaggcgagttgtgtccgtgtgtgtggtacacccgttgaacgcttgagactaagcagagtttgcaagaggctggcgccagcagcagcaccattcatgggcaatcattggaatgtgtgcggttgtgcgcaatttaaggccacttgcatgagttgcttagaattcttccacgaagcaagtaccgccatttgacacagtacctcgtctatctgaaagctctccttgtccatctgattggtgcctagtctgcgcttcctgcctcttaacggaggcttggatgcagggtgtcagttgtgtgagtggctaaacccctctccacggctgctgctaggcttaaatatcagtgccagtgcgctgagcgccgaagcctcagatgcaaggtcgagtagtgtgcactgttgggtaaccagctctgcacgctgctgtgaaatatccagcggctcacgccgcttgggtacttcattctctaaccccaacttacaagccaaaatcacgatgatttaccctttaagtgttacctagtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacactatgcaaaggcctgttccccatgtcgtgtcgatcattcagtcatcgactcgtcaggccacttgtaacactactttcaactttgttattgaactgcccatacgagcgtaaggagagagcttaccagtttgctggtactcgattcactttgcgtgtagatttgccgcacttgctgtggtagtctaatgtagggggcttctgacactgcctgtcagtcaagcacctgagcactgtgtcacatcgatgcaccaggcgcttaaagctgtgcgcgtctccattacaatcagactcggatgaagccctcgtaacgatgaactgtgtttacactaaaactttgcatactgcacattgatttgtgcagttgttgttaactttcgacctcagatcaagcgagatta

SEQ ID NO: 44 - DNA, 1265 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccagaagtaaccaaaatgggcttatcatgttaggcgagttgtgtccgtgtgtgtggtacacccgttgaacgcttgagactaagcagagtttgcaagaggctggcgccagcaacagcaccattcatgggcaatcattggaatgtgtgcggttgtgtgcaatttaaggccacttgcatgagttgcttagaattcttccacgaagcaagtaccaccatttgacacagtacctcgtctatctgaaagctctccttgtccatctgattggtgcctagtctgcgcttcctgcctcttaacggaggcttggatgcagggtgtcagttgtgtgagtggctaaacccctctccacggctgctgctaggcttaaatatcagtgccagtgcgctgagcgccgaagcctcagatgcaaggtcgagtagtgtgcactgttgggtaaccagctctgcacgctgctgtgaaatatccagcggctcacgccgcttgggtacttcattctctaaccccaacttacaagccaaaatcacgatgatttaccctttaagtgttacctagtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacaccatgcaaaggcctgttccccatgtcgtgtcgatcattcagtcatcgactcgtcaggccacttgtaacactactttcaactttgttattgaactgcccatacgagcgtaaggagagagcttaccagtttgctggtactcgattcactttgcgtgtagatttgccgcacttgttgtggtagtctaatgtagggggcttctgacactgcctgtcagtcaagcacctgagcactgtgtcacatcgatgcaccaagcgcttaaagctgtgcgcgtctccattacaatcagactcagatgaagcactcgtaacgatgaactgtgtttacactaaaactttgcatactgcacattgatttgtgcagttgttgttaactttcgacctcagatcaagcgagatta

SEQ ID NO: 45 - DNA, 1266 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccaaatgtaaccaaaatgggcttatcatgttaggccgagttgtgtccgtgtgtgtggtacacccgttgaacgcttgaagctaagcagagtttgcaagaggctggcgccagcagcagcaccattcatgggcaatcattggaatgtgtgcggttgtgcgcaatttaaggccacttgcatgagttgcttagaatttctttacaaagcaagtaccaccatttgacacagtacctcgtctatctgaaagctctccttgtctatctgattggtgcctagtctgcgcttcctgcctcttaacggaggcttggatgcagggtgtcagttgtgtgagtggctaaacccctctccacggctgctgctaggcttaaatatcagtgccagtgcgctgagcgccgaagcctcagatgcaaggtcgagtagtgtgcactgttgggtaaccagctttgcacgctgctgtgaaatatccagcggctcacgccgcttgggtacttcattctctaacctcaacttacaagccaaaatcacgatgatttaccctttaagtgttacctcgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacaccatgcaaaggcctgttccccatgtcgtgtcgatcattcagtcatcgactcgtcaggccacttgtaacactactttcaactttgttattgaactgcccatacgagcgtaaggagagagcttaccagtttgctggtactcgattcactttgcgtgtagatttgccgcacttgttgtggtagtctaatgtagggggcttctgacactgcctgtcagtcaagcacctgagcactgtgtcacatcgatgcaccaagcgcttaaagctgtgcgcgtctccattacaatcagactcagatgaagcactcgtaacgatgaactgtgtttacactaaaactttgcatactgcacattgatttgtgcagttgttgttaactttcgacctcagatcaagcgagatta

SEQ ID NO: 46 - DNA, 1259 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccaaatgtaaccaaaatgggcttatcatgttaggccgagttgtgtccgtgtgtgtggtacacccgttgaacgcttgaagctaagcagagtttgcaagaggctggcgccagcagcaccattcatgggcaatcaatggaatgtgtgctgttgtgcgcaatttaaggccacttgcatgagttgcttagaattcttccacgaagcaagtaccaccacttgacgcagtacctcgtctatctgaaagctctccttgtctatctgattggtgcctagtctgcgtttcctgccttctaacagaggcttggatgcagggtgtcagttgtgtgagtggctaagcccctctccacggctgctgctaggcttaaatatcagtgccagtgcgctgagcgccgaagcctcagatgcaaggtcgagtagtgtgcaccgttgggtaaccagctctgcacgctgctgtgaaatatccagcggctcacgtcgcttgggtacttcattctctaaccccaacttacaagccaaaatcacgatgatttaccctttaagtgttacctcgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacaccatgcaaaggcctgttccccatgtcgtgtcgatcattcagtcatcgactcgtcaggccacttgtaacactactttcaactttgttattgaactgcccatacgagcgtaaggagagagcctaccagtttgctggtactcgattcactttgcgtgtagatttgccgcacttgctgtggtagtctaatgtagggggcttctgacactacctgtcagtcaagcacctgagcactgtgtcacatcgatgccaagcgcttaaagctgtgcgcgtctccattacaatcagactcagatgaagcactcgtaacgatgaactgtgtttacactaaaactttgcatactgcgcacatttgtgcagttgttgttaactttcgacctcagatcaagcgagatta

SEQ ID NO: 47 - DNA, 1265 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccaaatgtaaccaaaatgggcttatcatgttaggccgagttgtgtccgtgtgtgtggtacacccgttgaacgcttgaagctaagcagagtttgcaagaggctggcgctagcagcagcaccattcatgggcaatcattggaatgtgtgcggttgtgcgcaatttaaggccacttgcatgagttgcttagaattcttctacgaagcaagtaccaccacttgacgcagtacctcgtctatctgaaagctctccttgtccatctgattggtgcctagtctgcgcttcctgcttcctaacggaggcttggatgcagggtgtcagttgtgtgagtggctaaaccccgctcggcagctgctgctaggcttaaatatcagtgccagtgcgctgagcgccgaagcctcagatgcaaggtcgagtagtgtgcactgttgggtaaccagctctgcacgctgctgtgaaatatccagcggctcacgccgcttgggtacttcattctctaaccccaacttacaagccaaaatcacgatgatttaccctttaagtgttacctagtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacactatgcaaaggcctgttccccatgtcgtgtcgatcattcagtcatcgactcgtcaggccacttgtaacactactttcaactttgttattgaactgcccatacgagcgtaaggagagagcctaccagtttgctggtactcgattcactttgcgtgtagatttgccgcacttgctgtggtagtctaatgtagggggcttctgacactacctgtcagtcaagcacctgagcactgtgtcgcatcgatgcaccaagcgcttaaagctgtgcgcgtctccaattacaatcagactcggatgaagtactcgtaacgatgaactgtgtttacactaaaactttgcatactgcgcacatttgtgcagttgttgttaactttcgacctcagatcaagcgagatta

SEQ ID NO: 48 - DNA, 1265 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccaaatgtaatcaaaatgggcttatcatgttaggcgagatgtgtccgtgtgtgtggtacacccgttgaacgcttgagactaagcagagtttgcaagaggctggcgccagcagcagcaccattcatgggcaatcattggaatgtgtgcggttgtgcgcaatttaaggccacttgcatgagttgcttagaattcttccacgaagcaagtaccaccgcttgacgcagtacctcgtctatctgaaagctctccttgtccatctgattggtgcctagtctgcgcttcctgccttctaacagaggcttggatgcagggtgtcagttgtgtgagtggctaagcccctctctacggctgctgctaggcttaaatatcagtgccagtgcgctgagcgccgaagcctcagatgcaaggtcgagtagtgtgcaccgttgggtaaccagctctgcacgctgctgtgaaatatccagcggctcacgccgcttgggtacttcattctctaaacccaacttacaagccaaaatcacgatgatttaccctttaagtgttacctcgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacaccatgcaaaggcctgttccccatgtcgtgtcgatcattcagtcatcgactcgtcaggccacttgtaacactactttcaactttgttattgaactgcccatacgagcgtaaggagagagcttaccagtttgctggtactcgattcactttgcgtgtagatttgccgcacttgttgtggtagtctaatgtagggggcttctgacactgcctgtcagtcaagcacctgagcactgtgtcacatcgatgcaccaagcgcttaaagctgtgcgcgtctccattacaatcagactcagatgaagcactcgtaacgatgaactgtgtttacactaaaactttgcatactgcacattgatttgtgcagttgttgttaactttcgacctcagatcaagcgagatta

SEQ ID NO: 49 - DNA, 1263 bp, Tyrophagus putrescentiae
gtttccgtaggtgaacctgcggaaggatcattaacggatcgacagaagaagcgaaggagttcagctctttcactaccactgtcactgtatccaaacctttttgcttgaacgcaaattgcccgttaccaaatgtaaccaaaaatgggcttatcatgttaggcgagttgtgtccgcgtgtgtggtacacccgttgaacgcttgaagctaagcagagtttgcaagaggctggcgccagcagcaccattcatgggcaatcattggaatgtgtgcggttgtgcgcaatttaaggccacttgcatgagttgcttagaattcttccacgaagcaagtaccaccatttgacacagtacctcgtctatctgaaagctctccttgtccatctgattggtgcctagtctgcgcttcctgcctcttaacggaggcttggatgcagggtgtcagttgtgtgagtggctaaaccccgctcggcagctgctgctaggcttaaatatcagtgccagtgcgctgagcgccgaagcctcagatgcaaggtcgagtagtgtgcaccgttgggtaaccagctctgcacgctgctgtgaaatatccagcggctcacgtcgcttgggtacttcattctctaaccccaacttacaagccaaaatcacgatgatttaccctttaagtgttacctcgtgtaaacttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacaccatgcaaaggcctgttccccatgtcgtgtcgatcattcagtcatcgactcgtcaggccacttgtaacactactttcaactttgttattgaactgcccatacgagcgtaaggagagagcttaccagtttgctggtactcgattcactttgcgtgtagatttgccgcacttgttgtggtagtctaatgtagggggcttctgacactgcctgtcagtcaagcacctgagcactgtgtcacatcgatgcaccaagcgcttaaagctgtgcgcgtctccattacaatcagactcggatgaagccctcgtaacgatgaactgtgtttacactaaaactttgcatactgcacattgatttgtgcagttgttgttaactttcgacctcagatcaagcgagatta

SEQ ID NO: 50 - DNA, 1261 bp, Tyrophagus putrescentiae
gtttccgtaggtgaacctgcggaaggatcattaacggatcgacagaagaagcgaaggagttcagctctttcactgccactgtcactgtatccaaacctttttgcttgaacgcaaattgcccgttaccaaatgtaaccaaaaatgggcttatcatgttaggcgagttatgtccgtgtgtgtggtacacccgttgaacgcttgaagctaagcaaagtttgcaagaggctggcgccagcagcaccattcatgggcaatcattggaatgtgtgcggttgtgcgcaatttaaggccacttgcatgagttgcttagaattcttccacgaagcaagtaccaccatttgacacagtacctcgtctatctgaaagctctccttgtccatctgattggtgcctagtctgcgcttcctgcctcttaacggaggcttggatgcagggtgtcagttgtgtgagtggctaaacccctctctacggctgctgctaggcttaaatatcagtgccagtgcgctgagcgccgaagcctcagatgcaaggtcgagtagtgtgcaccgttgggtaaccagctctgcacgctgctgtgaaatatccagcggttcacgccgtttgggtacttcattctctaaccccaacttacaagccaaaatcacgatgatttaccctttaagtgttacctcgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagccgaggttatacctcggcttcgtttgtctgagcgtcgttaatatgccaaacaccatgcaaaggcctgttccccatgtcgtgtcgatcattcagtcatcgactcgtcaggccacttgtaacacttctttcaactttgttattgaactgcccatacgagcgtagggagagagcctaccagtttgctggtactcgattcactttgcgtgtagatttgccgcacttgctgtggtagtctaatgtagggggcttctgacactgcctgtcagtcaagcacctgagcactgtgtcgcatcgatgccaagcgcttaaagctgtgcgcgtctccattacaatcagactcagatgaagcactcgtaacgatgaactgtgtttacactaaaactttgcatactgcacattgatttgtgcagttgttgttaactttcgacctcagatcaagcgagatta

SEQ ID NO: 51 - DNA, 1331 bp, Tyrophagus fanetzhangorum
gtttccgtaggtgaacctgcggaaggatcattaacggatcgacagaagctgaaagccgtctgctgttgtgctcttgcagtgcatcatcatcatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttacaagaggctggcgccaatagcatcaccatttgtgggcaatcattggaatggttgcagttgcgcgcattttaaggctaattgtatgaattgcttagaacttccttgaaaagcaggtgtactaactagccaacagtgctcgtctatctgaaagctctccttgttcatctgattggtgcctagtctgcgcttcctgccttctaacgacggcttggatgcagggtgtcagttgagtgtgtgtgggtgttaaagcctccactgctcaattgctgctaggcttaaatatcagtgccagtgcgctgagcgccgaagcctcagatgcaaggtcgagtagtgtgcagttgagcaatcaacggtgcactgctgtgaaatatccagcggatgctgctgcttcccaaatttttgggtggcgcacgccgttttgggtacttcattctctaaccccaacttacaagccaaaatcatgatgatttaccctttaagttttaccttgtgtaagcttaaacaatgacaaaattacaactgttagtggtggatcactcggcacgctgatcgaggaagaacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccattcagtcgtcgactcgtcaggccacttgtaacactactttcaacattgttatgtaaatgcccatacgagcgtaagaagagagttgcttaccagtttgccggtactcgattcactttgcgtgtagatttgccgcagaccaattctgcagtagtctaatgtagggggcttctgacactacctgtcagtttagcacctgagcactgtgtcacatcgctgctgcgcatcaaaactgtgcaaatttctcactattacattcagactcggatgaagcactcgtaacgtgaattgtgtttacacgctaaaactttgcatactttgtgcagatgtgttgttaactttcgacctcagatcaagcgagatta

SEQ ID NO: 52 - DNA, 1331 bp, Tyrophagus fanetzhangorum
gtttccgtaggtgaacctgcggaaggatcattaacggatcgacagaagctgaaagccgtctgctgttgtgctcttgcagtgcatcatcatcatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttacaagaggctggcgccaatagcatcaccatttgtgggcaatcattggaatggttgcagttgcgcgcattttaaggctaattgtatgaattgcttagaacttccttgaaaagcaggtgtactaactagccaacagtgctcgtctatctgaaagctctccttgttcatctgattggtgcctagtctgcgcttcctgccttctaacgacggcttggatgcagggtgtcagttgagtgtgtgtgggtgttaaagcctccactgctcaattgctgctaggcttaaatatcagtgccagtgcgctgagcgccgaagcctcagatgcaaggtcgagtagtgtgcagttgagcaatcaacggtgcactgctgtgaaatatccagcggatgctgctgcttcccaaatttttgggtggcgcacgccgttttgggtacttcattctctaaccccaacttacaagccaaaatcatgatgatttaccctttaagttttaccttgtgtaagcttaaacaatgacaaaattacaactgttagtggtggatcactcggcacgctgatcgaggaagaacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccattcagtcgtcgactcgtcaggccacttgtaacactactttcaacattgttatgtaaatgcccatacgagcgtaagaagagagttgcttaccagtttgccggtactcgattcactttgcgtgtagatttgccgcagaccaattctgcagtagtctaatgtagggggcttctgacactatctgtcagtttagcacctgagcactgtgtcacatcgctgctgcgcatcaaaactgtgcaaatttctcactattacattcagactcggatgaagcactcgtaacgtgaattgtgtttacacgctaaaactttgcatactttgtgcagatgtgttgttaactttcgacctcagatcaagcgagatta

SEQ ID NO: 53 - DNA, 1332 bp, Tyrophagus fanetzhangorum
gtttccgtaggtgaacctgcggaaggatcattaacggatcgacagaagctgaaagccgtctgctgttgtgctcttgcagtgcatcatcatcatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttacaagaggctggcgccaatagcatcaccatttgtgggcaatcattggaatggttgcagttgcgcgcattttaaggctaattgtatgaattgcttagaacttccttgaaaagcaggtgtactaactagccaacagtgctcgtctatctgaaagctctccttgttcatctgattggtgcctagtctgcgcttcctgccttctaacgacggcttggatgcagggtgtcagttgagtgtgtgtgggtgttaaagcctccactgctcaattgctgctaggcttaaatatcagtgccagtgcgctgagcgccgaagcctcagatgcaaggtcgagtagtgtgcagttgagcaatcaacggtgcactgctgtgaaatatccagcggatgctgctgcttcccaaatttttgtgtggcgcacgccgttttgggtacttcattctctaaccccaacttacaagccaaaatcatgatgatttaccctttaagttttaccttgtgtaagcttaaacaatgacaaaattacaactgttagtggtggatcactcggcacgctgatcgaggaaggacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccattcagtcgtcgactcgtcaggccacttgtaacactactttcaacattgttattgaaatgcccatacgagcgtaagaagagagttgcttaccagtttgccggtactcgattcactttgcgtgtagatttgccgcagaccaattctgcggtagtctaatgtagggggcttctgacactacctgtcagtttagcacctgagcactgtgtcacatcgttgctgcgcatcaaaactgtgcaaatttctcactattacattcagactcggatgaagcactcgtaacgtgaattgtgtttacacgctaaaactttgcatactttgtgtgcagttgtttgttaactttcgacctcagatcaagcgagatta

SEQ ID NO: 54 - DNA, 1329 bp, Tyrophagus fanetzhangorum
gtttccgtaggtgaacctgcggaaggatcattaacggatcgacagaagctgaaagccgtctgctgttgtgctcttgcagtgcatcatcatcatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttgcaagaggctggcgccaatagcatcaccattcgtgggcaatcatcggaatggttgcagttgcgcgcattttaaggccaattgtatgaattgcttagaacttccttgaaaagcaggtgtactaactagccaacagtgctcgtttatctgaaagctctccttgtccatctgattggtgcctagtctgcgcttcctgccttctaacgacggcttggatgcagggtgtcagttgagtgtgtgcgggtgttaaagcctccactgctcaactgctgctaggcttaaatatcagtgccagtgcgctgagcgccgaagcctcagatgcaaggtcgagtagtgtgcagttgagcaatcaacggtgcactgctgtgaaatatccagcggatgctgctgctccccaaatttttgggttgcgcacgccgttttgggtacttcattctctaaccccaacttacaagccaaaatcatgatgatttaccctttaagttttaccttgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccatgcagtcgtcgactcgtcaggccacttgtaacactactttcaacattgttatgcaaatgcccatacgagcgtaagaagagagttgcttaccagtttgccggtactcgattcactttgcgtgtagatttgccgcagaccaattctgcggtagtctaatgtagggggcttctgacactacctgtcagtttagcacctgagcactgtgtcacatcgctgctgcgcatcaaaactgtgcaaatttctcactattacattcagactcggatgaagcacttgtaacgtgaattgtgtttacactaaaactttgcatactttgtgcagatgtgttgttaactttcgacctcagatcaagcgagatta

SEQ ID NO: 55 - DNA, 1332 bp, Tyrophagus fanetzhangorum
gtttccgtaggtgaacctgcggaaggatcattaacggatcgacagaagctgaaagccgtctgctgttgtgctcttgcagtgcatcatcatcatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttgcaagaggctggcgccaatagcatcaccattcgtgggcaatcatcggaatggttgcagttgcgcgcattttaaggccaattgtatgaattgcttagaacttccttgaaaagcaggtgtactaactagccaacagtgctcgtttatctaaaagctctccttgtccatctgattggtgcctagtctgcgcttcctgccttctaacgacggcttggatgcagggtgtcagttgagtgtgtgcgggtgttaaagcctccactgctcaactgctgctaggcttaaatatcagtgccagtgcgctgagcgccgaagcctcagatgcaaggtcgagtagtgtgcagttgagcaatcaacggtgcactgctgtgaaatatccagcggatgctgctgctccccaaatttttgggttgcgcacgccgttttgggtacttcattctctaaccccaacttacaagccgaaatcatgatgatttaccctttaagttttaccttgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccattcagtcgtcgactcgtcaggccacttgtaacactactttcaacattgttattgaaatgcccatacgagcgtaagaagagagttgcttaccagtttgccggtactcgattcactttgcgtgtagatttgccgcagatcaattctgcggtagtctaatgtagggggcttctgacactacctgtcagtttagcacctgagcactgtgtcacatcgctgctgcgcatcaaaactgtgcaaatttctcactattacattcagactcggatgaagcactcgtaacgtgaattgtgtttacgctaaaactttgcatactaaaactgtgcagttgtttgttaactttcgacctcagatcaagcgagatta

SEQ ID NO: 56 - DNA, 1329 bp, Tyrophagus fanetzhangorum
gtttccgtaggtgaacctgcggaaggatcattaacggatcgacagaagctgaaagccgtctgttgttgtgctcttgcagtgcatcatcattatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttgcaagaggctggcgccaatagcatcaccattcgtgggcaatcatcggaatggttgcagttgcgcgcattttaaggccaattgtatgaattgcttagaacttccttgaaaagcaggtgtactaactagccaacagtgctcgtctatctgaaagctctccttgtccatctgattggtgcctagtctgcgcttcctgccttctaacgacggcttggatgcagggtgtcagttgtgtgtgtgtgggtgttaaagcctctactactcaattgctgctaggcttaaatatcagtgccagtgcgctgagcgccgaagcctcagatgcaaggtcgagtagtgtgcagttgagcaatcaacggcgcactgctgtgaaatatccagcggatgctgctgctctccgaattttcgggtggagcacgccgttttgggtacttcattctctaaccccaacttacaagccaaaatcatgatgatttaccctttaagttttaccttgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccattcagtcgtcgactcgtcaggccatttgtaacactactttcaacattgttattgaaatgcccatacgagcgtaagaagagagttgcttaccagtttgccggtactcgattcactttgcgtgtagatttgccgcagaccaattctgcgatagtctaatgtagggggcttctgacactacctgtcagtttagcgcctgagcactgtgtcacatcgctgctgcgcatcaaaactgtgcaaatttctcactattacattcagactcggatgaagcactcgtaacgtgaattgtgtttacactaaaactttgcatactttgtgcagatgtgttgttaactttcgacctcagatcaagcgagatta

SEQ ID NO: 57 - DNA, 1329 bp, Tyrophagus fanetzhangorum
gtttccgtaggtgaacctgcggaaggatcattaacggatcgacagaagctgaaagccgtctgttgttgtgctcttgcagtgcatcatcattatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttgcaagaggctggcgccaatagcatcaccattcgtgggcaatcatcggaatggttgcagttgcgcgcattttaaggccaattgtatgaattgcttagaacttccttgaaaagcaggtgtactaactagccaacagtgctcgtctatctgaaagctctccttgtccatctgattggtgcctagtctgcgcttcctgccttctaacgacggcttggatgcagggtgtcagttgtgtgtgtgtgggtgttaaagcctctactactcaattgctgctaggcttaaatatcagtgccagtgcgctgagcgccgaagcctcagatgcaaggtcgagtagtgtgcagttgagcaatcaacggcgcactgctgtgaaatatccagcggatgctgctgctctccgaattttcgggtggagcacgccgttttgggtacttcattctctaaccccaacttacaagccaaaatcatgatgatttaccctttaagttttaccttgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccattcagtcgtcgactcgtcaggccatttgtaacactactttcaacattgttattgaaatgcccatacgagcgtaagaagagagttgcttaccagtttgccggtactcgattcactttgcgtgtagatttgccgcagaccaattctgcggtagtctaatgtagggggcttctgacactacctgtcagtttagcacctgagcactgtgtcacatcgctgctgcgcatcaaaactgtgcaaatttctcactattacattcagactcggatgaagcactcgtaacgtgaattgtgtttacactaaaactttgcatactttgtgcagatgtgttgttaactttcgacctcaggtcaagcgagatta

SEQ ID NO: 58 - DNA, 1329 bp, Tyrophagus fanetzhangorum
gtttccgtaggtgaacctgcggaaggatcattaacggatcgacagaagctgaaagccgtctgttgttgtgctcttgcagtgcatcatcattatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttgcaagaggctggcgccaatagcatcaccattcgtgggcaatcatcggaatggttgcggttgcgcgcattttaaggccaattgtatgaattgcttagaacttccttgaaaagcaggtgtactaactagccaacagtgctcgtctatctgaaagctctccttgtccatctgattggtgcctagtctgcgcttcctgccttctaacgacggcttggatgcagggtgtcagttgtgtgtgtgtgggtgttaaagcctctactactcaattgctgctaggcttaaatatcagtgccagtgcgctgagcgccgaagcctcagatgcaaggtcgagtagtgtgcagttgagcaatcaacggcgcactgctgtgaaatatccagcggatgctgctgctctccgaattttcgggtggagcacgccgttttgggtacttcattctctaaccccaacttacaagccaaaatcatgatgatttaccctttaagttttaccttgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccattcagtcgtcgactcgtcaggccatttgtaacactactttcaacattgttattgaaatgcccatacgagcgtaagaagagagttgcttaccagtttgccggtactcgattcactttgcgtgtagatttgctgcagaccaattctgcggtagtctaatgtagggggcttctgacactacctgtcagtttagcacctgagcactgtgtcacatcgttgctgcgcatcaaaactgtgcaaatttctcactattacattcagactcggatgaagcactcgtaacgtgaattgtgtttacactaaaactttgcatactttgtgcagatgtgttgttaactttcgacctcagatcaagcgagatta

SEQ ID NO: 59 - DNA, 1329 bp, Tyrophagus fanetzhangorum
gtttccgtaggtgaacctgcggaaggatcattaacggatcgacagaagctgaaagccgtctgctgttgtgctcttgcagtgcatcatcatcatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttgcaagaggctggcgccaatagcatcaccattcgtgggcaatcatcggaatggttgcagttgcgcgcattttaaggccaattgtatgaattgcttagaacttccttgaaaagcaggtgtactaactagccaacagtgctcgtctatctgaaagctctccttgtccatctgattggtgcctagtctgcgcttcctgccttctaacgacggcttggatgcagggtgtcagttgtgtgtgtgtgggtgttaaagcctctactactcaattgctgctaggcttaaatatcagtgccagtgcgctgagcgccgaagcctcagatgcaaggtcgagtagtgtgcagttgagcaatcaacggcgcactgctgtgaaatatccagcggatgctgctgctctccgaattttcgggtggagcacgccgttttgggtacttcattctctaaccccaacttacaagccaaaatcatgatgatttaccctttaagttttaccttgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagcctaggttataccttggcttcatttgtctgagcgtcgttaatatgccaaaccccatgcaaaggcctgctgcacttgcgtgcagtgtcgtgtcgaccattcagtcgtcgactcgtcaggccatttgtaacactactttcaacattgttattgaaatgcccatacgagcgtaagaagagagttgcttaccagtttgccggtactcgattcactttgcgtgtagatttgccgcagaccaattctgcggtagtctaatgtagggggcttctgacactacctgtcagtttagcacctgagcactgtgtcacatcgctgctgcgcatcaaaactgtgcaaatttctcactattacattcagactcggatgaagcactcgtaacgtgaattgtgtttacactaaaactttgcatactttgtgcagatgtgttgttaactttcgacctcagatcaagcgagatta

SEQ ID NO: 60 - DNA, 1332 bp, Tyrophagus fanetzhangorum
gtttccgtaggtgaacctgcggaaggatcattaacggatcgacagaagctgaaagccgtttgttgttgtgctcttgcggtgcatcatcattatcactttcactgccactgtcactgtatccaaacctttttgcttgaacgcaaattgcccgttaccaaaagctaaaaatgggcttatcatgttaggcgagagttgtgcgtgtggtacacactttgaacgcttgaagctaagcagagtttgcaagaggctggcgccaatagcatcaccattcgtgggcaatcatcggaatggttgcagttgcgcgcattttaaggccaattgtatgaattgcttagaacttccttgaaaagcaggtgtactaactagccaacattgctcgtctatctgaaagctctccttgttcatctgattggtgcctagtctgcgcttcctgccttctaacgacggcttggatgcagggtgtcagttgagtgtgtgcgggtgttaaagtctccactgctcaattgctgctaggcttaaatatcagtgccagtgcgctgagcgccgaagcctcagatgcaaggtcgagtagtgtgcagttgagcaatcaacggcgcactgctgtgaaatatccagcggatgctgctgcttcccgaattttcgggtggcgcacgccgttttgggtacttcattctctaaccccaacttacaagccaaaatcatgatgatttaccctttaagttttaccttgtgtaagcttaaacaatgacagaattacaactgttagtggtggatcactcggcacgctgatcgaggaagaacgcagctagctgcgttaatcggtataaaatgcaggacatgccgaatactcgactttcgaacgcatattgcagcctaggttataccttggcttcgtttgtctgagcgtcgttaatatgccaaaccccatgcaaaggcctgctgcacttgcgtgcagtgtcgtgtcgaccattcagtcgtcgactcgtcaggccacttgtaacactactttcaacattgttattgaaatgcccatacgagcgtaagaagagagttgcttaccagtttgccggtactcgattcactttgcgtgtagatttgccgcagatcaattctgcggtagtctaatgtagggggcttctgacactacctgtcagtttagcacctgagcactgtgtcacatcgctgctgcgcatcaaaactgtgcaaatttctcactattacattcagactcggatgaagcactcgtaacgtgaattgtgtttacgctaaaactttgcatactaaaactgtgcagttgtttgttaactttcgacctcagatcaagcgagatta

SEQ ID NO: 61 - DNA, 908 bp, Dermatophagoides pteronyssinus
gtttccgtaggtgaacctgcggaaggatcattatcggttttcttgagcattcatttgcttaaaagaaacattcgattatcaattcgaacgaatcgttgcccgttatcacaaacggatcgactggcaattgttgacagtgatgaggttattgaaagctctgaaattgtatcctggttttcgtgttgggcgacatggatgaaaacaatgggagaatttcttaatttaggagctaaaccgaatcatgtcaactttgtcggcgattctccttgatgcctgttggtgcctagcctacagttcctgtttgtcctcgggacagatgtggatgtagtgtgtcgctttgttcttgcaacaagtgccgctaggtttaaatactggattcgtcatcgaacgaatgccggagccacaggttcaaggtattttctttttcatttatgaaaaacttcaagccagaaacattgttacaaaacaaattgtgattctcgaaagaattgcaaaaaacaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattatgaccaaacaaaatgggatagatctgtttcgtcgtgcatacgtcgtcaggtcatttccaaacatttgatatgctgacttttgtggtgaagaaggctttgtagcacattcactcacaagaggtatttagcgtttccagctaaacacctcgaatgtgtgccttgcttaaccaaaatatagtcgcaaatcattgtccaaaacaaaacgtctaatttcgacctcagatcaagcgagaata

SEQ ID NO: 62 - DNA, 908 bp, Dermatophagoides pteronyssinus; identical to SEQ ID NO: 61

SEQ ID NO: 63 - DNA, 909 bp, Dermatophagoides pteronyssinus
gtttccgtaggtgaacctgcggaaggatcattatcggctttcttgagcattcattttgcttaaaagaaacattcgattatcaattcgaacgaatcgttgcccgctatcacaaacggattgactggcaattgttgacattgatgaggttattgaaagctctgaaattgtatcctggttttcatgttgggcggcatggatgaaaacaatgggagaatttcttaatttaggagctggaccgaatcatgttaactttgttggcgattctccttgatgcctgttggtgcctagcctacagttcctgtttgtcctcgggacagatgtggatgtagtgtgtcgctttgttcttgaaacaagtgccgctaggtttaaatactggattcgtcatcgaacggatgccggagccacaggttcgaggtattttctttttcatttctgaaaaacattaagccagaaacatcgatacaaagcaaattgtgattctgcaaagaattgcaaaaaacaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattatgaccaaacaaaatggaatagatctgtgtcgtcgtgcatacgtcgtcaggtcatttccaaacatttgatatgctaacttttgtggtgaagaaggctttgtagcacattcactcacaagaggtatttagcgtttccagctaaacaccttgaatgtgtgccttgcataaccaaagtatagttgcaaatcattgtacaaatcaaaacgtctaatttcgacctcagatcaagcgagaata

SEQ ID NO: 64 - DNA, 906 bp, Dermatophagoides pteronyssinus
gtttccgtaggtgaacctgcggaaggatcattatcggttttcttgagcaatttatttgcttaaaagaaacatacgattatcaattcgaacgaaaagttgcccgttatcacaaatggatcgactggcaattgttgacactgatgaggttaatgaaagctctgaaattgtatcctggttttcatgttgggcaacatggatgaaaacaatgggagaatttcttaatttaggagctggatcgaatcatctcaactttgttggcgattctccttgatgcctgttggtgcctagcctacagttcctgtttgtcctcgggacagatgtggatgtagtgtgtcgctttgttcttgaaacaagtgccgctaggtttaaatactggattcgtaattgaacgaatgccggagccacaggttcaaggtattttctttttcatttatgaaaaacattaagccagaaacattgatacaaagcaaattgtgattctgcaaagaattgcaaaaaacaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattatgaccaaacaaaatgggatagatctgtttcgtcgtgcaaacgtcgtcggatcatttccaaacatttgatatgctgacttttgtggtgaagaaggctttgtagcacattcactcacaaaaggtatttagcgtttccagctaaacaccttgaatgtgtgccttgtataaccaaagtatagtcgcaaatcattgtccaataaaagtctaatttcgacctcagatcaagcgagaata

SEQ ID NO: 65 - DNA, 909 bp, Dermatophagoides pteronyssinus
gtttccgtaggtgaacctgcggaaggatcattatcggttttcttgagcaatttatttgcttaaaagaaacatacgattatcaattcgaacgaaaagttgcccgttatcacaaatggatcgactggcaattgttgacactgatgaggttaatgaaagctctgaaattgtatcctggttttcatgttgggcaacatggatgaaaacaatgggagaatttcttaatttaggagctggatcgaatcatctcaactttgttggcgattctccttgatgcctgttggtgcctagcctacagttcctgtttgtcctcgggacagatgtggatgtagtgtgtcgctttgttcttgaaacaagtgccgctaggtttaaatactggattcgtaattgaatgaatgccggagccacaggttcaaggtattttctttttcatttatgaaaaacattaagccagaaacattgatacaaagcaaattgtgattctgcaaagaattgcaaaaaacaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattatgaccaaacaaaatgggatagatctgtttcgtcgtgcaaacgtcgtcggatcatttccaaacatttgatatgctaacttttgtggtgaagaaggctttgtagcacattcactcacaagaggtatttagcgtttccagctaaacaccttgaatgtgtgccttgcataaccaaagtatagtcgcaaatcattgtacaaatcaaaacgtctaatttcgacctcagatcaagcgagaata

SEQ ID NO: 66 - DNA, 906 bp, Dermatophagoides pteronyssinus
gtttccgtaggtgaacctgcggaaggatcattatcggttttcttgagcaatttatttgcttaaaagaaacatacgattatcaattcgaacgaaaagttgcccgttatcacaaatggatcgactggcaattgttgacactgatgaggttaatgaaagctctgaaattgtatcctggttttcatgttgggcaacatggatgaaaacagtgggagaatttcttaatttaggagctggatcgaatcatctcaactttgttggcgattctccttgatgcctgttggtgcctagcctacagttcctgtttgtcctcgggacagatgtggatgtagtgtgtcgctttgttcttgaaacaagtgccgctaggtttaaatactggattcgtaattgaacgaatgccggagccacaggttcaaggtattttctttttcatttatgaaaaacattaagccagaaacattgatacaaagcaaattgtgattctgcaaagaattgcaaaaaacaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattatgaccaaacaaaatgggatagatctgtttcgtcgtgcaaacgtcgtcggatcatttccaaacatttgatatgctgacttttgtggtgaagaaggctttgttgcacattcactcacaagaggtatttagcgtttccagctaaacaccttgaatgtgtgccttgtataaccagtcaaaatggttgacaaataatgtaccaatcgtctaatttcgacctcagatcaagcgagaata

SEQ ID NO: 67 - DNA, 910 bp, Dermatophagoides pteronyssinus
gtttccgtaggtgaacctgcggaaggatcattatcggttttcttgagcatttatttgcccaaaagaaacattcgattatcaattcgaacgaatcgttgcccgttatcacaaacggatcgactggcaattgttgacattgatgaggttattgaaagctctgaaattgtatcctggttttcgtgttgggcgacatggatgaaaacaatgggaaaatttcttaatttgggagctggaccgaatcatgtcaactttgttggcgattctccttgatgcctgttggtgcctagcctacagttcctgtttgtcctcgggacaaatgtggatgtagtgtgtcactttgttgtttttgcaacaagtgccgctaggtttaaatactggattcgtcatcgtacgaatgccggagccacaggttcaaggtattttctttttcatttatgaaaaacattaagccagaaacattgttacaaaccaaattgtgattctgcaaagaattgcaaaaaacaaattaactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattatgaccaaacaaaatgggatagatttgtttcgtcgtgcatacgtcgtcaggtcatttccaaacatttgatatgctgacttttgtggtgaagaaggctttgtagcacattcactcacaaaaggtatttagcgtttccagctaaacaccttgaatgtgtgccttgtataaccaatcaaaatggttgacaaatcaatgtccaataaaagtctaatttcgacctcagatcaagcgagaata

SEQ ID NO: 68 - DNA, 908 bp, Dermatophagoides pteronyssinus
gtttccgtaggtgaacctgcggaaggatcattatcggttttcttgagcatttatttgcccaaaagaaacattcgattatcaattcgaacgaatcgttgcccgttatcacaaacggatcgactggcaattgttgacattgatgaggttattgaaagctctgaaattgtatcctggttttcgtgttgggcgacatggatgaaaacaatgggaaaatttcttaatttgggagctggaccgaatcatgtcaactttgttggcgattctccttgatgcctgttggtgcctagcctacagttcctgtttgtcctcgggacaaatgtggatgtagtgtgtcgctttgttgtttttgcaacaagtgccgctaggtttaaatactggattcgtcatcgtacgaatgccggagccacaggttcaaggtattttctttttcatttatgaaaaacattaagccagaaacattgttacaaaccaaattgtggttctgcaaagaattgcaaaaaacaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattatgaccaaaaaaatgggatagatctgtttcgtcgtgcatacgtcgtcaggtcatttccaaacattcgatatgctgacttttgtggtgaagaaggctttgtagcacgttcattcacaagaggtatttagcgtttccagctaaacacctcgaatgtgtgccttgtataaccggtcaaaatggttgacaaatcaatgtaccaatcgtctaatttcgacctcagatcaagcgagaata

SEQ ID NO: 69 - DNA, 909 bp, Dermatophagoides pteronyssinus
gtttccgtaggtgaacctgcggaaggatcattatcgattttcttgagcattcattttgcccaaaagaaacattcgattatcaattcgaacgaatcgttgcccgttatcacaaacggatcgactggcaattgttgacactgatgaggttattgaaagctctgaaattgtatcctggttttcgtgttgggcgacatggatgaaaacaatgggagaatttcttgatttaggggttggaccgaatcatgtcaactttgttggcgattctccttgatgcttgttggtgcctagcctacagttcctgtttgtcctcgggacaaatgtggatgtagtgtgtcgctttgttgtttttgcaacaagtgccgctaggtttaaatactggattcgtcatggaacgaatgccggagccacaggttcaaggtattttctttttcatttatgaaaaacattaagccagaaacattgttacaaaccaaattgtgattctacaaagaattgcaaaaaacaagaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttttcaaattatgaccaaacaaaatgggatagatctgtttcgtcgtgtaaacgtcgtcggatcatttccaaacatttgatatgctgacttttgtggtgaagaaggctttgtagcacattcactcacaaaaggtatttagcgtttccagcttaacaccttgaatgtgtgccttgtataaccaaagtatagtcgcaaatcattgtccaataaaagtctaatttcgacctcagatcaagcgagaata

SEQ ID NO: 70 - DNA, 910 bp, Dermatophagoides pteronyssinus
gtttccgtaggtgaacctgcggaaggatcattatcggttttcttgagcatttatttgcccaaaagaaacattcgattatcaattcgaacgaaaagttgcccgttatcacaaatgggtcgactggcaattgttgacattgattaggttattgaaagctctgaaattgtatcctggttttcatgttgggcgacatggatgaaagcaatgggacaatttcttaattaaggagtaagaccgaatcatgtcaactttgtcggcgattctccttgatgcctgttggtgcctagcctacagttcctgtttgtcctcgggacagatgtggatgtagtgtgtcgctttgttgtttttgcaacaagtgccgctaggtttaaatactggattcgtcatcgaacgaatgccggagccacaggttcaaggtatttttttttcatttaagaaaaacttaaagccagaaacattgttacaaaacaagttgtgattctcgaaagaattgcaataaacaaatataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattatgaccaaacaaaatggaatagatctgtgtcgtcgtgcatacgtcgtcaggtcatttccaaacatttgatatgctaacttttgtggtgaagaaggctttgtagcacattcactcacaagaggtatttagcgtttccagctaaacaccttgaatgtgtgccttgcataaccaaagtatagtcgcaaatcattgtacaaatcaaaacgtctaatttcgacctcagatcaagcgagaata

SEQ ID NO: 71 - DNA, 904 bp, Euroglyphus maynei
gtttccgtaggtgaacctgcggaaggatcattatcggtttttcgtggcattcatttgctgtcggaaacattcgattatcaatttgaacgacagttgcccgttatcacaaatggaacgactggcaattgttgtgattgaggttgtcattgaatgctctgaagttttgtcctggtctttatgttgggcaacatggataaagacgtcgggtgaacttcttaatttggagcctgacaattatcaatgtgtaactttgccggcgtttctccttgacgcctgttggtgcctagcctacagttcctgtccatcctcgggatggtcgtggatgtagtgtgtcgcttcaacaaagtgccgctaggtttaaatactggattcgaattattcgaatgtcggagccacaggtacaaggtatctctttttcatttatgaaaaacattgagccagaatcattgtatcatttaatttgtgattctctacgtagaattgcaaacaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccgaatcgtcgtgatgtgttaaattgcgtcgtcggatcatttccaaacatttgatatgctgactcttgtggtgaagaaggctttgtcgcacattcactcaggaggtatttagcgtttccagctaaacgcccggaatgtgtgccttttgctactttcaagtcaacagatcattgttccaaaacaaatgtctaatttcgacctcagatcaagcgagacta

SEQ ID NO: 72 - DNA, 904 bp, Euroglyphus maynei
gtttccgtaggtgaacctgcggaaggatcattatcggtttttcgtggcattcatttgctgtcggaaacattcgattatcaatttgaacgacagttgcccgttatcacaaatggaacgactggcaattgttgtgattgaggttgtcattgaatgctctgaagttttgtcctggtctttatgttgggcaacatggataaagacgtcgggtgaacttcttaatttggagcctgacaattatcaatgtgcaactttgccggcgtttctccttgacgcctgttggtgcctagcctacagttcctgtccatcctcgggatggtcgtggatgtagtgtgtcgcttcaacaaagtgccgctaggtttaaatactggattcgaattattcgaatgtcggagccacaggtacaaggtatctctttttcatttatgaaaaacattgagccagaatcattgtatcatttaatttgtgattctctacgtagaattgcaaacaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccgaatcgtcgtgatgtgttaaattgcgtcgtcggatcatttccaaacatttgatatgctgactcttgtggtgaagaaggctttgtcgcacattcactcaggaggtatttagcgtttccagctaaacgcccggaatgtgtgccttttgctactttcaagtcaacagatcattgttccaaaacaaatgtctaatttcgacctcagatcaagcgagacta

SEQ ID NO: 73 - DNA, 901 bp, Euroglyphus maynei
gtttccgtaggtgaacctgcggaaggatcattatcggtttttcgtggcattcatttgctgtcggaaacattcgattatcaatttgaacgacagttgcccgttatcacaaatggaacaactggcaattgttgtgattgaggttgtcattgaatgctctgaagttgtatcctggtctttatgttgggcaacatggataaagacgtcgggtgaacttctcaattggagcctgacaatcatcaatgtgcaactttgccggcgtttctccttgacgcctgttggtgcctagcctacagttcctgtccatcctcgggatggtcgtggatgtagtgtgtcgctttatcaaagtgccgctaggtttaaatactggattcgaattattcgaatgtcggagccacaggtacaaggtatttctttttcatttatgaaaaacattgagccagaatcattgtatcattgaatttgtgattctctacgtagaattgcaaacaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccgaatcgtcgtaatattaaattgcgtcgtcggatcatttccaaacatttgatatgctgactcttgtggtgaagaaggctttgtcgcacattcactcaggaggtatttagcgtttccagctaaacgcccggaatgtgtgccttttgctactttcaagtcaacagatcattgttccaaaacaaatgtctaatttcgacctcagatcaagcgagacta

SEQ ID NO: 74 - DNA, 903 bp, Euroglyphus maynei
gtttccgtaggtgaacctgcggaaggatcattatcggtttttcgtggcattcatttgctgtcggaaacattcgattatcaatttgaacgacagttgcccgttatcacaaatggaacgactggcaattgttgtgattgaggttgtcattgaatgctctgaagttgtatcccggtctttatgttgggcaacatggataaagacaccgggtgaacttctcaattaggagcctgacaattatcaatgtgcaactttgtcggcgtttctccttgacgcctgttggtgcctagcctacagttcctgtccatcctcgggatggtcgtggatgtagtgtgtcgcttcatcaaagtgccgctaggtttaaatactggattcgaattattcgaatgtcggagccacaggtacaaggtatttctttttcatttatgaaaaacattgagccagaatcattgtatcattgaatttgtgattctctacgcagaattgcaaacaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccgaatcgtcgtgatgtgttaaattgcgtcgtcggatcatttccaaacatttgatatgctgactcttgtggtgaagaaggctttgtcgcacattcactcaggaggtatttagcgtttccagctaaacgcccggaatgtgtgccttttgctactttcaagtcaacagatcattgttccaaaacaaatgtctaatttcgacctcagatcaagcgagacta

SEQ ID NO: 75 - DNA, 902 bp, Euroglyphus maynei
gtttccgtaggtgaacctgcggaaggatcattatcggtttttcgtggcattcatttgctgtcggaaacattcgattatcaatttgaacgtcagttgcccgttatcacaaatggaacgactggcaattgttgtgattgaggttgtcattgaatgctctggagttgtatcctggtctttacgtagggcaacatggataaagacgtcgggtgaacttcttaaattggagcctgacaattatcaatgtgcaactttgccggcgtttctccttgaagcctgttggtgcctagcctacagttcctgtccatcctcgggatgggcgtggatgtagtgtgtcgcttcaacaaagtgccgctaggtttaaatactggattcgaattattcgaatgtcggagccacaggtacaaggtatttctttttcatttatgaaaaacattgagccagaatcattgtatcattgaatttgtgattctctacgtagaattgcaaacaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccgaatcgtcgtgatgttaaattgcgtcgtcgaatcatttccaaacatttgatatgctgactcttgtggtgaagaaggctttgtcgcacattcactcaggaggtatttagcgtttccagctaaacgcccggaatgtgtgccttttgctactttcaagtcaacagatcattgttgcaaaacaaatgtctaatttcgacctcagatcaagcgagacta

SEQ ID NO: 76 - DNA, 902 bp, Euroglyphus maynei
gtttccgtaggtgaacctgcggaaggatcattatcggtttttcgtggcattcatttgctgtcggaaacattcgattatcaatttgaacgtcagttgcccgttatcacaaatggaacgactggcaattgttgtgattgaggttgtcattgaatgctctggagttgtatcctggtctttacgtagggcaacatggataaagacgtcgggtgaacttcttaaattggagcctgacaattatcaatgtgcaactttgccggcgtttctccttgacgcctgttggtgcctagcctacagttcctgtccatcctcgggatgggcgtggatgtagtgtgtcgcttcaacaaagtgccgctaggtttaaatactggattcgaattattcgaatgtcggagccacaggtacaaggtatttctttttcatttatgaaaaacattgagccagaatcattgtatcattgaatttgtgattctctacgtagaattgcaaacaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccgaatcgtcgtgatgttaaattgcgtcgtcggatcatttccaaacatttgatatgctgactcttgtggtgaagaaggctttgtcgcacattcactcaggaggtatttagcgtttccagctaaacgcccggaatgtgtgccttttgctactttcaagtcaacagatcattgttgcaaaacaaatgtctaatttcgacctcagatcaagcgagacta

SEQ ID NO: 77 - DNA, 903 bp, Euroglyphus maynei
gtttccgtaggtgaacctgcggaaggatcattatcggtttttcgtggcattcatttgctgtcggaaacattcgattatcaatttgaacgtcagttgcccgttatcacaaatggaacgactggcaattgttgtgattgaggttgtcattgaatgctctggagttgtatcctggtctttacgtagggcaacatggataaagacgtcgggtgaacttcttaaattggagcctgacaattatcaatgtgcaactttgtcggcgtttctccttgacgcctgttggtgcctagcctacagttcctgtccatcctcgggatggtcgtggatgtagtgtgtcgcttcaacaaagtgccgctaggtttaaatactggattcgaattattcgaatgtcggagccacaggtacaaggtatttctttttcatttatgaaaaacattgagccagaatcattgtatcattgaatttgtgattctctacgtagaattgcaaacaaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccgaatcgtcgtgatgtcaaattgcgtcgtcggatcatctccaaacatttgatatgctgactcttgtggtgaagaaggctttgtcgcacattcactcaggaggtatttagcgtttccagctaaacgcccggaatgtgtgccttttgctactttcaagtcaacagatcattgttccaaaacaaatgtctaatttcgacctcagatcaagcgagacta

SEQ ID NO: 78 - DNA, 900 bp, Euroglyphus maynei
gtttccgtaggtgaacctgcggaaggatcattatcggtttttcgacagttttgctgacgaaaacattcgattatcaacttgaacgacagttgcccgttatcacaaatggaacgactggcaattgttgtgattgaggttgtcattgaatgctctgaagttttgtcctggtctttatgttgggcaacatggataaagacatcgggtgaacttctcaattgggagcctgacaattatcaatgtgcaactttgccggcgtttctccttgacgcctgttggtgcctagcctacagttcctgtccatcctcgggatgggcgtggatgtagtgtgtcgcttcaacaaagtgccgctaggtttaaatactggattcgaattattcgaatgtcggagccacaggtacaaggtatttctttttcatttatgaaaaacattgagccagaatcattgtatcatttaatttgtgattctctacgtagaattgcaaacaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccgaatcgtcgtgatgtgttaaattgcgtcgtcggatcatttccaaacatttgatatgctgactcttgtggtgaagaaggctttgtcgcacattcactcaggaggtatttagcgtttccagctaaacgcccggaatgtgtgccttttgctactttcaagtcaacagatcattgttccaaaacaaatgtctaatttcgacctcagatcaagcgagacta

SEQ ID NO: 79 - DNA, 899 bp, Euroglyphus maynei
gtttccgtaggtgaacctgcggaaggatcattatcggtttttcgtggcattcatttgctgtcggaaacattcgattatcaatttgaacgacagttgcccgttatcacaaatggaacaactggcaattgttgtgattgaggttgtcattgaatgctctgaagttgtatcctggtctttatgtggggcaacatggataaagacatcgggtgaacttctcaattaggagcctgacgattatcaatgtgcaactttggcggcgtttctccttgacgcctgttggtgcctagcctacagttcctgtccatcctcgggatgggcgtggatgtagtgtgtcgctttatcaaagtgccgctaggtttaaatactggattcgaattattcgaatgtcggagccgcaggtacgaggtatttctttttcatttatgaaaaacattgagccagaatcattgtatcattgaatttgtgattctctacgtagaattgcaaacaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccggatcgtcgtgattgaattgcgtcgtcggatcatttccaaacatttgatatgctgactcttgtggtgaagaaggctttgtcgcacattcactcaggaggtatttagcgtttccagctaaacgccctgaatgtgtgccttttgctactttcaagtcaacagatcattgttccaaaaaaatgtctaatttcgacctcagatcaagcgagacta

SEQ ID NO: 80 - DNA, 895 bp, Euroglyphus maynei
gtttccgtaggtgaacctgcgggaggatcattatcggttattcgacagttttgctgacgaaaacattcgattatcaatttgaacgacagttgcccgttatcacaaatggaacgactggcaattgttgtgattgaggttgtcattgaatgctctgaagttgtatcctggtctttatgtggggcaacatggataaagacatcgggtgaacttctcaattaggagcctgacgattatcaatgtgcaactttggcggcgtttctccttgacgcctgttggtgcctagcctacagttcctgcccatcctcgggatgggcgtggatgtagtgtgtcgctttatcaaagtgccgctaggtttaaatactggattcgaattattcgaatgtcgaagccgcaggtacgaggtatttctttttcatttatgaaaaacattgagccagaatcattgtatcattgaatttgtgattctctacgtagaattgcaaacaaaaaaaataactactgccagtggtggatcactcggctcgctggtcgaggaagaacgcagctagctgcgttaatcggtgtgaaatgcaggacactccgatcactcgacattcgaacgcacattgcagccattggatagccgatggcttcgtttgtctgagcgtcgttatcaaattgtgacaaatcaaatgcggtaggtccggatcgtcgtgattgaattgcgtcgtcggatcatttccaaacatttgatatgctgactcttgtggtgaagaaggctttgtcgcacattcactcaggaggtatttagcgtttccagctaaacgcccggaatgtgtgccttttgctactttcaagtcaacagatcattgttccaaaaaaatgtctaatttcgacctcagatcaagcgagacta

SEQ ID NO: 81 - DNA, 1220 bp, Glycyphagus domesticus
gtttccgtaggtgaacctgcggaaggatcattaacggattgtttgtcttaatttttgcaatttacttgtaatattaaaacgagcatcaatatccgaacctttcaaaaaaatcgaacgacagttgcccgttatcaaaaatgggttgactggcgagcgattatgattgtaatctaaggtgtaaaatgcatcgaaagcttaatgttgcaattgtgcataatgtgttgaagcaggttaaatgaacgtttatatgatctgggaatactttgggcaaccaaatacgaagtatccaatattttaaaggatccaaacggtgctgctcggccaatcatgcaaatgtgtaatattgatttgtttattcaagtcggctggcatttcacttaccttgattaataattgtgtcgcttgttggcaactctgctcatatcttgtcctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacttgtgctgatgatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgagaattcgtcaggttcaattgatacactgattgtcaatattatgtacgcccaaaaatgcgtattgaagctgtttcgcatattgcaacaaacattaatttgcttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacataattctcaattccaagtgtgtatgggctaattttttagcgcatatgttttttttgtaaactttcgacctcagatcaagcgagatta

SEQ ID NO: 82 - DNA, 1220 bp, Glycyphagus domesticus
gtttccgtaggtgaacctgcggaaggatcattaacggattgtttgtcttgattttcaatttatcattgtttatcatacacgagcatcaatatccgaacctttcaaaaaaatcgaacgatagttgcccgttatcaaaaatgggttgactggcgagcgattatgattgtaatctaaggtgtaaaatgcatcgaaagcttaatgttgcaattgtgcataatgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacctaccttgattaataattgtgtcgcttgttggcaactctgctcatatcttgtcctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacttgtgctgatgatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgagaattcgtcaggttcaattgatacactgattgtcaatattatgtacgcccaaaaatgcgtattgaagctgtttcgcatattgcaacaaacattaatttgtttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacataattctcaattccaagtgtgtatgggctaattttttagcgcatatgttttttttgtaaactttcgacctcagatcaagcgagatta

SEQ ID NO: 83 - DNA, 1218 bp, Glycyphagus domesticus
gtttccgtaggtgaacctgcggaaggatcattaacggattgtttgtcttgattttcaatttatcattgtttatcatacacgagcatcaatatccgaacctttcaaaaaaatcgaacgatagttgcccgttatcaaaaatgggttgactggcgagcgatcgattatgaatcgagtgttgtggatgcatcgaaagcttaatgttgcaattgtgcataccgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacttaccttgattaataattgtgtcgcttgttggcaactctgctcatatcttgttctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacattcgtgtgctgataatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgagaattcgtcaggttcaattgatacactgattgtcaatattatgtacgcccaaaaatgcgtattgaagctgtttcgcatattgcaacaattaattttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacatagctaaaacaatccaagtgtgtatgggctaattttttagcgcatatgttttttttgtaaactttcgacctcagatcaagcgagatta

SEQ ID NO: 84 - DNA, 1218 bp, Glycyphagus domesticus
gtttccgtaggtgaacctgcggaaggatcattaacggattgtttgttttgattttcaatttatcattgtttatcatacacgagcatcaatatccgaacctttcaaaaaaatcgaacgatagttgcccgttatcaaaaatgggttgactggcgagcgatcgattatgaatcgagtgttgtggatgcatcgaaagcttaatgttgcaattgtgcataccgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacttaccttgattaataattgtgtcgcttgttggcaactctgctcatatcttgttctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacattcgtgtgctgataatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgagaattcgtcaggttcaattgatacactgattgtcaatattatgtacgcccaaaaatgcgtattgaagctgtttcgcatattgcaacaattaattttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacatagctaaaacaatccaagtgtgtatgggctaattttttagcgcatatgttttttttgtaaactttcgacctcagatcaagcgagatta

SEQ ID NO: 85 - DNA, 1220 bp, Glycyphagus domesticus
gtttccgtaggtgaacctgcggaaggatcattaacggattgtttgtcttgattttcaatttatcattgtttatcatacacgagcatcaatatccgaacctttcaaaaaaatcgaacgatagttgcccgttatcaaaaatgggttgactggcgagcgatcgattatgaatcgagtgttgtgaatgcatcgaaagcttaatgttgcaattgtgcataccgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacttaccttgattaataattgtgtcgcttgttggcaactctgctcatatcttgttctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacttgtgctgataatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgagaattcgtcaggttcaattgatacactgattgtcaatattatgtacgcccaaaaatgcgtattgaagctgtttcgcatattgcaacaaacattaatttgtttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacatagctaaaacaatccaagtgtgtatgggctaattttttagcgcatatgttttttttgtaaactttcgacctcagatcaagcgagatta

SEQ ID NO: 86 - DNA, 1214 bp, Glycyphagus domesticus
gtttccgtaggtgaacctgcggaaggatcattaacggattgtttgtcttgattttcaatttatcattgtttatcatacacgagcatcaatatccgaacctttcaaaaaaatcgaacgatagttgcccgttatcaaaaatgggttgactggcgagcgatcgattatgaatcgagtgttgtgaatgcatcgaaagcttaatgttgcaattgtgcataccgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacttaccttgattaataattgtgtcgcttgttggcaactctgctcatatcttgttctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacttgtgctgataatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagtgtcgttaattaataatcaaacatcaaaagacctgtttgtcgtattcctcgattacttgagaattcgtcaggttcaattgatacactgattgtcaatattatgtacgcccaaaaatgcgtattgaagctgtttcgcatattgcaacaattaattttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacatagctaaaacaatccaagtgtgtatgggctaattttttagcgcatatgttttttttgtaaactttcgacctcagatcaagcgagatta

SEQ ID NO: 87 - DNA, 1220 bp, Glycyphagus domesticus
gtttccgtaggtgaacctgcggaaggatcattaacggattgtttgttttgattttcaatttatcattgtttatcatacacgagcatcaatatccgaacctttcaaaaaaatcgaacgatagttgcccgttatcaaaaatgggttgactggcgagcgatcgattatgaatcgagtgttgtgaatgcatcgaaagcttaatgttgcaattgtgcataccgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacttaccttgattaataattgtgtcgcttgttggcaactctgctcatatcttgttctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacttgtgctgataatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgagaattcgtcaggttcaattgatacactgattgtcaatattatgtacgcccaaaaatgcgtattgaagctgtttcgcatattgcaacaaacattaatttgtttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacatagctaaaacaatccaagtgtgtatgggctaattttttagcgcatatgttttttttgtaaactttcgacctcagatcaagcgagatta

SEQ ID NO: 88 - DNA, 1220 bp, Glycyphagus domesticus
gtttccgtaggtgaacctgcggaaggatcattaacggattgtttgtcttgattttcaatttatcattgtttatcatacacgagcatcaatatccgaacctttcaaaaaaatcgaacgatagttgcccgttatcaaaaatgggttgactggcgagcgatcgattatgaatcgagtgttgtgaatgcatcgaaagcttaatgttgcaattgtgcataccgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacttaccttgattaataattgtgtcgcttgttggcaactctgctcatatcttgttctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacttgtgctgataatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgagaattcgtcaggttcaattgatacactgattgtcaatattatgtacgcccaagaatgcgtattgaagctgtttcgcatattgcaacaaacattaatttgtttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacatagctaaaacaatccaagtgtgtatgggctaattttttagcgcatatgttttttttgtaaactttcgacctcagatcaagcgagatta

SEQ ID NO: 89 - DNA, 1220 bp, Glycyphagus domesticus
gtttccgtaggtgaacctgcggaaggatcattaacggattgtttgtcttgatttacaacgttacaattgtttatcatacacgagcatcaatatccgaacctttcaaaaaaatcgaacgacagttgcccgttatcaaaaatgggttgactggcgagcgatcgattatgaatcgagtgttgtgaatgcatcgaaagcttaatgttgcaattgtgcataccgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacttaccttgattaataattgtgtcgcttgttggcaactctgctcatatcttgttctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacttgtgctgataatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgagaattcgtcaggttcaattgatacactgattgtcaatattatgtacgcccaaaaatgcgtattgaagctgtttcgcatattgcaacaaacattaatttgtttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacataattctcaattccaagtgtgtatgggctaattttttagcgcatatgttttttttgtaaactttcgacctcagatcaagcgagatta

SEQ ID NO: 90 - DNA, 1223 bp, Glycyphagus domesticus
gtttccgtaggtgaacctgcggaaggatcattaacggattgtttgtcttgatttacaacgttacaattgtttatcatacacgagcatcaatatccgaacctttcaaaaaaatcgaacgacagttgcccgttatcaaaaatgggctgactggcgagcgatcgattatgaatcgagtgttgtgaatgcatcgaaagcttaatgttgcaattgtgcataacgtgttgaagcaggttaaatgaacgtttatatgatctgggaatactttgggcaaccaaatacgaagtatccaatattttaaaggatccaaacgttgctgctcggccaatcatgcaaatgtgtaatattgatttgtttattcaagtcggctggcatttcacttaccttgattgtaataattgtgtcgcttgttggcaactctgctcatatcttgtcctggtgtctagtccgctattcctaacaaattaaaaacttgtatggatgcggtgtgtcagtctattgttgactgttgctaggcttaaatatcgttagcttaactgttgacgaagccgcaagtatggggaaaccttttctttacttaaaccaaactttaagctaataccattaacatttatggaattgtattacctattggatgcacttgtgctgatgatagagatacaaaagagataaatactactgccagtggtggatcactcggctcgctggtcgaggaagaacgcagctagctgcgttaaccggtgtgaaatgcaggacacgccgagcactcgacattcgaacgcacattgcagtcatgggccatcctatgacttcgtttgtctgagcgtcgttaattaataatcaaacatcaaaagacctgtttgtcgtattcctcgattacttgggaattcgtcaggttcaattgatacactgattgtcaatattatgtacgcccaaaaatgcgtattgaagctgtttcgcatattgcaacaaacattaatttgtttgttcaatgttgtgatcgggttctgtgcacgcgtttaattacgcagggcttttggcacatcatgtcaattgcttgaaattgcactagaatattccaaactgatcatacaactgatgacaatatgtgcttctgatacgatactttggtgaaacatagctaaaacaatccaagtgtgtatgggctaattttttagcgcatatgttttttttgtaaactttcgacctcagatcaagcgagatta

SEQ ID NO: 91 - DNA, 1301 bp, Lepidoglyphus destructor
gtttccgtaggtgaacctgcggaaggatcattaacggattgtttattctttcattgcatttcacatgcatgttggaacaaagcaaaaatatccgaacctttcaaacaaatcgaacgaaagttgcccgttatcaaaaatgggttgactgatgaacgatgtgcgatgtaaccgcgtgcatgcatggtatcgagaaatgtgaaaagatgtgcaatgtatcgaaagcttgatgttttgcttgcttgattgctgatgatgttcgaatcaattgctagtgtttgtgcagaataaacaagacatttttgagaatactttgggcaaccgaattatacgaagtattcaatcaaaaatgttgctgcttaatgcttgattgatgaacgcgcatcacatcacagaagtagacatcgattcaattgaatcggctggcattgctatgacattccataattcctcaattccatcttcatgcgatatgattggtgatcgtttgttggcaactctgctcatatcttgcttggtgcctagtccgctgttcctgctttccgtaaggattgtcgtggatgcggtgtgtcagctatatgctgttgctaggcttaaatatcgtttcgcaattatttgggaaacgaagccgcaagtatggggaaaaccttttctttacttaaaccaaactttaagctaataccattaacatttatggaattgtattgtcttgaaataatttttaattattaaagcgatgcaaaagagataaatactactgccagtggtggatcactcggctcgctggtcgaggaagaacgcagctagctgcgttaaccggtgtgaaatgcaggacacgccgagcactcgacattcgaacgcacattgcagtcatgggtcatcctatgacttcgtttgtctgagcgtcgttaattattaaatcaaacatcaaaagacccgtttcgtatcttgtcgtttaattcgatgattcgtcgggtttaattgatacacgctgattgtcacacactacgaatgtgtatgccccaaaattcgtatcgaagctttatcaagcatattgcatcgatcgggttctgtgcacgcgtttaattacgcgcagggcttatggcacaacatgccattatgcctggattgcaacagaatattccaaaccgatcataactgacaatatgtgcttctgatacatttgaaatttggtggtatttgttctgtatgttgtgttctttcattttgtgtgattgattgtaattattgtgattacaattgatattgtttgtaaactttcgacctcagatcaagcgagatta

SEQ ID NO: 92 - DNA, 1301 bp, Lepidoglyphus destructor
gtttccgtaggtgaacctgcggaaggatcattaacggattgtttattctttcattgcatttcacatgcatgttggaacaaagcaaaaatatccgaacctttcaaacaaatcgaacgaaagttgcccgttatcaaaaatgggttgactgatgaacgatgtgcgatgtaaccgcgtgcatgcatggtatcgagaaatgtgaaaagatgtgcaatgtatcgaaagcttgatgttttgcttgcttgattgctgatgatgttcgaatcaattgctagtgtttgtgcagaataaacaagacatttttgagaatactttgggcaaccgaattatacgaagtattcaatcaaaaatgttgctgcttaatgcttgattgatgaacgcgcatcacatcacagaagtagacatcgattcaattgaatcggctggcattgctatgacattccataattcctcaattccatcttcatgcgatatgattggtgatcgtttgttggcaactctgctcatatcttgcttggtgcctagtccgctgttcctgctttccgtaaggattgtcgtggatgcggtgtgtcagctatatgctgttgctaggcttaaatatcgtttcgcaattatttgggaaacgaagccgcaagtatggggaaaaccttttctttacttaaaccaaactttaagctaataccattaacatttatggaattgtattgtcttgaaataatttttaattattaaagcgatgcaaaagagataaatactactgccagtggtggatcactcggctcgctggtcgaggaagaacgcagctagctgcgttaaccggtgtgaaatgcaggacacgccgagcactcgacattcgaacgcacattgcagtcatgggtcatcctatgacttcgtttgtctgagcgtcgttaattattaaatcaaacatcaaaagacccgtttcgtatcttgtcgtttaattcgatgattcgccgggtttaattgatacacgctgattgtcacacactacgaatgtgtatgccccaaaattcgtatcgaagctttatcaagcatattgcatcgatcgggttctgtgcacgcgtttaattacgcgcagggcttatggcacaacatgccattatgcctggattgcaacagaatattccaaaccgatcataactgacaatatgtgcttctgatacatttgaaatttggtggtatttgttctgtatgttgtgttctttcattttgtgtgattggttgtaattattgtgattacaattgatattgtttgtaaactttcgacctcagatcaagcgagatta

SEQ ID NO: 93 - DNA, 1301 bp, Lepidoglyphus destructor; identical to SEQ ID NO: 91

SEQ ID NO: 94 - DNA, 1301 bp, Lepidoglyphus destructor; identical to SEQ ID NO: 91

SEQ ID NO: 95 - DNA, 1302 bp, Lepidoglyphus destructor
gtttccgtaggtgaacctgcggaaggatcattaacggattgtttattctttcattgcatttcacatgcatgttggaacaaagcaaaaatatccgaacctttcaaacaaatcgaacgaaagttgcccgttatcaaaaatgggttgactgatgaacgatgtgcgatgtaaccgcgtgcatgcatggtatcgagaaatgtgaaaagatgtgcaatgtatcgaaagcttgatgttttgcttgcttgattgctgatgatgttcgaatcaattgctagtgtttgtgcagaataaacaagacatttttgagaatactttgggcaaccgaattatacgaagtattcaatcaaaaatgttgctgcttaatgcttgattgatgaacgcgcatcacatcacagaagtagacatcgattcaattgaatcggctggcattgctatgacattccataattcctcaattccatcttcatgcgatatgattggtgatcgtttgttggcaactctgctcatatcttgcttggtgcctagtccgctgttcctgctttccgtaaggattgtcgtggatgcgggtgtgtcagctatatgctgttgctaggcttaaatatcgtttcgcaattatttgggaaacgaagccgcaagtatggggaaaaccttttctttacttaaaccaaactttaagctaataccattaacatttatggaattgtattgtcttgaaataatttttaattattaaagcgatgcaaaagagataaatactactgccagtggtggatcactcggctcgctggtcgaggaagaacgcagctagctgcgttaaccggtgtgaaatgcaggacacgccgagcactcgacattcgaacgcacattgcagtcatgggtcatcctatgacttcgtttgtctgagcgtcgttaattattaaatcaaacatcaaaagacccgtttcgtatcttgtcgtttaattcgatgattcgtcgggtttaattgatacacgctgattgtcacacactacgaatgtgtatgccccaaaattcgtatcgaagctttatcaagcatattgcatcgatcgggttctgtgcacgcgtttaattacgcgcagggcttatggcacaacatgccattatgcctggattgcaacagaatattccaaaccgatcataactgacaatatgtgcttctgatacatttgaaatttggtggtatttgttctgtatgttgtgttctttcattttgtgtgattgattgtaattattgtgattacaattgatattgtttgtaaactttcgacctcagatcaagcgagatta

SEQ ID NO: 96 - DNA, 1301 bp, Lepidoglyphus destructor
gtttccgtaggtgaacctgcggaaggatcattaacggattgtttattctttcattgcatttcacatgcatgttggaacaaagcaaaaatatccgaacctttcaaacaaatcgaacgaaagttgcccgttatcaaaaatgggttgactgatgaacgatgtgcgatgtaaccgcgtgcatgcatggtatcgagaaatgtgaaaagatgtgcaatgtatcgaaagcttgatgttttgcttgcttgattgctgatgatgttcgaatcaattgctagtgtttgtgcagaataaacaagacatttttgagaatactttgggcaaccgaattatacgaagtattcaatcaaaaatgttgctgcttaatgcttgattgatgaacgcgcatcacatcacagaagtagacatcgattcaattgaatcggctggcattgctatgacattccataattcctcaattccatcttcatgcgatatgattggtgatcgtttgttggcaactctgctcatatcttgcttggtgcctagtccgctgttcctgctttccgtaaggattgtcgtggatgcggtgtgtcagctatatgctgttgctaggcttaaatatcgtttcgcaattatttgggaaacgaagccgcaagtatggggaaaaccttttctttacttaaaccaaactttaagctaataccattaacatttatggaattgtattgtcttgaaataatttttaattattaaagagatgcaaaagagataaatactactgccagtggtggatcactcggctcgctggtcgaggaagaacgcagctagctgcgttaaccggtgtgaaatgcaggacacgccgagcactcgacattcgaacgcacattgcagtcatgggtcatcctatgacttcgtttgtctgagcgtcgttaattattaaatcaaacatcaaaagacccgtttcgtatcttgtcgtttaattcgatgattcgtcgggtttaattgatacacgctgattgtcacacactacgaatgtgtatgccccaaaattcgtatcgaagctttatcaagcatattgcatcgatcgggttctgtgcacgcgtttaattacgcgcagggcttatggcacaacatgccattatgcctggattgcaacagaatattccaaaccgatcataactgacaatatgtgcttctgatacatttgaaatttggtggtatttgttctgtatgttgtgttctttcattttgtgtgattgattgtaattattgtgattacaattgatattgtttgtaaactttcgacctcagatcaagcgagatta

SEQ ID NO: 97 - DNA, 1303 bp, Lepidoglyphus destructor
gtttccgtaggtgaacctgcggaaggatcattaacggattgtttattctttcattgcatttcacatgcatgttggaacaaagcaaaaatatccgaacctttcaaacaaatcgaacgaaagttgcccgttatcaaaaatgggttgactgatgaacgatgtgcgatgtaaccgcgtgcatgcatggtatcgagaaatgtgaaaagatgtgcaatgtatcgaaagcttgatgttttgcttgcttgattgctgatgatgttcgaatcaattgctagtgtttgtgcagaataaacaagacatttttgagaatactttgggcaaccgaattatacgaagtattcaatcaaaaatgttgctgcttaatgcttgattgatgaacgcgcatcacatcacagaagtagacatcgattcaattgaatcggctggcattgctatgacattccataattcctcaattccatcttcatgcgatatgattggtgatcgtttgttggcaactctgctcatatcttgcttggtgcctagtccgctgttcctgctttccgtaaggattgtcgtggatgcggtgtgtcagctatatgctgttgctaggcttaaatatcgtttcgcaattatttgggaaacgaagccgcaagtatggggaaaaccttttctttacttaaaccaaactttaagctaataccattaacatttatggaattgtattgtcttgaaataatttttaattattaaagcgatgcaaaagagataaatactactgccagtggtggatcactcggctcgctggtcgaggaagaacgcagctagctgcgttaaccggtgtgaaatgcaggacacgccgagcactcgacattcgaacgcacattgcagtcatgggtcatcctatgacttcgtttgtctgagcgtcgttaattattaaatcaaacatcaaaagacccgtttcgtatcttgtcgtttaattcgatgattcgtcgggtttaattgatacacgctgattgtcacacactacgaatgtgtatgccccaaaattcgtatcgaagctttatcaagcatattgcatcgatcgggttctgtgcacgcgtttaattacgcgcagggcttatggcacaacatgccattatgcctggattgcaacagaatattccaaaccgatcataactgacaatatgtgcttctgatacatttgaaatttggtggtatttgttctgtatgttgtgttctttcatttgttgtgtaattgattgtaattattgtgattacaattgatattgtttgtaaactttcgacctcagatcaagcgagatta

SEQ ID NO: 98 - DNA, 1301 bp, Lepidoglyphus destructor
gtttccgtaggtgaacctgcggaaggatcattaacggattgtttattctttcattgcatttcacatgcatgttggaacaaagcaaaaatatccgaacctttcaaacaaatcgaacgaaagttgcccgttatcaaaaatgggttgactgatgaacgatgtgcgatgtaaccgcatgcatgcatggtatcgagaaatgtgaaaagatgtgcaatgtatcgaaagcttgatgttttgcttgcttgattgctgatgatgttcgaatcaattgctagtgtttgtgcagaataaacaagacatttttgagaatactttgggcaaccgaattatacgaagtattcaatcaaaaatgttgctgcttaatgcttgattgatgaacgcgcatcacatcacagaagtagacatcgattcaattgaatcggctggcattgctatgacattccataattcctcaattccatcttcatgcgatatgattggtgatcgtttgttggcaactctgctcatatcttgcttggtgcctagtccgctgttcctgctttccgtaaggattgtcgtggatgcggtgtgtcagctatatgctgttgctaggcttaaatatcgtttcgcaattatttgggaaacgaagccgcaagtatggggaaaaccttttctttacttaaaccaaactttaagctaataccattaacatttatggaattgtattgtcttgaaataatttttaattattaaagagatgcaaaagagataaatactactgccagtggtggatcactcggctcgctggtcgaggaagaacgcagctagctgcgttaaccggtgtgaaatgcaggacacgccgagcactcgacattcgaacgcacattgcagtcatgggtcatcctatgacttcgtttgtctgagcgtcgttaattattaaatcaaacatcaaaagacccgtttcgtatcttgtcgtttaattcgatgattcgtcgggtttaattgatacacgctgattgtcacacactacgaatgtgtatgccccaaaattcgtatcgaagctttatcaagcatattgcatcgatcgggttctgtgcacgcgtttaattacgcgcagggcttatggcacaacatgccattatgcctggattgcaacagaatattccaaaccgatcataactgacaatatgtgcttctgatacatttgaaatttggtggtatttgttctgtatgttgtgttctttcatcttgtgtgattgattgtaattattgtgattacaattgatattgtttgtaaactttcgacctcagatcaagcgagatta

SEQ ID NO: 99 - DNA, 1297 bp, Lepidoglyphus destructor
gtttccgtaggtgaacctgcggaaggatcattaacggattgtttattctttcattgcatttcatgcatgttggaacaaagcaaaaatatccgaacctttcaaacaaatcgaacgaaagttgcccgttatcaaaaatgggttgactgatgaacgatgtgcgatgtaaccgcgtgcatgcatggtatcgagaaatgtgaaaagatgtgcaatgtatcgaaagcttgatgttttgcttacttgattgctgatgatgttcgaatcaattgctagtgtttgtgcagaataaacgagacatttttgagaatactttgggcaaccgaattatacgaagtattcaatcaaaaatgttgctgcctaatgcttgattgatgaacgcatcacatcacagaagtagacatcgattcaattgaatcggctggcattgctatgacattccataacctcaattccatcttcatgcgatatgattggtgatcgtttgttggcaactctgctcatatcttgcttggtgcctagtccgctgttcctgctttccgtaaggactgtcgtggatgcggtgtgtcagctatatgctgttgctaggcttaaatatcgtttcgcaatattacaattgggatacggagccgcaagtatggggaaaaccttttctttacttaaaccaaactttaagctaataccattaacatttatggaattgtattgtcttgaaataatttttaattattaaagcgatgcaaaagagataaatactactgccagtggtggatcactcggctcgctggtcgaggaagaacgcagctagctgcgttaaccggtgtgaaatgcaggacacgccgagcactcgacattcgaacgcacattgcagtcatgggtcatcctatgacttcgtttgtctgagcgtcgttaattattaaatcaaacatcaaaagacccgtttcgtatcttgtcgtttattcgatgattcgtcgggtttaattgatacacgctgattgtcgcacactacgaatgtgtatgccccaaaattcgtatcgaagctttatcaattgcatattgcatcgatcgggttctgtgcacgcgtttaattacgcgcagggcttatggcacaacatgccattatgcctggattgcaacagaatattccaaaccgatcataactgacaatatgtgcttctgatacatttgaaatttggtggtatttgttctgtatgttgtgttctttcatttgttgtgtaattgattgtaattttttacaattgatattgtttgtaaactttcgacctcagatcaagcgagatta

SEQ ID NO: 100 - DNA, 1297 bp, Lepidoglyphus destructor
gtttccgtaggtgaacctgcggaaggatcattaacggattgtttattctttcattgcatttcatgcatgttggaacaaagcaaaaatatccgaacctttcaaacaaatcgaacgaaagttgcccgttatcaaaaatgggttgactgatgaacgatgtgcgatgtaaccgcgtgcatgcatggtatcgagaaatgtgaaaagatgtgcaatgtatcgaaagcttgatgttttgcttacttgattgctgatgatgttcgaatcaattgctagtgtttgtgcagaataaacgagacatttttgagaatactttgggcaaccgaattatacgaagtattcaatcaaaaatgttgctgcctaatgcttgattgatgaacgcatcacatcacagaagtagacatcgattcaattgaatcggctggcattgctatgacattccataacctcaattccatcttcatgcgatatgattggtgatcgtttgttggcaactctgctcatatcttgcttggtgcctagtccgctgttcctgctttccgtaaggactgtcgtggatgcggtgtgtcagctatatgctgttgctaggcttaaatatcgtttcgcaatattacaattgggatacggagccgcaagtatgaggaaaaccttttctttacttaaaccaaactttaagctaataccattaacatttatggaattgtattgtcttgaaataatttttaattattaaagcgatgcaaaagagataaatactactgccagtggtggatcactcggctcgctggtcgaggaagaacgcagctagctgcgttaaccggtgtgaaatgcaggacacgccgagcactcgacattcgaacgcacattgcagtcatgggtcatcctatgacttcgtttgtctgagcgtcgttaattattaaatcaaacatcaaaagacccgtttcgtatcttgtcgtttattcgatgattcgtcgggtttaattgatacacgctgattgtcgcacactacgaatgtgtatgccccaaaattcgtatcgaagctttatcaattgcatattgcatcgatcgggttctgtgcacgcgtttaattacgcgcagggcttatggcacaacatgccattatgcctggattgcaacagaatattccaaaccgatcataactgacaatatgtgcttctgatacatttgaaatttggtggtatttgttctgtatgttgtgttctttcatttgttgtgtaattgattgtaattttttacaattgatattgtttgtaaactttcgacctcagatcaagcgagatta

SEQ ID NO: 101 - DNA, 19 bp, Tyrophagus fanetzhogorum
gacagaagctgaaagccgt

SEQ ID NO: 102 - DNA, 23 bp, Lepidoglyphus destructor
gatgttcgaatcaattgctagtg

SEQ ID NO: 103 - DNA, 20 bp, Glycophagus destructor
gcataccgtgttgaagcagg

SEQ ID NO: 104 - DNA, 21 bp, Dermatophagoides pteronyssinus
gatcgactggcaattgttgac

SEQ ID NO: 105 - DNA, 19 bp, Tyrophagus putrescentiae
cgccatttgacacagtacc

SEQ ID NO: 106 - DNA, 19 bp, Blomia tropicalis
tgtgtgtgggggattttgc

SEQ ID NO: 107 - DNA, 23 bp, Eroglyphus maynei
gagcctgacaattatcaatgtgc

SEQ ID NO: 108 - DNA, 19 bp, Dermatophagoides microceras
cgggatgaacgtgtggatg

SEQ ID NO: 109 - DNA, 18 bp, Acarus siro
gtcggttacggtcaaacg

SEQ ID NO: 110 - DNA, 24 bp, Dermatophagoides farinae
gaaacaattgaattgtgattctgc

SEQ ID NO: 111 - DNA, 18 bp, Artificial sequence
  Synthetic universal primer
tgcgttcgaangtcgagt

SEQ ID NO: 112 - DNA, 10 bp, Artificial Sequence
  Generic sequence
aggatcatta

SEQ ID NO: 113 - DNA, 10 bp, Artificial Sequence
  Gereric sequence
ctgyyagtgg

SEQ ID NO: 114 - DNA, 10 bp, Artificial Sequence
  Generic sequence
tgagcgtcgt

SEQ ID NO: 115 - DNA, 9 bp, Artificial Sequence
  Generic sequence
cgacctcag

SEQ ID NO: 116 - DNA, 20 bp, Artificial Sequence
  example sequence to illustrate sequence identiy
gcataccgtgttgaagcagg

SEQ ID NO: 117 - DNA, 20 bp, Artificial Sequence
  Example sequence to illustrate sequence identity
aaataccgtgttgaagcaaa

SEQ ID NO: 118 - DNA, 24 bp, Artificial Sequence
  DNA primer
agaggaagtaaaagtcgtaacaag

SEQ ID NO: 119 - DNA, 20 bp, Artificial Sequence
  DNA primer
atatgcttaaattcagcggg

SEQ ID NO: 120 - DNA, 18 bp, Artificial Sequence
  DNA primer
tgcgttcgaawgtcgagt

SEQ ID NO: 121 - DNA, 24 bp, Artificial Sequence
  DNA primer
agaggaagtaaaagtcgtaacaag

SEQ ID NO: 122 - DNA, 24 bp, Artificial Sequence
  DNA primer
gctttcaataacctcatcagtgtc

SEQ ID NO: 123 - DNA, 23 bp, Artificial Sequence
  DNA primer
ccatcactaaaggacagaaccgc

SEQ ID NO: 124 - DNA, 22 bp, Artificial Sequence
  DNA primer
ctccagcaatcgaattatgctc